(19) United States
(12) Patent Application Publication
(10) Pub. No.: US 2025/0312491 A1
(43) Pub. Date: Oct. 9, 2025

(54) BASE EDITING OF TRANSTHYRETIN GENE

(71) Applicant: Beam Therapeutics Inc., Cambridge, MA (US)

(72) Inventors: Alexandra C. Chadwick, Cambridge, MA (US); Kallanthottahil G. Rajeev, Cambridge, MA (US)

(73) Assignee: Beam Therapeutics Inc., Cambridge, MA (US)

(21) Appl. No.: 19/204,091

(22) Filed: May 9, 2025

Related U.S. Application Data
(63) Continuation of application No. PCT/US2023/079329, filed on Nov. 10, 2023.
(60) Provisional application No. 63/383,394, filed on Nov. 11, 2022.

(51) Int. Cl. A61K 48/00; C12N 9/22; C12N 9/78; C12N 15/11; C12N 15/88

(52) U.S. Cl. CPC A61K 48/0058; C12N 9/226; C12N 9/78; C12N 15/11; C12N 15/88; C12Y 305/04002; C12N 2310/20; C12N 2310/315; C12N 2310/321; C12N 2310/351; C12N 2320/32; C12N 2320/34

(57) ABSTRACT

Provided herein are compositions for gene modification related to base editor systems, and methods of using the same to treat or prevent conditions associated with the extracellular deposition in various tissues of amyloid fibrils formed by the aggregation of misfolded transthyretin (TTR) proteins. Such conditions include, but are not limited to, polyneuropathy due to hereditary transthyretin amyloidosis (hATTR-PN) and hereditary cardiomyopathy due to transthyretin amyloidosis (hATTR-CM), both associated with autosomal dominant mutations of the TTR gene, and an age-related cardiomyopathy associated with wild-type TTR proteins (ATTRwt), also known as senile cardiac amyloidosis.

### CROSS-REFERENCE TO RELATED APPLICATIONS

This application is a continuation under 35 U.S.C. § 111(a) of PCT International Patent Application No. PCT/US2023/079329, filed Nov. 10, 2023, designating the United States and published in English, which claims priority to and the benefit of U.S. Provisional Application No. 63/383,394, filed Nov. 11, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Nov. 10, 2023, is named 180802-046902US_SL.xml and is 5,147,724 bytes in size.

### BACKGROUND

Transthyretin (TTR) is a 55-kDa transport protein, for both thyroxine (T4) and retinol-binding protein, that circulates in soluble form in the serum and cerebrospinal fluid (CSF) of healthy humans. Under normal conditions, the TTR protein circulates as a homotetramer. Hereditary transthyretin amyloidosis (hATTR) is a disease due to mutations in the gene encoding TTR. Autosomal dominant mutations destabilize the TTR tetramer and enhance dissociation into monomers, resulting in misfolding, aggregation, and the subsequent extracellular deposition of TTR amyloid fibrils in different sites. This multisystem extracellular deposition of amyloid results in dysfunction of different organs and tissues. In particular, polyneuropathy (ATTR-PN) and cardiomyopathy (ATTR-CM) due to transthyretin amyloidosis are severe disorders associated with significant morbidity and mortality.

Since TTR is mainly produced by the liver, an early therapeutic approach for the treatment of hATTR amyloidosis was liver transplantation. Other therapies include the administration of oral drugs that act as kinetic stabilizers of TTR tetramers (such as tafamidis and diflunisal) and the suppression of TTR protein synthesis with gene-silencing drugs such as small interfering RNAs (siRNAs) (patisiran) and antisense oligonucleotides (inotersen).

The invention here recognizes that a gene editing approach for the treatment of transthyretin amyloidosis, including both polyneuropathy and cardiomyopathy, has the potential to deliver a once and done treatment with superior results to existing treatments.

### SUMMARY

Provided herein are compositions for gene modification or editing and methods of using the same to treat or prevent conditions associated with the extracellular deposition in various tissues of amyloid fibrils formed by the aggregation of misfolded transthyretin (TTR) proteins. Such conditions include, but are not limited to, polyneuropathy due to hereditary transthyretin amyloidosis (hATTR-PN) and hereditary cardiomyopathy due to transthyretin amyloidosis (hATTR-CM), both associated with autosomal dominant mutations of the TTR gene, and an age-related cardiomyopathy associated with wild-type TTR proteins (ATTRwt), also known as senile cardiac amyloidosis. Compositions and methods directed to editing the TTR gene using an editing system such as one comprising a base editor and guide RNAs are disclosed.

In some aspects, provided herein is a base editor system for modifying a target Transthyretin (TTR) gene, comprising a guide RNA, comprising a sequence defined by

(SEQ ID NO: 11)
mG*mC*mC*AUCCUGCCAAGAAUGAGmGUUUUAGmAmGmCmUmAGmAmAmAm
UmAmGmCmAmAGUUmAAmAAmUAmAmGmGmCmUmAGUmCmCGUUAmUmCA
AmCmUmUGmAmAmAmAmAmGmUmGGmCmAmCmCmGmAmGmUmCmGmGmU
mGmCmU*mU*mU*mU (GA521),

wherein A is adenosine, C is cytidine, G is guanosine, U is uridine, mA* is 2′-O-methyladenosine, mC* is 2′-O-methylcytidine, mG* is 2′-O-methylguanosine, mU* is 2′-O-methyluridine, and wherein nucleotides represented in bold are linked by a phosphorothioate (PS) backbone linkage,
the guide RNA directing the base editor system to effect a nucleobase alteration in the TTR gene.

In some aspects, provided herein is a guide RNA, comprising a sequence defined by

(SEQ ID NO: 11)
mG*mC*mC*AUCCUGCCAAGAAUGAGmGUUUUAGmAmGmCmUmAGmAmAmAm
UmAmGmCmAmAGUUmAAmAAmUAmAmGmGmCmUmAGUmCmCGUUAmUmCA
AmCmUmUGmAmAmAmAmAmGmUmGGmCmAmCmCmGmAmGmUmCmGmGmU
mGmCmU*mU*mU*mU (GA521),

wherein A is adenosine, C is cytidine, G is guanosine, U is uridine, mA* is 2′-O-methyladenosine, mC* is 2′-O-methylcytidine, mG* is 2′-O-methylguanosine, mU* is 2′-O-methyluridine, and wherein nucleotides represented in bold are linked by a phosphorothioate (PS) backbone linkage,
the guide RNA directing a base editor system to effect a nucleobase alteration in the TTR gene.

An engineered, non-naturally occurring base editing system for modifying a target Transthyretin (TTR) gene, comprising (a) a guide RNA molecule having a sequence defined by

(SEQ ID NO: 11)
mG*mC*mC*AUCCUGCCAAGAAUGAGmGUUUUAGmAmGmCmUmAGmAmAmAm
UmAmGmCmAmAGUUmAAmAAmUAmAmGmGmCmUmAGUmCmCGUUAmUmCA
AmCmUmUGmAmAmAmAmAmGmUmGGmCmAmCmCmGmAmGmUmCmGmGmU
mGmCmU*mU*mU*mU,

and (b) a codon-optimized nucleic acid encoding a Cas9 protein fused to a deaminase, wherein the Cas9 protein fusion is capable of binding to the guide RNA and of editing the target TTR sequence complementary to the guide RNA.

In some embodiments, the deaminase comprises a cytosine deaminase or an adenine deaminase.

In some embodiments, the Cas9 protein is a catalytically impaired Cas9 protein.

In some embodiments, the Cas9 protein is a dead Cas9 or nickase Cas9.

In some embodiments, the cytosine or adenine deaminase is a deoxycytosine or a deoxyadenosine deaminase.

In some embodiments, the Cas9 is fused to ABE8.8.

In some embodiments, provided herein is a lipid nanoparticle (LNP) comprising the system described herein.

In some embodiments, the LNP comprises an ionizable amino lipid, a neutral helper lipid, a PEG-Lipid, a sterol lipid and/or a GalNAc lipid.

In some embodiments, the ionizable amino lipid is VL422 or LP-01, the neutral helper lipid is DSPC, the PEG lipid is PEG₂₀₀₀-DMG, the sterol lipid is cholesterol and the GalNAc lipid is DSG-PEG-Lys-tris (GalNAc).

In some embodiments, the ionizable lipid is LP-01 (or CIN16645) defined by the structure,

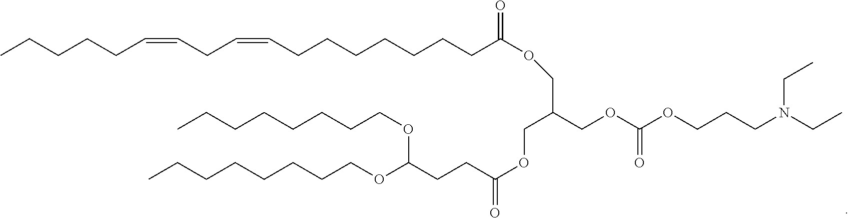 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments, the LNP comprises an N:P ratio of between about 1:40 to about 1:1.

In some embodiments, the LNP comprises an N:P ratio of about 1:6.

In some embodiments, provided is a pharmaceutical composition comprising the LNP as described herein.

In some aspects, provided herein is a method of editing a TTR gene in a cell, the method comprising contacting the cell with an LNP comprising (a) a guide RNA molecule having a sequence defined by

(SEQ ID NO: 11)
mG*mC*mC*AUCCUGCCAAGAAUGAGmGUUUUAGmAmGmCmUmAGmAmAmAm
UmAmGmCmAmAGUUmAAmAAmUAmAmGmGmCmUmAGUmCmCGUUAmUmCA
AmCmUmUGmAmAmAmAmAmGmUmGGmCmAmCmCmGmAmGmUmCmGmGmU
mGmCmU*mU*mU*mU,

and

(b) a base editor system comprising a codon-optimized nucleic acid encoding a Cas9 protein fused to a deaminase, wherein the Cas9 protein fusion is capable of binding to the guide RNA and of editing the target nucleic acid sequence complementary to the guide RNA.

In some embodiments, provided herein is a method of treating a disease or disorder, comprising administering to a subject in need thereof, the pharmaceutical composition described herein.

In some embodiments, the disease or disorder is hereditary transthyretin amyloidosis, cardiomyopathy, polyneuropathy or senile cardiac amyloidosis.

In some embodiments, the pharmaceutical composition is administered by a route selected from intravenous, intradermal, transdermal, intranasal, intramuscular, subcutaneous, transmucosal or oral.

In some embodiments, the LNP is delivered to liver.

### BRIEF DESCRIPTION OF THE FIGURES

FIGS. 1A, 1B, and 1C. A general schematic of a gene editor complexed with a gRNA targeting a gene of interest. Cas9 protein, guide RNA, Spacer sequence, protospacer sequence, and PAM (protospacer adjacent motif) are identified (FIG. 1A). FIG. 1A discloses SEQ ID NO: 5760. Additionally, schematics of general principles of base editing with cytosine base editors (CBE) (FIG. 1B) and adenine base editors (ABE) (FIG. 1C) are illustrated.

FIG. 2. Alteration of splice donor sites resulting from base editing. Top panel represents normal splicing of RNA transcribed from a gene. Bottom panel represents splicing that may result from transcription of a gene that has a disrupted splice site due to editing.

FIG. 3. Map of the human TTR gene (hTTR gene), shows the location of various restriction enzyme recognition sites, Exons 1-4, and the single guide RNAs GA457, GA459, GA460, and GA461 specified in Table 1.

FIG. 4. Nucleotide sequence of the human TTR gene (UniProtKB—P02766 (TTHY_HUMAN)) from the reference human genome (GRCh38) is shown and depicts the region on the gene where guides GA457, GA459, GA460, and GA461 are located. FIG. 4 discloses SEQ ID NO: 5761.

FIGS. 5A, 5B, and 5C. A schematic showing TTR guides and editing locations for GA457 (FIG. 5A), GA460 (FIG. 5B), and GA461 (FIG. 5C). Human genomic DNA (gDNA) sequences are labeled in black. Guide sequences are highlighted in grey above. Genomic exon sequences are in uppercase letters and intron sequences are in lowercase letters. The main position targeted by ABE editing is labeled with a black arrow. FIGS. 5A, 5B, and 5C disclose SEQ ID NOS 1, 5762, 4, 5763, 5 and 5764, respectively, in order of appearance.

FIG. 6. is a graph representing the percent splice editing in human hepatocytes using ABE editing with single guide RNAs GA457, GA459, GA460, and GA461 guide RNAs. The three TTR guide RNAs GA457, GA460, and GA461 show high activity in human hepatocytes. Each of the guides employ the identical tracr sequence and differ only by their RNA spacer sequence which corresponds to specified DNA protospacer sequences on the targeted TTR gene.

FIG. 7 is a flowchart of the ONE-seq protocol for determining candidate off-target sites.

FIG. 8 is a schematic diagram comparison of GA519 and GA457 hybridized to NHP and Human TTR exon 1. FIG. 8 discloses SEQ ID NOS 1, 5765, 2 and 5766, respectively, in order of appearance.

FIG. 9 is a schematic diagram showing a comparison of GA520 and GA460 hybridized to NHP and Human TTR exon 3. FIG. 9 discloses SEQ ID NOS 5767-5768 and 5767-5768, respectively, in order of appearance.

FIG. 10 is a bar graph showing hepatic editing of TTR gene by LNP1 and LNP2 in Non-Human Primates (NHPs) as described in the Examples.

FIG. 11 is a bar graph showing serum TTR protein changes as measured by ELISA in NHP treated with LNP1 and LNP2 as described in the Examples.

FIG. 12 is a bar graph showing serum TTR protein changes as measured by mass spectrometry in NHP treated with LNP1 and LNP2 as described in the Examples.

FIGS. 13A and 13B are a bar graphs showing serum Alanine Aminotransferase (ALT), FIG. 13A, and serum Aspartate Aminotransferase (AST), FIG. 13B, concentrations in NHP treated with LNP1 and LNP2 as described in the Examples.

FIGS. 14A and 14B are a bar graphs showing serum Lactate Dehydrogenase (LDH), FIG. 14A, and serum Glutamate Dehydrogenase (GDH), FIG. 14B, concentrations in NHP treated with LNP1 and LNP2 as described in the Examples.

FIGS. 15A and 15B are a bar graphs showing serum Gamma-Glutamyl Transferase (GGT), FIG. 15A, and serum Alkaline Phosphatase (AP), FIG. 15B, concentrations in NHP treated with LNP1 and LNP2 as described in the Examples.

FIG. 16 is a bar graph showing serum total bilirubin concentrations in NHP treated with LNP1 and LNP2 as described in the Examples.

FIG. 17 is a bar graph showing serum creatine kinase concentrations in NHP treated with LNP1 and LNP2 as described in the examples.

FIG. 18 shows bar graphs of serum cytokine concentrations (MCP-1, upper left panel; IL-6, upper right panel; IP-10, lower left panel; and IL-1RA, lower right panel) over time in NHP treated with LNP1 and LNP2 as described in the Examples.

FIGS. 19A and 19B are plots of plasma pharmacokinetic profiles of iLipid (FIG. 19A) and PEG lipids (FIG. 19B) in NHP treated with LNP1 and LNP2 as described in the Examples.

FIG. 20 is a bar graph showing hepatic editing of TTR gene by LNP3 in NHPs as described in the Examples.

FIG. 21 is a plot showing serum TTR protein changes measured by ELISA in NHP treated with LNP3 as described in the Examples.

FIG. 22 is a plot showing serum TTR protein changes measured by liquid chromatography-mass spectrometry in NHP treated with LNP3 as described in the Examples.

FIGS. 23A and 23B are bar graphs showing serum Alanine Aminotransferase (ALT), FIG. 23A, and serum Aspartate Aminotransferase (AST), FIG. 23B, concentrations in NHP treated with LNP3 as described in the Examples.

FIGS. 24A and 24B are bar graphs showing serum Lactate Dehydrogenase (LDH), FIG. 24A, and serum Glutamate Dehydrogenase (GDH), FIG. 24B, concentrations in NHP treated with LNP3 as described in the Examples.

FIGS. 25A and 25B are bar graphs showing serum Gamma-Glutamyl Transferase (GGT), FIG. 25A, and serum Alkaline Phosphatase (AP), FIG. 25B, concentrations in NHP treated with LNP3 as described in the Examples.

FIG. 26 is a bar graph showing serum total bilirubin concentrations in NHP treated with LNP2 as described in the Examples.

FIG. 27 is a bar graph showing serum creatine kinase concentrations in NHP treated with LNP3 as described in the examples.

FIGS. 28A and 28B are plots of plasma pharmacokinetic profiles of iLipid (FIG. 28A) and PEG lipids (FIG. 28B) in NHP treated with LNP1 and LNP2 as described in the Examples.

FIG. 29 is a graph that shows percent base editing in primary human hepatocytes at various doses total RNA (ng/TA/ml) where GA521 was provided as the guide RNA. GA521 showed sustained base editing of greater than 40% in primary human hepatocytes.

### DETAILED DESCRIPTION OF ILLUSTRATIVE EMBODIMENTS

Provided herein are compositions for gene modification or editing and methods of using the same to treat or prevent conditions associated with the extracellular deposition in various tissues of amyloid fibrils formed by the aggregation of misfolded transthyretin (TTR) proteins. Such conditions include, but are not limited to, polyneuropathy due to hereditary transthyretin amyloidosis (hATTR-PN) and hereditary cardiomyopathy due to transthyretin amyloidosis (hATTR-CM), both associated with autosomal dominant mutations of the TTR gene, and an age-related cardiomyopathy associated with wild-type TTR proteins (ATTRwt), also known as senile cardiac amyloidosis. Compositions and methods directed to editing the TTR gene using an editing system such as one comprising a base editor and guide RNAs are disclosed.

### Definitions

The following presents definitions of some terms presented throughout this disclosure. In some instances, terms are defined in areas of this specification other than in this “Definitions” section.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated, the term “about” means within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

An article, composition, method, or the like that comprises one or more elements may consist of the one or more elements or may consist essentially of the one or more elements. As used in this specification and claim(s), “consisting of” (and any form of consisting of, such as “consists of” and “consist of”) means including and limited to. As used in this specification and claim(s), an article, composition, method, or the like “consisting essentially of” (and any form of consisting essentially of, such as “consists essentially of” and “consist essentially of”) means the article, composition, method, or the like includes the specified enumerated elements; such as components, compounds, materials, steps, or the like, and may include additional elements that do not materially affect the basic and novel characteristics of the article, composition, method, or the like.

Reference in the specification to “some embodiments,” “an embodiment,” “one embodiment,” “embodiments” or “other embodiments” means that a particular feature, structure, or characteristic described in connection with the embodiments is included in at least some embodiments, but not necessarily all embodiments, of the present disclosures.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages or modified sugar residues, or non-canonical/chemically-modified nucleobases and combinations thereof, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs).

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally-occurring bases, sugars and intersugar (backbone) linkages. The terms “polynucleotide” and “oligonucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases. It should be understood that the terms “polynucleotide” and “oligonucleotide” can also include polymers or oligomers comprising both deoxy and ribonucleotide combinations or variants thereof in combination with backbone modifications, such as those described herein.

The “nucleic acid” described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The nucleic acid described herein may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. Backbone modifications can include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) prevents nuclease recognition and degradation. Backbone modifications can also include peptide bonds instead of phosphorous in the backbone structure (e.g., N-(2-aminoethyl)-glycine units linked by peptide bonds in a peptide nucleic acid), or linking groups including carbamate, amides, and linear and cyclic hydrocarbon groups. Oligonucleotides with modified backbones are reviewed in Micklefield, Curr. Med. Chem., 8 (10): 1157-79, 2001 and Lyer et al., Curr. Opin. Mol. Ther., 1 (3): 344-358, 1999. Nucleic acid molecules described herein may contain a sugar moiety that comprises ribose or deoxyribose, as present in naturally occurring nucleotides, or a modified sugar moiety or sugar analog. Modified sugar moieties include, but are not limited to, 2′-O-methyl, 2′-O-methoxyethyl, 2′-O-aminoethyl, 2′-Flouro, N3′→P5′ phosphoramidate, 2′dimethylaminooxyethoxy, 2′ 2′dimethylaminoethoxyethoxy, 2′-guanidinium, 2′-O-guanidinium ethyl, carbamate modified sugars, and bicyclic modified sugars. 2′-O-methyl or 2′-O-methoxyethyl modifications promote the A-form or RNA-like conformation in oligonucleotides, increase binding affinity to RNA, and have enhanced nuclease resistance. Modified sugar moieties can also include having an extra bridge bond (e.g., a methylene bridge joining the 2′-O and 4′-C atoms of the ribose in a locked nucleic acid) or sugar analog such as a morpholine ring (e.g., as in a phosphorodiamidate morpholino).

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The present disclosure encompasses isolated or substantially purified nucleic acid molecules and compositions containing those molecules. As used herein, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

As used herein, the terms “protein,” “polypeptide,” and “peptide” are used interchangeably and refer to a polymer of amino acid residues linked via peptide bonds and which may be composed of two or more polypeptide chains. The terms “polypeptide,” “protein,” and “peptide” refer to a polymer of at least two amino acid monomers joined together through amide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant. Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

“A subject in need thereof” refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has hereditary transthyretin amyloidosis (hATTR). In some embodiments, the subject has cardiomyopathy due to transthyretin amyloidosis (ATTR-CM). In some embodiments, the subject has polyneuropathy due to transthyretin amyloidosis (ATTR-PN). In some embodiments, the subject has wild-type ATTR (ATTRwt), the age-related deposition of wild type TTR protein (formerly known as senile amyloidosis).

“Administering” and its grammatical equivalents as used herein can refer to providing one or more replication competent recombinant adenovirus or pharmaceutical compositions described herein to a subject or a patient. By way of example and without limitation, “administering” can be performed by intravenous (i.v.) injection, sub-cutaneous (s.c.) injection, intradermal (i.d.) injection, intraperitoneal (i.p.) injection, intramuscular (i.m.) injection, intravascular injection, intracerebroventricular (i.c.v.) injection, intrathecal (i.t.) injection, infusion (inf.), oral routes (p.o.), topical (top.) administration, or rectal (p.r.) administration. One or more such routes can be employed.

The term “parenteral” as used herein includes subcutaneous, intracutaneous, intravenous, intramuscular, intraarticular, intraarterial, intrasynovial, intrasternal, intracerebroventricular, intrathecal, intralesional, and intracranial injection or infusion techniques. Parenteral administration can be, for example, by bolus injection or by gradual perfusion over time. In addition, it can be administered to the subject via injectable depot routes of administration such as using 1-, 3-, or 6-month depot injectable or biodegradable materials and methods.

The terms “treat,” “treating,” or “treatment,” and its grammatical equivalents as used herein, can include alleviating, abating, or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. “Treating” may refer to administration of a composition comprising a nanoparticle, such as a lipid nanoparticle (LNP), to a subject after the onset, or suspected onset, of a disease or condition. “Treating” includes the concepts of “alleviating,” which refers to lessening the frequency of occurrence or recurrence, or the severity, of any symptoms or other ill effects related to a disease or condition and/or the side effects associated with the disease or condition. The term “treating” also encompasses the concept of “managing” which refers to reducing the severity of a particular disease or disorder in a patient or delaying its recurrence, e.g., lengthening the period of remission in a patient who had suffered from the disease. The term “treating” further encompasses the concept of “prevent,” “preventing,” and “prevention.” It is appreciated that, although not precluded, treating a disorder or condition does not require that the disorder, condition, or symptoms associated therewith be completely eliminated.

As used herein, the terms “prevent,” “preventing,” “prevention,” and the like, refer to reducing the probability of developing a disease or condition in a subject, who does not have, but is at risk of or susceptible to developing a disease or condition.

The term “ameliorate” as used herein can refer to decrease, suppress, attenuate, diminish, arrest, or stabilize the development or progression of a disease.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” may be an acid or base salt that is generally considered in the art to be suitable for use in contact with the tissues of human beings or animals without excessive toxicity, irritation, allergic response, or other problem or complication. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985).

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

As used herein, a spacer sequence of a guide nucleic acid is considered to be “homologous” to a protospacer sequence of a target nucleic acid if a base editor of a base editor system comprising the spacer sequence is capable of making a modification to a base within the target nucleic acid. A spacer sequence that is homologous to a protospacer sequence may be identical or substantially identical to the protospacer sequence.

As used herein, a nucleic acid sequence that is “substantially identical” to another nucleic acid sequence is a nucleotide sequence that has 70% or more sequence identity to the other nucleic acid sequence.

For purposes of percent sequence identity between an RNA sequence (e.g., spacer) and a DNA sequence (e.g., target gene protospacer), uracil bases in the RNA are to be considered identical to thymine bases in DNA sequences.

As used herein “sequence identity” refers to the extent to which two optimally aligned nucleic acid sequences are invariant throughout a window of alignment of components, e.g., nucleotides. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) nucleic acid (or its complementary strand) as compared to a test (“subject”) nucleic acid (or its complementary strand) when the two sequences are optimally aligned. Percent sequence identity may be determined, when the compared sequences are aligned for maximum correspondence, as measured using a sequence comparison algorithm described below and as known in the art, or by visual inspection.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGR Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100.

“Percent identity” may also be determined using BLASTX version 2.0 for translated nucleotide sequences and BLASTN version 2.0 for polynucleotide sequences. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands.

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001.

In some embodiments, a first nucleotide sequence that is homologous to a second nucleotide sequence may hybridize to the complimentary sequence of the second nucleotide sequence under stringent conditions or highly stringent conditions. “Stringent hybridization conditions” and “stringent hybridization wash conditions” in the context of nucleic acid hybridization are sequence dependent and are different under different environmental parameters. An extensive guide to the hybridization of nucleic acids is found in Tijssen, Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleotide sequences which have more than 100 complementary residues on a filter in a Southern or Northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook and Russel, Molecular Cloning: A laboratory Manual, 3^{rd} ed., Cold Spring Harbor Laboratory Press, 2001 for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

For any method disclosed herein that includes discrete steps, the steps may be conducted in any feasible order. And, as appropriate, any combination of two or more steps may be conducted simultaneously.

All headings throughout are for the convenience of the reader and should not be used to limit the meaning of the text that follows the heading, unless so specified.

### Transthyretin Protein and Gene

Transthyretin (TTR), originally known as prealbumin, is a 55-kDa transport protein for both thyroxine (T4) and retinol-binding protein, that circulates in soluble form in the serum and cerebrospinal fluid (CSF) of healthy humans. TTR is understood to be primarily synthesized in the liver. Under normal conditions, TTR circulates as a homotetramer with a central channel. The wild-type TTR monomer is 147 amino acids in length and has the amino acid sequence below:

(SEQ ID NO: 23)
MASHRLLLLC LAGLVFVSEA GPTGTGESKC PLMVKVLDAV RGSPAINVAV
HVFRKAADDT WEPFASGKTS ESGELHGLTT EEEFVEGIYK VEIDTKSYWK
ALGISPFHEH AEVVFTANDS GPRRYTIAAL LSPYSYSTTA VVTNPKE.

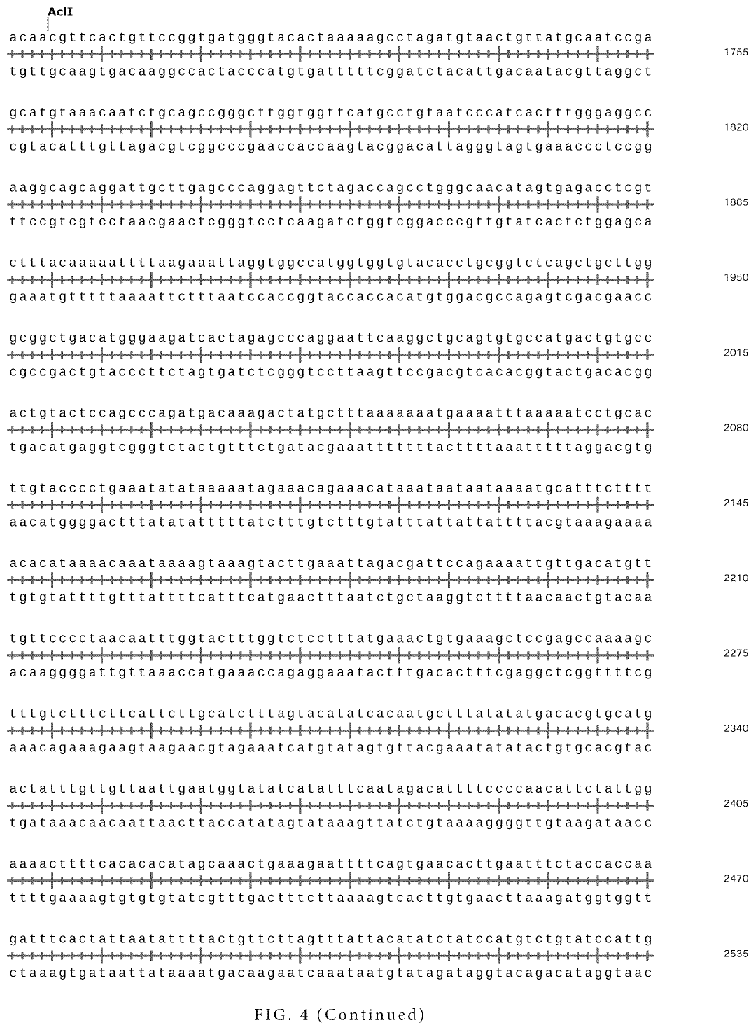

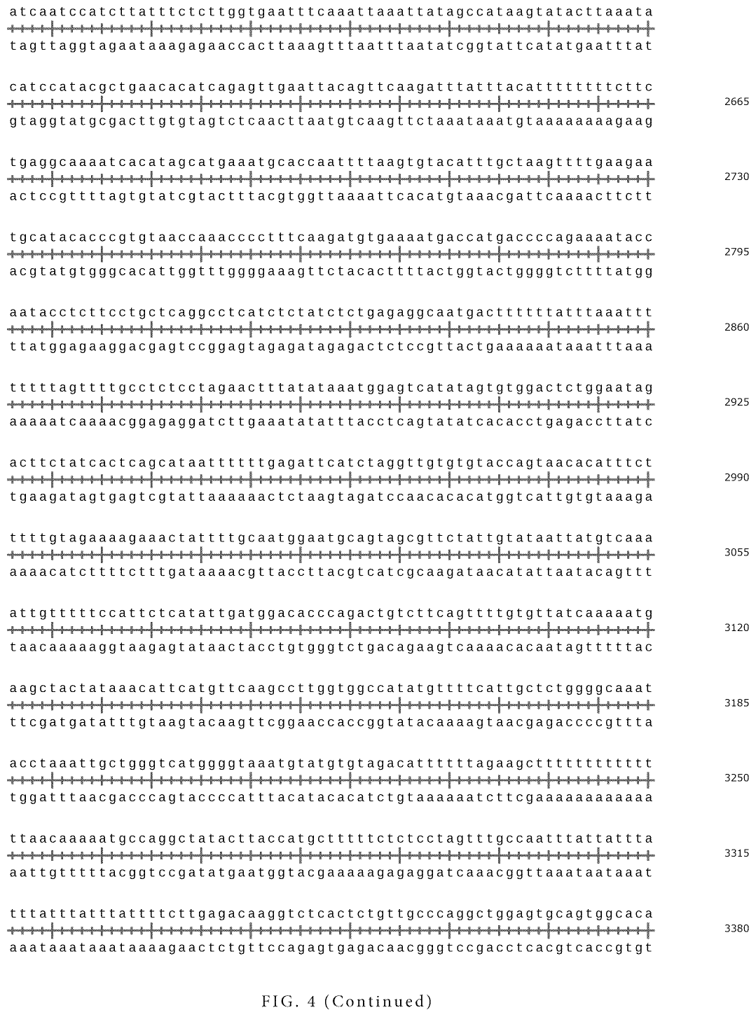

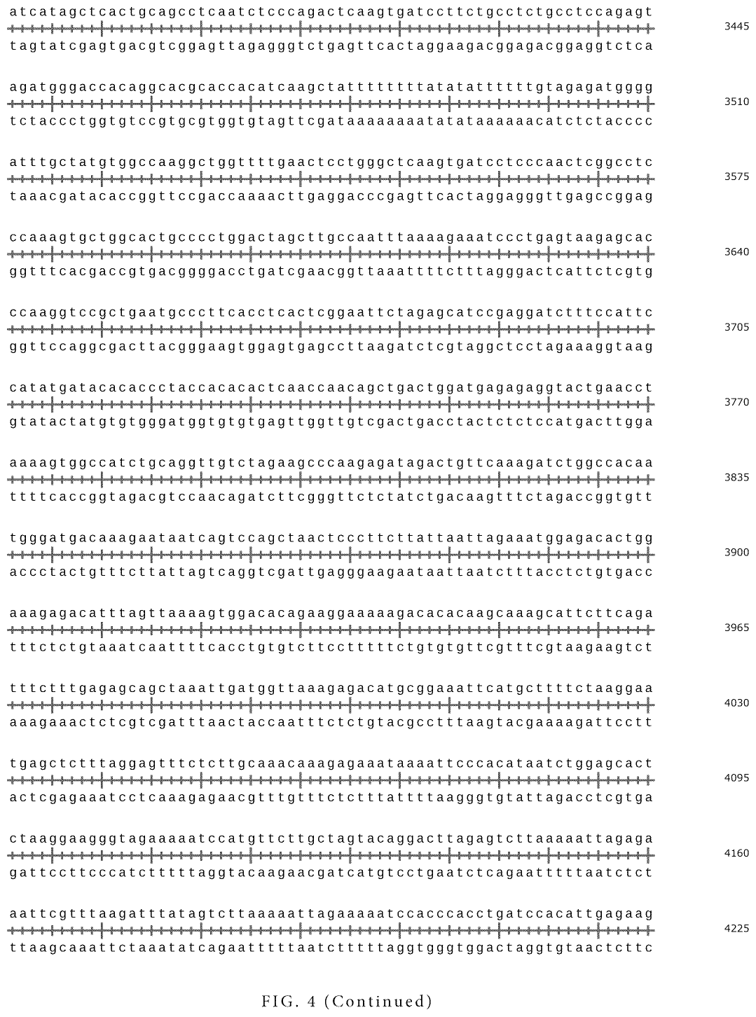

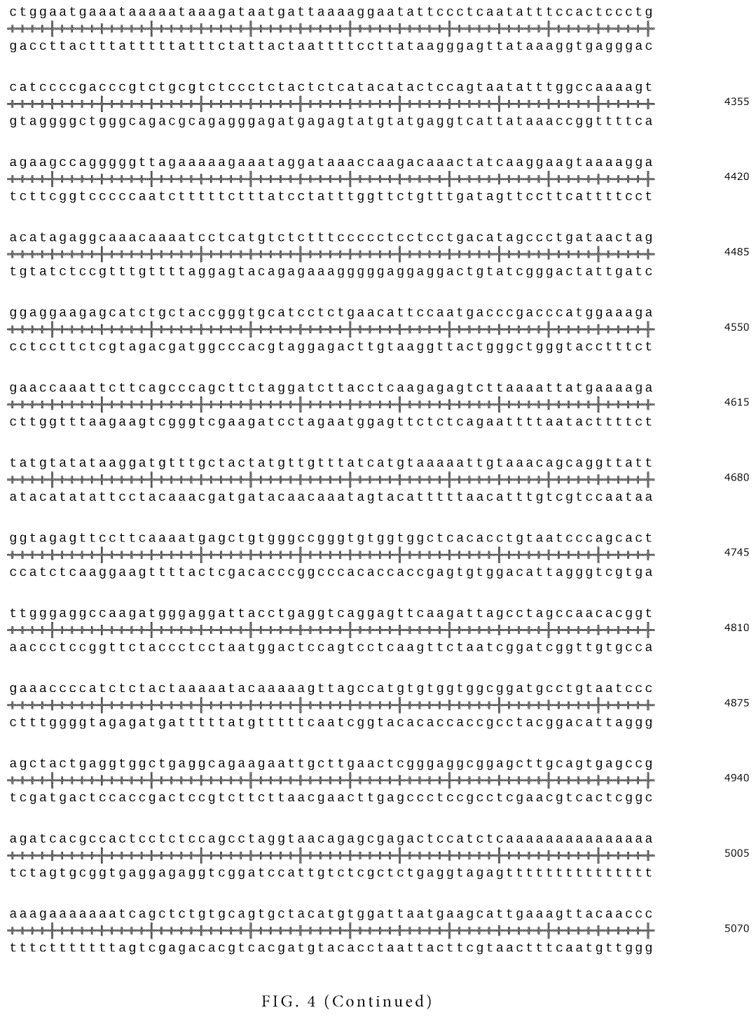

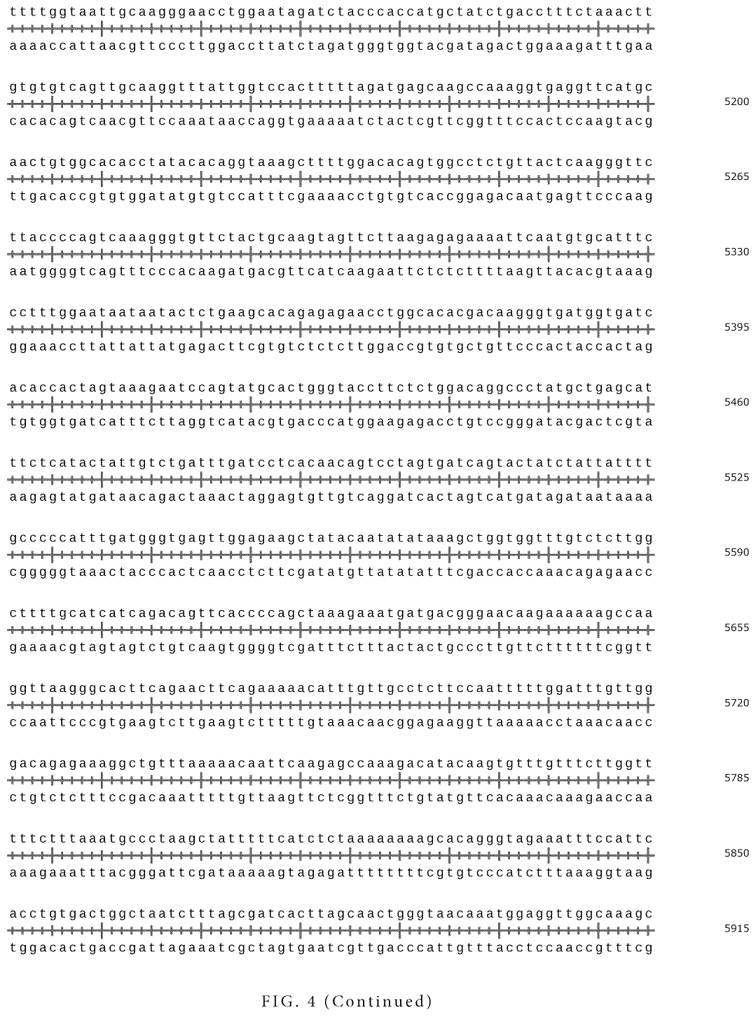

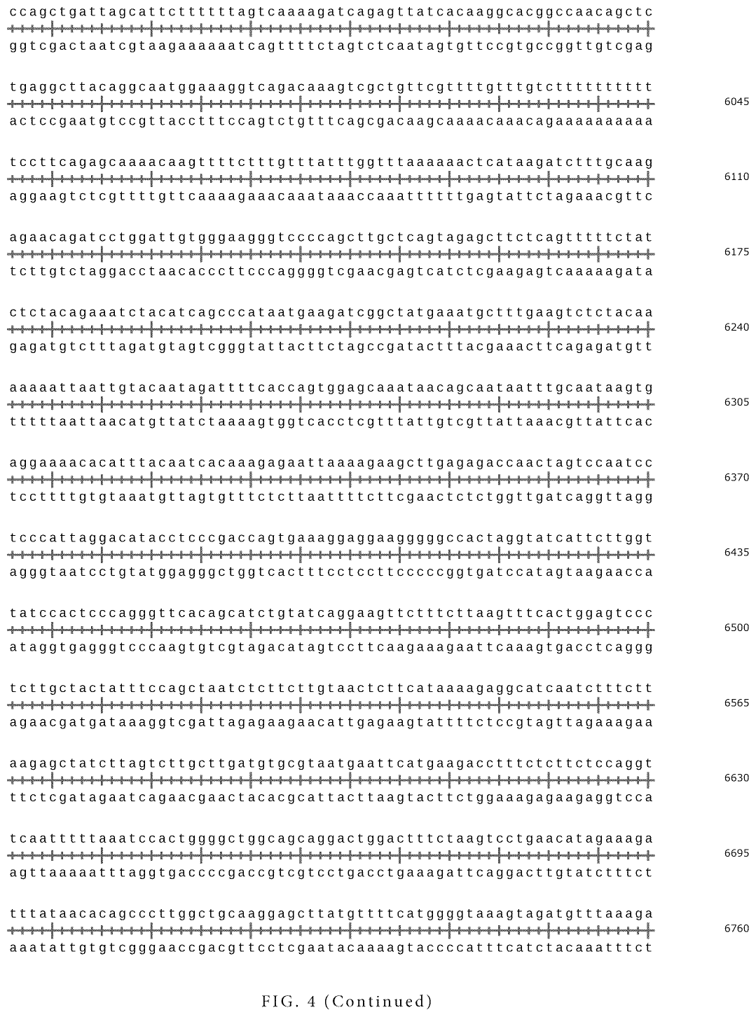

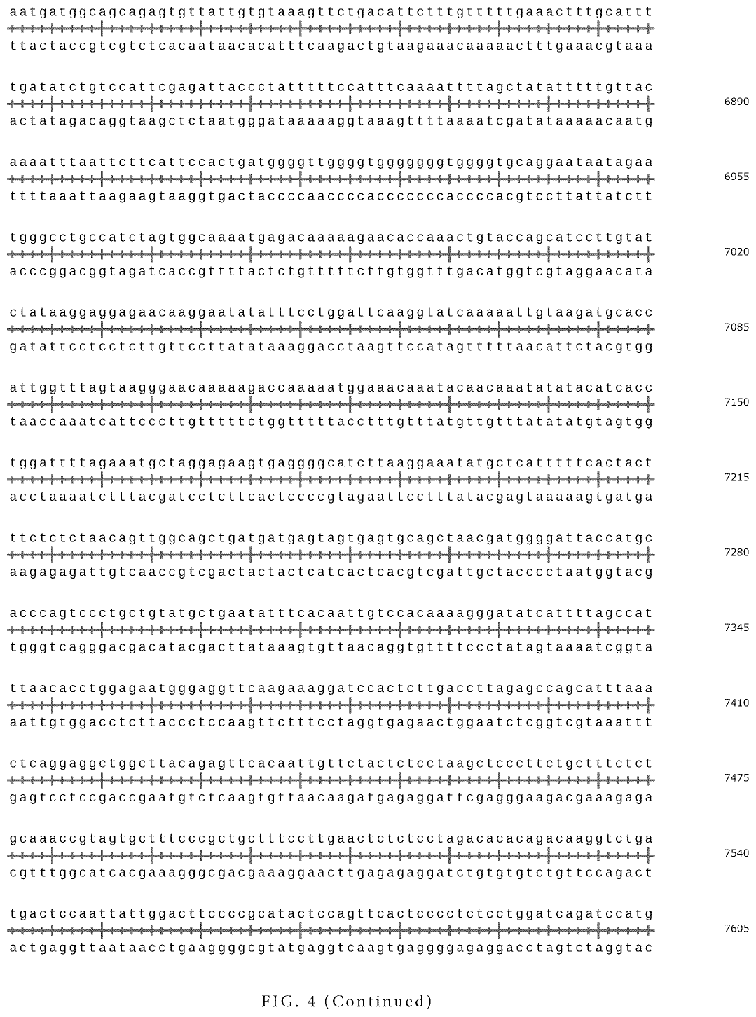

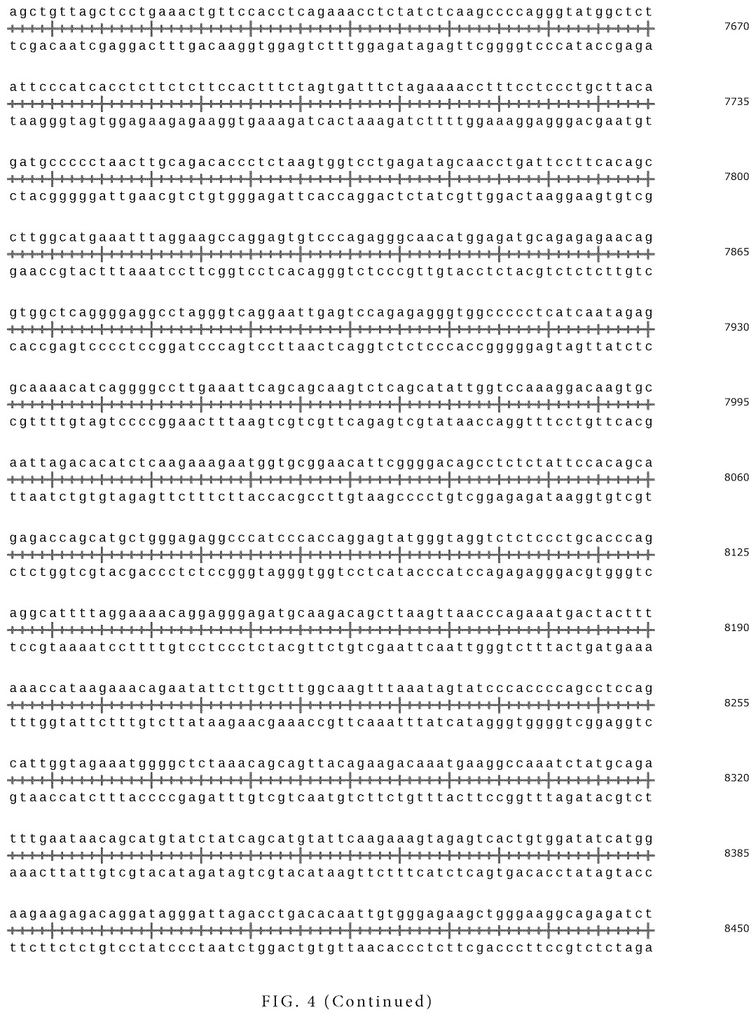

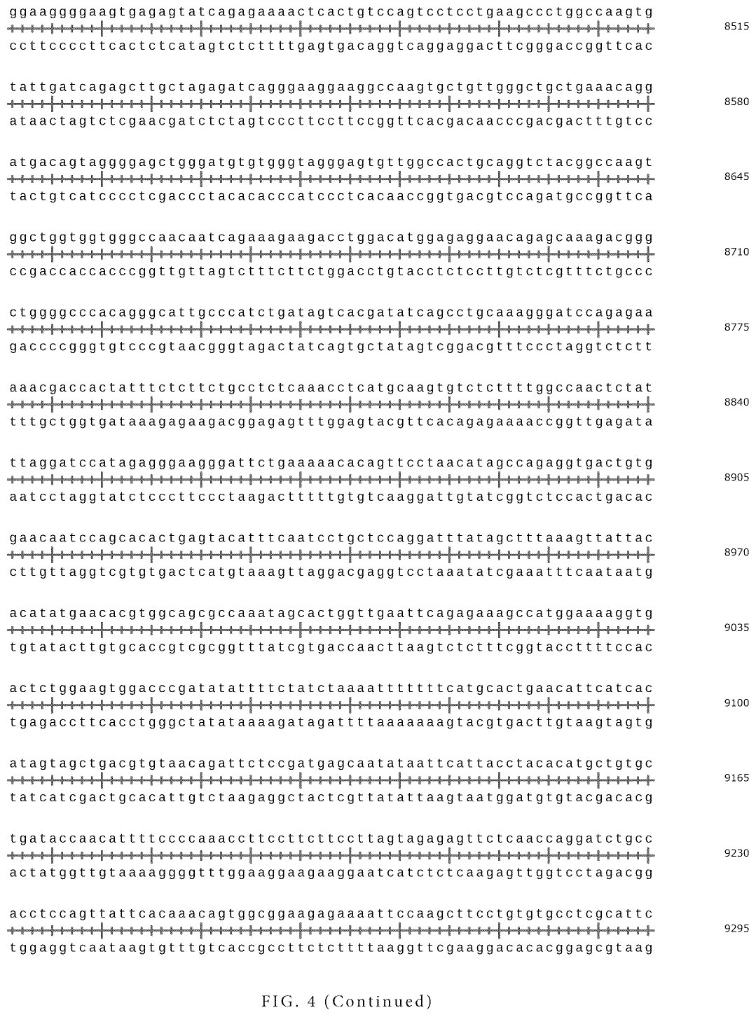

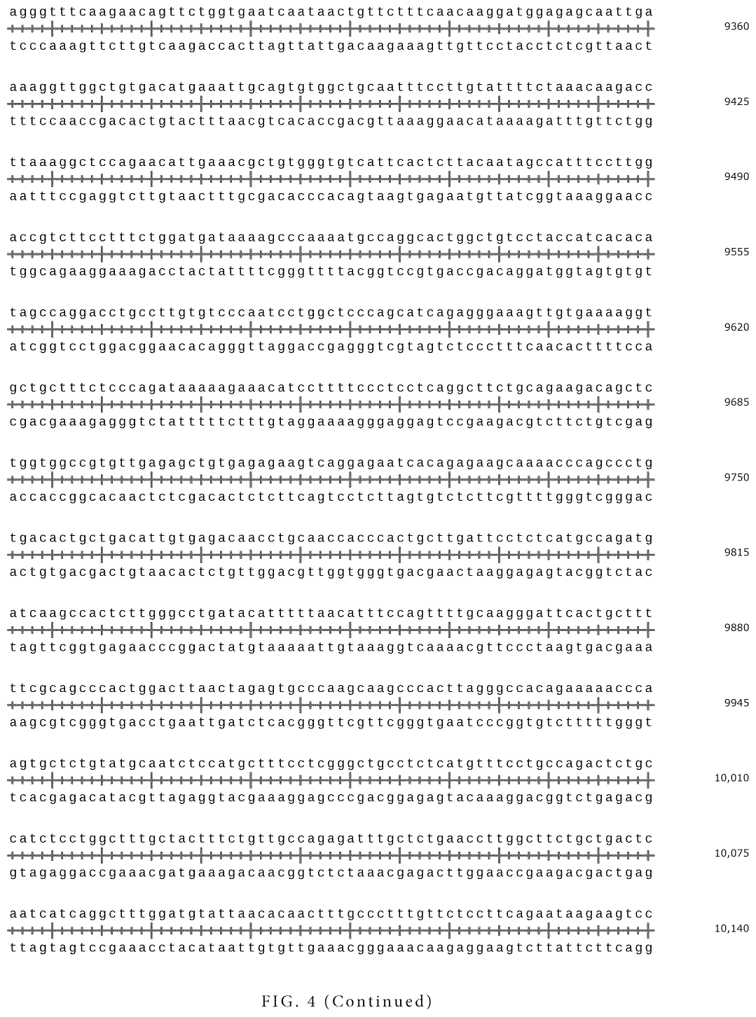

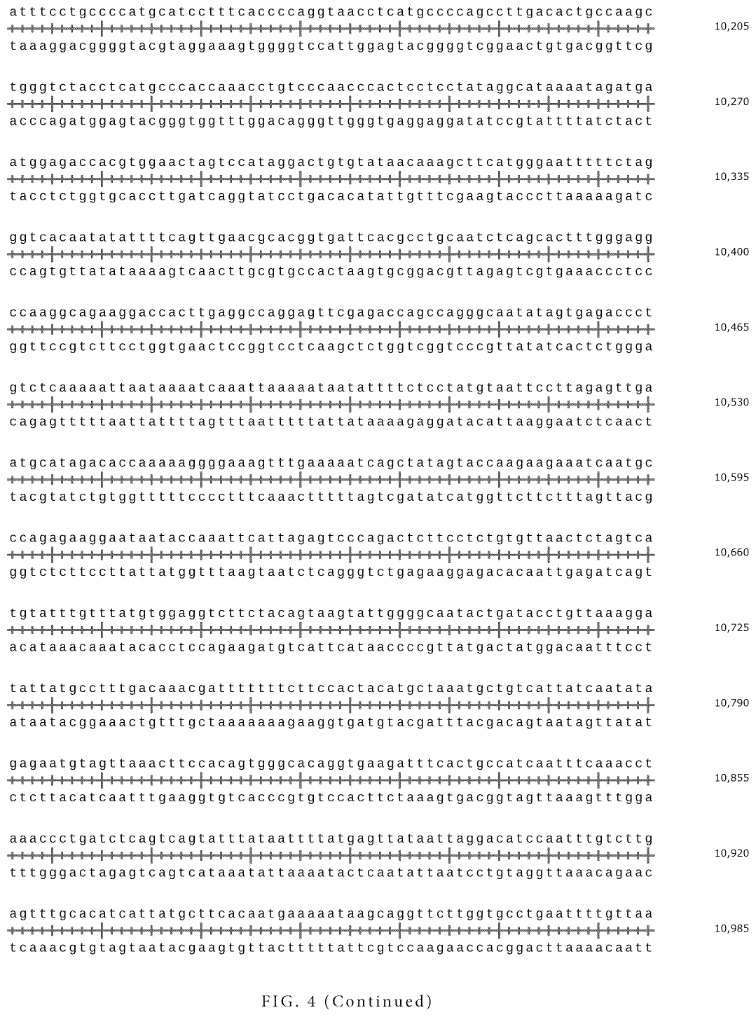

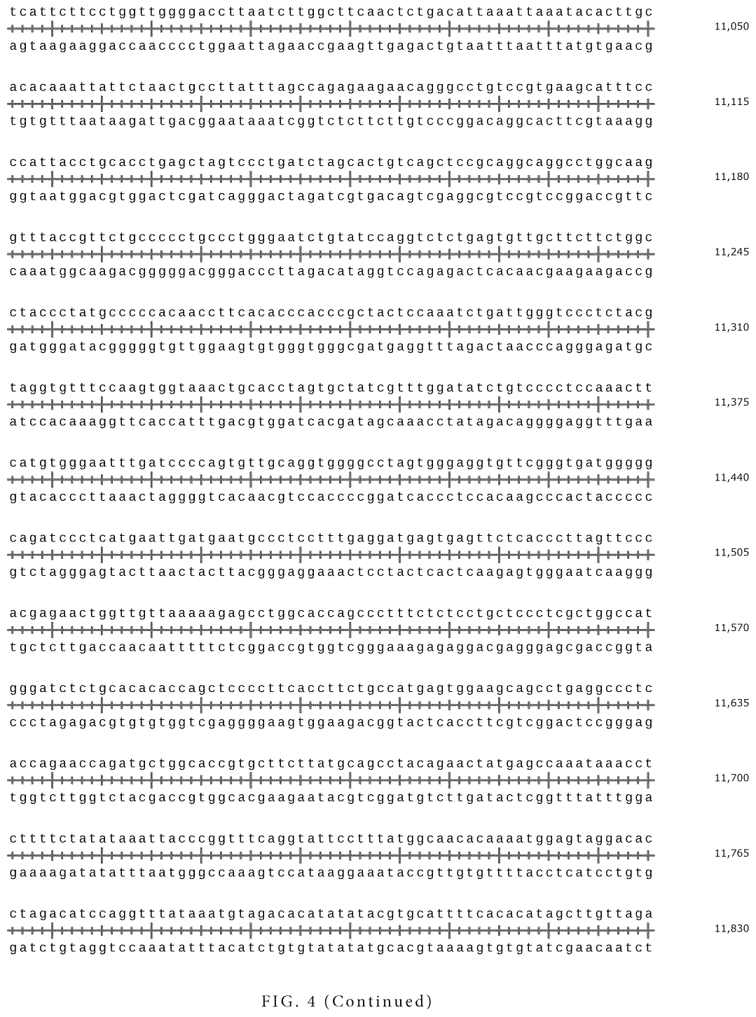

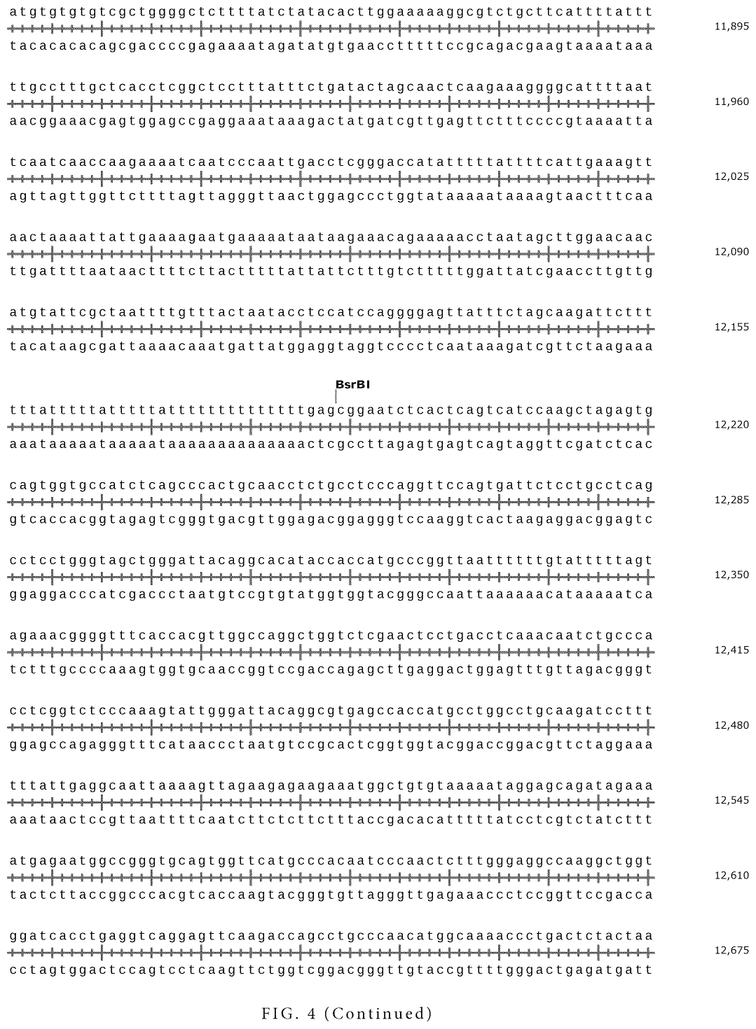

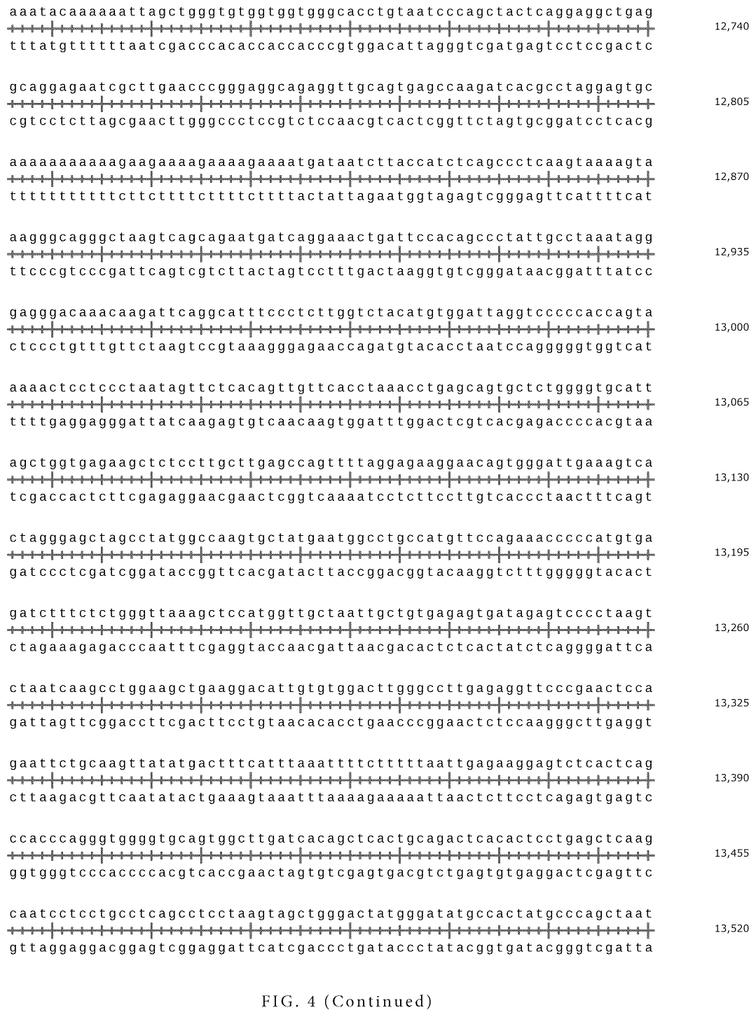

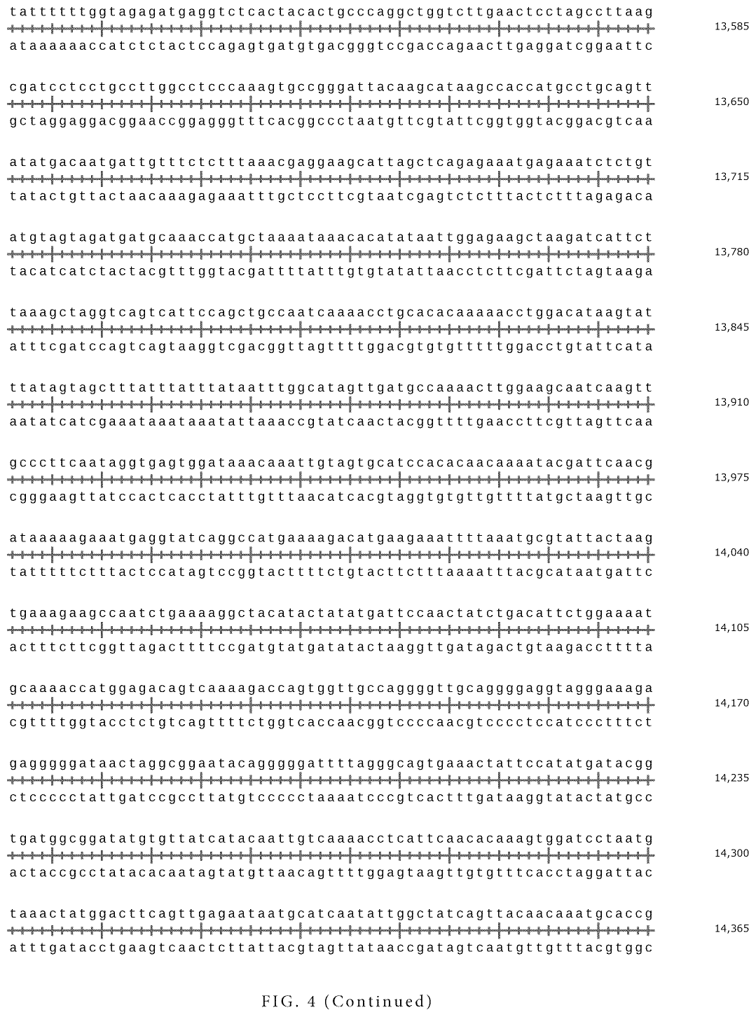

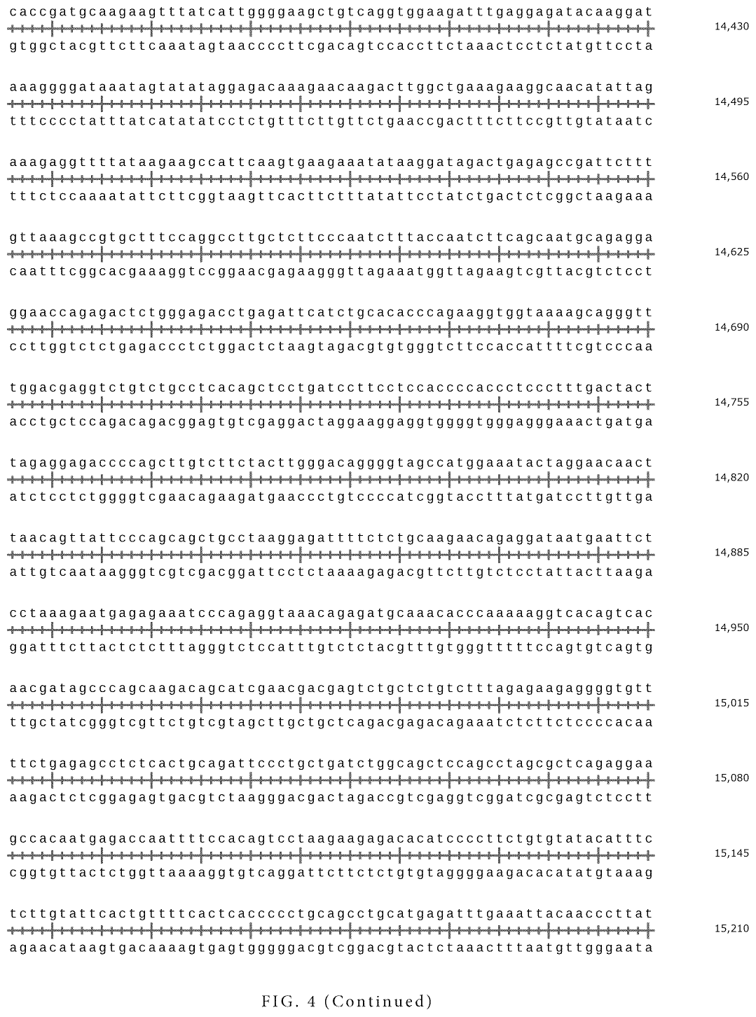

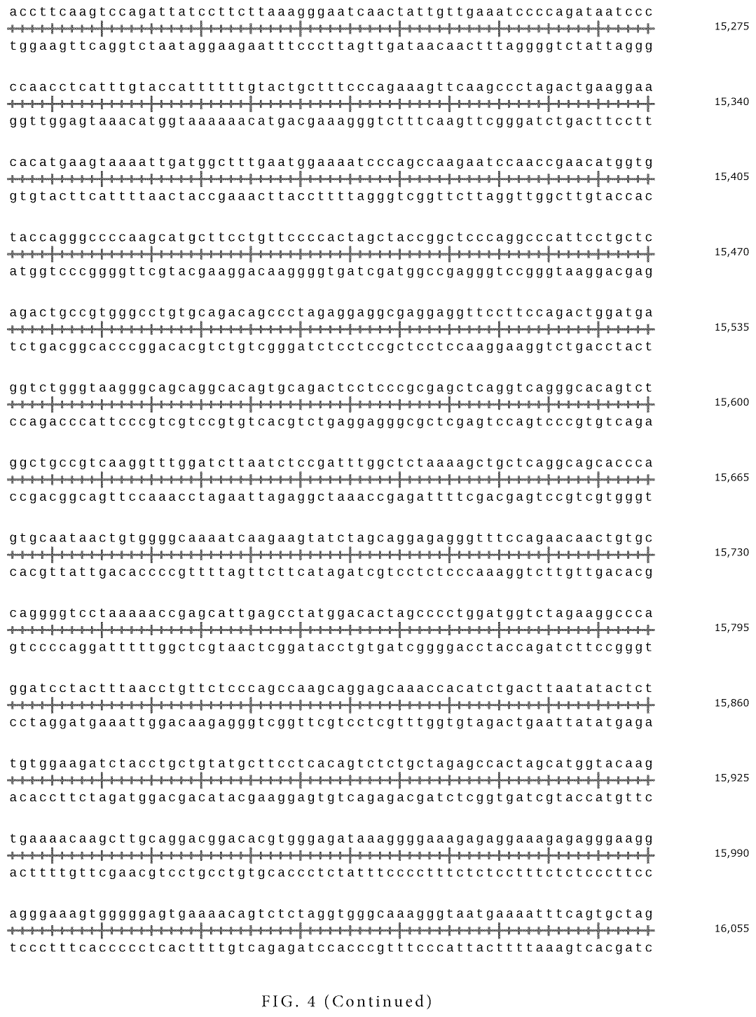

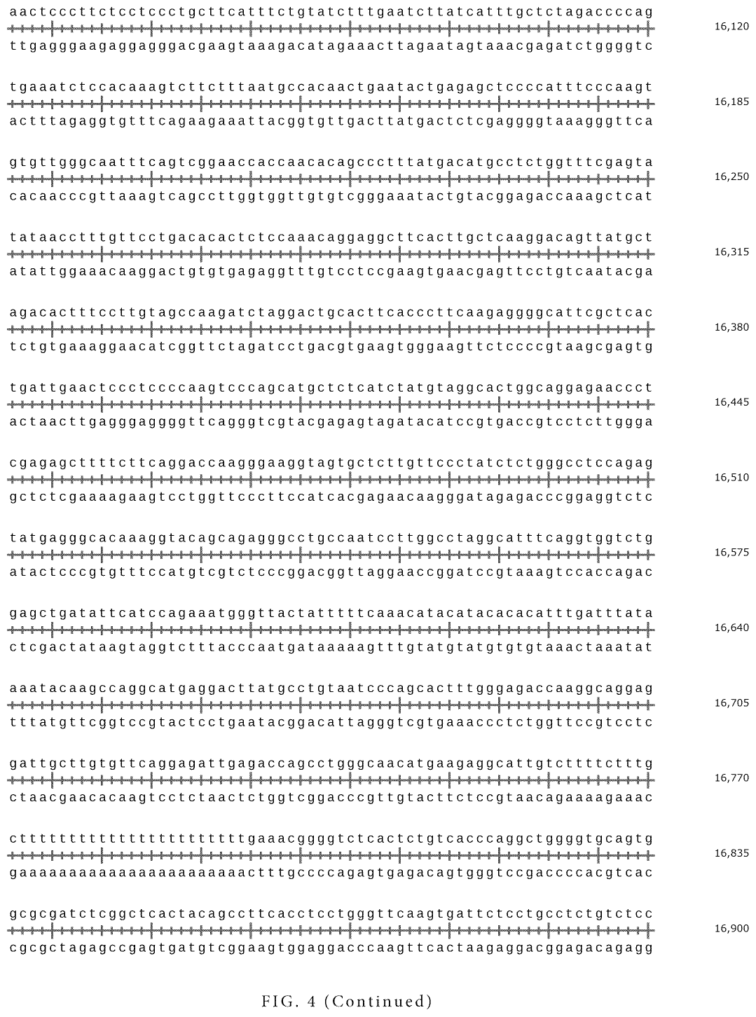

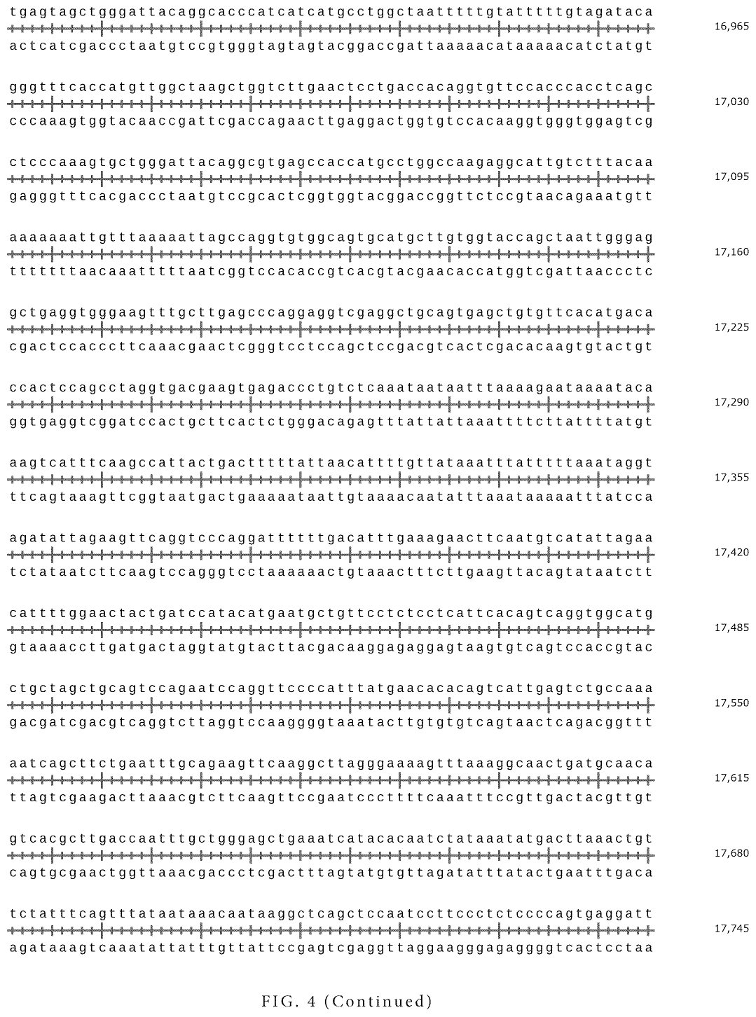

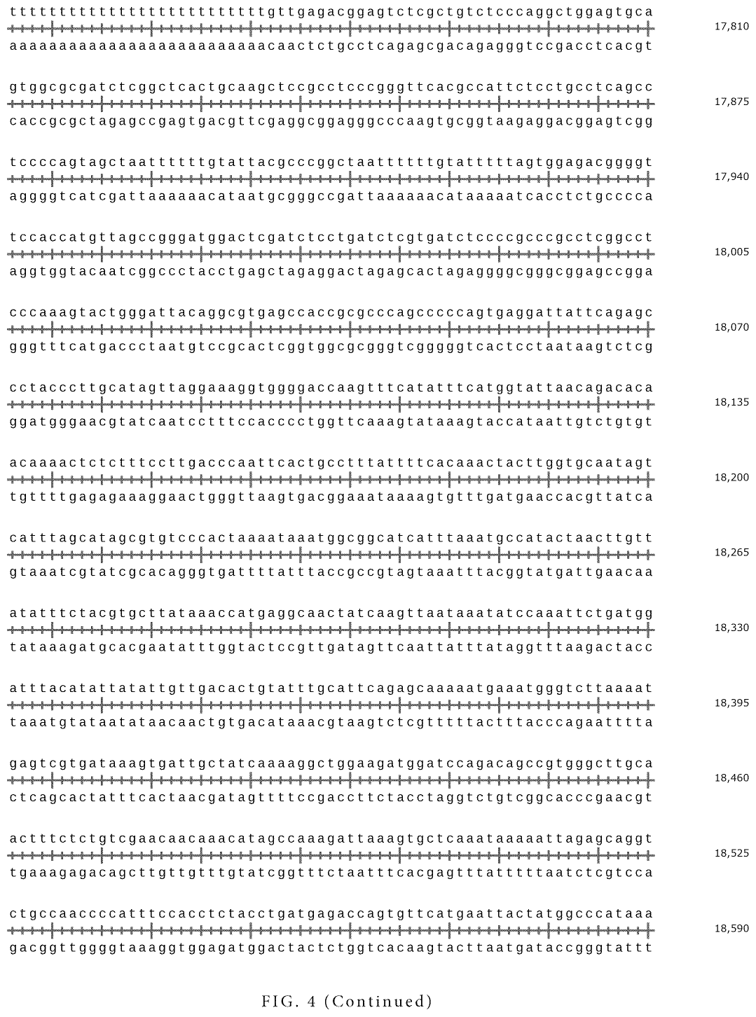

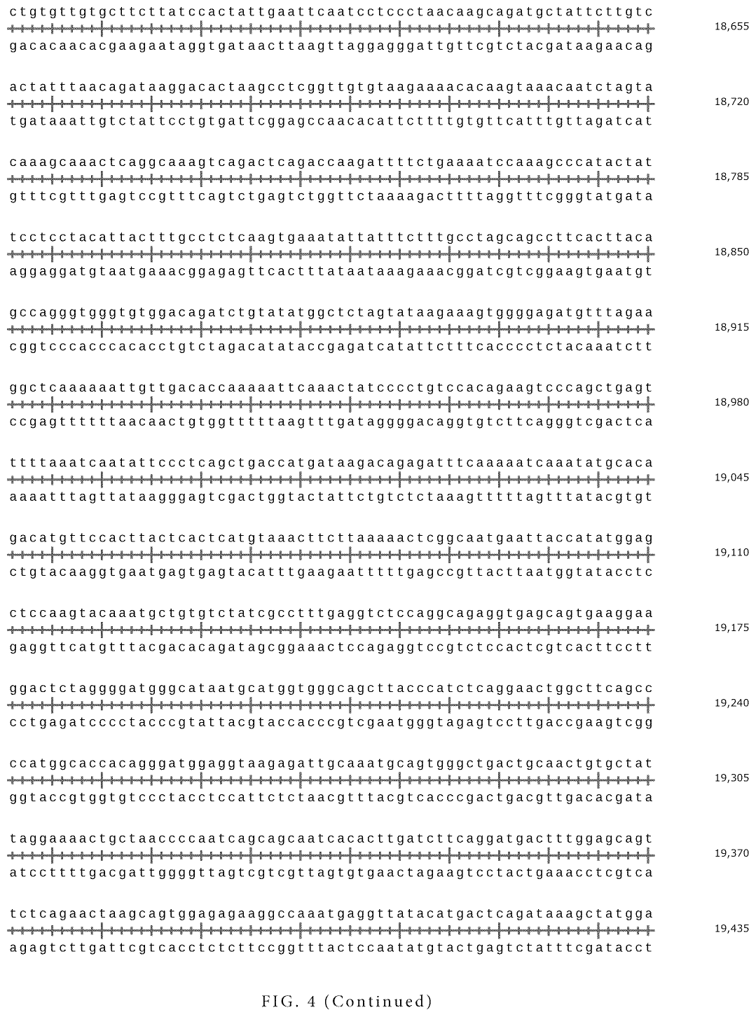

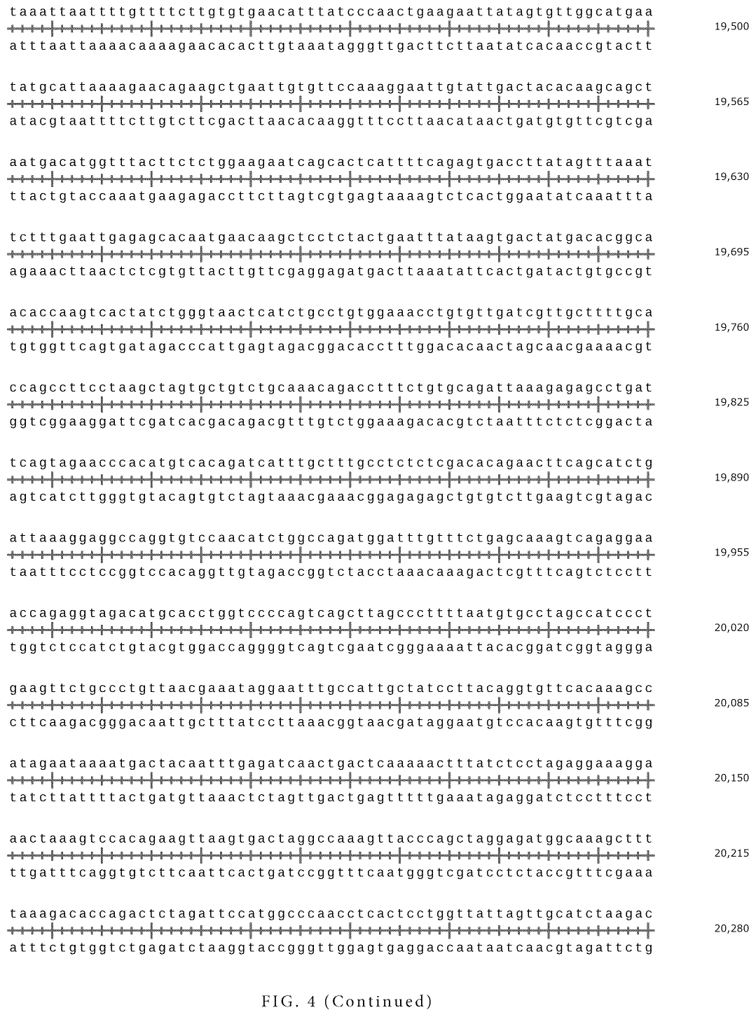

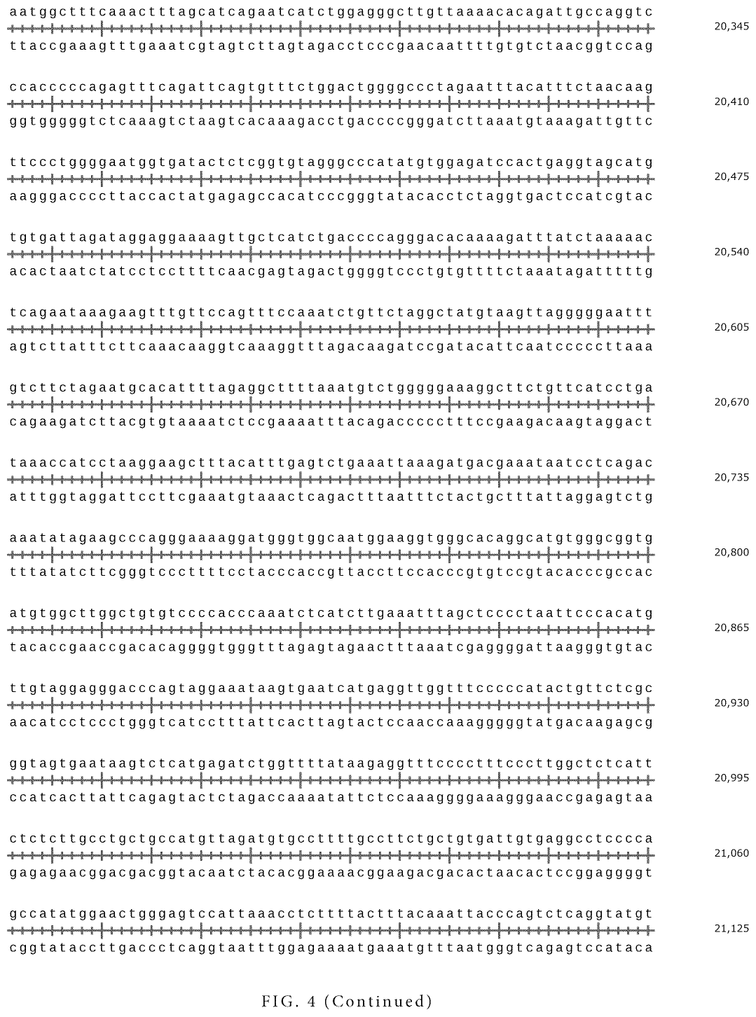

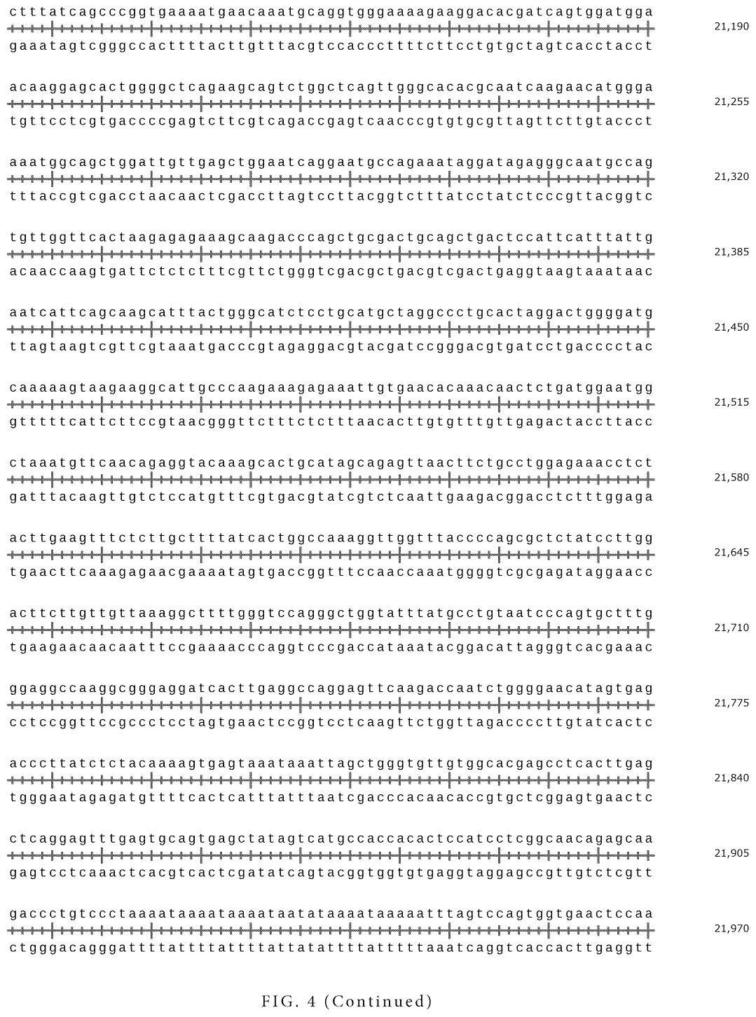

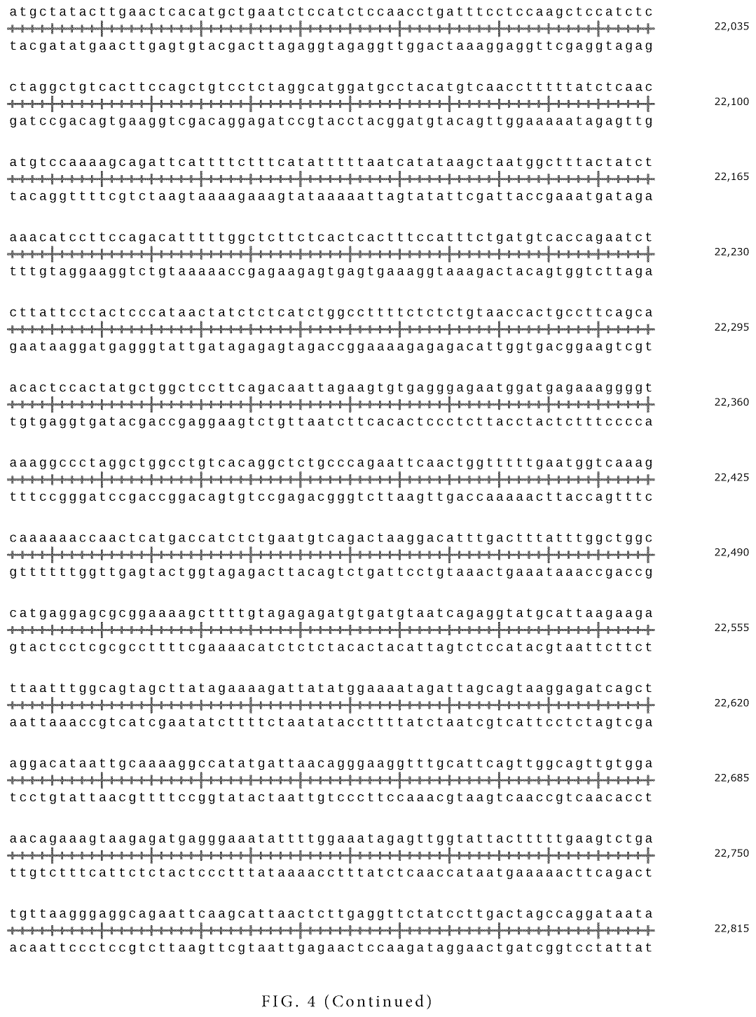

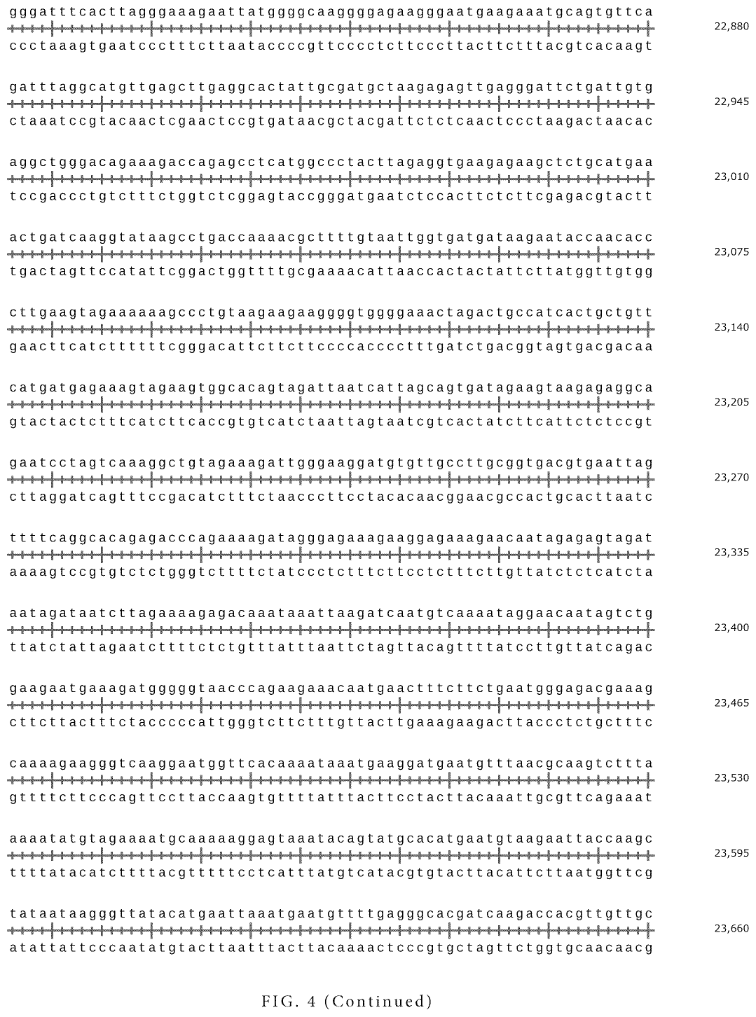

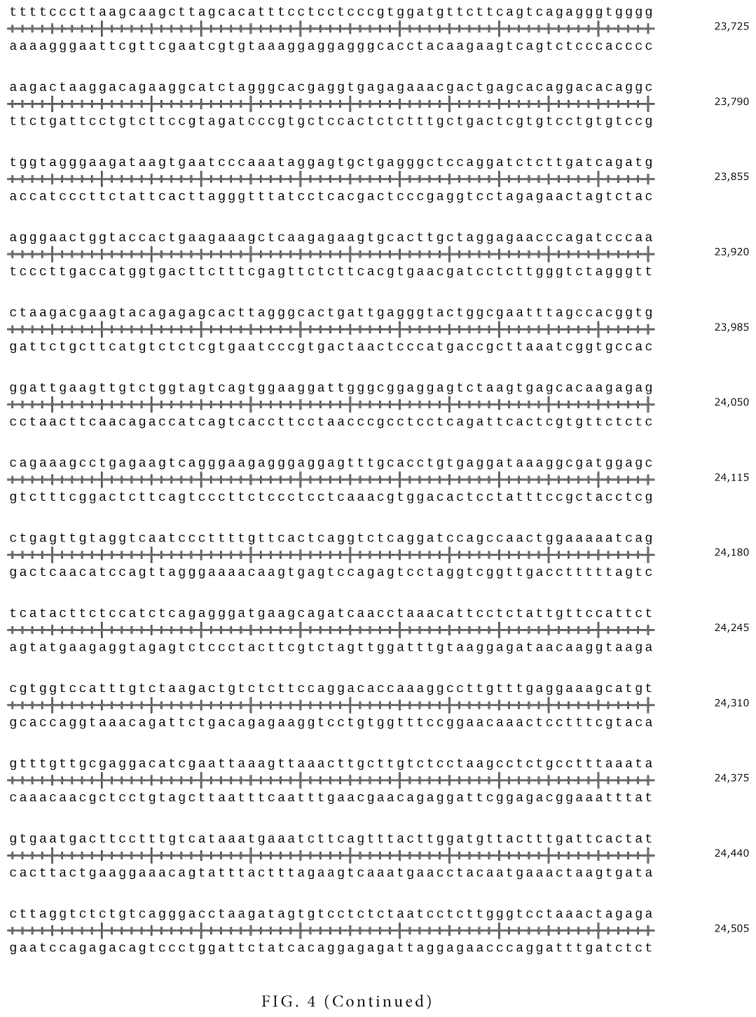

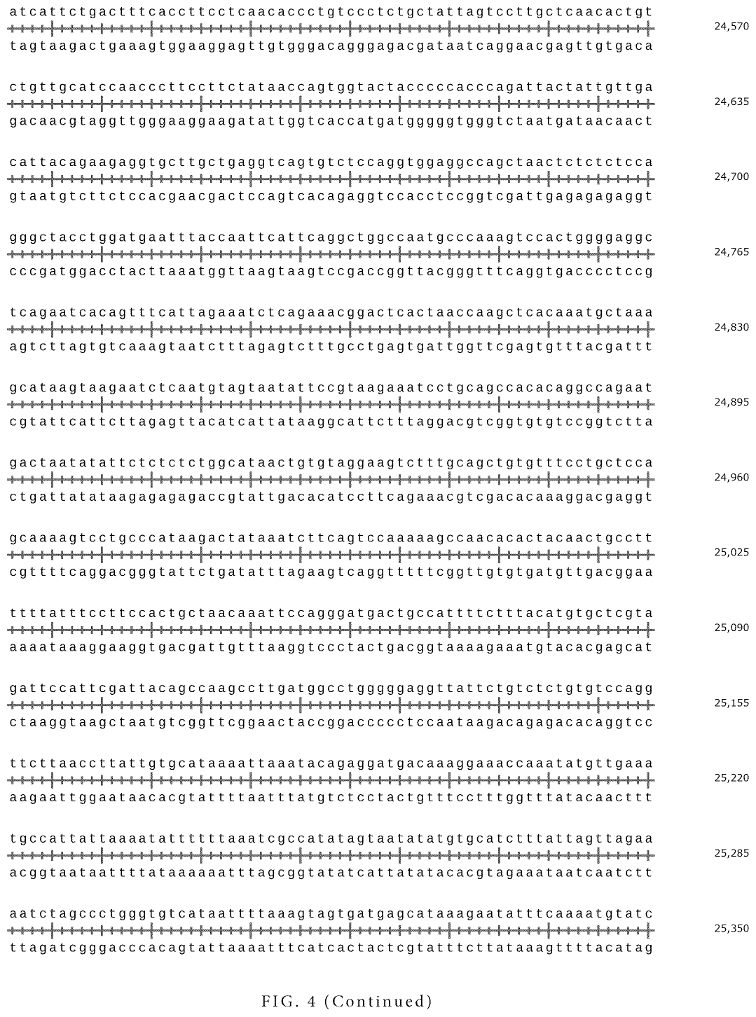

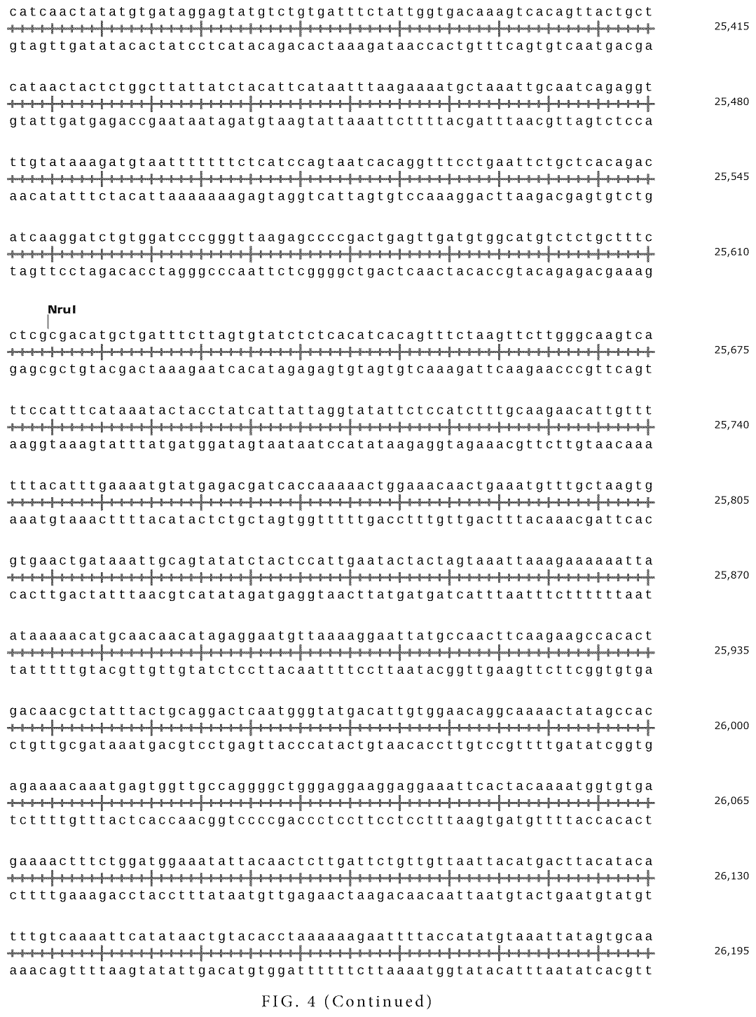

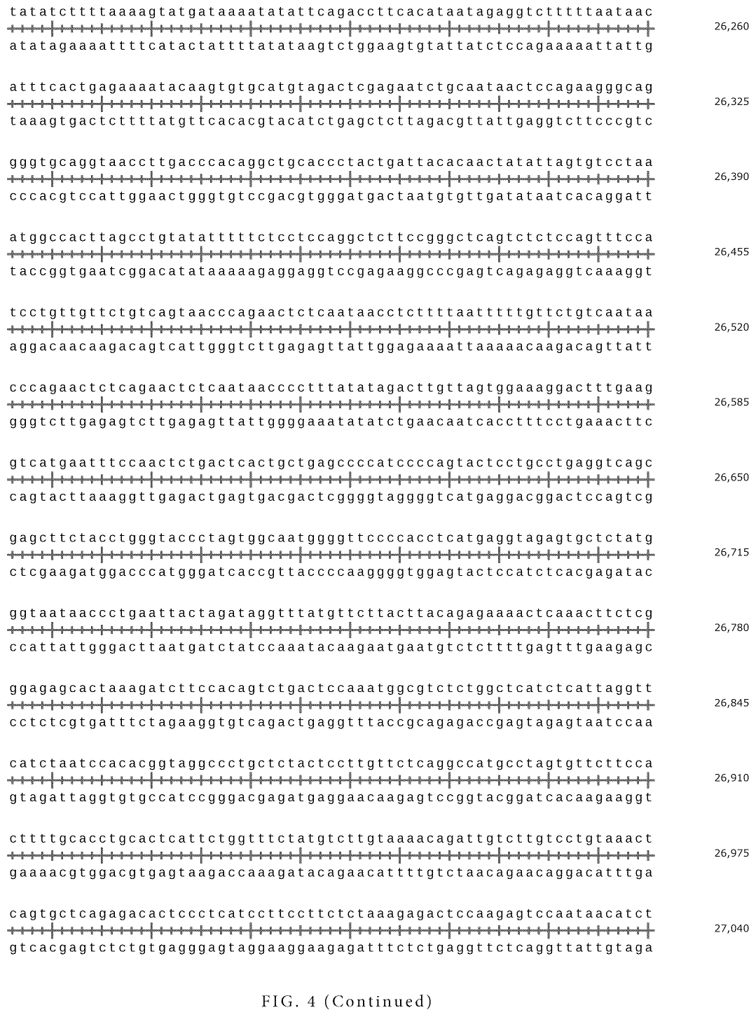

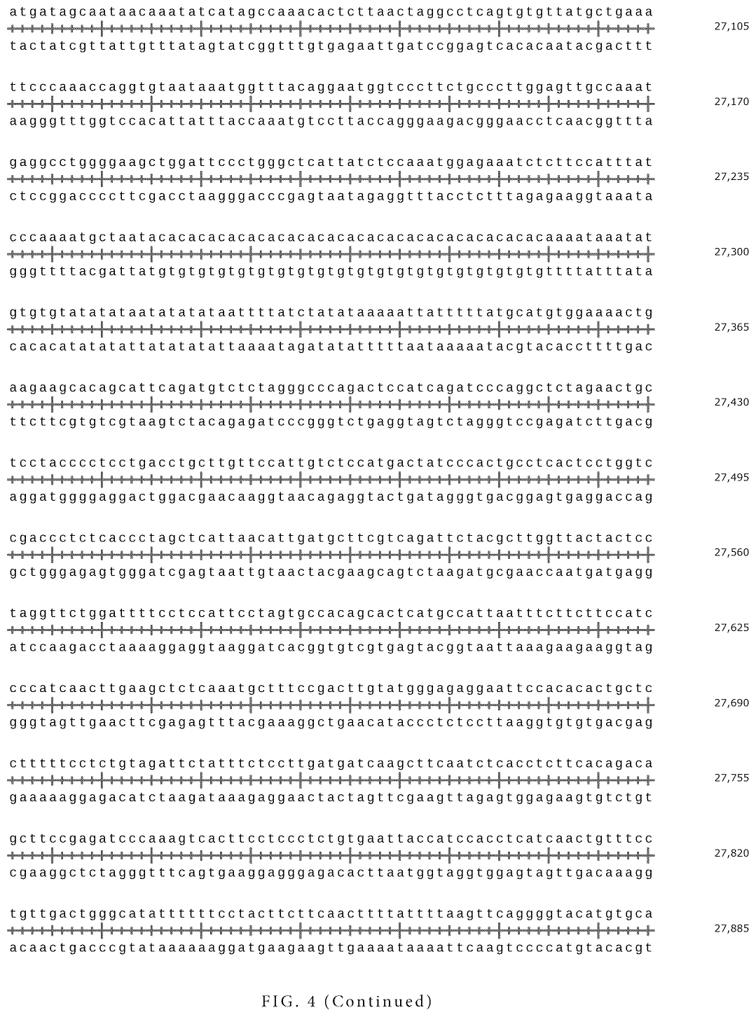

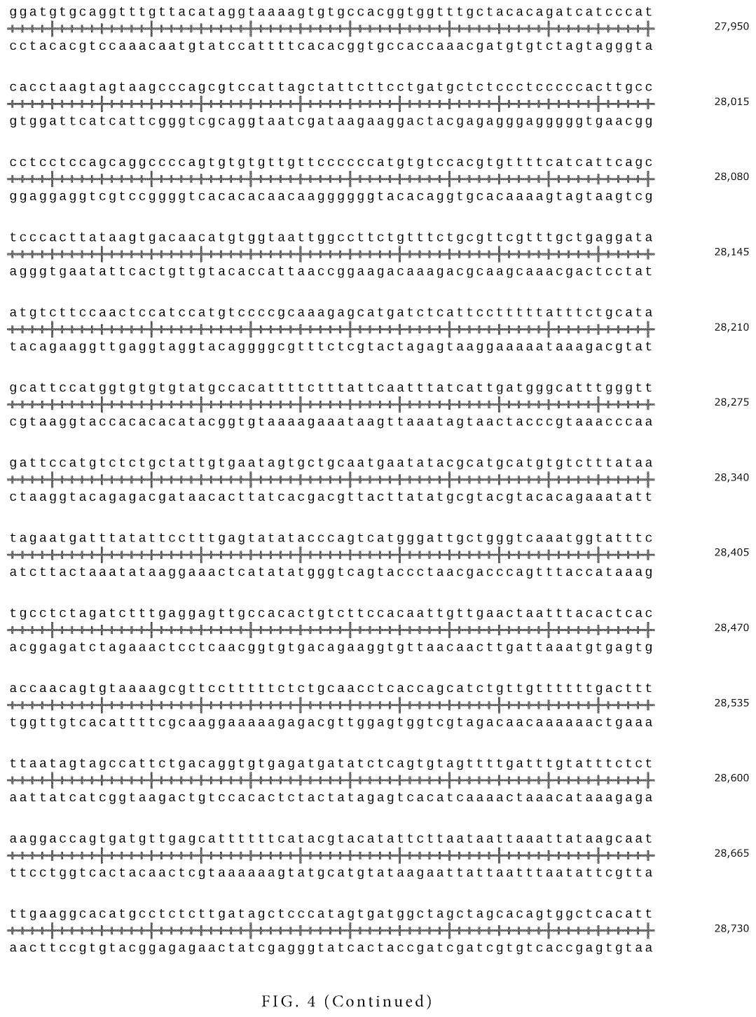

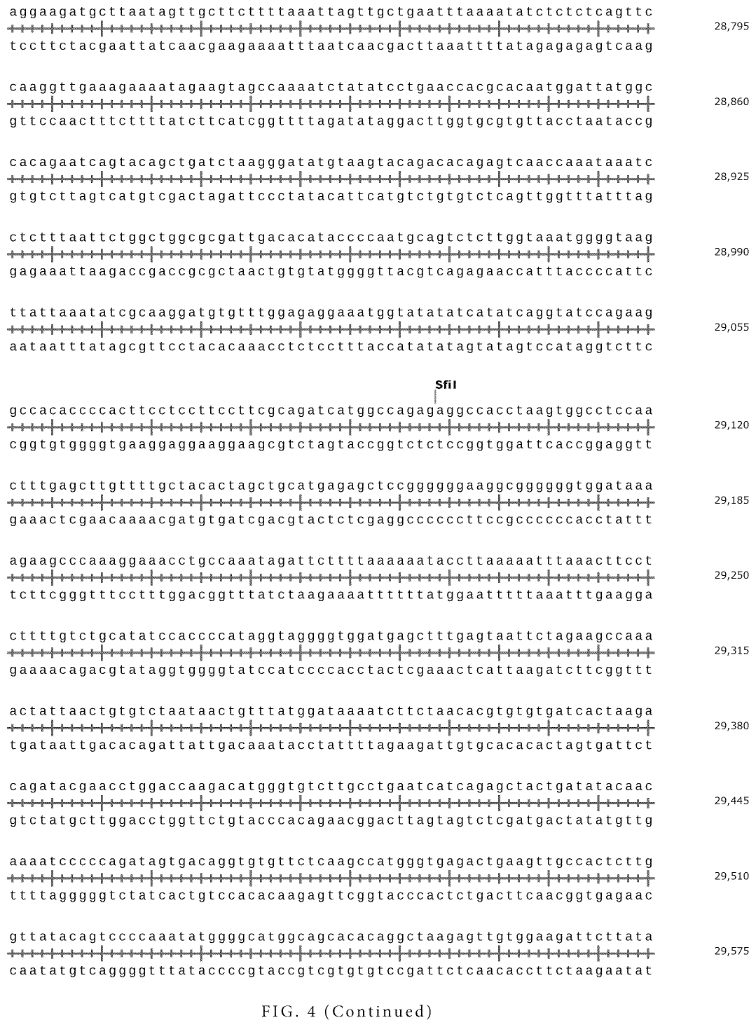

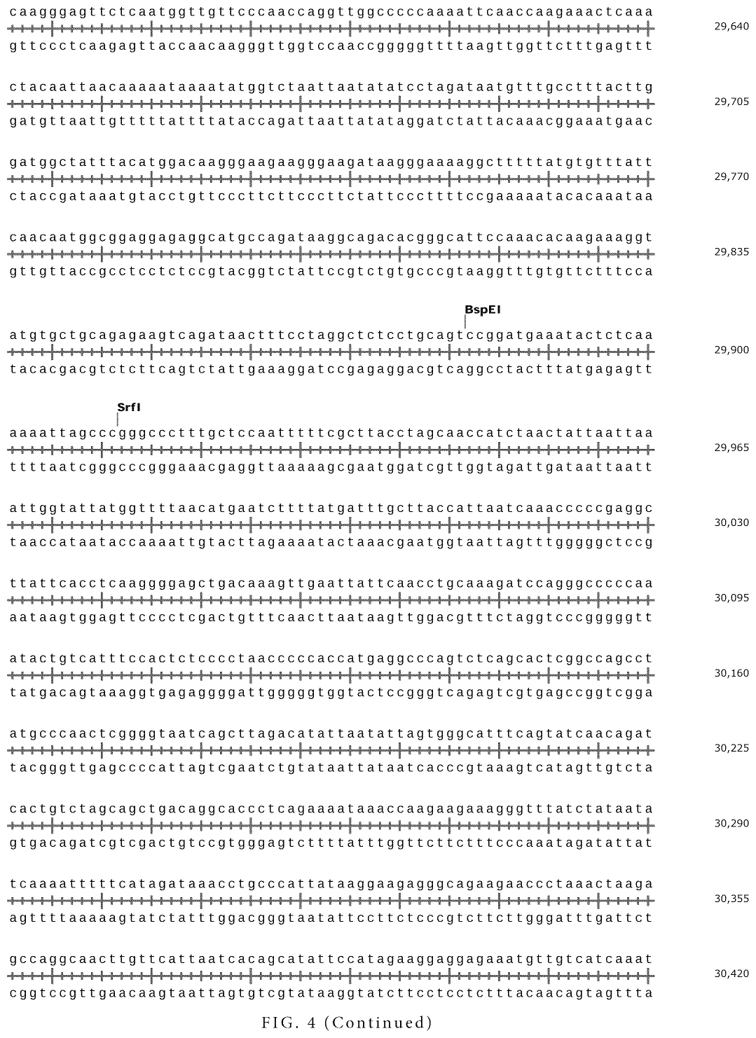

The TTR gene, composed of four exons, is located on chromosome 18 at 18q12.1. The full sequence of the human TTR gene is shown in FIG. 4 and is also available at UniProtKB—P02766 (TTHY_HUMAN). Over 120 TTR variants have so far been identified, the great majority of which are pathogenic. The most common pathogenic variant consists of a point mutation leading to replacement of valine by methionine at position 30 of the mature protein. This Val30Met mutation is responsible for hATTR amyloidosis and is the most frequent amyloidogenic mutation worldwide, accounting for about 50% of TTR variants.

Hereditary transthyretin amyloidosis (hATTR) is a disease caused by mutations in the TTR gene. Autosomal dominant mutations destabilize the TTR tetramer and enhance dissociation into monomers, resulting in misfolding, aggregation, and the subsequent extracellular deposition of TTR amyloid fibrils in different tissue sites. This multisystem extracellular deposition of amyloid (amyloidosis) results in dysfunction of different organs and tissues. In particular, polyneuropathy due to transthyretin amyloidosis (ATTR-PN) and cardiomyopathy due to transthyretin amyloidosis (ATTR-CM) are severe disorders associated with significant morbidity and mortality.

When there is clinical suspicion for hATTR-PN, diagnosis is typically done by tissue biopsy with staining for amyloid, amyloid typing (using immunohistochemistry or mass spectrometry), and/or TTR gene sequencing. When there is clinical suspicion for ATTR-CM, the key diagnostic tools are either endomyocardial biopsy (with tissue staining and amyloid typing by immunohistochemistry or mass spectrometry) or 99mtechnetium-pyrophosphate scan. Both of these approaches can provide a diagnosis of ATTR-CM. TTR gene sequencing can be used to differentiate between the hATTR-CM (mutation positive) and ATTRwt-CM (mutation negative).

The compositions described herein include a spacer having a nucleotide sequence that functions as a guide to direct a gene editing protein (e.g., a base editor) to alter the TTR gene, for example by introducing one or more nucleobase alterations in the TTR gene. These point mutations may be used to disrupt gene function, by the introduction of a missense mutation(s) that results in production of a less functional, or non-functional protein, thus silencing the TTR gene. Alternatively, it is contemplated herein that corrections to one or more point mutation(s) may be made using a gene editing protein to alter a mutated gene to correct the underlying mutation causing the dysfunction in the TTR gene or otherwise mitigate against dysfunction of the gene.

### Gene Editing/Gene Modification

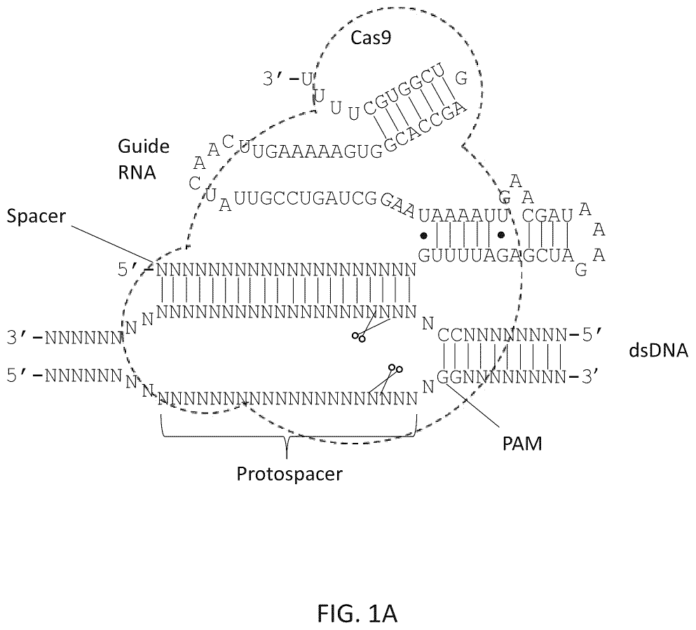

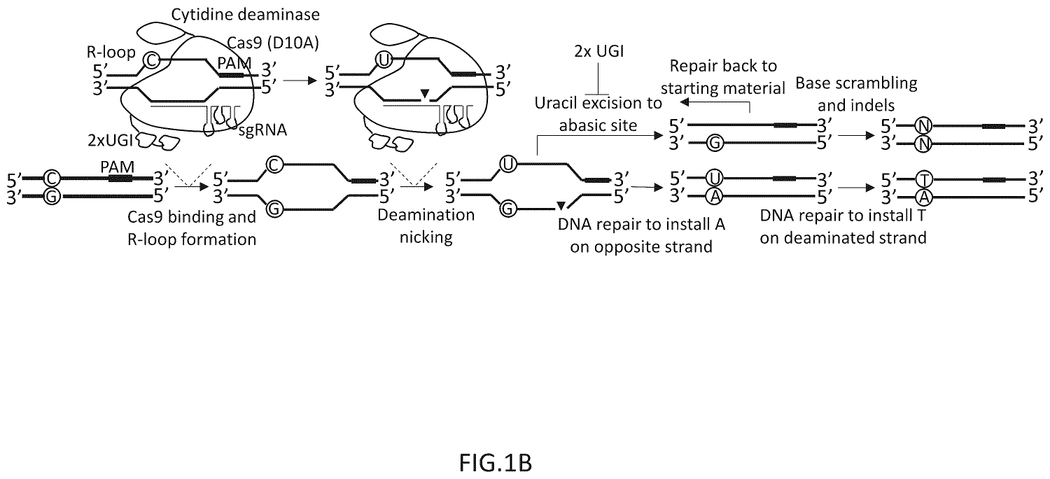

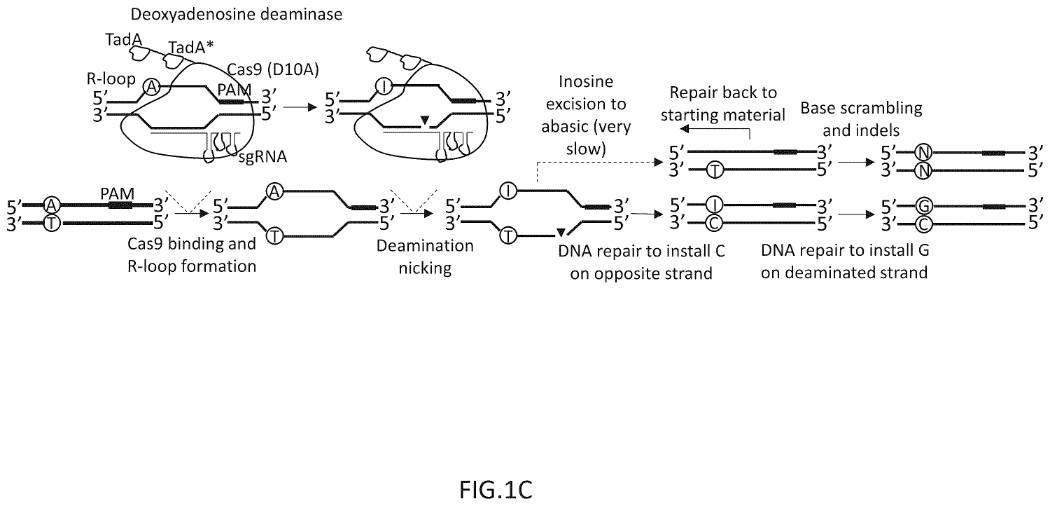

The term “gene editing” or “gene modification” and its grammatical equivalents as used herein refers to genetic engineering in which one or more nucleotides are inserted, replaced, or removed from a genome. Gene editing can be performed using a nuclease (e.g., a natural-existing nuclease or an artificially engineered nuclease). Gene modification can include introducing a double stranded break, a non-sense mutation, a frameshift mutation, a splice site alteration, or an inversion in a polynucleotide sequence, e.g., a target polynucleotide sequence. FIG. 1A depicts a crispr Cas9 protein which is an RNA-guided endonuclease that can be used to impart a double-stranded break at a site-specific location in DNA or a gene. Gene modification can also be accomplished using other editors, such as base editors.

### Base Editors

A base editor (BE) or nucleobase editor (NBE) refers to an agent comprising a polypeptide that is capable of making a modification to a base (e.g., A, T, C, G, or U) within a nucleic acid sequence (e.g., DNA or RNA). A base editor may comprise a macromolecule or macromolecular complex that is capable of converting a nucleobase in a polynucleic acid sequence into another nucleobase (e.g., a transition or transversion) in one location or two or more locations within a base editing window. A base editor may comprise a combination of (a) a nucleotide-, nucleoside-, or nucleobase-converting enzyme and (b) a nucleic acid binding protein that can be programmed to bind to a specific nucleic acid sequence. The nucleic acid binding protein may be catalytically inactivated or impaired such that it does not cleave a single stranded nucleic acid target or such that it nicks or cleaves at most one strand of a double stranded nucleic acid target.

A base editor may comprise a polynucleotide programmable DNA binding domain fused or linked to a domain having base editing activity, resulting in a base editor fusion protein. The base editor fusion protein may comprise one or more linkers, for example, peptide linkers between the domains. In some embodiments, the domain having base editing activity is linked to the guide RNA (e.g., via an RNA binding motif on the guide RNA and an RNA binding domain fused to the deaminase).

In some embodiments, a base editor is a class of modular programmable proteins comprising a deaminase domain fused to a catalytically impaired CRISPR-Cas enzyme. Adenine base editors (ABEs) convert A:T to G:C base pairs and cytosine base editors (CBEs) convert C:G to T:A base pairs, through hydrolytic deamination and subsequent cellular processing without generating double-stranded DNA breaks. The respective deaminases of base editors are directed to the site of interest by a guide RNA (gRNA) within the D10A nickase Cas9 (nCas9). Cytidine deaminase enzyme of a CBE directs conversion of cytosine to uridine, thereby effecting a C→T (or G→A) substitution (see FIG. 1B). “Cytidine deaminase” is used herein to refer to a deaminase enzyme that acts on deoxycytidine, on cytidine, or both on deoxycytidine and on cytidine to convert cytosine to uridine. Cytidine deaminase and cytosine deaminase may be used interchangeably herein. Where the goal is to disrupt a gene in vivo for a therapeutic purpose, cytosine base editors (CBE) are capable of potentially being able to directly introduce stop codons into the coding sequence of the gene (nonsense mutations) by altering specific codons for glutamine (CAG→TAG, CAA→TAA), arginine (CGA→TGA), and tryptophan (TGG→TAG/TAA/TGA, with editing of cytosines on the antisense strand).

In comparison, the adenosine deaminase enzyme of an ABE directs conversion of adenosine to inosine, thereby effecting a A→G (or T→C) substitution (see FIG. 1B). “Adenosine deaminase” is used herein to refer to a deaminase enzyme that acts on deoxyadenosine, on adenosine, or both on deoxyadenosine and on adenosine to convert adenine to hypoxanthine or, alternatively, adenosine to inosine. Because the structure of inosine is the similar to guanosine (inosine does not include an exocyclic amino group of guanosine), inosine tends to behave as guanosine. Inosine is eventually replaced by guanosine through subsequent cellular processing. Accordingly, adenosine deaminase effects an A→G (or T→C) substitution. Adenosine deaminase and adenine deaminase may be used interchangeably herein. Adenine base editors (ABE) cannot directly introduce stop codons, as there are no A→G changes that result in nonsense mutations.

Adenine base editors can be used, for example, to disrupt gene function by editing the start codon, from either ATG→GTG or ATG→ACG. A second strategy by which adenine base editors can disrupt gene function is by editing splice sites, whether splice donors at the 5′ ends of introns or splice acceptors at the 3′ ends of introns. Splice site disruption can result in the inclusion of intronic sequences in messenger RNA (mRNA), potentially introducing nonsense, frameshift, or in-frame mutations that result in premature stop codons or in insertion/deletion of amino acids that disrupt protein activity, or in the exclusion of exonic sequences, potentially introducing nonsense, frameshift, or in-frame indel mutations.

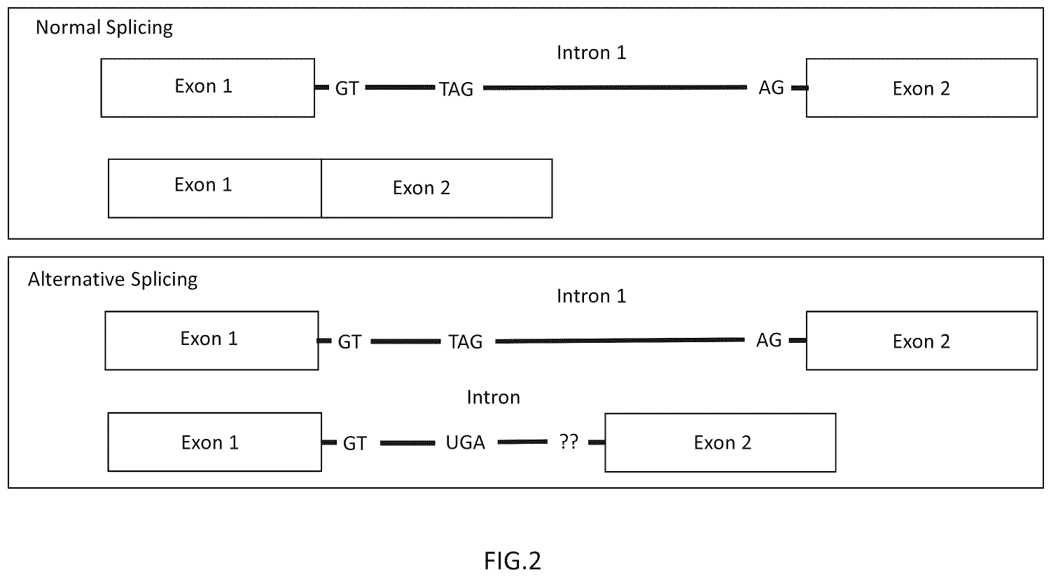

As shown in FIG. 2, canonical splice donors comprise the DNA sequence GT on the sense strand, whereas canonical splice acceptors comprise the DNA sequence AG. Alteration of the sequence disrupts normal splicing. Splice donors can be disrupted by adenine base editing of the complementary base in the second position in the antisense strand (GT→GC), and splice acceptors can be disrupted by adenine base editing of the first position in the sense strand (AG→GG).

Adenine base editors (ABE) include, but are not limited to, ABE8.8 (Gaudelli et al., Nat Biotechnol. 2020 July; 38 (7): 892-900. doi: 10.1038/s41587-020-0491-6. Epub 2020 Apr. 13)).

In embodiments, an adenine base editor is encoded by mRNA comprising the sequence of MA004 mRNA shown in Table 11. In embodiments, the adenine base editor is encoded by mRNA comprising a sequence having 50% or more sequence identity to MA004 mRNA shown in Table 11, 60% or more sequence identity to MA004 mRNA shown in Table 11, 70% or more sequence identity to MA004 mRNA shown in Table 11, 75% or more sequence identity to MA004 mRNA shown in Table 11, 80% or more sequence identity to MA004 mRNA shown in Table 11, 85% or more sequence identity to MA004 mRNA shown in Table 11, 90% or more sequence identity to MA004 mRNA shown in Table 11, 95% or more sequence identity to MA004 mRNA shown in Table 11, 96% or more sequence identity to MA004 mRNA shown in Table 11, 97% or more sequence identity to MA004 mRNA shown in Table 11, 98% or more sequence identity to MA004 mRNA shown in Table 11, or 99% or more sequence identity to MA004 mRNA shown in Table 11.

ABE7.10 (Gaudelli et al., Nature. 2017 Nov. 23; 551(7681):464-471. doi: 10.1038/nature24644), and other ABE variants containing Streptococcus pyogenes Cas9. The CRISPR Journal, Volume 4, Number 2, 2021 pp. 169-177 and Supplementary Figures S1-s9, Supplementary Data S4-S6, and Supplementary Table S1-S2 discloses additional inlaid base editors (IBEs) variants.

In some embodiments, a base editor may convert C:G to G:C base pairs. Examples of such base editors are disclosed in (a) Chen et al. Programmable C:G to G:C genome editing with CRISPR-Cas9-directed base excision repair proteins. Nat Commun 12, 1384 (2021). doi:10.1038/s41467-021-21559-9. Epub 2021-March-02; (b) Kurt, I. C., Zhou, R., Iyer, S. et al. CRISPR C-to-G base editors for inducing targeted DNA transversions in human cells. Nat Biotechnol 39, 41-46 (2021). doi: 10.1038/s41587-020-0609-x. Epub 2020-July-20; and (c) Zhao, D., Li, J., Li, S. et al. Glycosylase base editors enable C-to-A and C-to-G base changes. Nat Biotechnol 39, 35-40 (2021). doi: 10.1038/s41587-020-0592-2. Epub 2020-July-20. Such base editors may comprise a Cas9 nickase and a cytidine deaminase. Such base editors may further comprise a uracil-DNA glycosylase, a DNA repair protein such as XRCC1, DNA ligase S, or DNA binding and ligase domains of DNA polymerase β.

In some embodiments, a base editor may convert C:G to A:T base pairs. An example of such a base editor is disclosed in Zhao, D., Li, J., Li, S. et al. Glycosylase base editors enable C-to-A and C-to-G base changes. Nat Biotechnol 39, 35-40 (2021). doi: 10.1038/s41587-020-0592-2. Epub 2020-July-20. Such base editors may comprise a Cas9 nickase and a cytidine deaminase. Such base editors may further comprise a uracil-DNA glycosylase.

The term “base editor system” refers to a system for editing a nucleobase of a target nucleotide sequence. In various embodiments, the base editor system comprises (1) a polynucleotide programmable nucleotide binding domain (e.g., Cas9); (2) a deaminase domain (e.g., an adenosine deaminase or a cytidine deaminase) for deaminating said nucleobase; and (3) one or more guide polynucleotide (e.g., guide RNA). In some embodiments, the base editor system comprises a base editor fusion protein comprising (1) and (2), or a polynucleotide (e.g., mRNA) encoding the base editor fusion protein comprising (1) and (2). In some embodiments, the polynucleotide programmable nucleotide binding domain is a polynucleotide programmable DNA binding domain. In some embodiments, the base editor is an adenine or adenosine base editor (ABE). In some embodiments, the base editor is a cytosine base editor (CBE).

Genome-editing systems include clustered regularly interspaced short palindromic repeats (CRISPR)/Cas system. Exemplary guide nucleotide sequence-programmable DNA-binding proteins include, but are not limited to, Cas9 (e.g., dCas9 and nCas9), saCas9 (e.g., saCas9d, saCas9d, saKKH Cas9) CasX, CasY, Cpf1, C2c1, C2c2, C2c3, Argonaute, and any other suitable protein described herein, or suitable variants thereof.

Through the use of a guide RNA (gRNA) with a sequence homologous to that of a sequence of DNA in the target genome (known as the protospacer) adjacent to a specific protospacer-adjacent motif (PAM) comprising the sequence NGG (N is any standard base) in the DNA, Cas9 can be used to create a double-strand break (DSB) at the targeted sequence. Non-homologous end joining (NHEJ) at DSBs is capable of creating indels and potentially knocking out genes at genetic loci; likewise, homology-directed repair (HDR), with an introduced template DNA, may insert genes or modify the targeted sequence.

A variety of Cas9-based tools have been developed in recent years. Methods of using guide nucleotide sequence-programmable DNA-binding protein, such as Cas9, for site-specific cleavage (e.g., to modify a genome) have been described (see e.g., Cong et al., Science 339, 819-823 (2013); Mali et al., Science 339, 823-826 (2013); Hwang et al., Nature Biotechnology 31, 227-229 (2013); Jinek et al., eLife 2, e00471 (2013); Dicarlo et al., Nucleic Acids Research (2013); and Jiang et al., Nature Biotechnology 31, 233-239 (2013).

In 2016, Komor et al. described the use of CRISPR-Cas9 to convert a cytosine base to a thymine base without the introduction of a template DNA strand and without the need for DSBs (Komor et al., Nature, 2016, 533:420-4). After the cytidine deaminase domain of rat APOBEC1 was fused to the N-terminus of catalytically-dead Cas9 (dCas9) using the linker XTEN (resulting in a fusion protein called base editor 1, or BEI), conversion of cytosine to uracil was observed between position 4 and position 8 within the 20-nt protospacer region of DNA (or, to express it a different way, 13 to 17 nucleotides upstream of the PAM). Of note, any cytosine base within this “window” was amenable to editing, resulting in varied outcomes depending on how many and which cytosines were edited. After DNA replication or repair, each uracil was replaced by a thymine, completing the C to T base editing.

The next version of base editor (BE2) incorporated a uracil glycosylase inhibitor (UGI) fused to the C-terminus of dCas9 to help inhibit base excision repair of the uracil bases resulting from the cytidine deaminase activity (which otherwise would act to restore the original cytosine bases); this improved the efficiency of C to T base editing.

The next version of base editor (BE3) used a Cas9 nickase rather than dCas9; the nickase cut the unedited strand opposite the edited C to T bases, stimulating the removal of the opposing guanidine through eukaryotic mismatch repair. BE2 and BE3 base editing was observed in both human and murine cell lines. FIG. 1B is an illustration of BE3. The specificity of base editing has been further improved through the addition of mutations to the Cas9 nickase; in similar fashion, Cas9 has been mutated to narrow the width of the editing window from approximately 5 nucleotides to as little as 1-2 nucleotides (Rees et al., Nat Commun, 2017, 8:15790, Kim et al., Nat Biotechnol, 2017, 35:371-6).

By fusing Escherichia coli adenine tTNA deaminase TadA (ecTadA) to dCas9 and mutagenesis of the ecTadA domain in conjunction with selection for editing activity revealed that A106V and D108N mutations yielded a base editor capable of editing adenine to guanine in DNA, termed ABE7.10 (Gaudelli et al. Nature, 2017, 551:464-71).

Adenine 8.8-m (also referred to herein as ABE8.8) uses its core Streptococcus pyogenes nickase Cas9 (nSpCas9) protein with a guide RNA (gRNA) to engage a double-strand protospacer DNA sequence, flanked by an NGG protospacer-adjacent motif (PAM) sequence on its 3′ end. The protospacer sequence is specified via hybridization of the first 20 bases of the gRNA with a complementary sequence on the “target” DNA strand, leaving part of the other (“non-target”) strand in exposed single-strand form structure called the R-loop. Unlike Cas9 and Cas12, ABE8.8 does not make double-strand breaks in targeted DNA sequences. Rather, as illustrated in FIG. 1C, ABE8.8 uses an evolved deoxyadenosine deaminase domain—fused to nSpCas9—to chemically modify an adenosine nucleoside, contained in the single-strand DNA portion of the R-loop, into inosine and nicks the target DNA strand within the DNA: RNA heteroduplex of the R-loop. This nick biases DNA repair machinery to use the freshly deaminated strand as a template, enabling highly efficient transition mutation at the targeted site. The activity window of ABE8.8 typically ranges from positions 3 to 9 in the protospacer DNA sequence specified by the gRNA, 12 to 18 base pairs 5′ of the NGG PAM (positions 21 to 23), with peak editing observed at position 6 of the protospacer (Gaudelli et al., Nat Biotechnol. 2020 July; 38 (7): 892-900).

In some embodiments, the nucleic acid encoding the base editor fusion protein is a mRNA. In some embodiments, the mRNA generates the base editor fusion protein upon translation in the targeted cell or subject after the administration. In some embodiments, the base editor fusion protein forms a ribonucleoprotein (RNP) complex in the targeted cell or subject.

It should be appreciated that the fusion proteins of the present disclosure may comprise one or more additional features. For example, in some embodiments, the fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein comprises one or more His tags.

### Protospacers

The term “protospacer” or “target sequence” and its grammatical equivalents as used herein can refer to a PAM-adjacent nucleic acid sequence. A protospacer can be a nucleotide sequence within gene, genome, or chromosome that is targeted by a gRNA. In the native state, a protospacer is adjacent to a PAM (protospacer adjacent motif). The site of cleavage by an RNA-guided nuclease is within a protospacer sequence. For example, as illustrated in FIG. 1A, when a gRNA targets a specific protospacer, the Cas protein will generate a double strand break within the protospacer sequence, thereby cleaving the protospacer. Following cleavage, disruption of the protospacer can result though non-homologous end joining or homology-directed repair. Disruption of the protospacer can result in the deletion of the protospacer. Additionally, or alternatively, disruption of the protospacer can result in an exogenous nucleic acid sequence being inserted into or replacing the protospacer.

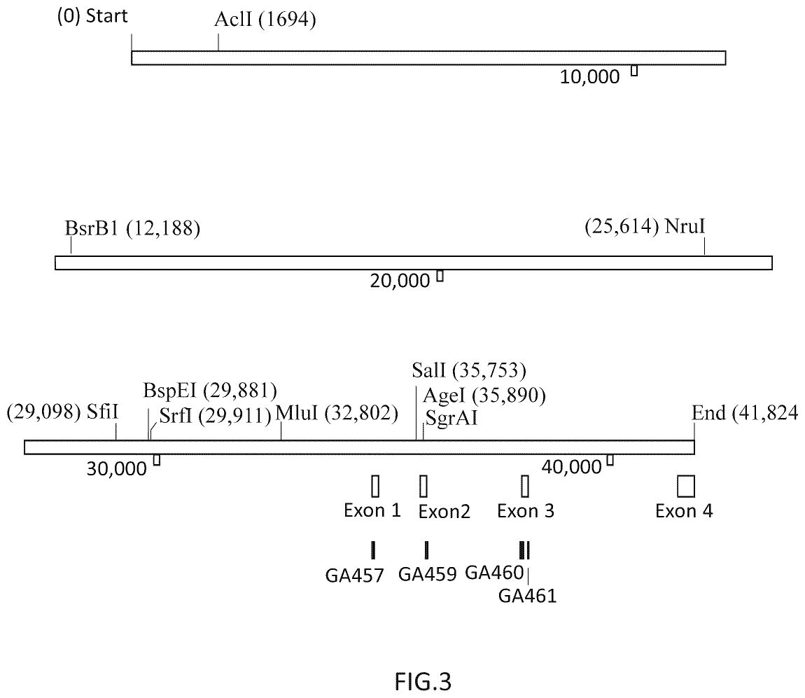

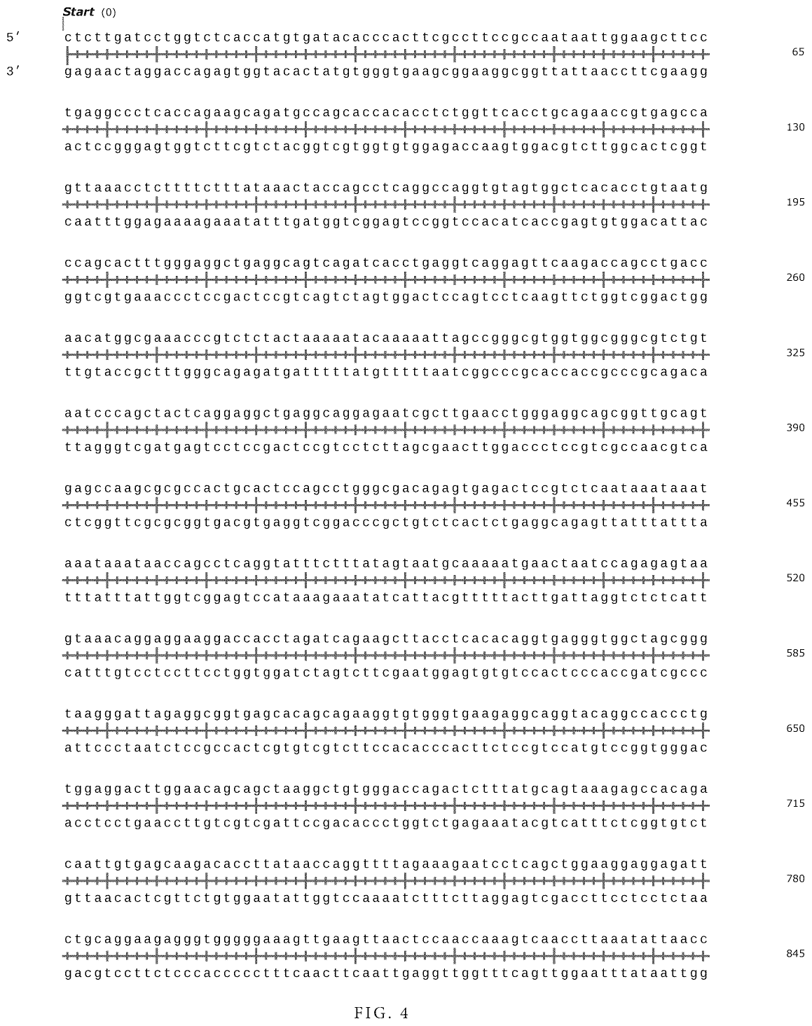

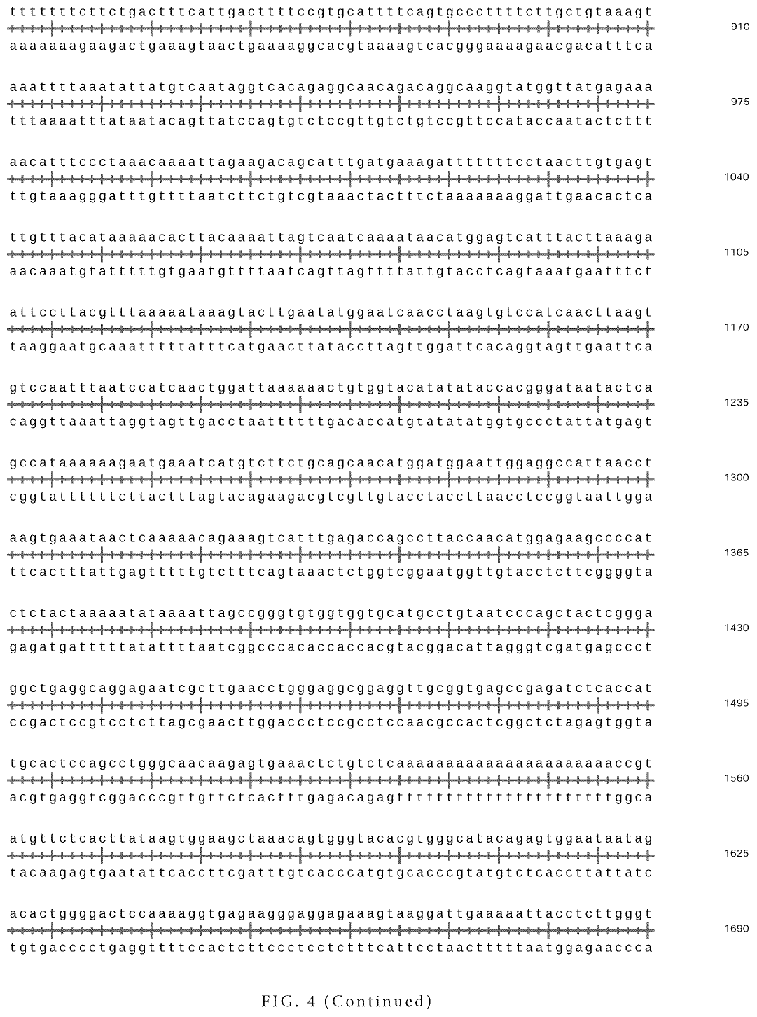

With the present disclosure, protospacer sequences were identified within the nucleotide sequence of the human TTR gene to be used as guide sequences that permit ABE8.8 (and other ABE variants containing Streptococcus pyogenes Cas9, such as ABE7.10, or another Cas protein that can use the NGG PAM) to either disrupt the start codon, or disrupt splice sites, whether donors or acceptors, via A→G editing within its editing window (roughly positions 4 to 7 in the 20-nt protospacer region of DNA). Four of the sequences shown in Table 1 were identified within the human TTR gene. The alignment of these four protospacer sequences on a map of the human TTR gene is shown in FIG. 3.

Protospacer, corresponding to guide RNA GA457, has the sequence 5′-GCCATCCTGCCAAGAATGAG-3′ (SEQ ID NO: 24) and is located at 34,879 to 34,898 bp of the human TTR gene.

Protospacer, corresponding to guide RNA GA459, has the sequence 5′-GCAACTTACCCAGAGGCAAA-3′ (SEQ ID NO: 25) and is located at 36,007 to 36,026 bp of the human TTR gene.

Protospacer, corresponding to guide RNA GA460, has the sequence 5′-TATAGGAAAACCAGTGAGTC-3′ (SEQ ID NO: 26) and is located at 38,106-38,125 bp of the human TTR gene.

Protospacer, corresponding to guide RNA GA461, has the sequence 5′-TACTCACCTCTGCATGCTCA-3′ (SEQ ID NO: 27) and is located at 38,234-38253 of the human TTR gene.

Protospacer, corresponding to guide RNA GA458, has the sequence 5′-GCCATCCTGCCAAGAACGAG-3′ (SEQ ID NO: 28) represents the sequence within the cynomolgus macaque TTR gene corresponding to the human protospacer sequence corresponding to guide RNA GA459.

A guide nucleic acid, (e.g., guide RNA) is about 15-100 nucleotides long and comprises a sequence of at least 10 contiguous nucleotides that is complementary to a target protospacer sequence. In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence.

In some embodiments, a guide polynucleotide is a DNA. In some embodiments, a guide polynucleotide is an RNA, also referred to herein as a guide RNA or gRNA. In some embodiments, a guide polynucleotide is a modified, artificial polynucleotide.

In some embodiments, a guide polynucleotide, including but not limited to, a gRNA, may be synthesized. The guide polynucleotide may comprise a spacer sequence configured to hybridize to the complementary sequence of a protospacer sequence as shown in Table 1 under, for example, conditions within a cell. The guide polynucleotide may comprise a spacer sequence that is homologous to a protospacer sequence as shown in Table 1. In some embodiments, the guide polynucleotide comprises a guide RNA comprising a spacer having a sequence homologous to the protospacer set forth in Table 1. In some embodiments, the guide RNA may have a sequence that comprises a guide RNA (gRNA) sequences set forth in Table 1.

The present disclosure includes a guide polynucleotide having a sequence at least about 75%, at least about 80%, at least about 85%, at least about 90%, or at least about 95% identical to the sequence 5′-GCCAUCCUGCCAAGAAUGAG-3′ (SEQ ID NO: 1) (GA457). The present disclosure includes a guide polynucleotide having the sequence 5′-GCCAUCCUGCCAAGAAUGAG-3′ (SEQ ID NO: 1) (GA457).

The present disclosure includes a modified guide polynucleotide having a sequence at least about 75%, at least about 80%, at least about 85%, at least about 90%, or at least about 95% identical to the sequence 5′-GCCAUCCUGCCAAGAAUGAG-3′ (SEQ ID NO: 1), wherein GCC are modified by methylation (GA521) (C is modified to 2′-O-methylcytidine, G is modified to 2′-O-methylguanosine). The present disclosure includes a modified guide polynucleotide having the sequence 5′-mGsmCsmCAUCCUGCCAAGAAUGAG-3′ (SEQ IQ NO: 1) (GA521), wherein mC: 2′-O-methylcytidine, mG: 2′-O-methylguanosine and s: phosphorothioate (PS) backbone linkage.

The present disclosure includes a guide polynucleotide having a sequence at least about 75%, at least about 80%, at least about 85%, at least about 90%, or at least about 95% identical to the sequence 5′-GCCAUCCUGCCAAGAACGAG-3′ (SEQ ID NO: 2) (GA458). The present disclosure includes a guide polynucleotide having the sequence 5′-GCCAUCCUGCCAAGAACGAG-3′ (SEQ ID NO: 2) (GA458).

The present disclosure includes a guide polynucleotide having a sequence at least about 75%, at least about 80%, at least about 85%, at least about 90%, or at least about 95% identical to the sequence 5′-GCAACUUACCCAGAGGCAAA-3′ (SEQ ID NO: 3) (GA459). The present disclosure includes a guide polynucleotide having the sequence 5′-GCAACUUACCCAGAGGCAAA-3′ (SEQ ID NO: 3) (GA459).

The present disclosure includes a guide polynucleotide having a sequence at least about 75%, at least about 80%, at least about 85%, at least about 90%, or at least about 95% identical to the sequence 5′-UAUAGGAAAACCAGUGAGUC-3′ (SEQ ID NO: 4) (GA460). The present disclosure includes a guide polynucleotide having the sequence 5′-UAUAGGAAAACCAGUGAGUC-3′ (SEQ ID NO: 4) (GA460).

The present disclosure includes a guide polynucleotide having a sequence at least about 75%, at least about 80%, at least about 85%, at least about 90%, or at least about 95% identical to the sequence 5′-UACUCACCUCUGCAUGCUCA-3′ (SEQ ID NO: 5) (GA461). The present disclosure includes a guide polynucleotide having the sequence 5′-UACUCACCUCUGCAUGCUCA-3′ (SEQ ID NO: 5) (GA461).

In some aspects, provided herein is a guide RNA, comprising a sequence defined by mG*mC*mC*AUCCUGCCAAGAAUGAGmGUUUUAGmAmGmCmUmAGmAmAmAmUm AmGmCmAmAGUUmAAmAAmUAmAmGmGmCmUmAGUmCmCGUUAmUmCAAmCm UmUGmAmAmAmAmAmGmUmGGmCmAmCmCmGmAmGmUmCmGmGmUmGmCmU*mU*mU*mU (GA521, (SEQ ID NO: 11), wherein A is adenosine, C is cytidine, G is guanosine, U is uridine, mA* is 2′-O-methyladenosine, mC* is 2′-O-methylcytidine, mG* is 2′-O-methylguanosine, mU* is 2′-O-methyluridine, and wherein nucleotides represented in bold are linked by a phosphorothioate (PS) backbone linkage.

Alternatively, GA521 is represented as mG*smC*smC*AUCCUGCCAAGAAUGAGmGsUsUsUsUsAsGsmAsmsGsmCsmUsm AsGsmAsmAsmAsmUsmAsmGsmCssmAsmAsGsUsUsmAsAsmAsAsmUsAsmAsmGsmGsm CsmUsmAsGsUsmCsmCsGsUsUsAsmUsmCsAsAsmCsmUsmUsGsmAsmAsmAsmAsmAsmG smUsmGsGsmCsmAsmCsmCsmGsmAsmGsmUsmCsmGsmGsmUsmGsmCsmU*smU*smU*smU (GA521) (SEQ ID NO: 11), wherein A is adenosine, C is cytidine, G is guanosine, U is uridine, mA* is 2′-O-methyladenosine, mC* is 2′-O-methylcytidine, mG* is 2′-O-methylguanosine, mU*is 2′-O-methyluridine, and wherein nucleotides are linked by a phosphorothioate (PS) backbone linkage represented by the letter ‘s’.

In some embodiments, mG*mC*mC*AUCCUGCCAAGAAUGAGmGUUUUAGmAmGmCmUmAGmAmAmAmUm AmGmCmAmAGUUmAAmAAmUAmAmGmGmCmUmAGUmCmCGUUAmUmCAAmCm UmUGmAmAmAmAmAmGmUmGGmCmAmCmCmGmAmGmUmCmGmGmUmGmCmU*mU*mU*mU (GA521,SEQ ID NO: 11), wherein A is adenosine, C is cytidine, G is guanosine, U is uridine, mA* is 2′-O-methyladenosine, mC* is 2′-O-methylcytidine, mG* is 2′-O-methylguanosine, mU* is 2′-O-methyluridine, and wherein nucleotides represented in bold are linked by a phosphorothioate (PS) backbone linkage.

Alternatively, GA521 is represented as mG*mC*mC*AUCCUGCCAAGAAUGAGmGsUsUsUsUsAsGsmAsmsGsmCsmUsmAsGsmA smAsmAsmUsmAsmGsmCssmAsmAsGsUsUsmAsAsmAsAsmUsAsmAsmGsmGsmCsmUsm AsGsUsmCsmCsGsUsUsAsmUsmCsAsAsmCsmUsmUsGsmAsmAsmAsmAsmAsmGsmUsmG sGsmCsmAsmCsmCsmGsmAsmGsmUsmCsmGsmGsmUsmGsmCsmU*mU*mU*mU (GA521) (SEQ ID NO: 11), wherein A is adenosine, C is cytidine, G is guanosine, U is uridine, mA* is 2′-O-methyladenosine, mC* is 2′-O-methylcytidine, mG* is 2′-O-methylguanosine, mU* is 2′-O-methyluridine, and wherein nucleotides represented in bold are linked by a phosphorothioate (PS) backbone linkage represented by the letter ‘s’.

In some embodiments, at least two or more of the tracrRNA nucleotides are linked by a phosphorothioate (PS) backbone linkage. In some embodiments, all the tracrRNA nucleotides are linked by a phosphorothioate (PS) backbone linkage. In some embodiments, only the tracrRNA nucleotides are linked by a phosphorothioate (PS) backbone linkage.

In some embodiments, only methyl modified nucleotides are linked by a phosphorothioate (PS) backbone linkage.

A guide polynucleotide may include at least three regions: a first region at the 5′ end that can be homologous to a target site in a chromosomal sequence (spacer region), a second internal region that can form a stem loop structure, and a third 3′ region that can be single-stranded. The second and third regions are considered a tracr sequence or region of the guide RNA and serves as a binding scaffold for the base editor or CRISPR/Cas protein, while the spacer regions serves to guide the protein to a specific target site. The acronym tracr refers to trans-activating crispr.

The second region of a gRNA may form a secondary structure. In some embodiments, a secondary structure formed by a gRNA can comprise a stem (or hairpin) and a loop. A length of a loop and a stem can vary. In some embodiments, a loop can range from about 3 to about 10 nucleotides in length. In some embodiments, a stem can range from about 6 to about 20 nucleotides in length. A stem can comprise one or more bulges of 1 to 10 nucleotides or about 10 nucleotides. In some embodiments, the overall length of a second region can range from about 16 to 60 nucleotides in length. In some embodiments, a loop can be about 4 nucleotides in length. In some embodiments, a stem can be about 12 in length.

The third region of gRNA at the 3′ end can be single-stranded. In some embodiments, a third region is not complementary to any chromosomal sequence in a cell of interest and is not complementary to the rest of a gRNA. The third region may have any suitable length. For example, the third region may be three or more or four or more nucleotides in length. In some embodiments, the length of a third region can vary, ranging from about 5 to about 60 nucleotides in length.

In some embodiments, the guide polynucleotide includes a spacer sequence that is homologous to a protospacer sequence of the TTR gene as shown in Table 1 with 0, 1, 2, 3, 4, or 5 mismatches. In some embodiments, the guide polynucleotide includes a spacer sequence homologous to a protospacer sequence of the TTR gene as shown in Table 1 with no mismatches.

In some embodiments, the length of a guide polynucleotide depends on the CRISPR/Cas component of the base editor system and components used. For example, different Cas proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 nucleotides in length. In some embodiments, the targeting sequence comprises 18-24 nucleotides in length. In some embodiments, the targeting sequence comprises 19-21 nucleotides in length. In some embodiments, such as those described in Table 1, the targeting sequence comprises 20 nucleotides in length.

In some embodiments, a guide polynucleotide includes a spacer sequences and otherwise conforms to the standard 100-nt Streptococcus pyogenes CRISPR gRNA sequence.

In some embodiments, the guide RNA is chemically modified. Chemically modified gRNAs may have increased stability when transfected into mammalian cells. For example, gRNAs can be chemically modified to comprise a combination of 2′-O-methylribosugar and phosphorothioate backbone modifications on at least one 5′ nucleotide and at least one 3′ nucleotide of each gRNA. In some cases, the three terminal 5′ nucleotides and three terminal 3′ nucleotides are chemically modified to comprise combinations of 2′-O-methylribosugar and phosphorothioate modifications.

The gRNAs described herein can be synthesized chemically, enzymatically, or a combination thereof. For example, the gRNA can be synthesized using standard phosphoramidite-based solid-phase synthesis methods. Alternatively, the gRNA can be synthesized in vitro by operably linking DNA encoding the gRNA to a promoter control sequence that is recognized by a phage RNA polymerase. Examples of suitable phage promoter sequences include, but are not limited to, T7, T3, SP6 promoter sequences, or variations thereof. In some embodiments, gRNA comprises two separate molecules (e.g., crRNA (which comprises the spacer) and tracrRNA). One molecule (e.g., crRNA) can be chemically synthesized and the other molecule (e.g., tracrRNA) can be enzymatically synthesized.

### Therapeutic Applications

The guide polynucleotides and compositions described herein may be administered to target cells or a subject in need thereof, in a therapeutically effective amount, to prevent or treat conditions related to transthyretin amyloidosis. In some embodiments, the subject has hereditary transthyretin amyloidosis (hATTR). In some embodiments, the subject has cardiomyopathy due to transthyretin amyloidosis (ATTR-CM). In some embodiments, the subject has polyneuropathy due to transthyretin amyloidosis (ATTR-PN). In some embodiments, the subject has wild-type ATTR (ATTRwt), the age-related deposition of wild type TTR protein (formerly known as senile amyloidosis).

With such administration, the guide polynucleotide directs the editor system (e.g., ABE editor system) to affect a nucleobase alteration in a TTR gene in the subject, editing the TTR gene to reduce or abolish the amount of full-length, functional protein being produced, thus treating the conditions. In some embodiments, the base alteration occurs in the cells of the liver (hepatocytes) in the subject.

For example, the gRNA and an adenosine base editor protein, which may be expressed in a cell wherein target gene editing is desired, such as, for example, a liver cell, thereby allowing contact of the target gene with the gRNA and the adenosine base editor protein. In some embodiments, the binding of the adenosine base editor protein to its target polynucleotide sequence in the target gene is directed by the guide RNA, wherein the spacer sequence of the gRNA hybridizes with a target polynucleotide sequence in a target gene e.g., a complimentary sequence to the protospacer. Thus, the guide RNA directs adenosine base editor protein to edit the target polynucleotide sequence (e.g., the protospacer sequence) in the target gene. In some embodiments, the guide RNA is co-introduced into a cell where editing is desired with the adenosine base editor protein or with a nucleic acid encoding the adenosine base editor protein.

In certain embodiments, adenine base editors may be used to disrupt gene function and/or expression by modifying nucleobases at splice sites of target genes. In some embodiments, an adenosine nucleobase editor as described herein may be used to disrupt a splice donor site at the 5′ end of an introns or a splice acceptor site at the 3′ ends of an intron. In some embodiments, splice site disruption results in the inclusion of intronic sequences in messenger RNA (mRNA)—potentially introducing nonsense, frameshift, or in-frame indel mutations that result in premature stop codons or in insertion/deletion of amino acids that disrupt protein activity—or in the exclusion of exonic sequences, which can also introduce nonsense, frameshift, or in-frame indel mutations.

Canonical splice donors comprise the DNA sequence GT on the sense strand, whereas canonical splice acceptors comprise the DNA sequence AG. In some embodiments, a base editor, such as an adenosine nucleobase editor as described herein can be used to generate alteration of the sequence disrupts normal splicing. In some embodiments, the adenosine base editor disrupts a complementary A on the antisense strand of the splice donor, causing an edit of GT→GC. In some embodiments, the adenosine base editor disrupts the A of the splice acceptor site on the sense strand, causing an edit of AG→GG.

In some embodiments, the methods and composition disclosed herein reduce or abolish the expression and/or function of the transthyretin protein encoded by the TTR gene. For example, the methods and composition disclosed herein may reduce expression and/or function of transthyretin by at least 2-fold, at least 3-fold, at least 4-fold, at least 5-fold, at least 6-fold, at least 7-fold, at least 8-fold, at least 9-fold, or at least 10-fold relative to a control.

In some embodiments, the method for treating or preventing a condition in a subject in need thereof as described herein includes administering (i) a guide polynucleotide and (ii) a nucleic acid encoding a base editor fusion protein to the subject.

In some embodiments, the method for treating or preventing a condition in a subject in need thereof as described herein includes administering a lipid nanoparticle (LNP) enclosing a (i) guide polynucleotide or a nucleic acid encoding the guide polynucleotide and/or (ii) a base editor fusion protein comprising a programmable DNA binding domain and a deaminase or a nucleic acid encoding the same. In some aspects, the (i) guide polynucleotide or nucleic acid encoding the same and (ii) the base editor fusion protein comprising a programmable DNA binding domain and a deaminase or a nucleic acid encoding the same are enclosed the same LNPs. In some aspects, they are enclosed in separate LNPs.

### Pharmaceutical Compositions

In some aspects, provided herein is a pharmaceutical composition comprising the base editor system as provided herein and a pharmaceutically acceptable carrier or excipient. In some aspects, provided herein, is a pharmaceutical composition for gene modification comprising a guide RNA as described herein and a base editor fusion protein or a nucleic acid sequence encoding the base editor fusion protein and a pharmaceutically acceptable carrier. Pharmaceutical compositions are formulated in a conventional manner using one or more pharmaceutically acceptable inactive ingredients that facilitate processing of the active compounds into preparations that can be used pharmaceutically. Suitable formulations for use in the present disclosure and methods of delivery are generally well known in the art. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition can be a mixture of a guide RNA as described herein or a nucleic acid sequence encoding the guide RNA and a base editor fusion protein or a nucleic acid sequence encoding the base editor fusion protein with one or more of other chemical components (i.e., pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration to an organism or a subject in need thereof.

The pharmaceutical compositions of the present disclosure can be administered to a subject using any suitable methods known in the art. The pharmaceutical compositions described herein can be administered to the subject in a variety of ways, including parenterally, intravenously, intradermally, intramuscularly, colonically, rectally, or intraperitoneally. In some embodiments, the pharmaceutical compositions can be administered by intraperitoneal injection, intramuscular injection, subcutaneous injection, or intravenous injection of the subject. In some embodiments, the pharmaceutical compositions can be administered parenterally, intravenously, intramuscularly, or orally.

In some embodiments, a pharmaceutical composition for gene modification includes a further therapeutic agent. The additional therapeutic agent may modulate different aspects of the disease, disorder, or condition being treated and provide a greater overall benefit than administration of the therapeutic agent alone. Therapeutic agents include, but are not limited to, a chemotherapeutic agent, a radiotherapeutic agent, a hormonal therapeutic agent, and/or an immunotherapeutic agent. In some embodiments, the therapeutic agent may be a radiotherapeutic agent. In some embodiments, the therapeutic agent may be a hormonal therapeutic agent. In some embodiments, the therapeutic agent may be an immunotherapeutic agent. In some embodiments, the therapeutic agent is a chemotherapeutic agent. Preparation and dosing schedules for additional therapeutic agents can be used according to manufacturers' instructions or as determined empirically by a skilled practitioner.

### Lipid Nanoparticle (LNP) Compositions

The pharmaceutical compositions for gene modification described herein may be encapsulated in lipid nanoparticles (LNP). As used herein, a “lipid nanoparticle (LNP) composition” or a “nanoparticle composition” is a composition comprising one or more described lipids. LNP compositions or formulations, as contemplated herein, are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition or formulation as contemplated herein may be a liposome having a lipid bilayer with a diameter of 500 nm or less. A LNP as described herein may have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 50 nm to 90 nm, from about 55 nm to 85 nm, from about 55 nm to 75 nm, from about 50 nm to about 80 nm, from about 60 nm to about 80 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. In one embodiment the mean diameter of the LNP is about 70 nm+/−20 nm, 70 nm+/−10 nm, 70 nm+/−5 nm. The LNPs described herein can be substantially non-toxic.

Lipid nanoparticles (LNPs) employ a non-viral drug delivery mechanism that is capable of passing through blood vessels and reaching hepatocytes [Am. J. Pathol. 2010, 176, 14-21]. Apolipoprotein E (ApoE) proteins are capable of binding to the LNPs post PEG-lipid diffusion from the LNP surface with a near neutral charge in the blood stream, and thereby function as an endogenous ligand against hepatocytes, which express the low-density lipoprotein receptor (LDLr) [Mol. Ther., 2010, 18, 1357-1364.]. Control the efficient hepatic delivery of LNP include: 1) effective PEG-lipid shedding from LNP surface in blood serum and 2) ApoE binding to the LNP. Endogenous ApoE-mediated LDLr-dependent LNP delivery route is unavailable or less effective path to achieve LNP-based hepatic gene delivery in patient populations that LDLr deficient.

Efficient delivery to cells requires specific targeting and substantial protection from the extracellular environment, particularly serum proteins. One method of achieving specific targeting is to conjugate a targeting moiety to an active agents or pharmaceutical effector such as a nucleic acid agent, thereby directing the active agent or pharmaceutical effector to particular cells or tissues depending on the specificity of the targeting moiety. One way a targeting moiety can improve delivery is by receptor mediated endocytotic activity. This mechanism of uptake involves the movement of nucleic acid agent bound to membrane receptors into the interior of an area that is enveloped by the membrane via invagination of the membrane structure or by fusion of the delivery system with the cell membrane. This process is initiated via activation of a cell surface or membrane receptor following binding of a specific ligand to the receptor. Receptor-mediated endocytotic systems include those that recognize sugars such as galactose, mannose, mannose-6-phosphate, peptides and proteins such as transferrin, asialoglycoprotein, vitamin B12, insulin and epidermal growth factor (EGF). Lipophilic moieties, such as cholesterol or fatty acids, when attached to highly hydrophilic molecules such as nucleic acids can substantially enhance plasma protein binding and consequently circulation half-life. Lipophilic conjugates can also be used in combination with the targeting ligands in order to improve the intracellular trafficking of the targeted delivery approach.

The Asialoglycoprotein receptor (ASGP-R) is a high-capacity receptor, which is abundant on hepatocytes. The ASGP-R shows a 50-fold higher affinity for N-Acetyl-D-Galactosylamine (GalNAc) than D-Gal. LNPs comprising receptor targeting conjugates, may be used to facilitate targeted delivery of the drug substances described herein. The LNPs may include one or more receptor targeting moiety on the surface or periphery of the particle at specified or engineered surface density ranging from relatively low to relatively high surface density. The receptor targeting conjugate may comprise a targeting moiety (or ligand), a linker, and a lipophilic moiety that is connected to the targeting moiety. In some embodiments, the receptor targeting moiety (or ligand) targets a lectin receptor. In some embodiments, the lectin receptor is asialoglycoprotein receptor (ASGPR). In some embodiments the receptor targeting moiety is GalNAc or a derivative GalNAc that targets ASGPR. In one aspect the receptor targeting conjugate comprises of one GalNAc moiety or derivative thereof. In another aspect, the receptor targeting conjugate comprises of two different GalNAc moieties or derivative thereof. In another aspect, the receptor targeting conjugate comprises of three different GalNAc moieties or derivative thereof. In another aspect, the receptor targeting conjugate is lipophilic. In some embodiments, the receptor targeting conjugate comprises one or more GalNAc moieties and one or more lipid moieties, i.e., GalNAc-Lipid. In some embodiments, the receptor targeting conjugate is a GalNAc-Lipid.

Described herein are (i) LNP compositions comprising an amino lipid, a phospholipid, a PEG lipid, a cholesterol, or a derivative thereof, a payload, or any combination thereof and (ii) LNP compositions comprising an amino lipid, a phospholipid, a PEG-lipid, a cholesterol, a GalNAc-Lipid or a derivative thereof, a payload, or any combination thereof. Each component is described in more detail below.

In the preparation of LNP compositions comprising the excipients amino lipid, phospholipid, PEG-Lipid and cholesterol, a desired molar ratio of the four excipients is dissolved in a water miscible organic solvent, ethanol for example. The homogenous lipid solution is then rapidly in-line mixed with an aqueous buffer with acidic pH ranging from 4 to 6.5 containing nucleic acid payload to form the lipid nanoparticle (LNP) encapsulating the nucleic acid payload(s). After rapid in-line mixing the LNPs thus formed undergo further downstream processing including concentration and buffer exchange to achieve the final LNP pharmaceutical composition with near neutral pH for administration into cell line or animal diseases model for evaluation, or to administer to human subjects.

For the preparation of GalNAc-LNP pharmaceutical composition the GalNAc-Lipid is mixed with the four lipid excipients in the water miscible organic solvent prior to the preparation of the GalNAc-LNP. The preparation of the GalNAc-LNP pharmaceutical composition then follow the same steps as described for the LNP pharmaceutical composition. The mol % of the GalNAc-Lipid in the GalNAc-LNP preparation ranges from 0.001 to 2.0 of the total excipients.

For both LNP and GalNAc-LNP preparation the payload comprises of a guide RNA targeting the TTR gene and an mRNA encoding a base editor protein. In some embodiments, the guide RNA to mRNA ratio in the acidic aqueous buffer and in the final formulation is 6:1, 5:1, 4:1, 3:1, 2.5:1, 2:1, 1.5:1, 1:1, 1:1.5, 1:2, 1:2.5, 1:3, 1:5 or 1:6 by wight. In some embodiment the said mRNA encodes adenosine base editor protein. In some other embodiments the said mRNA encodes cytosine or cytidine base editor protein.

In some embodiments, an LNP composition may be prepared as described in U.S. patent application Ser. No. 17/192,709, entitled COMPOSITIONS AND METHODS FOR TARGETED RNA DELIVERY, filed on 4 Mar. 2021, claiming the benefit of U.S. Provisional Patent Application Nos. 62/984,866 (filed on 4 Mar. 2020) and 63/078,982 (filed on 16 Sep. 2020), naming Kallanthottathil G. Rajeev as an inventor and Verve Therapeutics, Inc. as the applicant, which application is hereby incorporated herein by reference in its entirety.

### Amino Lipids

In some embodiments, the LNP composition comprises an amino lipid. In some embodiments, the cationic lipid is an ionizable lipid. In some embodiments, the amino lipid (e.g., an ionizable lipid) is a cationic lipid. In some embodiments, the amino lipid (e.g., an ionizable and/or cationic lipid) comprises one or more nitrogen atoms. Exemplary, non-limiting amino lipids suitable for the compositions described herein include those described herein.

### Formula (I)

In one aspect, disclosed herein is an amino lipid having the structure of Formula (I), or a pharmaceutically acceptable salt or solvate thereof,

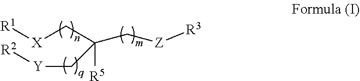

wherein
each of R¹ and R² is independently C₃-C₂₂ alkyl, C₃-C₂₂ alkenyl, C₃-C₈ cycloalkyl, —C₂-C₁₀ alkylene-L-R⁶, or

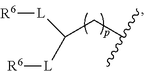

wherein each of the alkyl, alkylene, alkenyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—. —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—O, S, or a bond;
each of L is independently —C(═O)NR⁴—, —NR⁴C(═O)—, —C(═O)O—. —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;

R³ is —C₀-C₁₀ alkylene-NR⁷R⁸, —C₀-C₁₀ alkylene-heterocycloalkyl, or —C₀-C₁₀ alkylene-heterocycloaryl, wherein the alkylene, heterocycloalkyl and heterocycloalkyl is independently substituted or unsubstituted; each of R⁴ is independently hydrogen or substituted or unsubstituted C₁-C₆ alkyl;
R⁵ is hydrogen or substituted or unsubstituted C₁-C₆ alkyl;
each of R⁶ is independently substituted or unsubstituted C₃-C₂₂ alkyl or substituted or unsubstituted C₃-C₂₂ alkenyl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl;
p is an integer selected from 1 to 10; and
each of n, m, and q is independently 0, 1, 2, 3, 4, or 5.

In some embodiments of Formula (I), if the structure carries more than one asymmetric C-atom, each asymmetric C-atom independently represents racemic, chirally pure R and/or chirally pure S isomer, or a combination thereof.

In some embodiments, each of n, in, and q in Formula (I) is independently 0, 1, 2, or 3. In some embodiments, each of n, m, and q in Formula (I) is 1.

### Formula (Ia)

In some embodiments, the compound of Formula (I) has a structure of Formula (Ia), or a pharmaceutically acceptable salt or pharmaceutically acceptable solvate thereof:

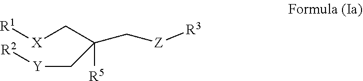

wherein
each of R¹ and R² is independently C₃-C₂₂ alkyl, C₃-C₂₂ alkenyl, C₃-C₈ cycloalkyl, —C₂-C₁₀ alkylene-L-R⁶, or

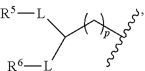

wherein each of the alkyl, alkylene, alkenyl, and cycloalkyl is independently substituted or unsubstituted;
each of X, Y, and Z is independently C(═O)NR⁴—, —NR⁴C(D)-, —C(═O)O—, —OC(═O)—, —OC(═O)O—, —NR⁴C(═O)O—, —OC(═O)NR⁴—, —NR⁴C═O)NR⁴—, —NR⁴C(═NR⁴)NR⁴. —C(═S)NR⁴—, —NR⁴C(═S)—, —C(E)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴— —C(═S)S—, —SC(═S)—, —SC(═S)O—, —NR⁴C(═S)S—, —SC(═S)NR⁴—, —C(═S)S—. —SC(═S)—, —SC(═O)S—, —SC(═S)S—. —NR⁴C(═S)S—, —SC(═S)NR⁴—, O, S, —C₁-C₁₀ alkylene-O—, or a bond, wherein the alkylene is substituted or unsubstituted;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—. —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴— —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S. —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloxy, wherein the alkylene, heterocycloalkyl and heterocycloaryl is independently substituted or unsubstituted;
each of R⁴ is independently hydrogen or substituted or unsubstituted C₁-C₆ alkyl;
R⁵ is hydrogen or substituted or unsubstituted C₁-C₆ alkyl;
each of R⁶ is independently substituted or unsubstituted C₃-C₂₂ alkyl or substituted or unsubstituted C₃-C₂₂ alkenyl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl; and
p is an integer selected from 1 to 10.

In some embodiments of Formula (Ia), if the structure carries more than one asymmetric C-atom, each asymmetric C-atom independently represents racemic, chirally pure R and/or chirally pure S isomer, or a combination thereof.

### Variations of Formula (I) and (Ia)

In some embodiments, R¹ and R² in Formula (I) and Formula (Ia) is independently C₃-C₂₂ alkyl, C₃-C₂₂ alkenyl, —C₂-C₁₀ alkylene-L-R⁶, or

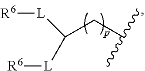

wherein each of the alkyl, alkylene, alkenyl, and cycloalkyl is independently substituted or unsubstituted. In some embodiments, R¹ and R² in Formula (I) and Formula (Ia) is independently C₁₀-C₂₀ alkyl, C₁₀-C₂₀ alkenyl. —C₈-C₇ alkylene-L-R⁶, or

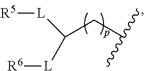

wherein each of the alkyl, alkylene, alkenyl, and cycloalkyl is independently substituted or unsubstituted. In some embodiments, R¹ in Formula (I) and Formula (Ia) is

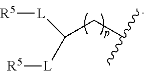

In some embodiments, each of L in Formula (I) and Formula (Ia) is independently O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted. In some embodiments, each of L in Formula (I) and Formula (Ia) is independently O, S, —C₁-C₃ alkylene-O—, —C₁-C₃ alkylene-C(═O)O—, —C₁-C₃ alkylene-OC(═O)—, or a bond, wherein the alkylene is substituted or unsubstituted. In some embodiments, each of L in Formula (I) and Formula (Ia) is independently O, S, —C₁-C₃ alkylene-O—, —C₁-C₃ alkylene-C(═O)O—, —C₁-C₃ alkylene-OC(═O)—, or a bond, wherein the alkylene is linear or branched unsubstituted alkylene.

In some embodiments, each of R⁶ in Formula (I) and Formula (Ia) is independently substituted or unsubstituted linear C₃-C₂₂ alkyl or substituted or unsubstituted linear C₃-C₂₂ alkenyl. In some embodiments, each of R⁶ in Formula (I) and Formula (Ia) is independently substituted or unsubstituted C₃-C₂₀ alkyl or substituted or unsubstituted C₃-C₂₀ alkenyl. In some embodiments, each of R⁶ in Formula (I) and Formula (Ia) is independently substituted or unsubstituted C₃-C₁₀ alkyl or substituted or unsubstituted C₃-C₁₀ alkenyl. In some embodiments, each of R⁶ in Formula (I) and Formula (Ia) is independently substituted or unsubstituted C₃-C₁₀ alkyl. In some embodiments, each of R⁶ in Formula (I) and Formula (Ia) is independently substituted or unsubstituted linear C₃-C₁₀ alkyl. In some embodiments, each of R⁶ in Formula (I) and Formula (Ia) is independently substituted or unsubstituted n-pentyl, n-hexyl, n-heptyl, n-octyl, n-nonyl, n-decyl, n-undecyl, or n-dodecyl. In some embodiments, each of R⁶ in Formula (I) and Formula (Ia) is independently substituted or unsubstituted n-octyl. In some embodiments, each of R⁶ in Formula (I) and Formula (Ia) is n-octyl.

In some embodiments, each of L in Formula (I) and Formula (Ia) is independently-C(═O)O—, —OC(═O)—, —C₁-C₁₀ alkylene-O—, or O. In some embodiments, each of. L in Formula (I) and Formula (Ia) is O. In some embodiments, each of L in Formula (I) and Formula (Ia) is —C₁-C₃ alkylene-O—. In some embodiments, p in Formula (I) and Formula (Ia) is 1, 2, 3, 4, or 5. In some embodiments, p in Formula (I) and Formula (Ia) is 2.

In some embodiments, R¹ in Formula (I) and Formula (Ia) is

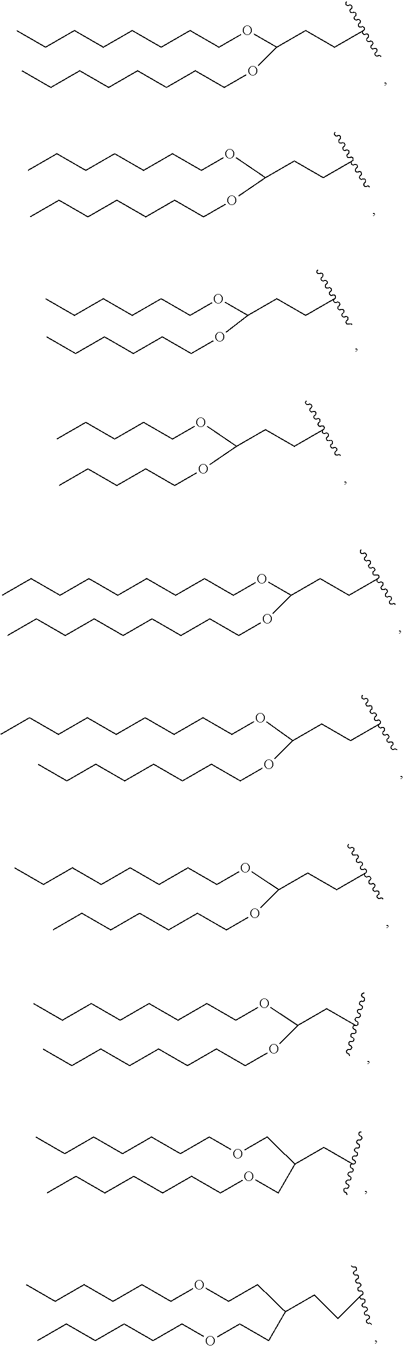

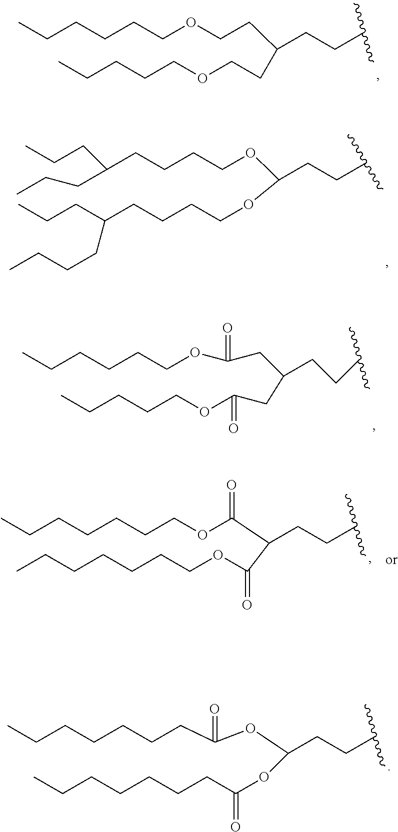 [SMILES: *CCC(CCOCCCCC)CCOCCCCCC ; *CCC(OCCCCC(CCC)CCC)OCCCCC(CCC)CCCC ; *CCC(CC(=O)OCCCCC)CC(=O)OCCCCCC ; *CCC(C(=O)OCCCCCCC)C(=O)OCCCCCCC ; *CCC(OC(=O)CCCCCCC)OC(=O)CCCCCCC]

In some embodiments R¹ in Formula (I) and Formula (Ia) is R².

In some embodiments, each of R⁴ in Formula (I) and Formula (Ia) is independently H or substituted or unsubstituted C₁-C₄ alkyl. In some embodiments, each of. R⁴ in Formula (I) and Formula (Ia) is independently substituted or unsubstituted linear C₁-C₄ alkyl. In some embodiments, each of R⁴ in Formula (1) and Formula (1a) is H. In some embodiments, each of R⁴ in Formula (I) and Formula (Ia) is independently H, —CH₃, —CH₂CH₃, —CH₂CH₂CH₃, or —CH(CH₃)₂.

In some embodiments, each of R⁴ in Formula (I) and Formula (Ia) is independently H or —CH₃. In some embodiments, each of R⁴ in Formula (I) and Formula (Ia) is —CH₃.

In some embodiments, X in Formula (I) and Formula (Ia) is —C(═O)O— or —OC(═O))—. In some embodiments, X in Formula (I) and Formula (Ia) is —C(═O)NR⁴— or —NR⁴C(═O)—. In some embodiments, X in Formula (I) and Formula (Ia) is —C(═O)N(CH₃)—, —N(CH₃)C(═O)—, —C(═O)NH—, or —NHC(═O)—. In some embodiments, X in Formula (I) and Formula (Ia) is —C(═O))NH—, —C(═O)N(CH₃)—. —OC(═O))—, —NHC(═O)—, —N(CH₃)C(═O))—, —C(═O)O—, —OC(═O)O—, —NHC(═O)O—, —N(CH₃)C(═O)O—, —OC(═O))NH—, —OC(═O)N(CH₃)—, —NHC(═O)NH—, —N(CH₃)C(═O))NH—, —NHC(═O)N(CH₃)—, —N(CH₃)C(═O)N(CH₃)—, NHC(═NH)NH—, —N(CH₃)C(═NH)NH—, —NHC(═NH)N(CH₃)—, —N(CH₃)C(═NH)N(CH₃)—, NHC(═NMe)NH—, —N(CH₃)C(═NMe)NH—, —NHC(═NMe)N(CH₃)—, or —N(CH₃)C(═NMe)N(CH₃)—.

In some embodiments. R² in Formula (I) and Formula (Ia) is C₃-C₂₂ alkyl, C₃-C₂₂ alkenyl, —C₂-C₁₀ alkylene-L-R⁶, or

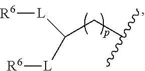

wherein each of the alkyl, alkylene, alkenyl, and cycloalkyl is independently substituted or unsubstituted. In some embodiments, R² in Formula (I) and Formula (Ia) is substituted or unsubstituted C₇-C₂₂ alkyl or substituted or unsubstituted C₃-C₂₂ alkenyl. In some embodiments, R² in Formula (I) and Formula (Ia) is substituted or unsubstituted linear C₇-C₂₂ alkyl or substituted or unsubstituted linear C₃-C₂₂ alkenyl. In some embodiments, R² in Formula (I) and Formula (Ia) is substituted or unsubstituted C₁₀-C₂₀ alkyl or substituted or unsubstituted C₁₀-C₂₀ alkenyl. In some embodiments, R² in Formula (I) and Formula (Ia) is unsubstituted C₁₀-C₂₀ alkyl. In some embodiments, R² in Formula (I) and Formula (Ia) is unsubstituted C₁₀-C₂₀ alkenyl. In some embodiments, R² in Formula (I) and Formula (Ia) is —C₂-C₁₀ alkylene-L-R⁶. In some embodiments, R² in Formula (I) and Formula (Ia) is —C₂-C₁₀ alkylene-C(═O)O—R⁶ or —C₂-C₁₀ alkylene-OC(═O)—R⁶.

In some embodiments, R² in Formula (I) and Formula (Ia) is

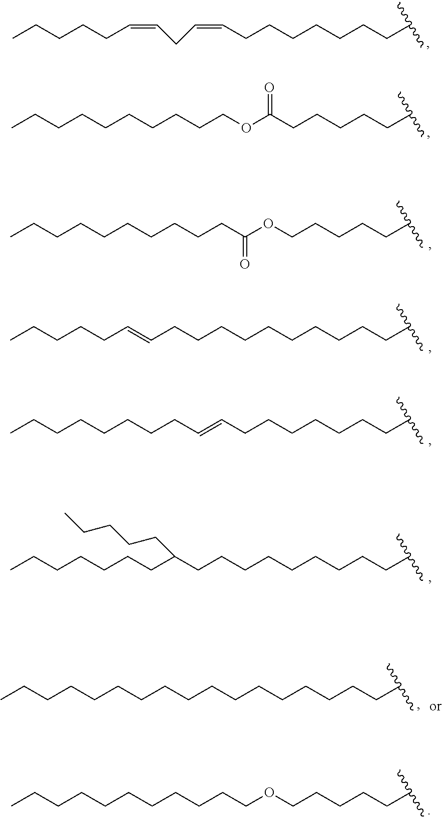

In some embodiments R¹ in Formula (I) and Formula (Ia) is R¹.

In some embodiments, Y in Formula (I) and Formula (Ia) is —C(═O)O— or —OC(═O)—. In some embodiments, Y in Formula (I) and Formula (Ia) is —C(═O)NR⁴— or —NR⁴C(═O)—. In some embodiments, Y in Formula (I) and Formula (Ia) is —C(═O)N(CH₃)—, —N(CH₃)C(═O)—, —C(═O)NH—, or —NHC(═O)—. In some embodiments, Y in Formula (I) and Formula (Ia) is —OC(═O)O—, —NR⁴C(═O)O—, —OC(═O)NR⁴—, or —NR⁴C(═O)NR⁴—. In some embodiments, Y in Formula (I) and Formula (Ia) is —OC(═O)O—, —NHC(═O)O—, —OC(═O)NH—, —NHC(═O)NH—, —N(CH₃)C(═O)O—. —OC(═O)N(CH₃)—, —N(CH₃)C(═O)N(CH₃)— or —N(CH₃)C(═O)NH—. In some embodiments, Y in Formula (I) and Formula (Ia) is —OC(═O)O—, —NHC(═O)O—, —OC(═O)NH—, or —NHC(═O)NH—.

In some embodiments, R³ in Formula (I) and Formula (Ia) is —C₀-C₁₀ alkylene-NR⁷R⁸ or —C₀-C₁₀ alkylene-heterocycloalkyl, wherein the alkylene and heterocycloalkyl is independently substituted or unsubstituted. In some embodiments, R³ in Formula (I) and Formula (Ia) is —C₀-C₁₀ alkylene-NR⁷R⁸. In some embodiments, R³ in Formula (I) and Formula (Ia) is —C₁-C₆ alkylene-NR⁷R⁸. In some embodiments, R³ in Formula (I) and Formula (Ia) is —C₁-C₄ alkylene-NR⁷R⁸. In some embodiments, R³ in Formula (I) and Formula (Ia) is —C₁-alkylene-NR⁷R⁸. In some embodiments, R³ in Formula (I) and Formula (Ia) is —C₂-alkylene-NR⁷R⁸. In some embodiments, R³ in Formula (I) and Formula (Ia) is —C₃-alkylene-NR⁷R⁸. In some embodiments, R³ in Formula (I) and Formula (Ia) is —C₄-alkylene-NR⁷R⁸. In some embodiments, R³ in Formula (I) and Formula (Ia) is —C₅-alkylene-NR⁷R⁸. In some embodiments, R³ in Formula (I) and Formula (Ia) is —C₀-C₁₀ alkylene-heterocycloalkyl. In some embodiments, R³ in Formula (I) and Formula (Ia) is —C₁-C₆ alkylene-heterocycloalkyl, wherein the heterocycloalkyl comprises 1 to 3 nitrogen and 0-2 oxygen. In some embodiments, R³ in Formula (I) and Formula (Ia) is —C₁-C₆ alkylene-heterocycloaryl.

In some embodiments, each of R⁷ and R⁸ in Formula (I) and Formula (Ia) is independently hydrogen or substituted or unsubstituted C₁-C₆ alkyl. In some embodiments, each of R⁷ and R⁸ is independently hydrogen or substituted or unsubstituted C₁-C₃ alkyl. In some embodiments, each of R⁷ and R⁸ is independently substituted or unsubstituted C₁-C₃ alkyl. In some embodiments, each of R⁷ and R⁸ is independently —CH₃, —CH₂CH₃, —CH₂CH₂CH₃, or —CH(CH₃)₂. In some embodiments, each of R and R⁸ is CH₃. In some embodiments, each of R⁷ and R⁸ is —CH₂CH₃.

In some embodiments, R⁷ and R⁸ in Formula (I) and Formula (Ia) taken together with the nitrogen to which they are attached form a substituted or unsubstituted C₂-C₆ heterocyclyl. In some embodiments, R⁷ and R⁸ taken together with the nitrogen to which they are attached form a substituted or unsubstituted C₂-C₆ heterocycloalkyl. In some embodiments, R⁷ and R⁸ taken together with the nitrogen to which they are attached form a substituted or unsubstituted 3-7 membered heterocycloalkyl.

In some embodiments, R³ in Formula (I) and Formula (Ia) is

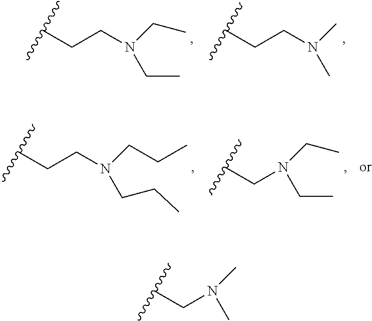

In some embodiments. R³ in Formula (I) and Formula (Ia) is

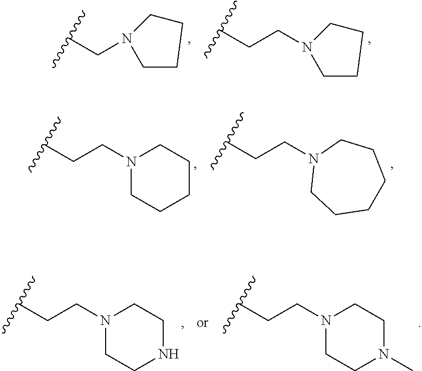

In some embodiments. R³ in Formula (1) and Formula (1a) is

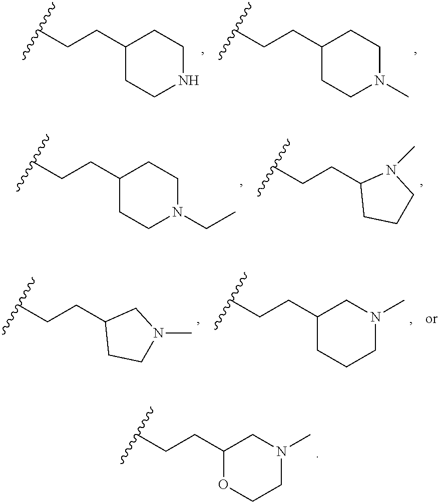

In some embodiments, Z in Formula (I) and Formula (Ia) is —C(═O)O— or —OC(═O)—.

In some embodiments, Z in Formula (I) and Formula (Ia) is —C(═O)NR⁴— or —NR⁴C(═O)—.

In some embodiments, Z in Formula (I) and Formula (Ia) is —C(═O)N(CH₃)—, —N(CH₃)C(═O)—, —C(═O)NH—, or —NHC(═O)—.

In some embodiments, Z in Formula (I) and Formula (Ia) is —OC(═O)O—, —NR⁴C(═O)O—, —OC(O)NR⁴—, or —NR⁴C(═O)NR⁴—.

In some embodiments, Z in Formula (I) and Formula (Ia) is —OC(═O)O—, —NHC(═O)O—, —OC(═O)NH—, —NHC(═O)NH—, —N(CH₃)C(═O)O—, —OC(═O)N(CH₃)—, —N(CH₃)C(═O)N(CH₃)—, —NHC(═O)N(CH₃)— or —N(CH₃)C(═O)NH—.

In some embodiments, Y in Formula (I) and Formula (Ia) is —OC(═O)O—, —NHC(═O)O—, —OC(═O)NH—, or —NHC(═O)NH—.

In some embodiments, R⁵ in Formula (I) and Formula (Ia) is hydrogen or substituted or unsubstituted C₁-C₃ alkyl.

In some embodiments, R⁵ in Formula (I) and Formula (Ia) is H, —CH₃, —CH—) CH₃, —CH₂CH₂CH₃, or —CH(CH₃)₂.

In some embodiments, R⁵ in Formula (I) and Formula (Ia) is H.

### Exemplary Lipids of WO2022140252

In another aspect, an amino lipid is according to any formula or structure, or a pharmaceutically acceptable salt or solvate thereof, as described in International Publication No. WO2022140252, which is hereby incorporated by reference in its entirety. In some embodiments, an amino lipid has a structure according to any of Formulae A′, A, I″, I′, I, II″, II′, II, III′, III, I″-a, I′-a, I-a, I″-a-i, I″-a-ii, I″-a-iii, I″-b, I′-b, I-b, I″-b-i, I″-b-ii, I″-b-iii, I″-c, I′-c, I-c, I″-c-i, I″-c-ii, I″-c-iii, I′-d, I-d, I′-d-i, II-a, II-a-i, III-a, and III-a-i of WO2022140252, or a pharmaceutically acceptable salt or solvate thereof. Exemplary amino lipids also include any of the lipids of Table 1 of WO2022140252, including any of the lipids represented by Examples 7-1 to 7-253 and Examples 8-1 to 8-106, or a pharmaceutically acceptable salt or solvate thereof.

In some embodiments, an amino lipid is according to Formula A′ of WO2022140252:

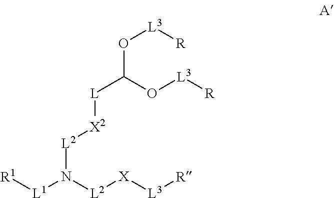

or its N-oxide, or a pharmaceutically acceptable salt thereof, wherein
L¹ is absent, C₁₋₆ alkylenyl, or C₂₋₆ heteroalkylenyl;
each L² is independently optionally substituted C₂₋₁₅ alkylenyl, or optionally substituted C₃₋₁₅ heteroalkylenyl;
L is C₁₋₁₀ alkylenyl, or C₂₋₁₀ heteroalkylenyl;
X² is —OC(O)—, —C(O)O—, or —OC(O)O—;
X is absent, —OC(O)—, —C(O)O—, or —OC(O)O—;
R″ is hydrogen,

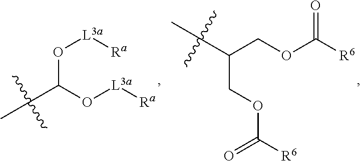

or an optionally substituted group selected from C₆₋₂₀ aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl;
each of R and R^{a} is independently hydrogen, or an optionally substituted group selected from C₆₋₂₀ aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl
each of L³ and L^{3a} is independently absent, optionally substituted C₁₋₁₀ alkylenyl, or optionally substituted C₂₋₁₀ heteroalkylenyl;
R¹ is hydrogen, optionally substituted phenyl, optionally substituted 3- to 7-membered cycloaliphatic, optionally substituted 3- to 7-membered heterocyclyl comprising 1-3 heteroatoms independently selected from nitrogen, oxygen, and sulfur, optionally substituted 5- to 6-membered monocyclic heteroaryl comprising 1-4 heteroatoms independently selected from nitrogen, oxygen, and sulfur, optionally substituted 8- to 10-membered bicyclic heteroaryl comprising 1-4 heteroatoms independently selected from nitrogen, oxygen, and sulfur, —OR², —C(O)OR², —C(O)SR², —OC(O)R², —OC(O)OR², —CN, —N(R²)₂, —C(O)N(R²)₂, —S(O)₂N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(O)₂R², —N(OR²)C(O)OR², —N(OR²)C(O)N(R²)₂, —N(OR²) C(S)N(R²)₂, —N(OR²) C(NR²)N(R²)₂, —N(OR²) C(CHR²)N(R²)₂, —C(NR²)N(R²)₂, —C(NR²)R², —C(O)N(R²)OR², —C(R²) N(R²)₂C(O)OR², —CR²(R³)₂, —OP(O)(OR²)₂, or —P(O)(OR²)₂; or
R¹ is

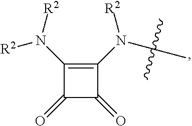

or a ring selected from 3- to 7-membered cycloaliphatic and 3- to 7-membered heterocyclyl comprising 1-3 heteroatoms independently selected from nitrogen, oxygen, and sulfur, wherein the cycloaliphatic or heterocyclyl ring is optionally substituted with 1-4 R² or R³ groups;
each R² is independently hydrogen, oxo, —CN, —NO₂, —OR⁴, —S(O)₂R⁴, —S(O)₂N(R⁴)₂, —(CH₂)ₙ—R⁴, or an optionally substituted group selected from C₁₋₆ aliphatic, phenyl, 3- to 7-membered cycloaliphatic, 5- to 6-membered monocyclic heteroaryl comprising 1-4 heteroatoms independently selected from nitrogen, oxygen, and sulfur, and 3- to 7-membered heterocyclyl comprising 1-3 heteroatoms independently selected from nitrogen, oxygen, and sulfur; or
   two occurrences of R², taken together with the atom(s) to which they are attached, form optionally substituted 4- to 7-membered heterocyclyl comprising 0-1 additional heteroatom selected from nitrogen, oxygen, and sulfur;
each R³ is independently —(CH₂)ₙ—R⁴; or
   two occurrences of R³, taken together with the atom(s) to which they are attached, form optionally substituted 5- to 6-membered heterocyclyl comprising 0-1 additional heteroatom selected from nitrogen, oxygen, and sulfur;
each R⁴ is independently hydrogen, —OR⁵, —N(R⁵)₂, —OC(O)R⁵, —OC(O)OR⁵, —CN, —C(O)N(R⁵)₂, —NR⁵C(O)R⁵, —OC(O)N(R⁵)₂, —N(R⁵)C(O)OR⁵, —NR⁵S(O)₂R⁵, —NR⁵C(O)N(R⁵)₂, —NR⁵C(S)N(R⁵)₂, —NR⁵C(NR⁵)N(R⁵)₂, or

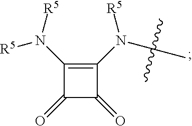

each R⁵ is independently hydrogen, or optionally substituted C₁₋₆ aliphatic; or
   two occurrences of R⁵, taken together with the atom(s) to which they are attached, form optionally substituted 4- to 7-membered heterocyclyl comprising 0-1 additional heteroatom selected from nitrogen, oxygen, and sulfur;
each R⁶ is independently C4-12 aliphatic; and
each n is independently 0 to 4.

In some embodiments, an amino lipid is according to Formula III-a of WO2022140252:

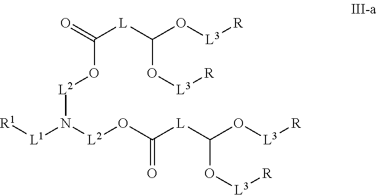

or its N-oxide, or a pharmaceutically acceptable salt thereof, wherein each of R, R¹, L, L¹, L², L³ is as defined therein for any of Formulae A′, A, III′, and III, and described in classes and subclasses above and herein, both singly and in combination. In embodiments of Formula III-a, each of R, R¹, L, L¹, L², L³ is as defined herein for Formula A′ above.

In some embodiments, an amino lipid is according to Formula III-a-i of WO2022140252:

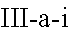

or its N-oxide, or a pharmaceutically acceptable salt thereof, wherein each of R, R¹, L, L¹, and L² is as defined therein for any of Formulae A′, A, III′, and III, and described in classes and subclasses above and herein, both singly and in combination. In embodiments of Formula III-a-i, each of R, R¹, L, L¹, and L² is as defined herein for Formula A′ above.

In some embodiments, an amino lipid is selected from any of the lipids described in Table 1 of WO2022140252, or its N-oxide, or a pharmaceutically acceptable salt thereof. In embodiments, an amino lipid is selected from the group consisting of:

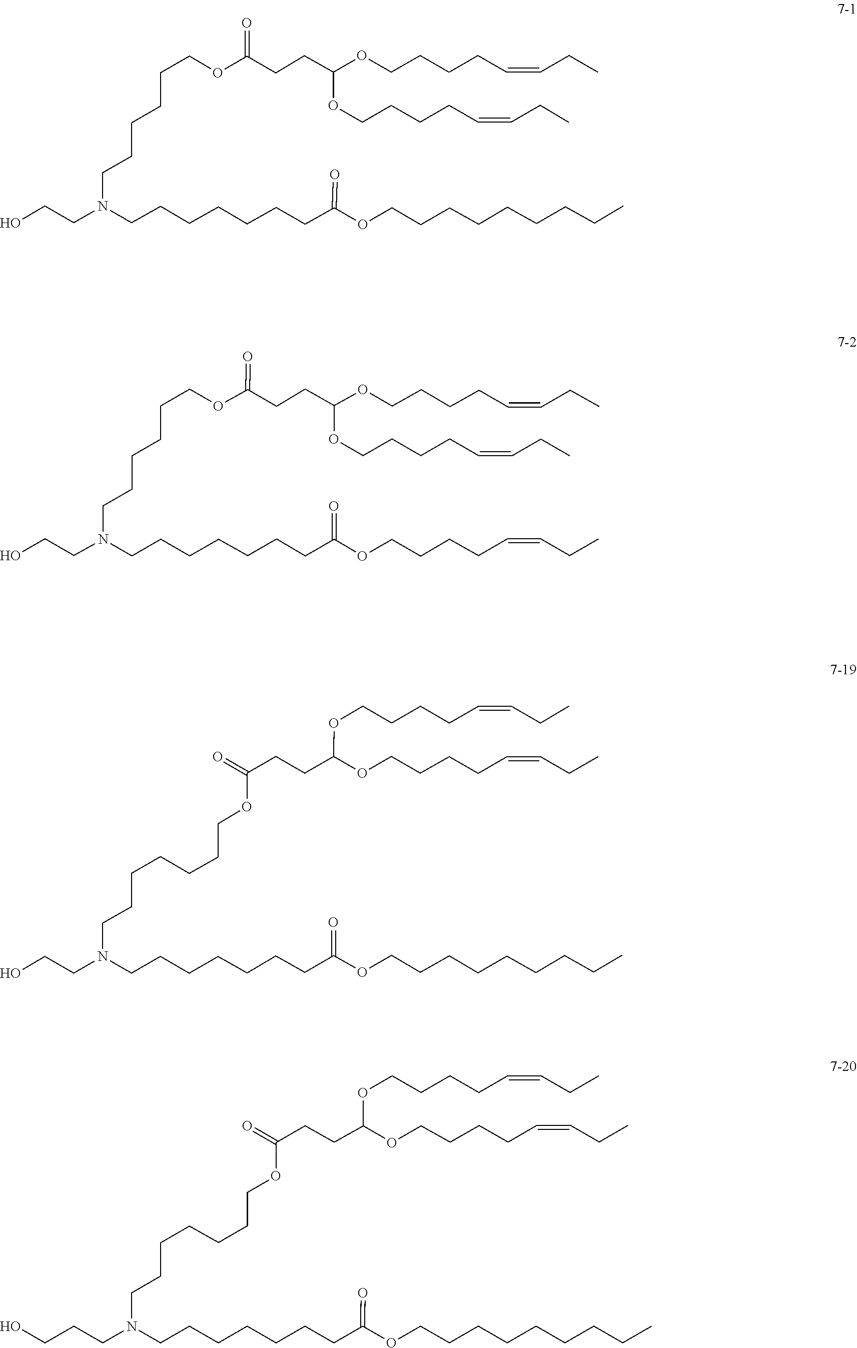 [SMILES: CC/C=C\CCCCOC(CCC(=O)OCCCCCCN(CCO)CCCCCCCC(=O)OCCCCCCCCC)OCCCC/C=C\CC ; CC/C=C\CCCCOC(=O)CCCCCCCN(CCO)CCCCCCOC(=O)CCC(OCCCC/C=C\CC)OCCCC/C=C\CC ; CC/C=C\CCCCOC(CCC(=O)OCCCCCCCN(CCO)CCCCCCCC(=O)OCCCCCCCCC)OCCCC/C=C\CC ; CC/C=C\CCCCOC(CCC(=O)OCCCCCCCN(CCCO)CCCCCCCC(=O)OCCCCCCCCC)OCCCC/C=C\CC]

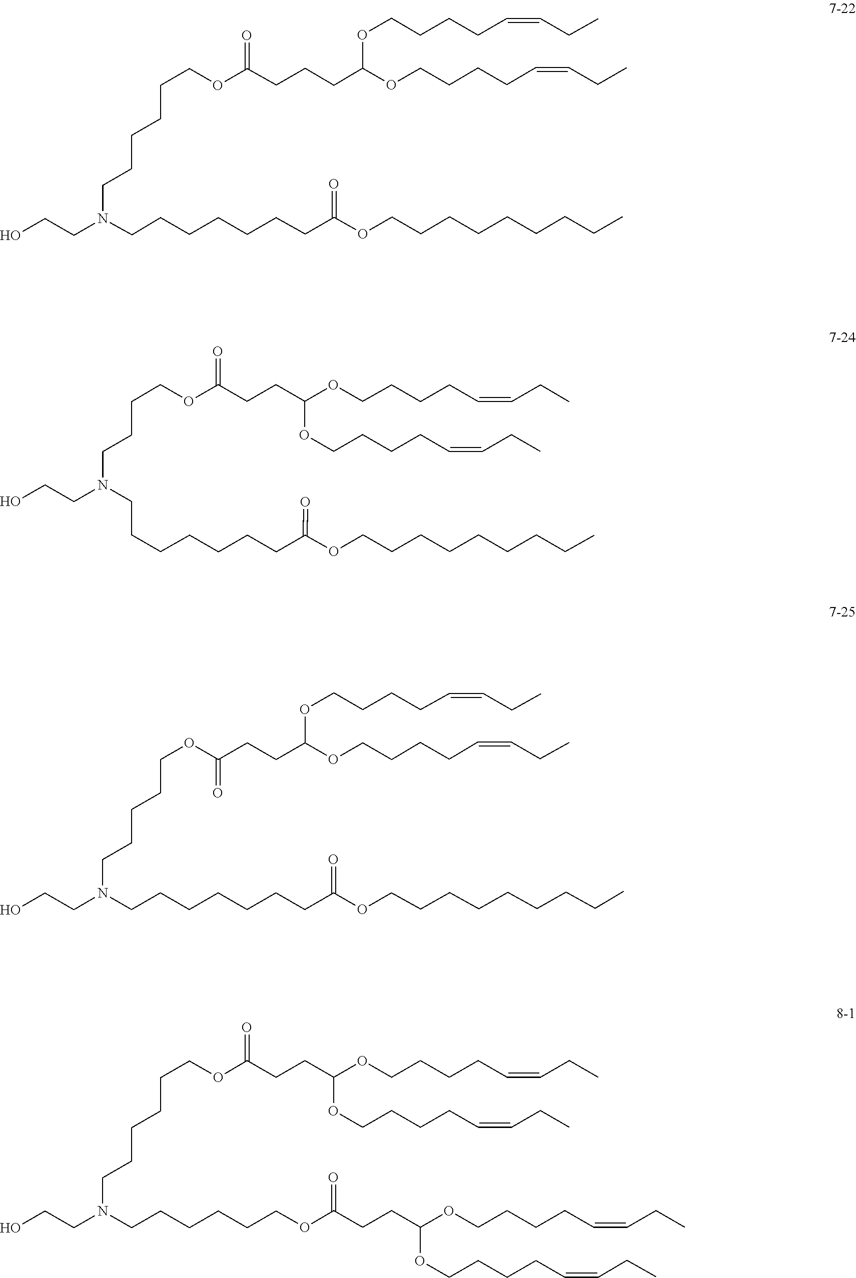 [SMILES: CC/C=C\CCCCOC(CCCC(=O)OCCCCCCN(CCO)CCCCCCCC(=O)OCCCCCCCCC)OCCCC/C=C\CC ; CC/C=C\CCCCOC(CCC(=O)OCCCCN(CCO)CCCCCCCC(=O)OCCCCCCCCC)OCCCC/C=C\CC ; CC/C=C\CCCCOC(CCC(=O)OCCCCCN(CCO)CCCCCCCC(=O)OCCCCCCCCC)OCCCC/C=C\CC ; CC/C=C\CCCCOC(CCC(=O)OCCCCCCN(CCO)CCCCCCOC(=O)CCC(OCCCC/C=C\CC)OCCCC/C=C\CC)OCCCC/C=C\CC]

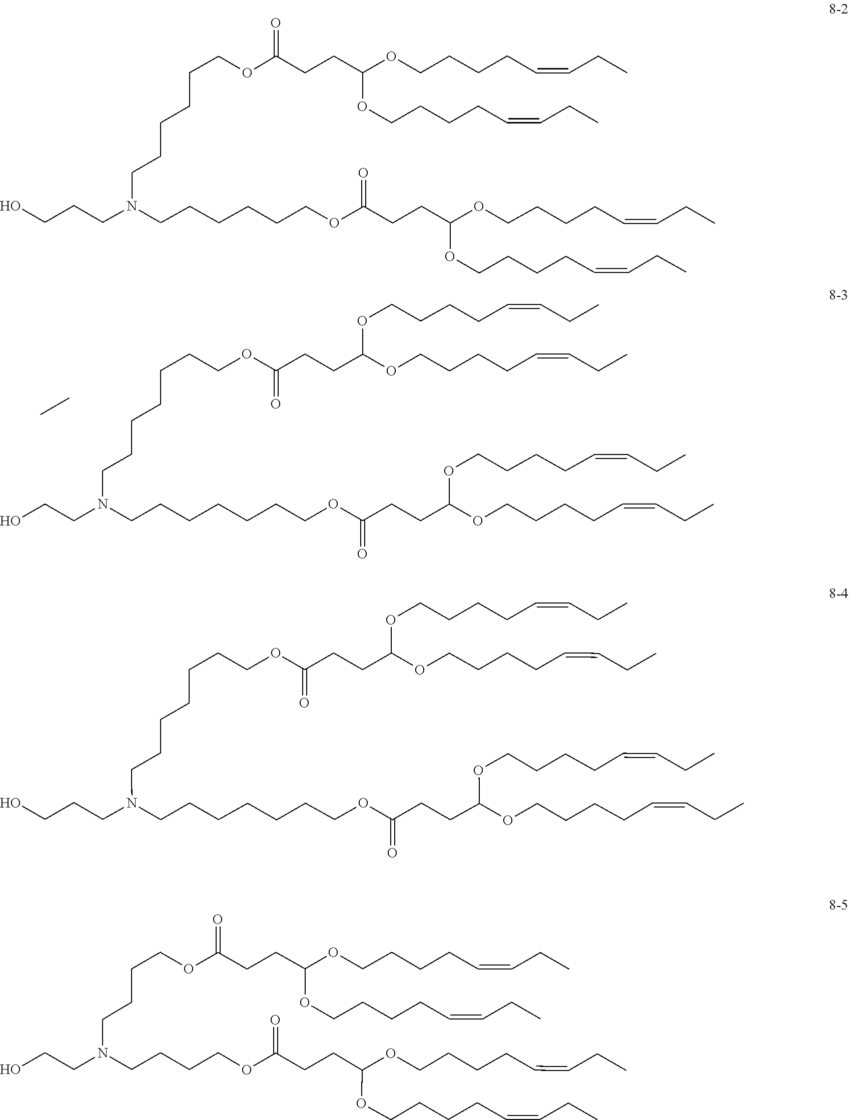

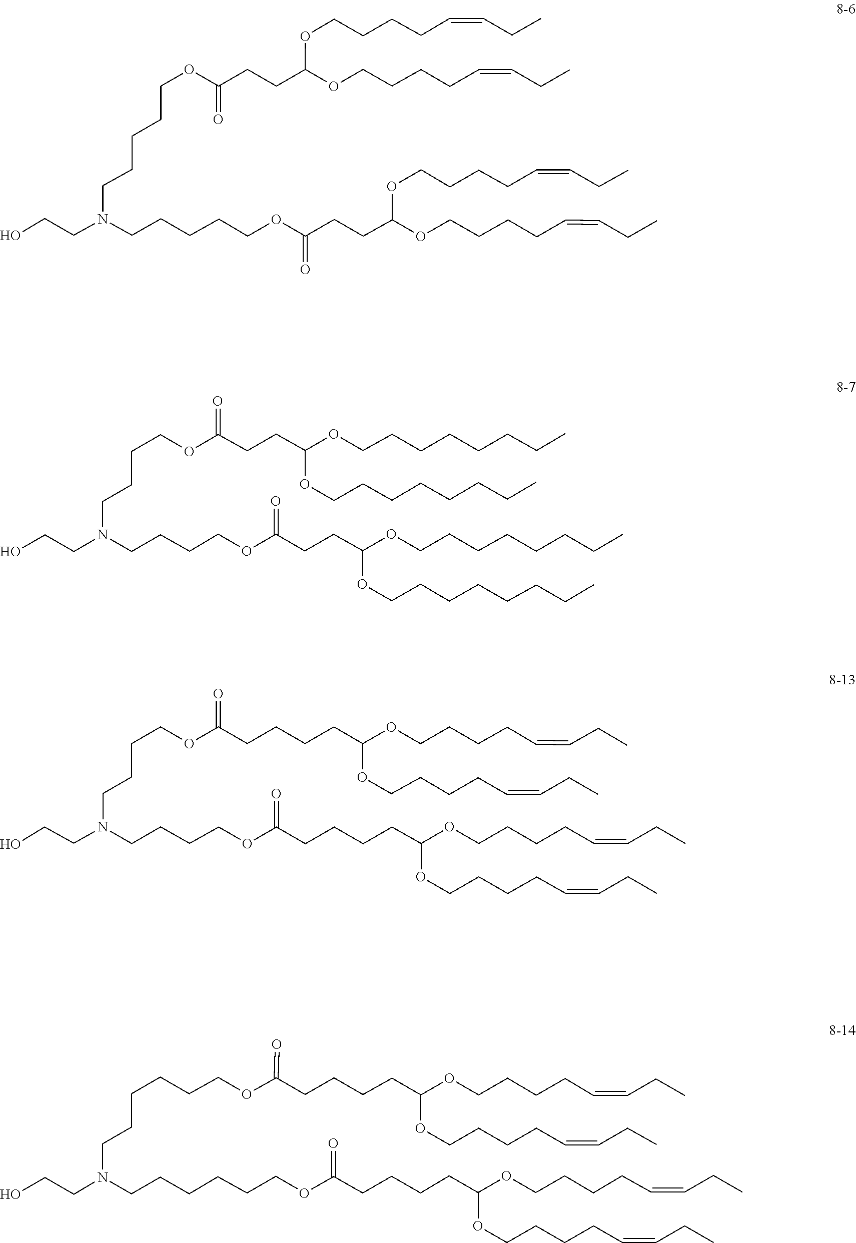 [SMILES: CC/C=C\CCCCOC(CCC(=O)OCCCCCN(CCO)CCCCCOC(=O)CCC(OCCCC/C=C\CC)OCCCC/C=C\CC)OCCCC/C=C\CC ; CCCCCCCCOC(CCC(=O)OCCCCN(CCO)CCCCOC(=O)CCC(OCCCCCCCC)OCCCCCCCC)OCCCCCCCC ; CC/C=C\CCCCOC(CCCCC(=O)OCCCCN(CCO)CCCCOC(=O)CCCCC(OCCCC/C=C\CC)OCCCC/C=C\CC)OCCCC/C=C\CC ; CC/C=C\CCCCOC(CCCCC(=O)OCCCCCCN(CCO)CCCCCCOC(=O)CCCCC(OCCCC/C=C\CC)OCCCC/C=C\CC)OCCCC/C=C\CC]

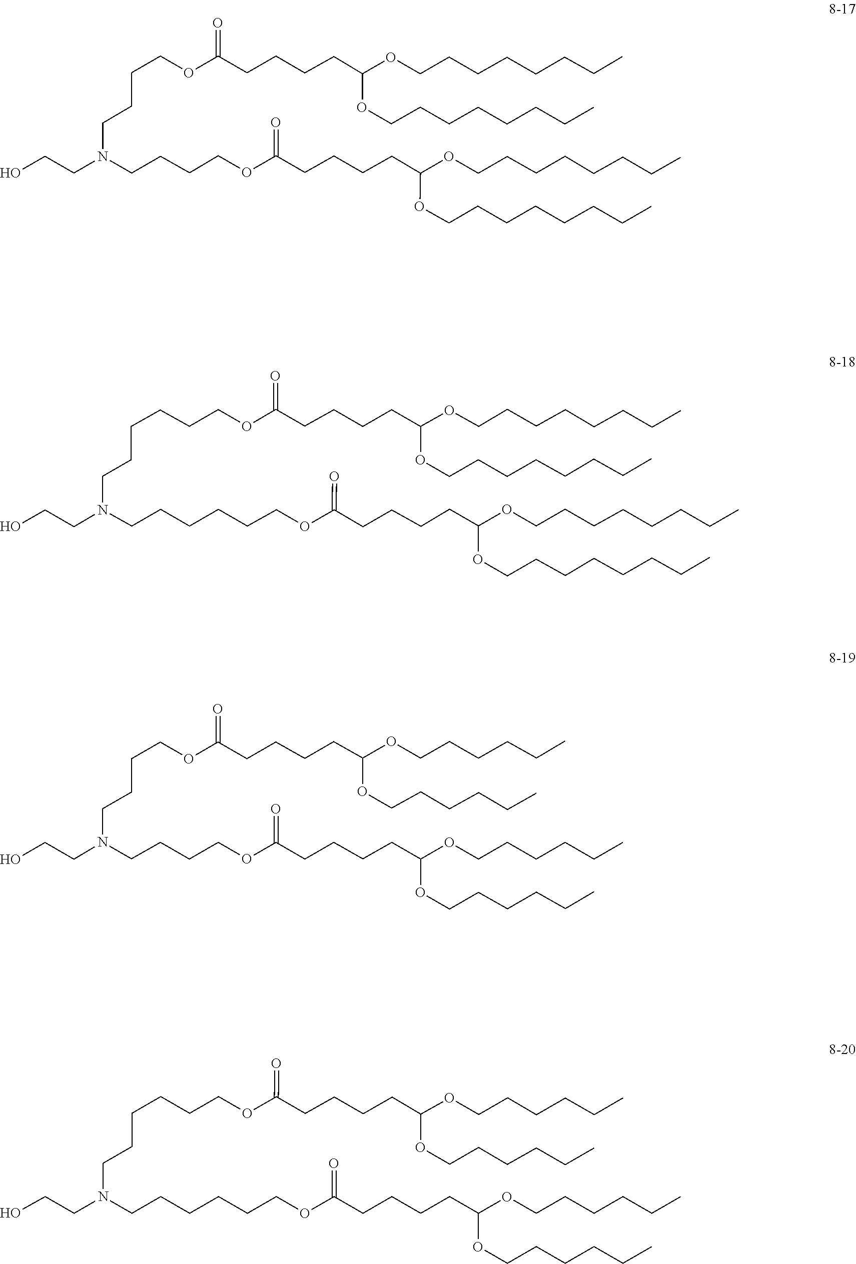 [SMILES: CCCCCCCCOC(CCCCC(=O)OCCCCN(CCO)CCCCOC(=O)CCCCC(OCCCCCCCC)OCCCCCCCC)OCCCCCCCC ; CCCCCCCCOC(CCCCC(=O)OCCCCCCN(CCO)CCCCCCOC(=O)CCCCC(OCCCCCCCC)OCCCCCCCC)OCCCCCCCC ; CCCCCCOC(CCCCC(=O)OCCCCN(CCO)CCCCOC(=O)CCCCC(OCCCCCC)OCCCCCC)OCCCCCC ; CCCCCCOC(CCCCC(=O)OCCCCCCN(CCO)CCCCCCOC(=O)CCCCC(OCCCCCC)OCCCCCC)OCCCCCC]

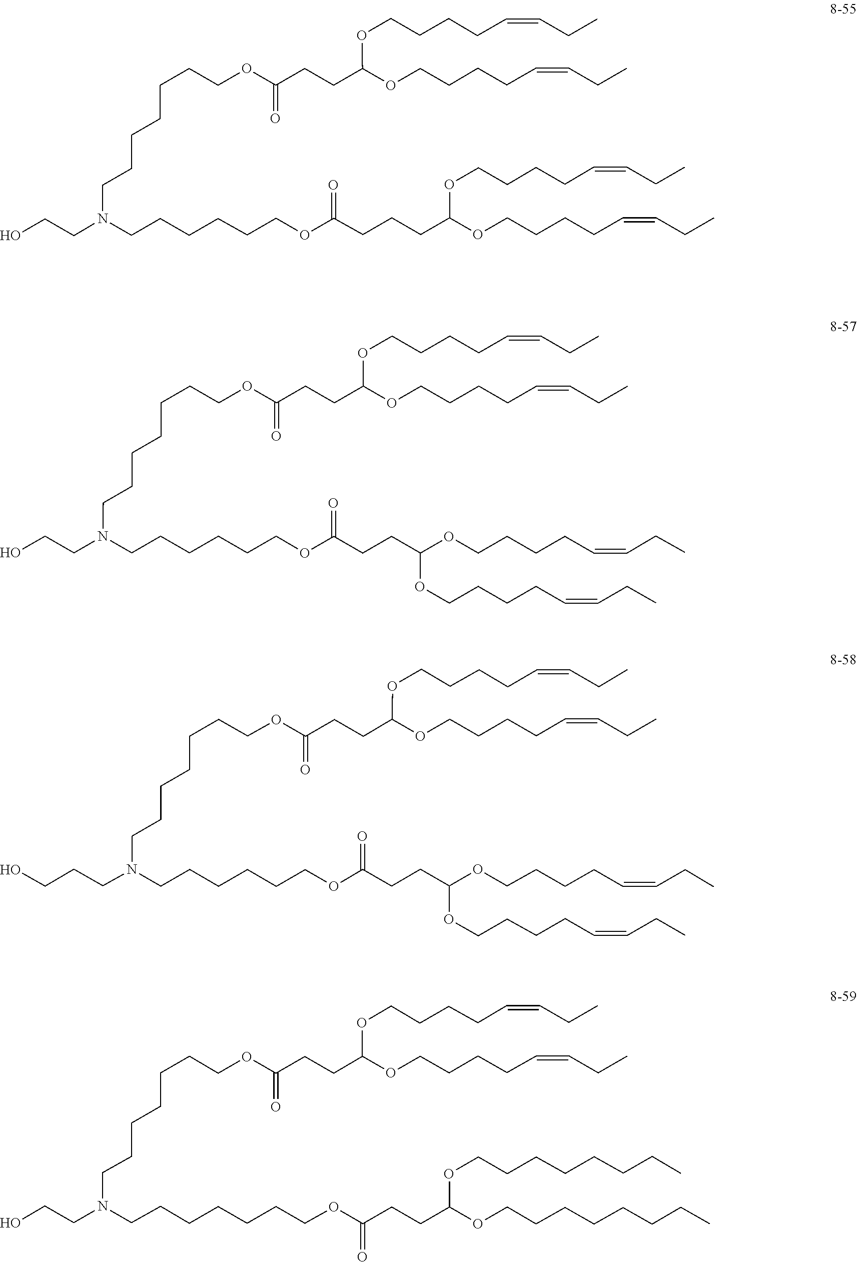 [SMILES: CC/C=C\CCCCOC(CCCC(=O)OCCCCCCN(CCO)CCCCCCCOC(=O)CCC(OCCCC/C=C\CC)OCCCC/C=C\CC)OCCCC/C=C\CC ; CC/C=C\CCCCOC(CCC(=O)OCCCCCCCN(CCO)CCCCCCOC(=O)CCC(OCCCC/C=C\CC)OCCCC/C=C\CC)OCCCC/C=C\CC ; CC/C=C\CCCCOC(CCC(=O)OCCCCCCCN(CCCO)CCCCCCOC(=O)CCC(OCCCC/C=C\CC)OCCCC/C=C\CC)OCCCC/C=C\CC ; CC/C=C\CCCCOC(CCC(=O)OCCCCCCCN(CCO)CCCCCCCOC(=O)CCC(OCCCCCCCC)OCCCCCCCC)OCCCC/C=C\CC]

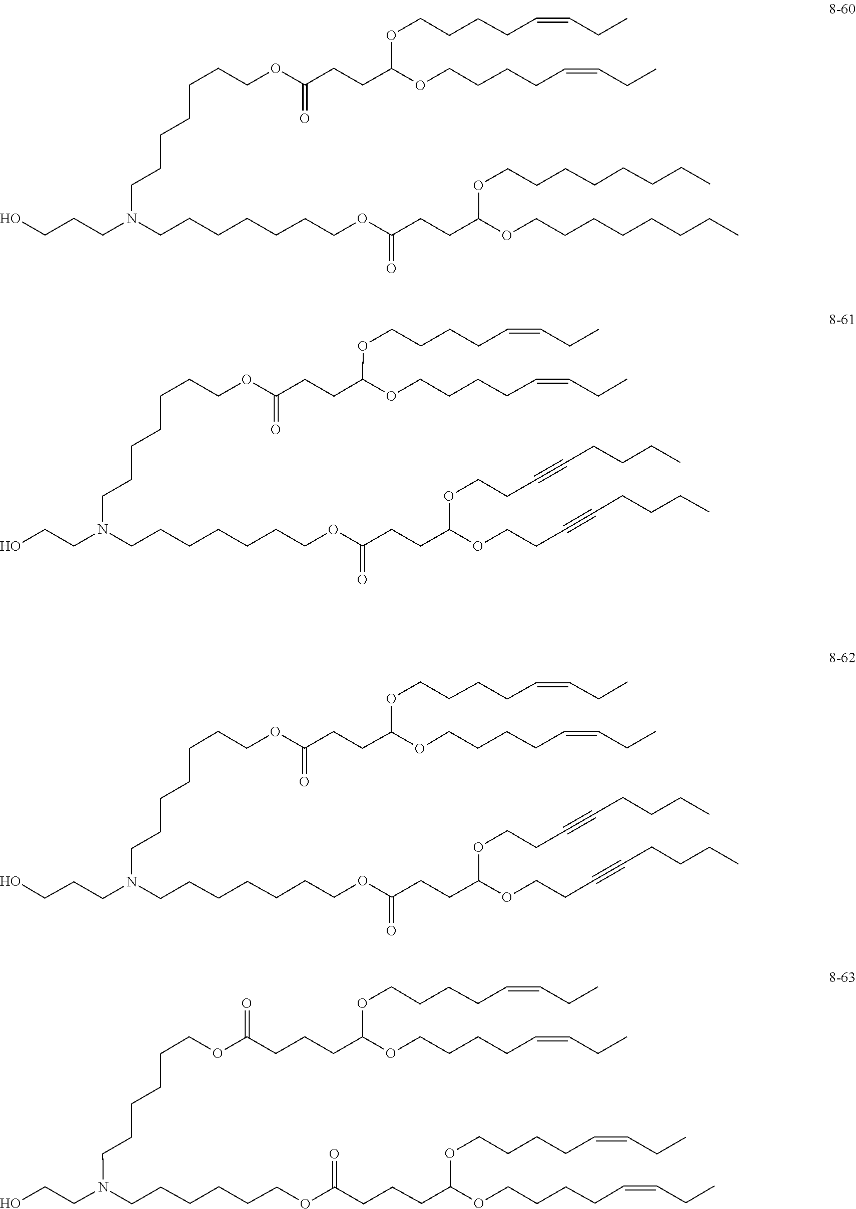 [SMILES: CC/C=C\CCCCOC(CCC(=O)OCCCCCCCN(CCCO)CCCCCCCOC(=O)CCC(OCCCCCCCC)OCCCCCCCC)OCCCC/C=C\CC ; CC/C=C\CCCCOC(CCC(=O)OCCCCCCCN(CCO)CCCCCCCOC(=O)CCC(OCCC#CCCCC)OCCC#CCCCC)OCCCC/C=C\CC ; CC/C=C\CCCCOC(CCC(=O)OCCCCCCCN(CCCO)CCCCCCCOC(=O)CCC(OCCC#CCCCC)OCCC#CCCCC)OCCCC/C=C\CC ; CC/C=C\CCCCOC(CCCC(=O)OCCCCCCN(CCO)CCCCCCOC(=O)CCCC(OCCCC/C=C\CC)OCCCC/C=C\CC)OCCCC/C=C\CC]

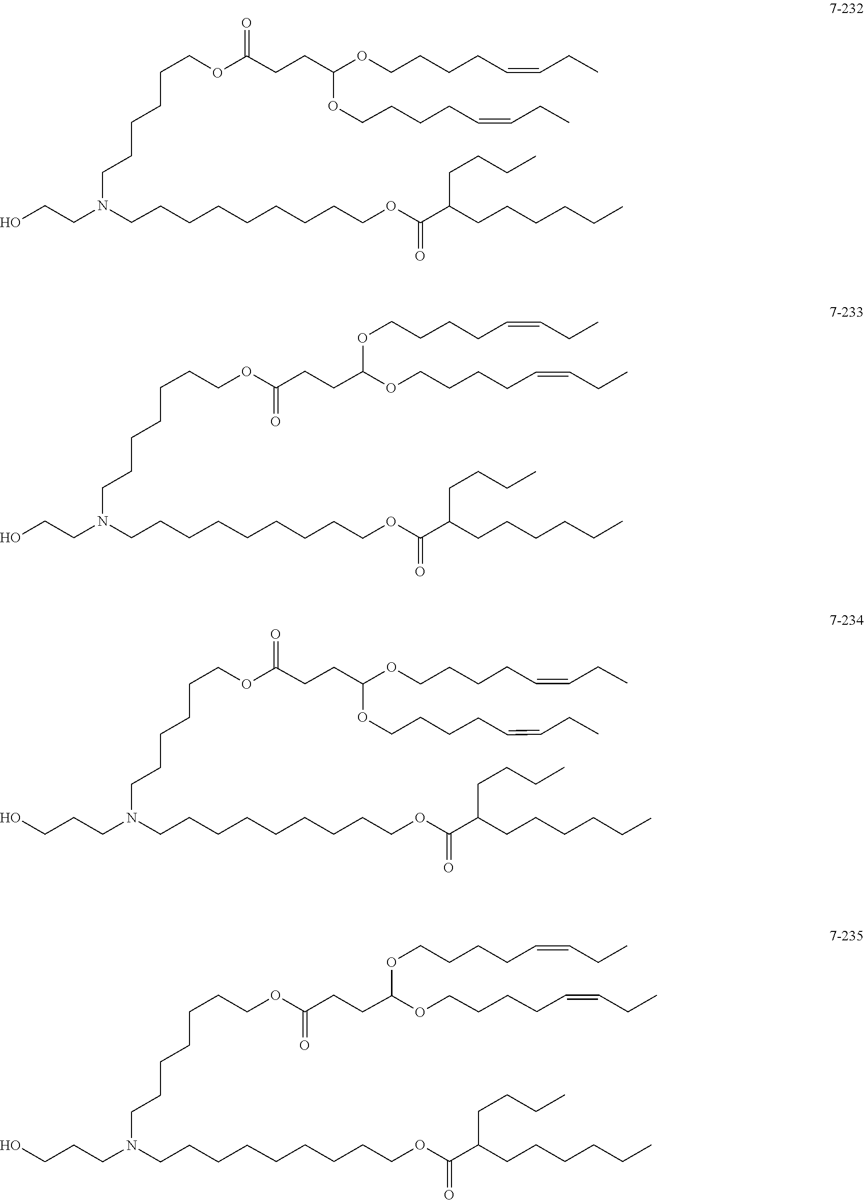 [SMILES: CC/C=C\CCCCOC(CCC(=O)OCCCCCCN(CCO)CCCCCCCCCOC(=O)C(CCCC)CCCCCC)OCCCC/C=C\CC ; CC/C=C\CCCCOC(CCC(=O)OCCCCCCCN(CCO)CCCCCCCCCOC(=O)C(CCCC)CCCCCC)OCCCC/C=C\CC ; CC/C=C\CCCCOC(CCC(=O)OCCCCCCN(CCCO)CCCCCCCCCOC(=O)C(CCCC)CCCCCC)OCCCC/C=C\CC ; CC/C=C\CCCCOC(CCC(=O)OCCCCCCCN(CCCO)CCCCCCCCCOC(=O)C(CCCC)CCCCCC)OCCCC/C=C\CC]

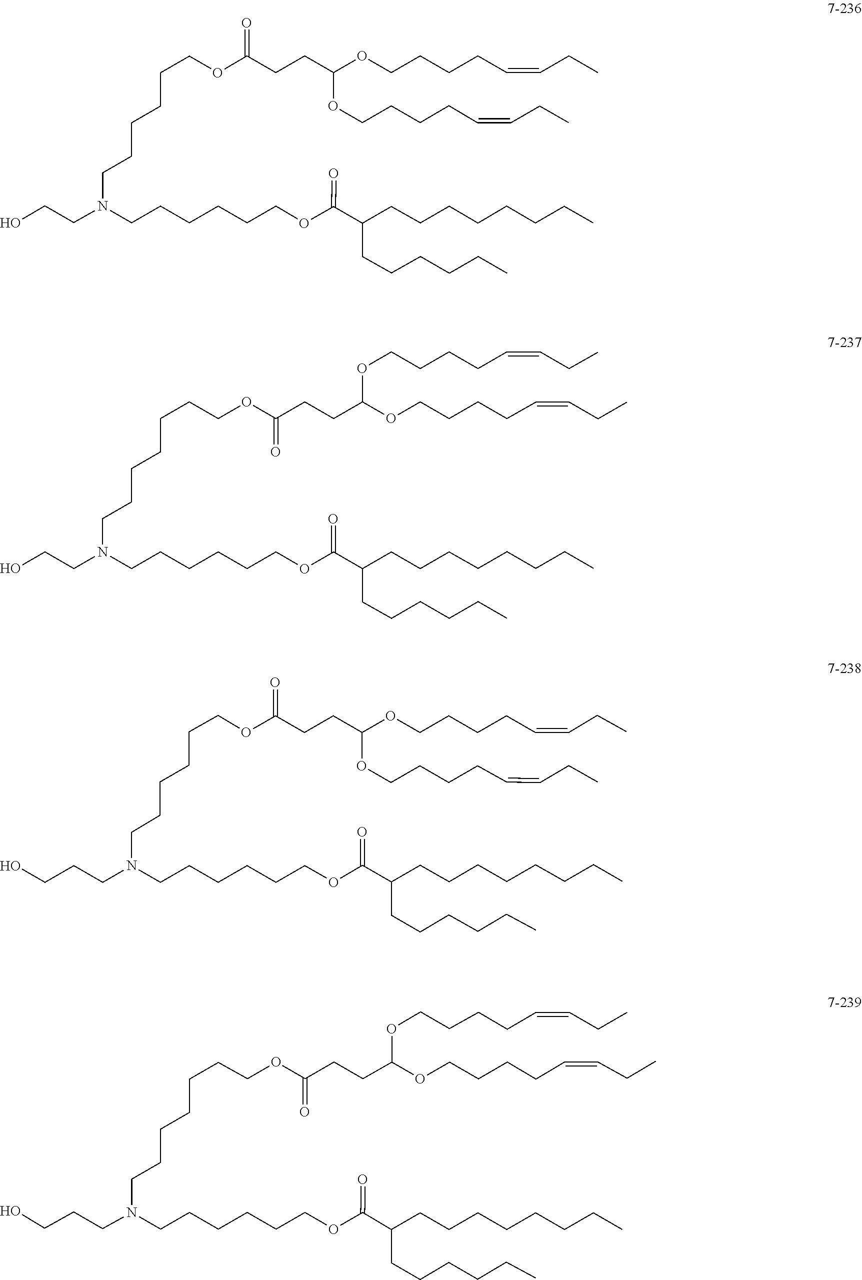 [SMILES: CC/C=C\CCCCOC(CCC(=O)OCCCCCCN(CCO)CCCCCCOC(=O)C(CCCCCC)CCCCCCCC)OCCCC/C=C\CC ; CC/C=C\CCCCOC(CCC(=O)OCCCCCCCN(CCO)CCCCCCOC(=O)C(CCCCCC)CCCCCCCC)OCCCC/C=C\CC ; CC/C=C\CCCCOC(CCC(=O)OCCCCCCN(CCCO)CCCCCCOC(=O)C(CCCCCC)CCCCCCCC)OCCCC/C=C\CC ; CC/C=C\CCCCOC(CCC(=O)OCCCCCCCN(CCCO)CCCCCCOC(=O)C(CCCCCC)CCCCCCCC)OCCCC/C=C\CC]

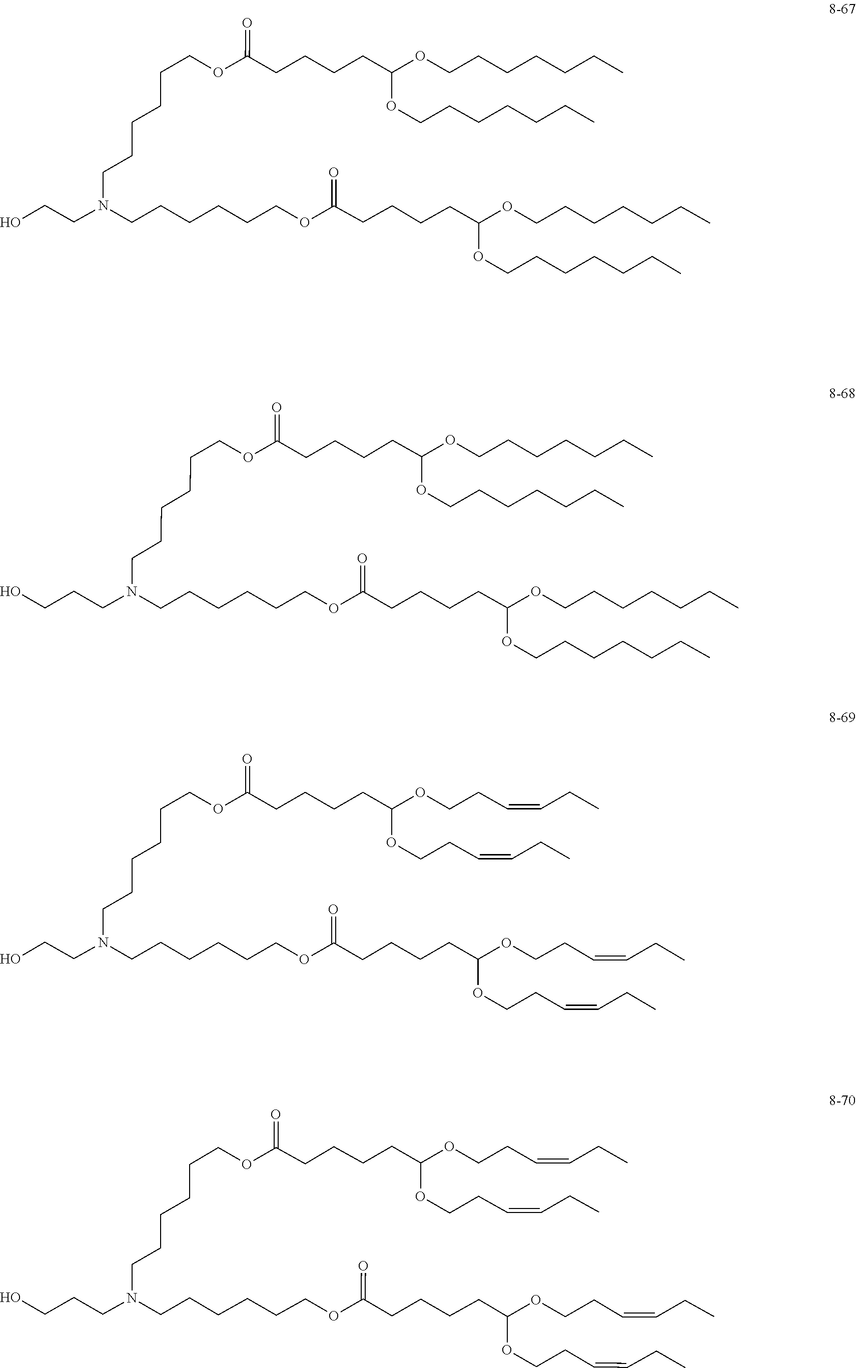 [SMILES: CCCCCCCOC(CCCCC(=O)OCCCCCCN(CCO)CCCCCCOC(=O)CCCCC(OCCCCCCC)OCCCCCCC)OCCCCCCC ; CCCCCCCOC(CCCCC(=O)OCCCCCCN(CCCO)CCCCCCOC(=O)CCCCC(OCCCCCCC)OCCCCCCC)OCCCCCCC ; CC/C=C\CCOC(CCCCC(=O)OCCCCCCN(CCO)CCCCCCOC(=O)CCCCC(OCC/C=C\CC)OCC/C=C\CC)OCC/C=C\CC ; CC/C=C\CCOC(CCCCC(=O)OCCCCCCN(CCCO)CCCCCCOC(=O)CCCCC(OCC/C=C\CC)OCC/C=C\CC)OCC/C=C\CC]

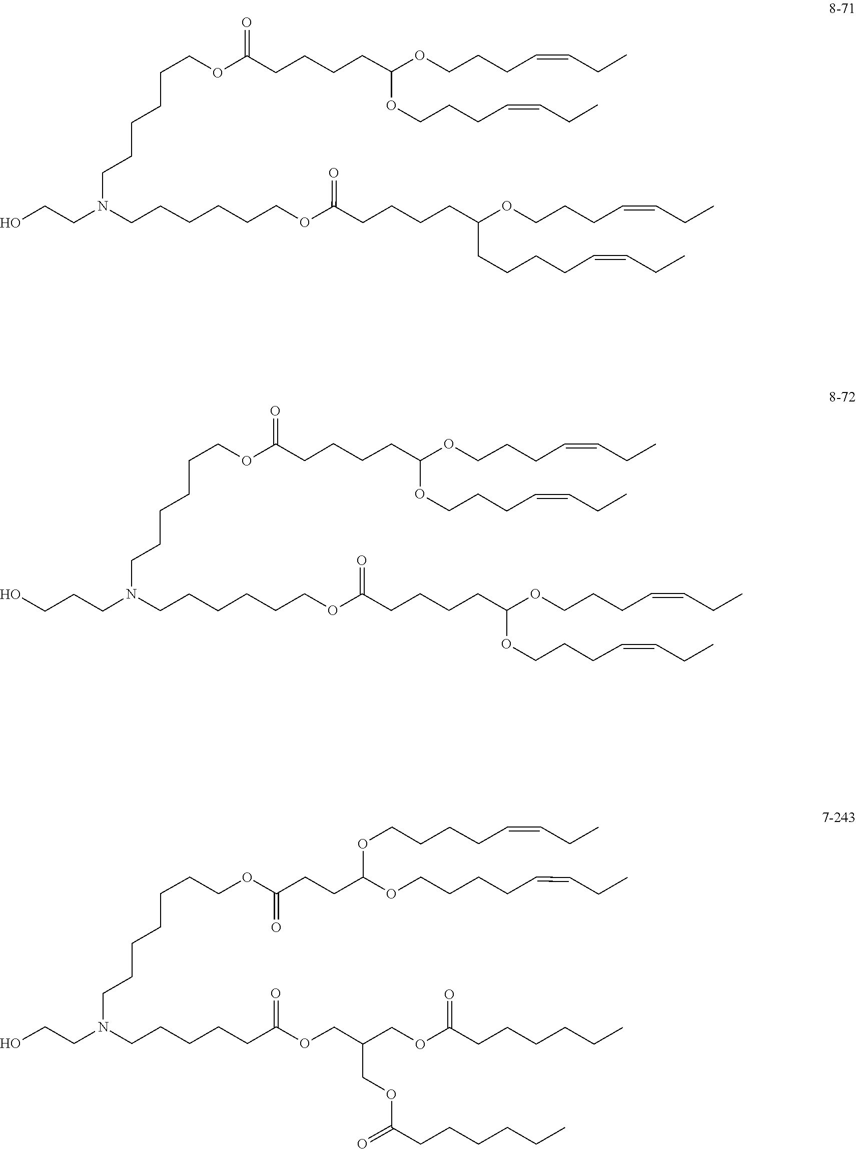 [SMILES: CC/C=C\CCCCC(CCCCC(=O)OCCCCCCN(CCO)CCCCCCOC(=O)CCCCC(OCCC/C=C\CC)OCCC/C=C\CC)OCCC/C=C\CC ; CC/C=C\CCCOC(CCCCC(=O)OCCCCCCN(CCCO)CCCCCCOC(=O)CCCCC(OCCC/C=C\CC)OCCC/C=C\CC)OCCC/C=C\CC ; CC/C=C\CCCCOC(CCC(=O)OCCCCCCCN(CCO)CCCCCC(=O)OCC(COC(=O)CCCCCC)COC(=O)CCCCCC)OCCCC/C=C\CC]

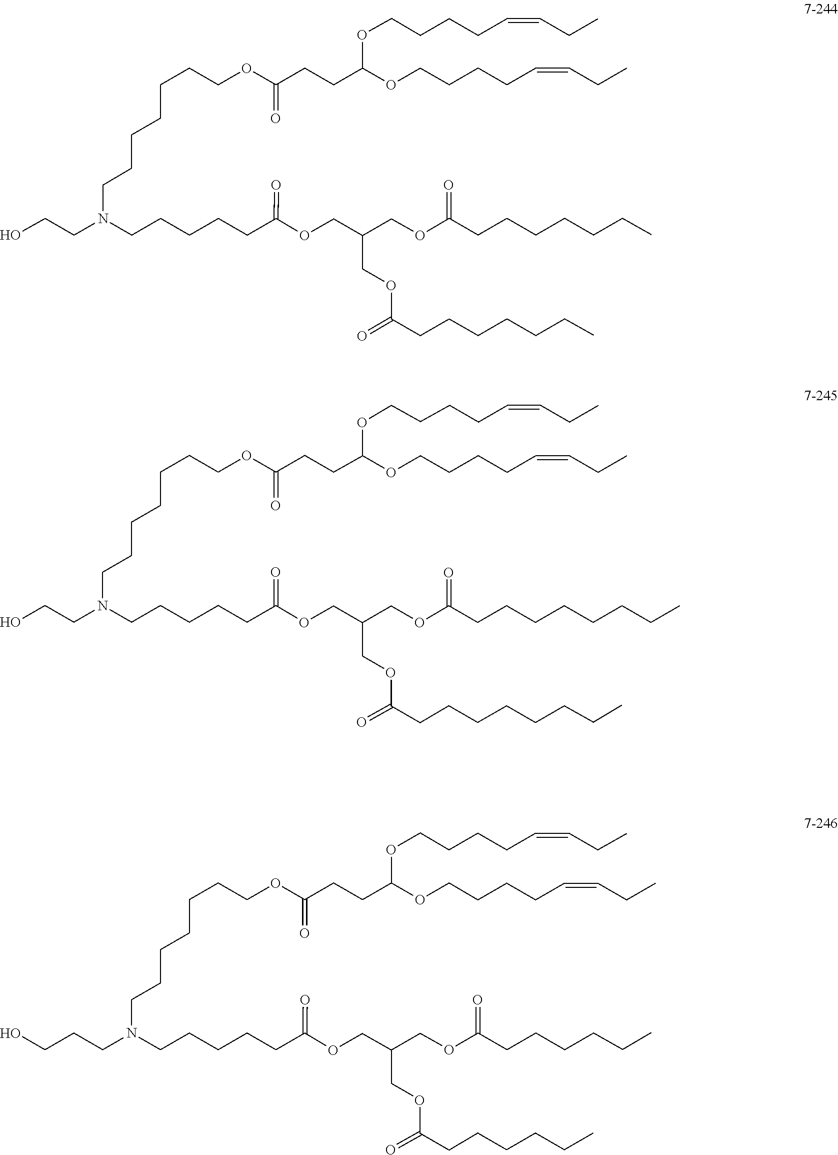 [SMILES: CC/C=C\CCCCOC(CCC(=O)OCCCCCCCN(CCO)CCCCCC(=O)OCC(COC(=O)CCCCCCC)COC(=O)CCCCCCC)OCCCC/C=C\CC ; CC/C=C\CCCCOC(CCC(=O)OCCCCCCCN(CCO)CCCCCC(=O)OCC(COC(=O)CCCCCCCC)COC(=O)CCCCCCCC)OCCCC/C=C\CC ; CC/C=C\CCCCOC(CCC(=O)OCCCCCCCN(CCCO)CCCCCC(=O)OCC(COC(=O)CCCCCC)COC(=O)CCCCCC)OCCCC/C=C\CC]

In some embodiments, an amino lipid is Example 7-1, or a pharmaceutically acceptable salt thereof. In some embodiments, an amino lipid is Example 7-2, or a pharmaceutically acceptable salt thereof. In some embodiments, an amino lipid is Example 7-19, or a pharmaceutically acceptable salt thereof. In some embodiments, an amino lipid is Example 7-20, or a pharmaceutically acceptable salt thereof. In some embodiments, an amino lipid is Example 7-22, or a pharmaceutically acceptable salt thereof. In some embodiments, an amino lipid is Example 7-24, or a pharmaceutically acceptable salt thereof. In some embodiments, an amino lipid is Example 7-25, or a pharmaceutically acceptable salt thereof. In some embodiments, an amino lipid is Example 8-1, or a pharmaceutically acceptable salt thereof. In some embodiments, an amino lipid is Example 8-2, or a pharmaceutically acceptable salt thereof. In some embodiments, an amino lipid is Example 8-3, or a pharmaceutically acceptable salt thereof. In some embodiments, an amino lipid is Example 8-4, or a pharmaceutically acceptable salt thereof. In some embodiments, an amino lipid is Example 8-5, or a pharmaceutically acceptable salt thereof. In some embodiments, an amino lipid is Example 8-13, or a pharmaceutically acceptable salt thereof. In some embodiments, an amino lipid is Example 8-14, or a pharmaceutically acceptable salt thereof. In some embodiments, an amino lipid is Example 8-17, or a pharmaceutically acceptable salt thereof. In some embodiments, an amino lipid is Example 8-18, or a pharmaceutically acceptable salt thereof. In some embodiments, an amino lipid is Example 8-19, or a pharmaceutically acceptable salt thereof. In some embodiments, an amino lipid is Example 8-20, or a pharmaceutically acceptable salt thereof. In some embodiments, an amino lipid is Example 8-55, or a pharmaceutically acceptable salt thereof. In some embodiments, an amino lipid is Example 8-57, or a pharmaceutically acceptable salt thereof. In some embodiments, an amino lipid is Example 8-58, or a pharmaceutically acceptable salt thereof. In some embodiments, an amino lipid is Example 8-59, or a pharmaceutically acceptable salt thereof. In some embodiments, an amino lipid is Example 8-60, or a pharmaceutically acceptable salt thereof. In some embodiments, an amino lipid is Example 8-61, or a pharmaceutically acceptable salt thereof. In some embodiments, an amino lipid is Example 8-62, or a pharmaceutically acceptable salt thereof. In some embodiments, an amino lipid is Example 8-63, or a pharmaceutically acceptable salt thereof. In some embodiments, an amino lipid is Example 7-232, or a pharmaceutically acceptable salt thereof. In some embodiments, an amino lipid is Example 7-233, or a pharmaceutically acceptable salt thereof. In some embodiments, an amino lipid is Example 7-234, or a pharmaceutically acceptable salt thereof. In some embodiments, an amino lipid is Example 7-235, or a pharmaceutically acceptable salt thereof. In some embodiments, an amino lipid is Example 7-236, or a pharmaceutically acceptable salt thereof. In some embodiments, an amino lipid is Example 7-237, or a pharmaceutically acceptable salt thereof. In some embodiments, an amino lipid is Example 7-238, or a pharmaceutically acceptable salt thereof. In some embodiments, an amino lipid is Example 7-239, or a pharmaceutically acceptable salt thereof. In some embodiments, an amino lipid is Example 8-67, or a pharmaceutically acceptable salt thereof. In some embodiments, an amino lipid is Example 8-68, or a pharmaceutically acceptable salt thereof. In some embodiments, an amino lipid is Example 8-69, or a pharmaceutically acceptable salt thereof. In some embodiments, an amino lipid is Example 8-70, or a pharmaceutically acceptable salt thereof. In some embodiments, an amino lipid is Example 8-71, or a pharmaceutically acceptable salt thereof. In some embodiments, an amino lipid is Example 8-72, or a pharmaceutically acceptable salt thereof. In some embodiments, an amino lipid is Example 7-243, or a pharmaceutically acceptable salt thereof. In some embodiments, an amino lipid is Example 7-244, or a pharmaceutically acceptable salt thereof. In some embodiments, an amino lipid is Example 7-245, or a pharmaceutically acceptable salt thereof. In some embodiments, an amino lipid is Example 7-246, or a pharmaceutically acceptable salt thereof.

### Exemplary Lipids of WO2022159472

In another aspect, an amino lipid is according to any formula or structure, or a pharmaceutically acceptable salt or solvate thereof, as described in International Publication No. WO2022159472, which is hereby incorporated by reference in its entirety. In some embodiments, an amino lipid has a structure according to any of Formulae I, II, III, IIIA, IIIB, IIIC, IV, V, VA, VI, VIA, VII, and VIIA of WO2022159472, or a pharmaceutically acceptable salt or solvate thereof. Exemplary amino lipids also include any of the lipids of Table 1 of WO2022159472, including any of the lipids represented by Examples 4-1 to 4-86, or a pharmaceutically acceptable salt or solvate thereof.

In some embodiments, an amino lipid is according to Formula I of WO2022159472:

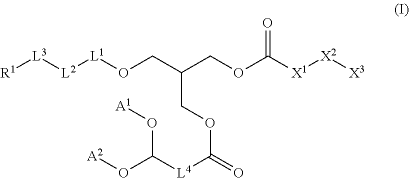

or a pharmaceutically acceptable salt thereof, wherein:
L¹ is a covalent bond, —C(O)—, or —OC(O)—;
L² is a covalent bond, an optionally substituted bivalent saturated or unsaturated, straight or branched C₁-C₁₂ hydrocarbon chain, or

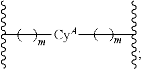

Cy^{A} is an optionally substituted ring selected from phenylene and 3- to 7-membered saturated or partially unsaturated carbocyclene;
each m is independently 0, 1, or 2;
L³ is a covalent bond, —C(O)—, —C(O)O—, —OC(O)—, —O—, or —OC(O)O—;
R¹ is

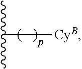

an optionally substituted saturated or unsaturated, straight or branched C₁-C₂₀ hydrocarbon chain wherein 1-3 methylene units are optionally and independently replaced with —O— or —NR—, or

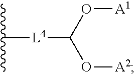

Cy^{B} is an optionally substituted ring selected from 3- to 12-membered saturated or partially unsaturated carbocyclyl, 1-adamantyl, 2-adamantyl,

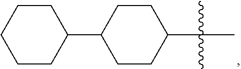 [SMILES: *C1CCC(C2CCCCC2)CC1]

sterolyl, and phenyl;
p is 0, 1, 2, or 3;
each L⁴ is independently a bivalent saturated or unsaturated, straight or branched C₁-C₆ hydrocarbon chain;
each A¹ and A² is independently an optionally substituted C₁-C₂₀ aliphatic or -L⁵-R⁵;
   or A¹ and A², together with their intervening atoms, may form an optionally substituted ring:

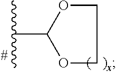

where
   x is selected from 1 or 2; and
   #represents the point of attachment to L⁴;
each L⁵ is independently a bivalent saturated or unsaturated, straight or branched C₁-C₂₀ hydrocarbon chain, wherein 1-3 methylene units are optionally and independently replaced with —O— or —NR—;
each R⁵ is independently an optionally substituted group selected from a 5- to 10-membered aryl ring or a 3- to 8-membered carbocyclic ring;
X¹ is a covalent bond, —O—, or —NR—;
X² is a covalent bond or an optionally substituted, bivalent saturated or unsaturated, straight or branched, C₁-C₁₂ hydrocarbon chain, wherein 1-3 methylene units are optionally and independently replaced with —O—, —NR—, or -Cy^{C}-;
Cy^{C} is an optionally substituted ring selected from 3- to 7-membered saturated or partially unsaturated carbocyclene, phenylene, 3- to 7-membered saturated or partially unsaturated heterocyclene having 1-3 heteroatoms independently selected from nitrogen, oxygen, and sulfur, and 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur; and
each R is independently hydrogen or an optionally substituted C₁-C₆ aliphatic group; provided that when L³ is a covalent bond, then R¹ must be

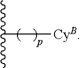

In some embodiments, an amino lipid is according to Formula VI of WO2022159472:

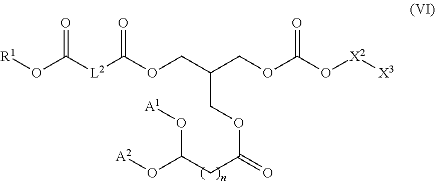

or a pharmaceutically acceptable salt thereof, wherein n is 1, 2, 3 or 4, and L², R¹, A¹, A², X¹, X², and X³ are as defined therein for Formula I and also described in classes and subclasses therein, both singly and in combination. In embodiments, L², R¹, A¹, A², X¹, X², and X³ are as defined herein for Formula I above.

In some embodiments, an amino lipid is according to Formula VIA of WO2022159472:

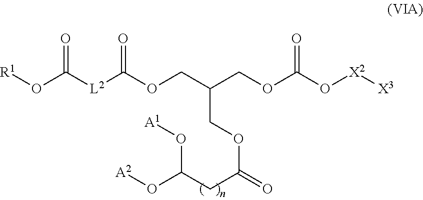

or a pharmaceutically acceptable salt thereof, wherein n is 1, 2, 3 or 4, and L², R¹, A¹, A², X², and X³ are as defined therein for Formula I and also described in classes and subclasses therein, both singly and in combination. In embodiments, L², R¹, A¹, A², X², and X³ are as defined herein for Formula I above.

In some embodiments, an amino lipid is selected from any of the lipids described in Table 1 of WO2022159472, or a pharmaceutically acceptable salt thereof. In embodiments, an amino lipid is selected from the group consisting of:

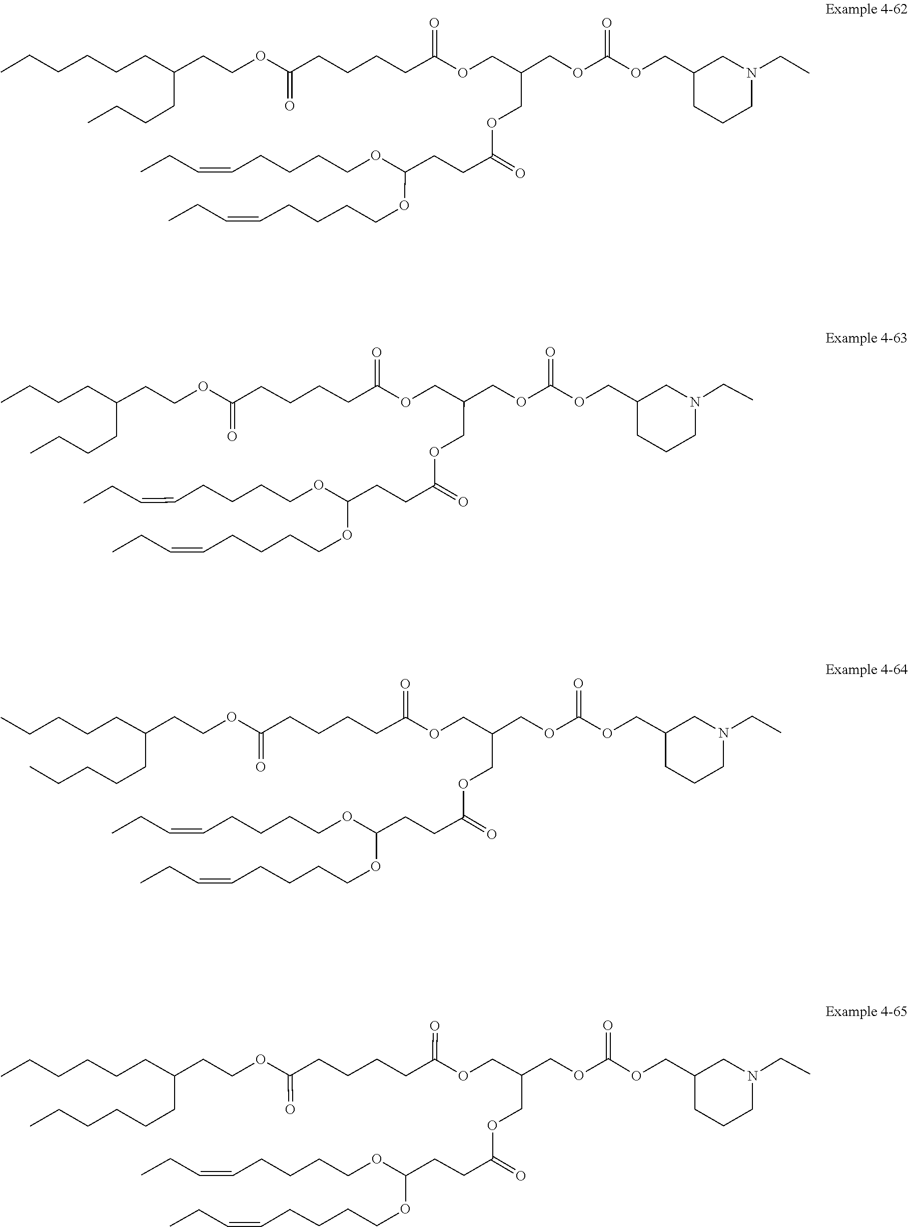

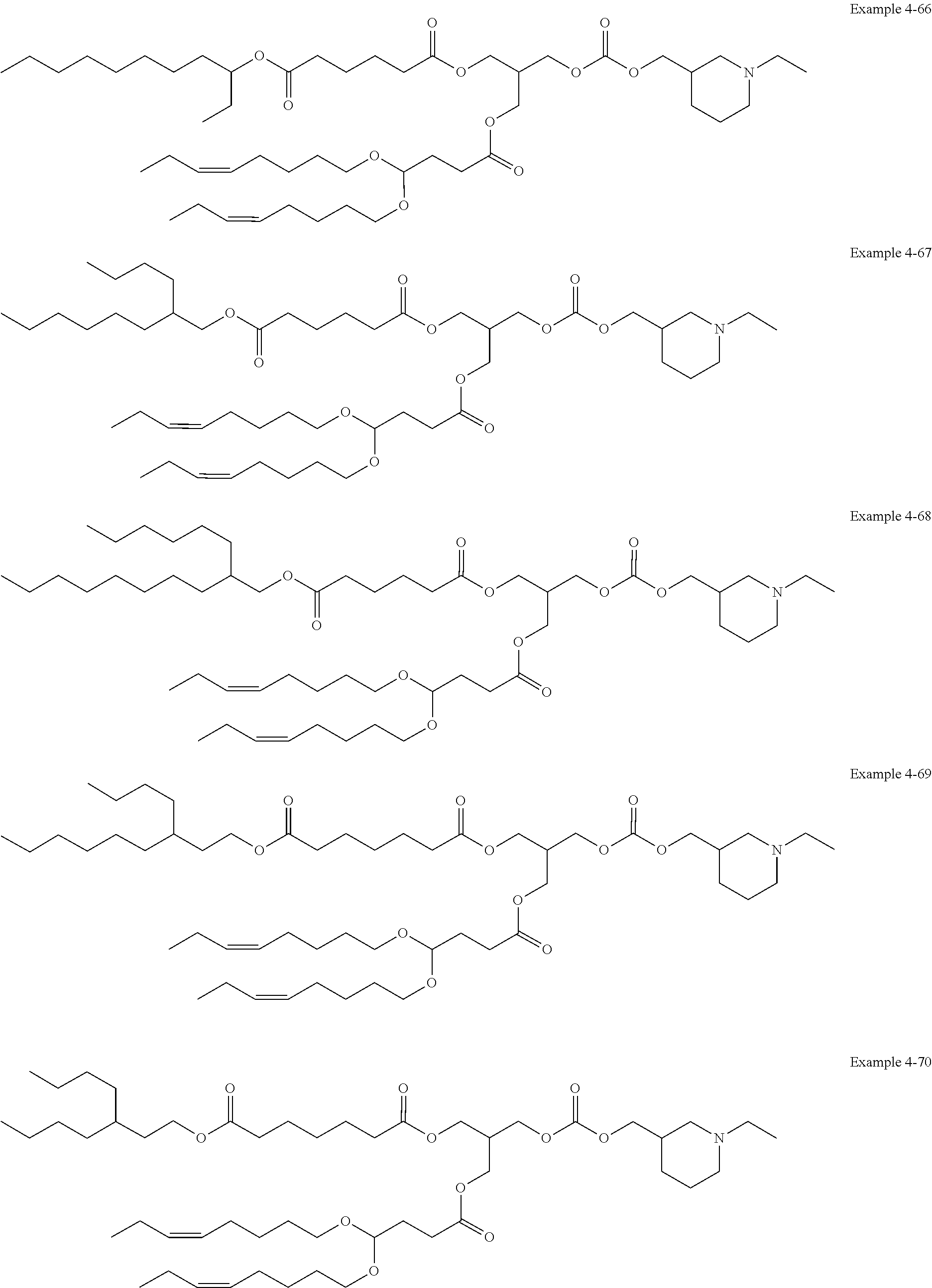

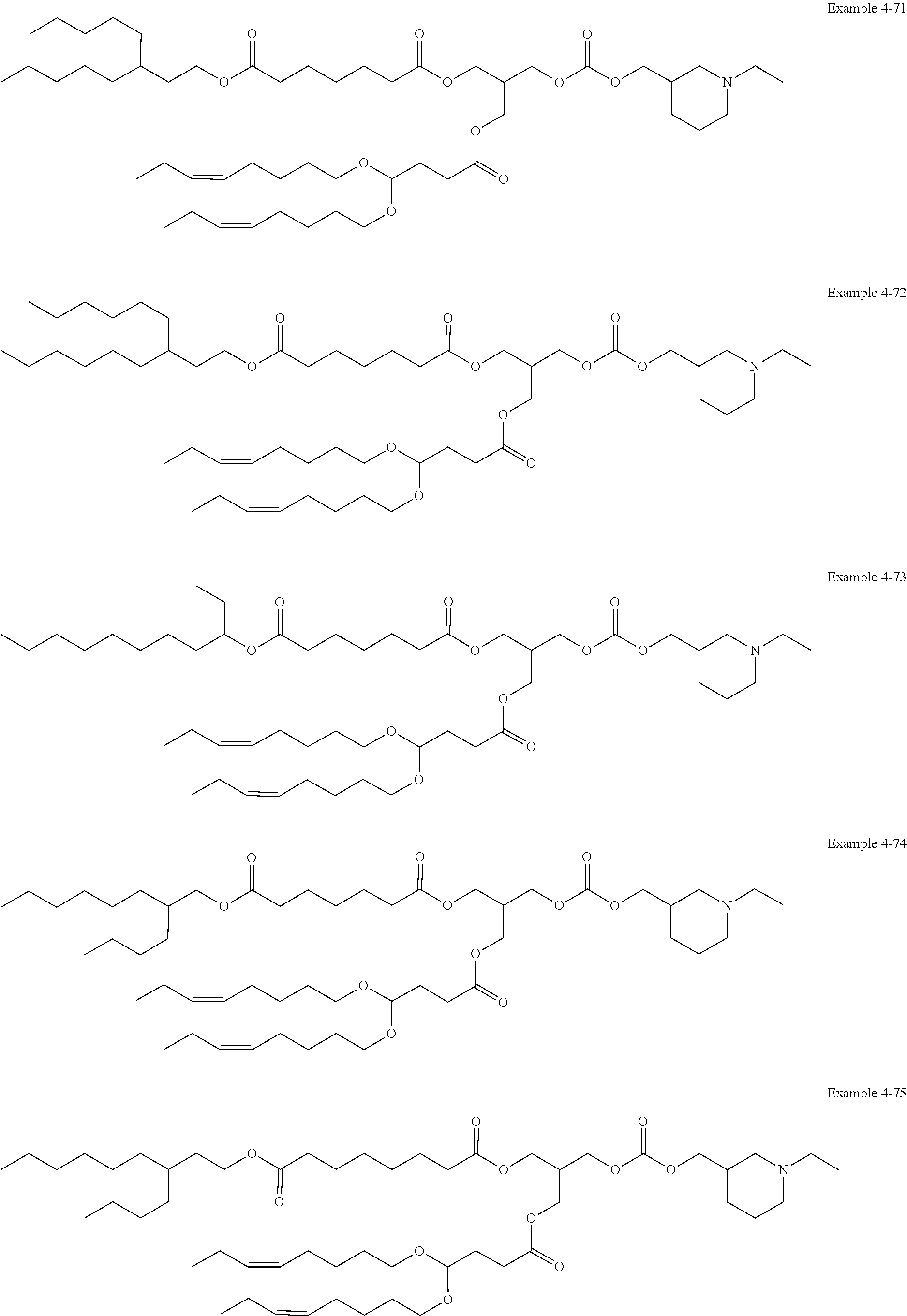

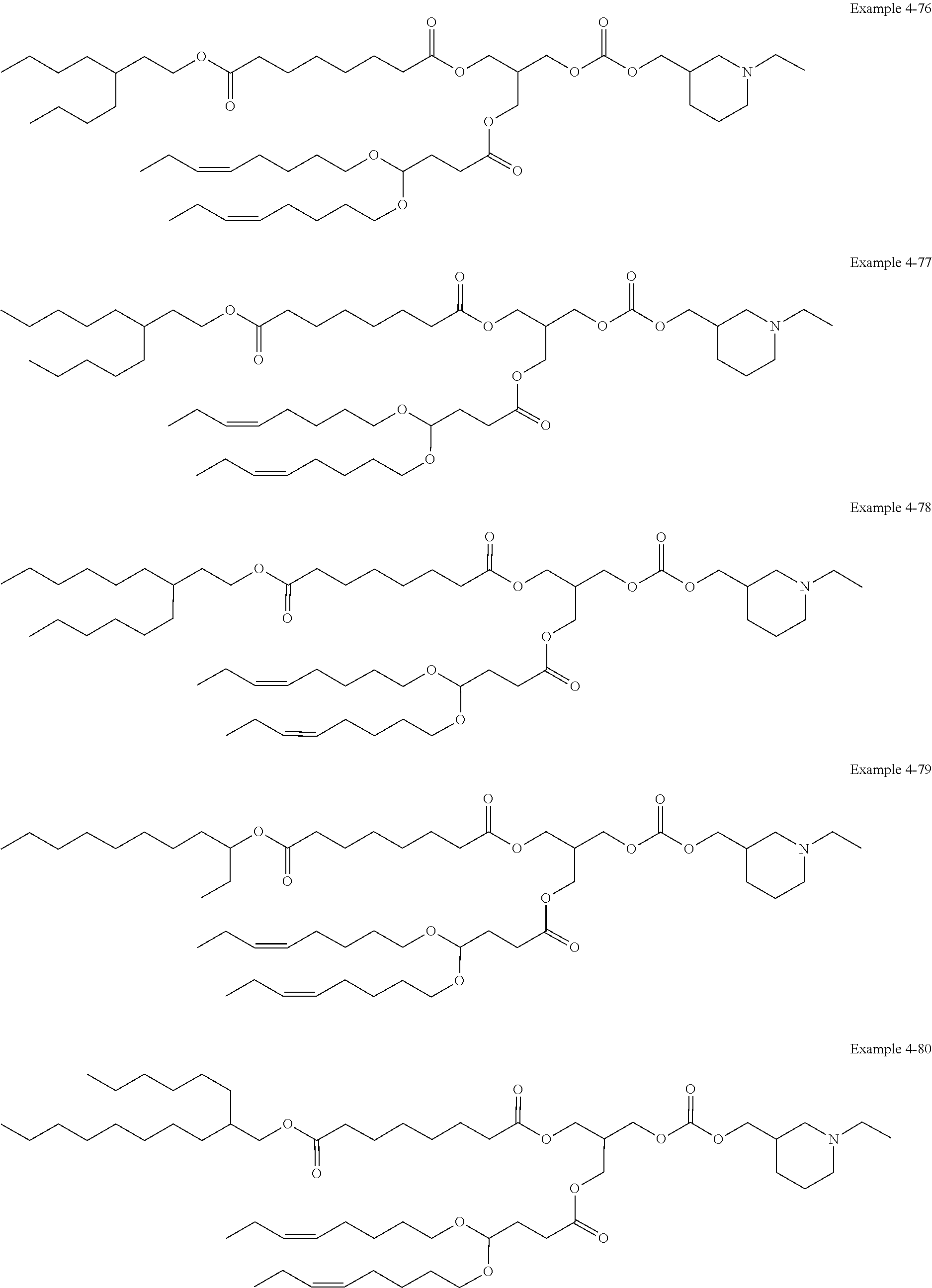

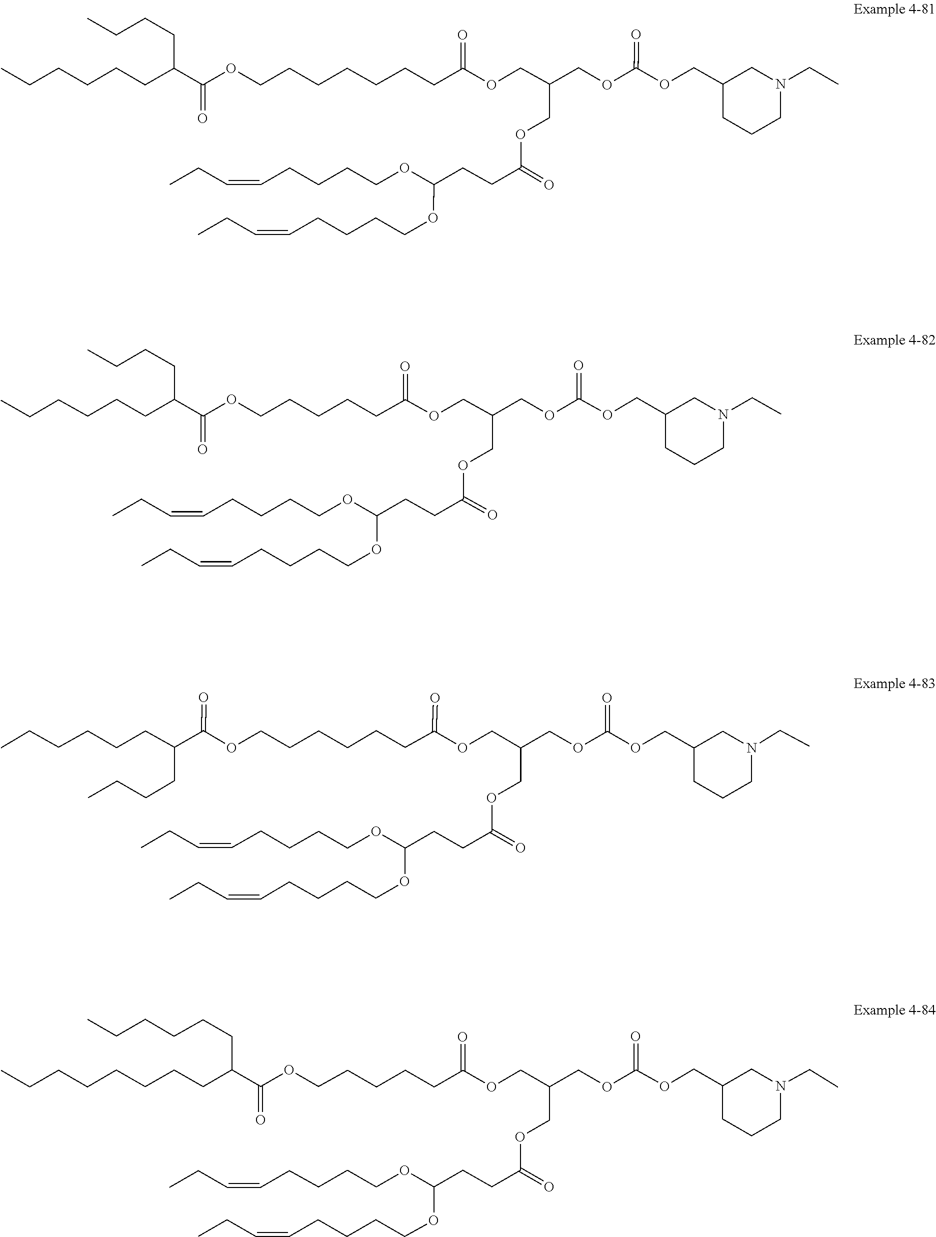

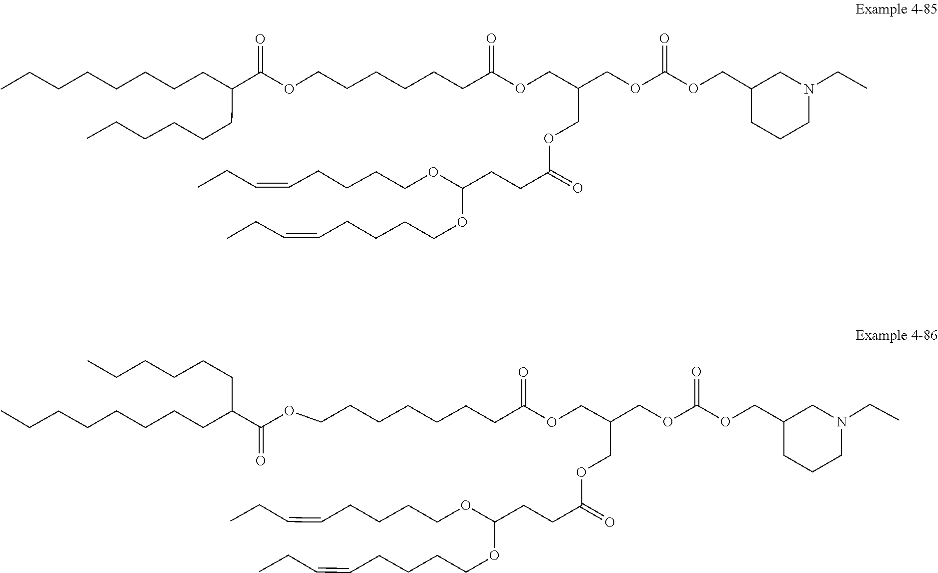

In some embodiments, an amino lipid is Example 4-62, or a pharmaceutically acceptable salt thereof. In some embodiments, an amino lipid is Example 4-63, or a pharmaceutically acceptable salt thereof. In some embodiments, an amino lipid is Example 4-64, or a pharmaceutically acceptable salt thereof. In some embodiments, an amino lipid is Example 4-65, or a pharmaceutically acceptable salt thereof. In some embodiments, an amino lipid is Example 4-66, or a pharmaceutically acceptable salt thereof. In some embodiments, an amino lipid is Example 4-67, or a pharmaceutically acceptable salt thereof. In some embodiments, an amino lipid is Example 4-68, or a pharmaceutically acceptable salt thereof. In some embodiments, an amino lipid is Example 4-69, or a pharmaceutically acceptable salt thereof. In some embodiments, an amino lipid is Example 4-70, or a pharmaceutically acceptable salt thereof. In some embodiments, an amino lipid is Example 4-71, or a pharmaceutically acceptable salt thereof. In some embodiments, an amino lipid is Example 4-72, or a pharmaceutically acceptable salt thereof. In some embodiments, an amino lipid is Example 4-73, or a pharmaceutically acceptable salt thereof. In some embodiments, an amino lipid is Example 4-74, or a pharmaceutically acceptable salt thereof. In some embodiments, an amino lipid is Example 4-75, or a pharmaceutically acceptable salt thereof. In some embodiments, an amino lipid is Example 4-76, or a pharmaceutically acceptable salt thereof. In some embodiments, an amino lipid is Example 4-77, or a pharmaceutically acceptable salt thereof. In some embodiments, an amino lipid is Example 4-78, or a pharmaceutically acceptable salt thereof. In some embodiments, an amino lipid is Example 4-79, or a pharmaceutically acceptable salt thereof. In some embodiments, an amino lipid is Example 4-80, or a pharmaceutically acceptable salt thereof. In some embodiments, an amino lipid is Example 4-81, or a pharmaceutically acceptable salt thereof. In some embodiments, an amino lipid is Example 4-82, or a pharmaceutically acceptable salt thereof. In some embodiments, an amino lipid is Example 4-83, or a pharmaceutically acceptable salt thereof. In some embodiments, an amino lipid is Example 4-84, or a pharmaceutically acceptable salt thereof. In some embodiments, an amino lipid is Example 4-85, or a pharmaceutically acceptable salt thereof. In some embodiments, an amino lipid is Example 4-86, or a pharmaceutically acceptable salt thereof.

### LNP Compositions Comprising Different Amino Lipids

In some embodiments, the LNP comprises a plurality of amino lipids having different formulas. For example, the LNP composition can comprise 2, 3, 4, 5, 6.7, 8, 9. 10, or more amino lipids. For another example, the LNP composition can comprise at least 2, at least 3, at least 4, at least 5, at least 6, at least 7, at least 9, at least 10, or at least 20 amino lipids. For yet another example, the LNP composition can comprise at most 2, at most 3, at most 4, at most 5, at most 6, at most 7, at most 9, at most 10, at most 20, or at most 30 amino lipids.

In some embodiments, the LNP composition comprises a first amino lipid. In some embodiments, the LNP composition comprises a first amino lipid and a second amino lipid. In some embodiments, the LNP composition comprises a first amino lipid, a second amino lipid, and a third amino lipid. In some embodiments, the LNP composition comprises a first amino lipid, a second amino lipid, a third amino lipid, and a fourth amino lipid. In some embodiments, the LNP composition does not comprise a fourth amino lipid. In some embodiments, the LNP composition does not comprise a third amino lipid. In some embodiments, a molar ratio of the first amino lipid to the second amino lipid is from about 0.1 to about 10. In some embodiments, a molar ratio of the first amino lipid to the second amino lipid is from about 0.20 to about 5. In some embodiments, a molar ratio of the first amino lipid to the second amino lipid is from about 0.25 to about 4. In some embodiments, a molar ratio of the first amino lipid to the second amino lipid is about 0.25, about 0.33, about 0.5, about 1, about 2, about 3, or about 4.

In some embodiments, a molar ratio of the first amino lipid:the second amino lipid:the third amino lipid is about 4:1:1. In some embodiments, a molar ratio of the first amino lipid:the second amino lipid:the third amino lipid is about 1:1:1. In some embodiments, a molar ratio of the first amino lipid:the second amino lipid:the third amino lipid is about 2:1:1. In some embodiments, a molar ratio of the first amino lipid:the second amino lipid:the third amino lipid is about 2:2:1. In some embodiments, a molar ratio of the first amino lipid:the second amino lipid:the third amino lipid is about 3:2:1. In some embodiments, a molar ratio of the first amino lipid:the second amino lipid:the third amino lipid is about 3:1:1. In some embodiments, a molar ratio of the first amino lipid:the second amino lipid:the third amino lipid is about 5:1:1. In some embodiments, a molar ratio of the first amino lipid:the second amino lipid:the third amino lipid is about 3:3:1. In some embodiments, a molar ratio of the first amino lipid:the second amino lipid:the third amino lipid is about 4:4:1.

### Additional Amino Lipid Embodiments

In some embodiments, the LNP composition comprises one or more amino lipids. In some embodiments, the one or more amino lipids comprise from about 40 mol % to about 65 mol % of the total lipid present in the particle. In some embodiments, the one or more amino lipids comprise about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, or about 65 mol % of the total lipid present in the particle. In some embodiments, the first amino lipid comprises from about 1 mol % to about 99 mol % of the total amino lipids present in the particle. In some embodiments, the first amino lipid comprises from about 16.7 mol % to about 66.7 mol % of the total amino lipids present in the particle. In some embodiments, the first amino lipid comprises from about 20 mol % to about 60 mol % of the total amino lipids present in the particle.

In some embodiments, the amino lipid is an ionizable lipid. An ionizable lipid can comprise one or more ionizable nitrogen atoms. In some embodiments, at least one of the one or more ionizable nitrogen atoms is positively charged. In some embodiments, at least 10 mol %, 20 mol %, 30 mol %, 40 mol %, 50 mol %, 60 mol %, 70 mol %, 80 mol %. 90 mol %, 95 mol %, or 99 mol % of the ionizable nitrogen atoms in the LNP composition are positively charged. In some embodiments, the amino lipid comprises a primary amine, a secondary amine, a tertiary amine, an imine, an amide, a guanidine moiety, a histidine residue, a lysine residue, an arginine residue, or any combination thereof. In some embodiments, the amino lipid comprises a primary amine, a secondary amine, a tertiary amine, a guanidine moiety, or any combination thereof. In some embodiments, the amino lipid comprises a tertiary amine.

In some embodiments, the amino lipid (e.g. an ionizable lipid) is a cationic lipid. In some embodiments, the cationic lipid is an ionizable lipid. In some embodiments, the amino lipid comprises one or more nitrogen atoms. In some embodiments, the amino lipid comprises one or more ionizable nitrogen atoms. Exemplary cationic and/or ionizable lipids include, but are not limited to, 3-(didodecylamino)-N1,N1,4-tri dodecyl-1-piperazineethane amine (KL10), N142-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleoyl-4-dimethylaminomethyl-[1,3]-dioxolane (DLin-K-DMA), heptatriaconta-6,9,28,31-tetraen-19-yl 4-(dimethylamino) butanoate (DLin-MC 3-DMA), 2,2-dilinoleoyl 1-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

In some embodiments, an amino lipid described herein can take the form of a salt, such as a pharmaceutically acceptable salt. All pharmaceutically acceptable salts of the amino lipid are encompassed by this disclosure. As used herein, the term “amino lipid” also includes its pharmaceutically acceptable salts, and its diastereomeric, enantiomeric, and epimeric forms.

In some embodiments, an amino lipid described herein, possesses one or more stereocenters and each stereocenter exists independently in either the R or S configuration. The lipids presented herein include all diastereomeric, enantiomeric, and epimeric forms as well as the appropriate mixtures thereof. The lipids provided herein include all cis. trans, syn, anti, entgegen (E), and zusammen (Z) isomers as well as the appropriate mixtures thereof. In certain embodiments, lipids described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In one aspect, stereoisomers are obtained by stereoselective synthesis.

In some embodiments, the lipids such as the amino lipids are substituted based on the structures disclosed herein. In some embodiments, the lipids such as the amino lipids are unsubstituted. In another embodiment, the lipids described herein are labeled isotopically (e.g., with a radioisotope) or by another other means, including, but not limited to, the use of chromophores or fluorescent moieties, bioluminescent labels, or chemiluminescent labels.

Lipids described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present lipids include isotopes of hydrogen, carbon, nitrogen, oxygen, sulfur, fluorine, and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled lipids described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the asymmetric carbon atom of the amino lipid is present in enantiomerically enriched form. In certain embodiments, the asymmetric carbon atom of the amino lipid has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the(S)- or (R)-configuration.

In some embodiments, the disclosed amino lipids can be converted to N-oxides. In some embodiments, N-oxides are formed by a treatment with an oxidizing agent (e.g., 3-chloroperoxybenzoic acid and/or hydrogen peroxides). Accordingly, disclosed herein are N-oxide compounds of the described amino lipids, when allowed by valency and structure, which can be designated as NO or N⁺—O⁻. In some embodiments, the nitrogen in the compounds of the disclosure can be converted to N-hydroxy or N-alkoxy. For example, N-hydroxy compounds can be prepared by oxidation of the parent amine by an oxidizing agent such as ra-CPBA. All shown nitrogen-containing compounds are also considered. Accordingly, also disclosed herein are N-hydroxy and N-alkoxy (e.g., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives of the described amino lipids.

In some embodiments, the one or more amino lipids comprise from about 40 mol % to about 65 mol % of the total lipid present in the particle.

### PEG-Lipids

In some embodiments, the described LNP composition includes one or more PEG-lipids. As used herein, a “PEG lipid” or “PEG-lipid” refers to a lipid comprising a polyethylene glycol component. Examples of suitable PEG-lipids also include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, the one or more PEG-lipids can comprise PEG-c-DOMG, PEG-DMG, PEG-DLPE, PEG-DMPE, PEG-DPPC, a PEG-DSPE lipid, or a combination thereof.

In some embodiments, PEG-lipid comprises from about 0.1 mol % to about 10 mol % of the total lipid present in the particle.

### Phospholipid

In some embodiments, the described LNP composition includes one or more phospholipids.

In some embodiments, the phospholipid comprises from about 5 mol % to about 15 mol % of the total lipid present in the particle.

### Cholesterol

In some embodiments, the LNP composition includes a cholesterol or a derivative thereof.

### GalNAc-Lipid

In some embodiments, the LNP composition includes a receptor targeting conjugate comprising a compound formula (V),

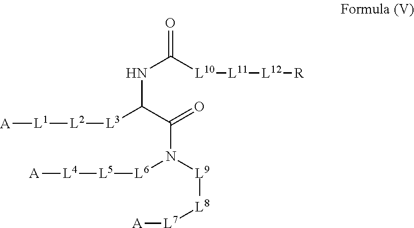

wherein,
a plurality of the A groups collectively comprise a receptor targeting ligand;
each L¹, L², L³, L⁴, L⁵, L⁶, L⁷, L⁸, L⁹, L¹⁰ and L¹² is independently substituted or unsubstituted C₁-C₁₂ alkylene, substituted or unsubstituted C₁-C₁₂ heteroalkylene, substituted or unsubstituted C₂-C₁₂ alkenylene, substituted or unsubstituted C₂-C₁₂ alkynylene, —(CH₂CH₂O)ₘ—, —(OCH₂CH₂)ₘ—, —O—, —S—, —S(═O)—, —S(═O)₂—, —S(═O)(═NR¹)—, —C(═O)—, —C(═N—OR¹)—, —C(═O)O—, —OC(═O)—, —C(═O)C(═O)—, —C(═O)NR¹—, —NR¹C(═O)—, —OC(═O)NR¹—, —NR¹C(═O)O—, —NR¹C(═O)NR¹—, —C(═O)NR¹C(═O)—, —S(═O)₂NR¹—, —NR¹S(═O)₂—, —NR¹—, or —N(OR¹)—;
L¹¹ is substituted or unsubstituted —(CH₂CH₂O)ₙ—, substituted or unsubstituted —(OCH₂CH₂)ₙ— or substituted or unsubstituted —(CH₂)ₙ—;
each R¹ is independently H or substituted or unsubstituted C₁-C₆alkyl;
R is a lipid, nucleic acid, amino acid, protein, or lipid nanoparticle;
m is an integer selected from 1 to 10; and
n is an integer selected from 1 to 200.

In some embodiments, each L¹, L⁴, and L⁷ is independently substituted or unsubstituted C₁-C₁₂ alkylene. In some embodiments, each L¹, L⁴, and L⁷ is independently substituted or unsubstituted C₂-C₆ alkylene. In some embodiments, each L¹, L⁴, and L⁷ is C₄ alkylene. In some embodiments, each L², L⁵, and L⁸ is independently —C(═O)NR¹—, —NR¹C(═O)—, —OC(═O)NR¹—, —NR¹C(═O)O—, —NR¹C(═O)NR¹—, or —C(═O)NR¹C(═O)—. In some embodiments, each L², L⁵, and L⁸ is independently —C(—O) NR¹— or —NR¹C(═O)—. In some embodiments, each L², L⁵, and L⁸ is —C(═O)NH—. In some embodiments, each L³, L⁶, and L⁹ is independently substituted or unsubstituted C₁-C₁₂ alkylene. In some embodiments, each L³ is substituted or unsubstituted C₂-C₆ alkylene. In some embodiments, L³ is C₄ alkylene. In some embodiments, each L⁶ and L⁹ is independently substituted or unsubstituted C₂-C₁₀ alkylene. In some embodiments, each L⁶ and L⁹ is independently substituted or unsubstituted C₂-C₆ alkylene. In some embodiments, each L⁶ and L⁹ is C₃ alkylene. In some embodiments, A binds to a lectin. In some embodiments, the lectin is an asialoglycoprotein receptor (ASGPR). In some embodiments, A is N-acetylgalactosamine (GalNAc) or

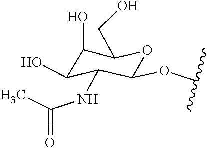 [SMILES: *OC1OC(CO)C(O)C(O)C1NC(C)=O]

or a derivative thereof. A is N-acetylgalactosamine (GalNAc)

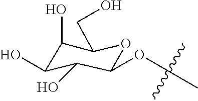 [SMILES: *OC1OC(CO)C(O)C(O)C1O]

or a derivative thereof.

In some embodiments, the receptor targeting conjugate comprises from about 0.001 mol % to about 20 mol % of the total lipid content present in the nanoparticle composition.

### Phosphate Charge Neutralizer

In some embodiments, the LNP described herein includes a phosphate charge neutralizer. In some embodiments, the phosphate charge neutralizer comprises arginine, asparagine, glutamine, lysine, histidine, cationic dendrimers, polyamines, or a combination thereof. In some embodiments, the phosphate charge neutralizer comprises one or more nitrogen atoms. In some embodiments, the phosphate charge neutralizer comprises a polyamine.

Suitable phosphate charge neutralizers to be used in LNP formulations, set forth below, for example include, but are not limited to, Spermidine and 1,3-propanediamine.

### Antioxidants

In some embodiments, the LNP described herein includes one or more antioxidants. In some embodiments, the one or more antioxidants function to reduce a degradation of the cationic lipids, the payload, or both. In some embodiments, the one or more antioxidants comprise a hydrophilic antioxidant. In some embodiments, the one or more antioxidants is a chelating agent such as ethylenediaminetetraacetic acid (EDTA) and citrate. In some embodiments, the one or more antioxidants comprise a lipophilic antioxidant. In some embodiments, the lipophilic antioxidant comprises a vitamin E isomer or a polyphenol. In some embodiments, the one or more antioxidants are present in the LNP composition at a concentration of at least 1 mM, at least 10 mM, at least 20 mM, at least 50 mM, or at least 100 mM. In some embodiments, the one or more antioxidants are present in the particle at a concentration of about 20 mM.

### Other Lipids

In some embodiments, the disclosed LNP compositions may comprise a helper lipid. In some embodiments, the disclosed LNP compositions comprise a neutral lipid. In some embodiments, the disclosed LNP compositions comprise a stealth lipid. In some embodiments, the disclosed LNP compositions comprises additional lipids. Neutral lipids can function to stabilize and improve processing of the LNPs.

“Helper lipids” can refer to lipids that enhance transfection (e.g., transfection of the nanoparticle (LNP) comprising the composition as provided herein, including the biologically active agent). The mechanism by which the helper lipid enhances transfection includes enhancing particle stability. In some embodiments, the helper lipid enhances membrane fusogenicity. Helper lipids can include steroids, sterols, and alkyl resorcinols. Helper lipids suitable for use in the present disclosure can include, but are not limited to, cholesterol, 5-heptadecylresorcinol, and cholesterol hemisuccinate. In some embodiments, the helper lipid is cholesterol. In some embodiments, the helper lipid may be cholesterol hemisuccinate.

“Stealth lipids” can refer to lipids that alter the length of time the nanoparticles can exist in vivo (e.g., in the blood). Stealth lipids can assist in the formulation process by, for example, reducing particle aggregation and controlling particle size. Stealth lipids used herein may modulate pharmacokinetic properties of the LNP. Stealth lipids suitable for use in a lipid composition of the disclosure can include, but are not limited to, stealth lipids having a hydrophilic head group linked to a lipid moiety. Stealth lipids suitable for use in a lipid composition of the present disclosure and information about the biochemistry of such lipids can be found in Romberg et al, Pharmaceutical Research, Vol. 25, No. 1, 2008, pg. 55-71 and I-Toekstra et al, Biochimica et Biophysica Acta 1660 (2004) 41-52. Additional suitable PEG lipids are disclosed, e.g., in WO 2006/007712.

In some embodiments, the stealth lipid is a PEG-lipid. In one embodiment, the hydrophilic head group of stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly N-(2-hydroxypropyl)methacrylamide]. Stealth lipids can comprise a lipid moiety. In some embodiments, the lipid moiety of the stealth lipid may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

The structures and properties of helper lipids, neutral lipids, stealth lipids, and/or other lipids are further described in WO2017173054A1, WO2019067999A1, US20180290965A1, US20180147298A1, US20160375134A1, U.S. Pat. Nos. 8,236,770, 8,021,686, 8,236,770B2, U.S. Pat. No. 7,371,404B2, U.S. Pat. No. 7,780,983B2, U.S. Pat. No. 7,858,117B2, US20180200186A1, US20070087045A1, WO2018119514A1, and WO2019067992A1, all of which are hereby incorporated by reference in their entirety.

### LNP Formulations

Particular formulation of a nanoparticle composition comprising one or more described lipids is described herein.

The described nanoparticle compositions are capable of delivering a therapeutic agent such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic agent included in a nanoparticle composition may also be selected based on the desired delivery target or targets. For example, a therapeutic agent may be selected for a particular indication, condition, disease, or disorder and/or for delivery to a particular cell, tissue, organ, or system or group thereof (e.g., localized, or specific delivery). In certain embodiments, a nanoparticle composition may include an mRNA encoding a polypeptide of interest capable of being translated within a cell to produce the polypeptide (e.g., base editor) of interest. Such a composition are capable of having specificity or affinity to a particular organ or cell type to facilitate drug substance delivery thereto, for example the liver or hepatocytes.

The amount of a therapeutic agent or drug substance (e.g., the mRNA that encodes for the base editor and the guide RNA) in an LNP composition may depend on the size, composition, desired target and/or application, or other properties of the nanoparticle composition. For example, the amount of an RNA comprised in a nanoparticle composition may depend on the size, sequence, and other characteristics of the RNA. The relative amounts of a therapeutic agent and other elements (e.g., lipids) in a nanoparticle composition may also vary. In some embodiments, the wt/wt ratio of the lipid component to a therapeutic agent in a nanoparticle composition may be from about 5:1 to about 60:1, such as about 5:1. 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic agent may be from about 10:1 to about 40:1. In certain embodiments, the wt/wt ratio is about 20:1. The amount of a therapeutic agent in a nanoparticle composition can be measured using absorption spectroscopy (e.g., ultraviolet-visible spectroscopy).

In some embodiments, an LNP formulation comprises one or more nucleic acids such as RNAs. In some embodiments, the one or more RNAs, lipids, and amounts thereof may be selected to provide a specific N/P ratio. The N/P ratio can be selected from about 1 to about 30. The N/P ratio can be selected from about 2 to about 12. In some embodiments, the N/P ratio is from about 0.1 to about 50. In some embodiments, the N/P ratio is from about 2 to about 8. In some embodiments, the N/P ratio is from about 2 to about 15, from about 2 to about 10, from about 2 to about 8, from about 2 to about 6, from about 3 to about 15, from about 3 to about 10, from about 3 to about 8, from about 3 to about 6, from about 4 to about 15, from about 4 to about 10, from about 4 to about 8, or from about 4 to about 6. In some embodiments, the N/P ratio is about 2, about 2.5, about 3, about 3.5, about 4, about 4.5, about 5, about 5.5, about 6, about 6.5, about 7, about 7.5, about 8, about 9, or about 10. In some embodiments, the N/P ratio is from about 4 to about 6. In some embodiments, the NIP ratio is about 4, about 4.5, about 5, about 5.5, or about 6.

As used herein, the “N/P ratio” is the molar ratio of ionizable (e.g., in the physiological pH range) nitrogen atoms in a lipid (or lipids) to phosphate groups in a nucleic acid molecular entity (or nucleic acid molecular entities), e.g., in a nanoparticle composition comprising a lipid component and an RNA. Ionizable nitrogen atoms can include, for example, nitrogen atoms that can be protonated at about pH 1, about pH 2, about pH 3, about pH 4, about pH 4.5, about pH 5, about pH 5.5, about pH 6, about pH 6.5, about pH 7, about pH 7.5, or about pH 8 or higher. The physiological pH range can include, for example, the pH range of different cellular compartments (such as organs, tissues, and cells) and bodily fluids (such as blood, CSF, gastric juice, milk, bile, saliva, tears, and urine). In certain specific embodiments, the physiological pH range refers to the pH range of blood in a mammal, for example, from about 7.35 to about 7.45. Similarly, for phosphate charge neutralizers that have one or more ionizable nitrogen atoms, the N/P ratio can refer to a molar ratio of ionizable nitrogen atoms in the phosphate charge neutralizer to the phosphate groups in a nucleic acid. In some embodiments, ionizable nitrogen atoms refer to those nitrogen atoms that are ionizable within a pH range between 5 and 14.

For the payload that does not contain a phosphate group, the N/P ratio can refer to a molar ratio of ionizable nitrogen atoms in a lipid to the total negative charge in the payload. For example, the N/P ratio of an LNP composition can refer to a molar ratio of the total ionizable nitrogen atoms in the LNP composition to the total negative charge in the payload that is present in the composition.

In some embodiments, the LNPs are formed with an average encapsulation efficiency ranging from about 50% to about 70%, from about 70% to about 90%, or from about 90% to about 100%. In some embodiments, the LNPs are formed with an average encapsulation efficiency ranging from about 75% to about 98%.

In another aspect, provided herein is a lipid nanoparticle (LNP) comprising the composition as provided herein. As used herein, a “lipid nanoparticle (LNP) composition” or a “nanoparticle composition” is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. In some embodiments, a LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 40-100 nm, 50-100 nm. 50-90 nm, 50-80 nm, 50-70 nm, 55-95 nm, 55-80 nm, 55-75 nm, 60-100 nm, 60-90 nm, 60-80 nm, 60-70 nm, 25-100 nm, 25-80 nm, or 40-80 nm.

In some embodiments, an LNP may be made from cationic, anionic, or neutral lipids. In some embodiments, an LNP may comprise neutral lipids, such as the fusogenic phospholipid 1,2-dioleoyl-sn-glycero-3-phosphoethanolamine (DOPE) or the membrane component cholesterol, as helper lipids to enhance transfection activity and nanoparticle stability. In some embodiments, an LNP may comprise hydrophobic lipids, hydrophilic lipids, or both hydrophobic and hydrophilic lipids. Any lipid or combination of lipids that are known in the art can be used to produce an LNP. Examples of lipids used to produce LNPs include, but are not limited to DOTMA (N [1-(2,3-dioleyloxy)propyl]-N,N,N-trimethylammonium chloride), DOSPA (N,N-dimethyl-N-([2-sperminecarboxamido]ethyl)-2,3-bis(dioleyloxy)-1-propaniminium pentahydrochloride), DOTAP (1,2-Dioleoyl-3-trimethylammonium propane), DMRIE (N-(2-hydroxyethyl)-N,N-dimethyl-2,3-bis(tetradecyloxy-1-propanaminiumbromide), DC-cholesterol (3β-[N—(N′,N′-dimethylaminoethane)-carbamoyl]cholesterol), DOTAP-cholesterol, GAP-DMORIE-DPyPE, and GL67A-DOPE-DMPE (1,2-Bis(dimethylphosphino) ethane)-polyethylene glycol (PEG). Examples of cationic lipids include, but are not limited to, 98N12-5, C12-200, DLin-KC2-DMA (KC2), DLin-MC3-DMA (MC3), XTC, MD1, and 7C1. Examples of neutral lipids include, but are not limited to, DPSC, DPPC (Dipalmitoylphosphatidylcholine), POPC (1-palmitoyl-2-oleoyl-sn-glycero-3-phosphocholine), DOPE, and SM (sphingomyelin). Examples of PEG-modified lipids include, but are not limited to, PEG-DMG (Dimyristoyl glycerol), PEG-CerC14, and PEG-CerC20. In some embodiments, the lipids may be combined in any number of molar ratios to produce an LNP. In some embodiments, the polynucleotide may be combined with lipid(s) in a wide range of molar ratios to produce an LNP.

The definitions of terms in the following eight paragraphs apply only the compounds of Formula (I), (Ia), and (V) above.

The term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioallcyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Exemplary substituents include amino, alkylamino, and the like.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances, one or more substituents having a double bond (e.g., “oxo” or “—O”) as the point of attachment may be described, shown, or listed herein within a substituent group, wherein the structure may only show a single bond as the point of attachment to the core structure of Formula (I). A person of ordinary skill in the art would understand that, while only a single bond is shown, a double bond is intended for those substituents.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CI-12-, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkenyl” refers to a type of alkyl group in which at least one carbon-carbon double bond is present. In one embodiment, an alkenyl group has the formula —C(R)═CR², wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “cycloalkyl” refers to a monocyclic or polycyclic non-aromatic radical, wherein each of the atoms forming the ring (i.e., skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one. spiro[2.2]pentyl, norbornyl and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

The term “heterocycle” or “heterocyclic” refers to heteroaromatic rings (also known as heteroaryls) and heterocycloalkyls (also known as heteroalicyclic groups) that includes at least one heteroatom selected from nitrogen, oxygen, and sulfur, wherein each heterocyclic group has from 3 to 12 atoms in its ring system, and with the proviso that any ring does not contain two adjacent O or S atoms. A “heterocyclyl” is a univalent group formed by removing a hydrogen atom from any ring atoms of a heterocyclic compound. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzofused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl. thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4Hpyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl 1,3-azabicyclo[4.1.0]heptanyl, 3 h-indolyl, indolin-2-onyl, isoindolin-1-onyl, isoindoline-1,3-dionyl, 3,4-dihydroisoquinolin-1 (2H)-onyl, 3,4-dihydroquinolin-2 (1H)-onyl, isoindoline-1,3-dithionyl, benzo[d]oxazol-2 (3H)-onyl, 1H-benzo[d]imidazol-2 (3H)-onyl, benzo[d]thiazol-2 (3H)-onyl, and quinolizinyl. Examples of aromatic heterocyclic groups are pyridinyl, imidazolyl, pyrimidinyl, pyrazolyl, triazolyl, pyrazinyl, tetrazolyl, futyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or Clinked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon, or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxothiomorpholinyl, 1,1-dioxo-thiomorpholinyl. The term heterocycloalkyl also includes all ring forms of carbohydrates, including but not limited to monosaccharides, disaccharides, and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted. As used herein, the term “teterocycloalkylene” can refer to a divalent heterocycloalkyl group.

The term “heteroaryl” refers to an aryl group that includes one or more ring heteroatoms selected from nitrogen, oxygen, and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

The definitions of terms in the following twenty-five paragraphs apply only the compounds of Formula A′, III-a, and III-a-I, 1, VI, and VIA above

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 carbon atoms. In some embodiments, aliphatic groups contain 1-4 carbon atoms. In some embodiments, aliphatic groups contain 1-3 carbon atoms, and in some embodiments, aliphatic groups contain 1-2 carbon atoms. In some embodiments, “carbocyclic” (or “cycloaliphatic” or “carbocycle” or “cycloalkyl”) refers to an optionally substituted monocyclic C₃-C₈ hydrocarbon, or an optionally substituted C₆-C₁₂ bicyclic hydrocarbon, that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “alkenyl” refers to an alkyl group, as defined herein, having one or more double bonds. In some embodiments, the term “alkenyl”, used alone or as part of a larger moiety, refers to an optionally substituted straight or branched hydrocarbon chain having at least one double bond and having (unless otherwise specified) 2-20, 2-18, 2-16, 2-14, 2-12, 2-10, 2-8, 2-6, 2-4, or 2-3 carbon atoms (e.g., C₂₋₂₀, C₂₋₁₈, C₂₋₁₆, C₂₋₁₄, C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

The term “alkylenyl” or “alkylene” refers to a bivalent alkyl group (i.e., a bivalent saturated hydrocarbon chain) that is a straight-chain (i.e., unbranched) or branched, substituted or unsubstituted. Any of the above mentioned monovalent alkyl groups may be an alkylenyl by abstraction of a second hydrogen atom from the alkyl. In some embodiments, an “alkylenyl” is a polymethylene group, i.e., —(CH₂)ₙ—, wherein n is a positive integer, preferably from 1 to 10, from 1 to 9, from 1 to 8, from 1 to 7, from 1 to 6, from 1 to 5, from 1 to 4, from 1 to 3, from 1 to 2, from 2 to 5, or from 4 to 8. A substituted alkylenyl is a polymethylene group in which one or more methylene hydrogen atoms are replaced with a substituent. Suitable substituents include those described below for a substituted aliphatic group.

As used herein, the term “alkynyl” refers to an alkyl group, as defined herein, having one or more triple bonds. In some embodiments, the term “alkynyl”, used alone or as part of a larger moiety, refers to an optionally substituted straight or branched chain hydrocarbon group having at least one triple bond and having (unless otherwise specified) 2-20, 2-18, 2-16, 2-14, 2-12, 2-10, 2-8, 2-6, 2-4, or 2-3 carbon atoms (e.g., C₂₋₂₀, C₂₋₁₈, C₂₋₁₆, C₂₋₁₄, C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

The term “aryl” refers to monocyclic and bicyclic ring systems having a total of six to fourteen ring members (e.g., C₆₋₁₄), wherein at least one ring in the system is aromatic and wherein each ring in the system contains three to seven ring members. The term “aryl” may be used interchangeably with the term “aryl ring”. In some embodiments, “aryl” refers to an aromatic ring system which includes, but is not limited to, phenyl, naphthyl, anthracyl and the like, which may bear one or more substituents. Unless otherwise specified, “aryl” groups are hydrocarbons.

As used herein, the term “bivalent” refers to a chemical moiety with two points of attachment. For example, a “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include but are not limited to:

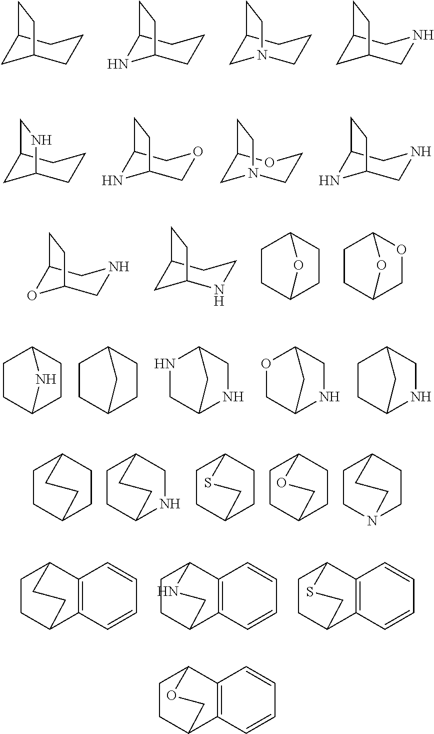

The terms “carbocyclyl,” “carbocycle,” and “carbocyclic ring” as used herein, refer to saturated or partially unsaturated cyclic aliphatic monocyclic, bicyclic, or polycyclic ring systems, as described herein, having from 3 to 14 members, wherein the aliphatic ring system is optionally substituted as described herein. Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₆-C₁₂ bicyclic hydrocarbon that is completely saturated or that contains one or more units of unsaturation, but which is not aromatic, that has a single point of attachment to the rest of the molecule. The term “cycloalkyl” refers to an optionally substituted saturated ring system of about 3 to about 10 ring carbon atoms. In some embodiments, cycloalkyl groups have 3-6 carbons. Exemplary monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

The term “haloaliphatic” refers to an aliphatic group substituted by one or more halogen atoms (e.g., one, two, three, four, five, six, or seven halo, such as fluoro, iodo, bromo, or chloro). In some embodiments, haloaliphatic groups contain 1-7 halogen atoms. In some embodiments, haloaliphatic groups contain 1-5 halogen atoms. In some embodiments, haloaliphatic groups contain 1-3 halogen atoms.

The term “haloalkyl” refers to an alkyl group substituted by one or more halogen atoms (e.g., one, two, three, four, five, six, or seven halo, such as fluoro, iodo, bromo, or chloro). In some embodiments, haloalkyl groups contain 1-7 halogen atoms. In some embodiments, haloalkyl groups contain 1-5 halogen atoms. In some embodiments, haloalkyl groups contain 1-3 halogen atoms.

The term “heteroalkylenyl” or “heteroalkylene”, as used herein, denotes an optionally substituted straight-chain (i.e., unbranched), or branched bivalent alkyl group (i.e., bivalent saturated hydrocarbon chain) having, in addition to carbon atoms, from one to five heteroatoms. The term “heteroatom” is described below. In some embodiments, heteroalkylenyl groups contain 2-10 carbon atoms wherein 1-3 carbon atoms are optionally and independently replaced with heteroatoms selected from oxygen, nitrogen, and sulfur. In some embodiments, heteroalkylenyl groups contain 2-8 carbon atoms wherein 1-3 carbon atoms are optionally and independently replaced with heteroatoms selected from oxygen, nitrogen, and sulfur. In some embodiments, heteroalkylenyl groups contain 4-8 carbon atoms, wherein 1-3 carbon atoms are optionally and independently replaced with heteroatoms selected from oxygen, nitrogen, and sulfur. In some embodiments, heteroalkylenyl groups contain 2-5 carbon atoms, wherein 1-2 carbon atoms are optionally and independently replaced with heteroatoms selected from oxygen, nitrogen, and sulfur. In yet other embodiments, heteroalkylenyl groups contain 1-3 carbon atoms, wherein 1 carbon atom is optionally and independently replaced with a heteroatom selected from oxygen, nitrogen, and sulfur. Suitable heteroalkylenyl groups include, but are not limited to —CH₂O—, —(CH₂)₂O—, —CH₂OCH₂—, —O(CH₂)₂—, —(CH₂)₃O—, —(CH₂)₂OCH₂—, —CH₂O(CH₂)₂—, —O(CH₂)₃—, —(CH₂)₄O—, —(CH₂)₃OCH₂—, —CH₂O(CH₂)₃—, —(CH₂)₂O(CH₂)₂—, —O(CH₂)₄—. Unless otherwise specified, Cₓ heteroalkylenyl refers to heteroalkylenyl having x number of carbon atoms prior to replacement with heteroatoms.

The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3 (4H)-one, and benzoisoxazolyl. The term “heteroaryl” may be used interchangeably with the terms “heteroaryl ring”, “heteroaryl group”, or “heteroaromatic”, any of which terms include rings that are optionally substituted.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The terms “heterocycle”, “heterocyclyl”, “heterocyclic radical”, and “heterocyclic ring” are used interchangeably herein, and refer to a stable 3- to 8-membered monocyclic, a 7- to 12-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, tetrahydropyranyl, dioxanyl, dioxolanyl, diazepinyl, oxazepinyl, thiazepinyl, morpholinyl, thiamorpholinyl, and

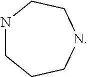

A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

As used herein, the term “linker” is used to refer to that portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element “L” has an overall structure of the general form S1-L′-S2, wherein S1 and S2 may be the same or different and represent two domains associated with one another by the linker. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger, P., et al. (1993) Proc. Natl. Acad. Sci. USA 90:6444-6448; Poljak, R. J., et al. (1994) Structure 2:1 121-1123).

The term “sterolyl,” as used herein, refers to a 17-membered fused polycyclic ring moiety that is either saturated or partially unsaturated and substituted with at least one hydroxyl group, and has a single point of attachment to the rest of the molecule at any substitutable carbon or oxygen atom. In some embodiments, a sterolyl group is a cholesterolyl group, or a variant or derivative thereof. In some embodiments, a cholesterolyl group is modified. In some embodiments, a cholesterolyl group is an oxidized cholesterolyl group (e.g., oxidized on the beta-ring structure or on the hydrocarbon tail structure). In some embodiments, a cholesterolyl group is an esterified cholesterolyl group. In some embodiments, a sterolyl group is a phytosterolyl group. Exemplary sterolyl groups include but are not limited to 25-hydroxycholesterolyl (25-OH), 20α-hydroxycholesterolyl (20α-OH), 27-hydroxycholesterolyl, 6-keto-5α-hydroxycholesterolyl, 7-ketocholesterolyl, 7β-hydroxycholesterolyl, 7α-hydroxycholesterolyl, 7β-25-dihydroxycholesterolyl, beta-sitosterolyl, stigmasterolyl, brassicasterolyl, and campesterolyl.

As described herein, compounds of this disclosure may be described as “substituted” or “optionally substituted”. That is, compounds may contain optionally substituted and/or substituted moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

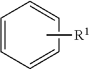

refers to at least

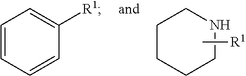

refers to at least

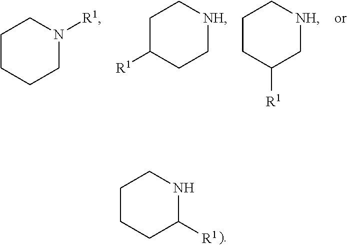

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents include halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O) (CH₂)₀₋₄SR^{∘}—, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —OSiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N3, —(CH₂)₀₋₂C(O)R^{●}, —(CH₂)₀₋₂C(O)OH, —(CH₂)₀₋₂C(O)OR^{●}, —(CH₂)₀₋₂C(O)NH₂, —(CH₂)₀₋₂C(O)NHR^{●}, —(CH₂)₀₋₂C(O)NR^{●}₂, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂)₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In some embodiments, suitable substituents on a substitutable nitrogen include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each Rt is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, amino lipids can contain at least one primary, secondary, or tertiary amine moiety that is protonatable (or ionizable) between pH range 4 and 14. In some embodiments, the amine moiety/moieties function as the hydrophilic headgroup of the amino lipids. When most of the amine moiety(ies) of an amino lipid (or amino lipids) in a nucleic acid-lipid nanoparticle formulation is protonated at physiological pH, then the nanoparticles can be termed as cationic lipid nanoparticle (cLNP). When most of the amine moiety(ies) of an amino lipid (or amino lipids) in a nucleic acid-lipid nanoparticle formulation is not protonated at physiological pH but can be protonated at acidic pH, endosomal pH for example, can be termed as ionizable lipid nanoparticle (iLNP). The amino lipids that constitute cLNPs can be generally called cationic amino lipids (cLipids). The amino lipids that constitute iLNPs can be called ionizable amino lipids (iLipids). The amino lipid can be an iLipid or a cLipid at physiological pH.

As used herein, LNP compositions or formulations are typically sized on the order of micrometers or smaller and may include a lipid bilayer. Nanoparticle compositions encompass lipid nanoparticles (LNPs), liposomes lipid vesicles), and lipoplexes nanoparticle composition a liposome having a lipid bilayer with a diameter of 500 nm or less. The LNPs described herein can have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. The LNPs described herein can be substantially non-toxic.

As used herein, a “phospholipid” can refer to a lipid that includes a phosphate moiety and one or more carbon chains, such as unsaturated fatty acid chains. A phospholipid may include one or more multiple (e.g., double or triple) bonds. In some embodiments, a phospholipid may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of an LNP to pass through the membrane, i.e., delivery of the one or more elements to a cell.

### Payload

The LNPs described herein can be designed to deliver a payload, such as one or more therapeutic agent(s) or drug substances(s) to a target cell or organ of interest. In some embodiments, a LNP described herein encloses one or more components of a base editor system as described herein. For example, a LNP may enclose one or more of a guide RNA, a nucleic acid encoding the guide RNA, a vector encoding the guide RNA, a base editor fusion protein, a nucleic acid encoding the base editor fusion protein, a programmable DNA binding domain, a nucleic acid encoding the programmable DNA binding domain, a deaminase, a nucleic acid encoding the deaminase, or all or any combination thereof. In some embodiments, the nucleic acid is a DNA. In some embodiments, the nucleic acid is a RNA, for example, a mRNA and/or a guide RNA. In some embodiments, the said nucleic acid(s) is/are chemically modified.

In some embodiments, the payload comprises one or more nucleic acid(s) (i.e., one or more nucleic acid molecular entities). In some embodiments, the nucleic acid is a single-stranded nucleic acid. In some embodiments, single-stranded nucleic acid is a DNA. In some embodiments, single-stranded nucleic acid is an RNA. In some embodiments, the nucleic acid is a double-stranded nucleic acid. In some embodiments, the double-stranded nucleic acid is a DNA. In some embodiments, the double-stranded nucleic acid is an RNA. In some embodiments, the double-stranded nucleic acid is a DNA-RNA hybrid. In some embodiments, the nucleic acid is a messenger RNA (mRNA), a microRNA, an asymmetrical interfering RNA (aiRNA), a small hairpin RNA (shRNA), an antisense oligonucleotide, or a Dicer-Substrate dsRNA. In some embodiments, the single-stranded nucleic acids form secondary structure, one or more stem-loops for example. In some other embodiments, the single stranded nucleic acids contain one or more stem-loops and single-stranded regions within the molecule.

### Kits

It is contemplated herein that the therapeutic agents or drug substances disclosed herein are part of a kit as described herein. Accordingly, one aspect of the disclosure relates to kits including the compositions comprising a single guide RNA as provided herein, the base editor system and complex as provided herein, the composition as provided herein, and/or the lipid nanoparticle formulations as provided herein for treating or preventing a condition. The kits can further include one or more additional therapeutic regimens or agents for treating or preventing a condition.

Also disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a composition as described herein, and optionally in addition with therapeutic regimens or agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In embodiments, a label is on or associated with the container. In one embodiment, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Dosing

The skilled artisan will appreciate that certain factors may influence the dosage and frequency of administration required to effectively treat a subject, including but not limited to the severity of the disease or disorder, previous treatments, the general characteristics of the subject including health, sex, weight and/or age of the subject, and other diseases present. Moreover, treatment of a subject with a therapeutically effective amount of the compositions can include a single treatment or can include a series of treatments. It will also be appreciated that the effective dosage of the composition of the disclosure used for treatment may increase or decrease over the course of a particular treatment. Changes in dosage may result and become apparent from the results of diagnostic assays as described herein. The therapeutically effective dosage will generally be dependent on the patient's status at the time of administration. The precise amount can be determined by routine experimentation but may ultimately lie with the judgment of the clinician, for example, by monitoring the patient for signs of disease and adjusting the treatment accordingly.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide may be appropriate.

The dosing regimen (including a composition disclosed herein) can vary over time. The particular dosage regimen, i.e., dose, timing, and repetition, will depend on the particular subject and that subject's medical history, as well as the properties of the polypeptide or the polynucleotide (such as the half-life of the polypeptide or the polynucleotide, and other considerations well known in the art).

The appropriate therapeutic dosage of a composition as described herein will depend on the specific agent (or compositions thereof) employed, the formulation and route of administration, the type and severity of the disease, whether the polypeptide or the polynucleotide is administered for preventive or therapeutic purposes, previous therapy, the subject's clinical history and response to the antagonist, and the discretion of the attending physician. Typically, the clinician will administer a polypeptide until a dosage is reached that achieves the desired result.

Administration of one or more compositions can be continuous or intermittent, depending, for example, upon the recipient's physiological condition, whether the purpose of the administration is therapeutic or prophylactic, and other factors known to skilled practitioners. The administration of a composition may be essentially continuous over a preselected period of time or may be in a series of spaced dose, e.g., either before, during, or after developing a disease.

The methods and compositions of the disclosure described herein including embodiments thereof can be administered with one or more additional therapeutic regimens or agents or treatments, which can be co-administered to the mammal. By “co-administering” is meant administering one or more additional therapeutic regimens or agents or treatments and the composition of the disclosure sufficiently close in time to enhance the effect of one or more additional therapeutic agents, or vice versa. In this regard, the composition of the disclosure described herein can be administered simultaneously with one or more additional therapeutic regimens or agents or treatments, at a different time, or on an entirely different therapeutic schedule (e.g., the first treatment can be daily, while the additional treatment is weekly). For example, in embodiments, the secondary therapeutic regimens or agents or treatments are administered simultaneously, prior to, or subsequent to the composition of the disclosure.

In embodiments, a polynucleotide encoding a base editor fusion protein and a guide RNA are administered to a subject. In embodiments, the polynucleotide encoding the base editor fusion protein is an mRNA. In embodiments, a LNP comprising such amounts of the polynucleotide encoding a base editor fusion protein and the guide RNA are administered to the subject. In embodiments, the subject is a primate. In embodiments, the subject is a non-human primate. In embodiments, the non-human primate is a cynomolgus monkey.

In embodiments, administration of the guide RNA and the polynucleotide encoding the base editor fusion protein to a non-human primate, such as a cynomolgus monkey results base alteration in 20% or more, 25% or more, 30% or more, 35% or more, 40% or more, 45% or more 50% or more, 55% or more, or 60% or more whole liver cells as measured by next generation sequencing. In embodiments, such base alteration percentages are achieved when the subject is administered a combined dose of the guide RNA and the polynucleotide encoding the base editor fusion protein. In embodiments, such doses are administered in LNPs. In embodiments, such administration results in reduced serum TTR levels.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Guides for Adenine Base Editing of the TTR Gene

With this example, gRNA sequences were identified that permit ABE8.8 (and other ABE variants containing Streptococcus pyogenes Cas9, such as ABE7.10, or another Cas protein that can use the NGG PAM) to either: 1) disrupt the start codon, or 2) disrupt splice sites, whether donors or acceptors, via A→G editing within its editing window (roughly positions 4 to 7 in the 20-nt protospacer region of DNA). Five sequences were identified throughout the human TTR gene (Table 1). gRNAs were synthesized matching each of the protospacer sequences and otherwise conforming to the standard 100-nt Streptococcus pyogenes CRISPR gRNA sequence, with each gRNA molecule having a minimal degree of chemical modifications (specified in Table 1). Each of the gRNAs was co-transfected with an equivalent amount of in vitro transcribed ABE8.8 mRNA (1:1 ratio by molecular weight) into primary human hepatocytes via MessengerMax reagent (Lipofectamine), using various dilutions (2500, 1250, 625 ng/RNA/mL) to assess for editing activity at different concentrations of test article.

TABLE 1
TTR Guides

| gRNA | | Protospacer | |
|---|---|---|---|
| ID | Species | (5′-3′) | gRNA sequence (5′-3′) |
| GA457 | Human | GCCATCCTG | gscscsAUCCUGCCAAGAAUGAGGUUUUAGAGCUAGAAA |
| | | CCAAGAATG | UAGCAAGUUAAAAUAAGGCUAGUCCGUUAUCAACUUGAA |
| | | AG (SEQ | AAAGUGGCACCGAGUCGGUGCUsususu (SEQ ID |
| | | ID NO : | NO: 11) |
| | | 24) | |
| GA519 | Cyno | GCCATCCTG | gscscsAUCCUGCCAAGAACGAGgUUUUAGagcuaGaaa |
| | | CCAAGAACG | uagcaaGUUaAaAuAaggcuaGUccGUUAucAAcuuGaa |
| | | AG (SEQ | aaagugGcaccgagucggugcuususus (SEQ ID |
| | | ID NO : | NO: 16) |
| | | 28) | |
| GA458 | Cyno | GCCATCCTG | gscscsAUCCUGCCAAGAACGAGGUUUUAGAGCUAGAAA |
| | | CCAAGAACG | UAGCAAGUUAAAAUAAGGCUAGUCCGUUAUCAACUUGAA |
| | | AG (SEQ | AAAGUGGCACCGAGUCGGUGCUsususu (SEQ ID |
| | | ID NO : | NO: 29) |
| | | 28) | |
| GA459 | Human | GCAACTTAC | gscsasACUUACCCAGAGGCAAAGUUUUAGAGCUAGAAA |
| | | CCAGAGGCA | UAGCAAGUUAAAAUAAGGCUAGUCCGUUAUCAACUUGAA |
| | | AA (SEQ | AAAGUGGCACCGAGUCGGUGCUsususu (SEQ ID |
| | | ID NO: | NO: 13) |
| | | 25) | |
| GA460 | Human/ | TATAGGAAA | usasusAGGAAAACCAGUGAGUCGUUUUAGAGCUAGAAA |
| | Cyno | ACCAGTGAG | UAGCAAGUUAAAAUAAGGCUAGUCCGUUAUCAACUUGAA |
| | | TC (SEQ | AAAGUGGCACCGAGUCGGUGCUsususu (SEQ ID |
| | | ID NO : | NO: 14) |
| | | 26) | |
| GA520 | Human/ | TATAGGAAA | usasusAGGAAAACCAGUGAGUCgUUUUAGagcuaGaaa |
| | Cyno | ACCAGTGAG | uagcaaGUUaAaAuAaggcuaGUccGUUAucAAcuuGaa |
| | | TC (SEQ | aaagugGcaccgagucggugcuususus (SEQ ID |
| | | ID NO : | NO: 17) |
| | | 26) | |
| GA461 | Human/ | TACTCACCT | usascsUCACCUCUGCAUGCUCAGUUUUAGAGCUAGAAA |
| | Cyno | CTGCATGCT | UAGCAAGUUAAAAUAAGGCUAGUCCGUUAUCAACUUGAA |
| | | CA (SEQ | AAAGUGGCACCGAGUCGGUGCUsususu (SEQ ID |
| | | ID NO: | NO: 15) |
| | | 27) | |
| GA521 | Human | GCCATCCTG | gscscsAUCCUGCCAAGAAUGAGmGUUUUAGmAmGmCmU |
| | | CCAAGAATG | mAGmAmAmAmUmAmGmCmAmAGUUmAAmAAmUAmAmGmG |
| | | AG (SEQ | mCmUmAGUmCmCGUUAmUmCAAmCmUmUGmAmAmAmAmA |
| | | ID NO : | mGmUmGGmCmAmCmCmGmAmGmUmCmGmGmUmGmCmU*m |
| | | 24) | U*mU*mU (SEQ ID NO: 11) |

Letters in the sequences:-A: adenosine; C: cytidine; G: guanosine; U: uridine; a: 2′-O-methyladenosine; c: 2′-O-methylcytidine; g: 2′-O-methylguanosine; u: 2′-O-methyluridine; and s: phosphorothioate (PS) backbone linkage.
Bold type in gRNA sequence denotes spacer sequence corresponding to Protospacer.
GA460 and GA520 have the same protospacer sequence but have different chemical modifications in the gRNA sequence.

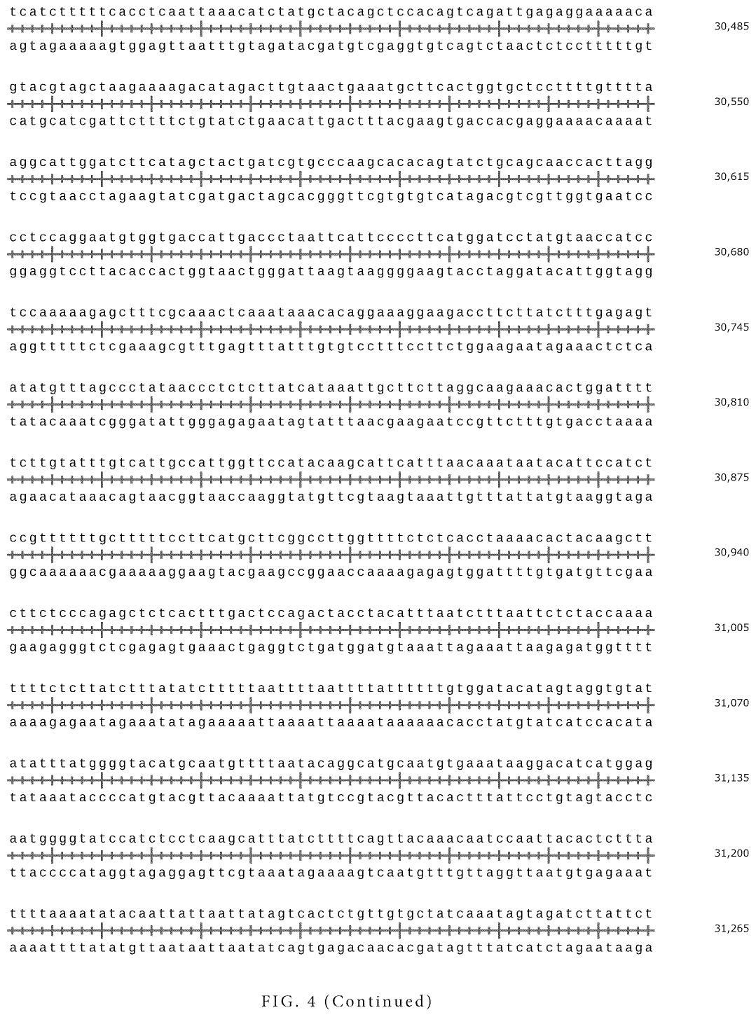

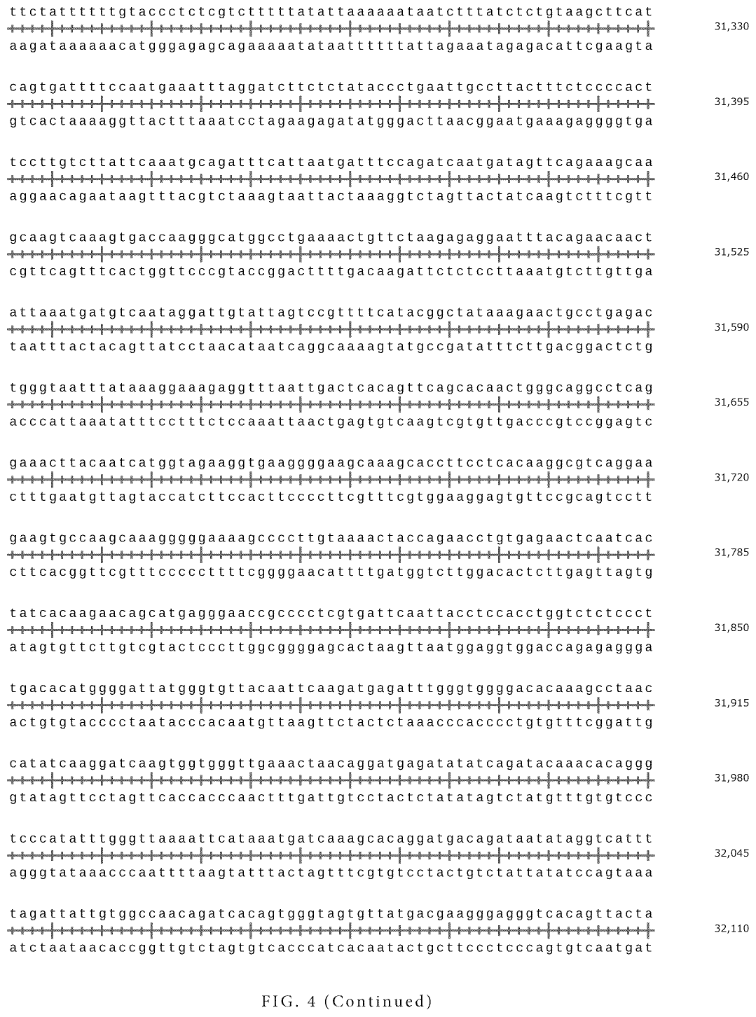

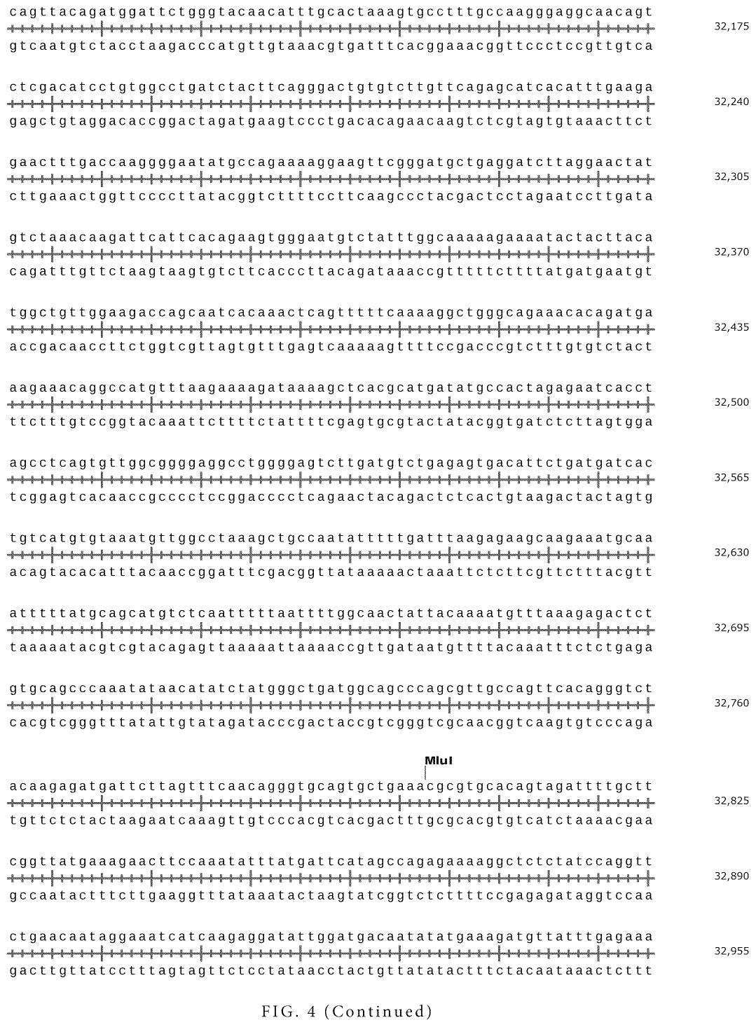

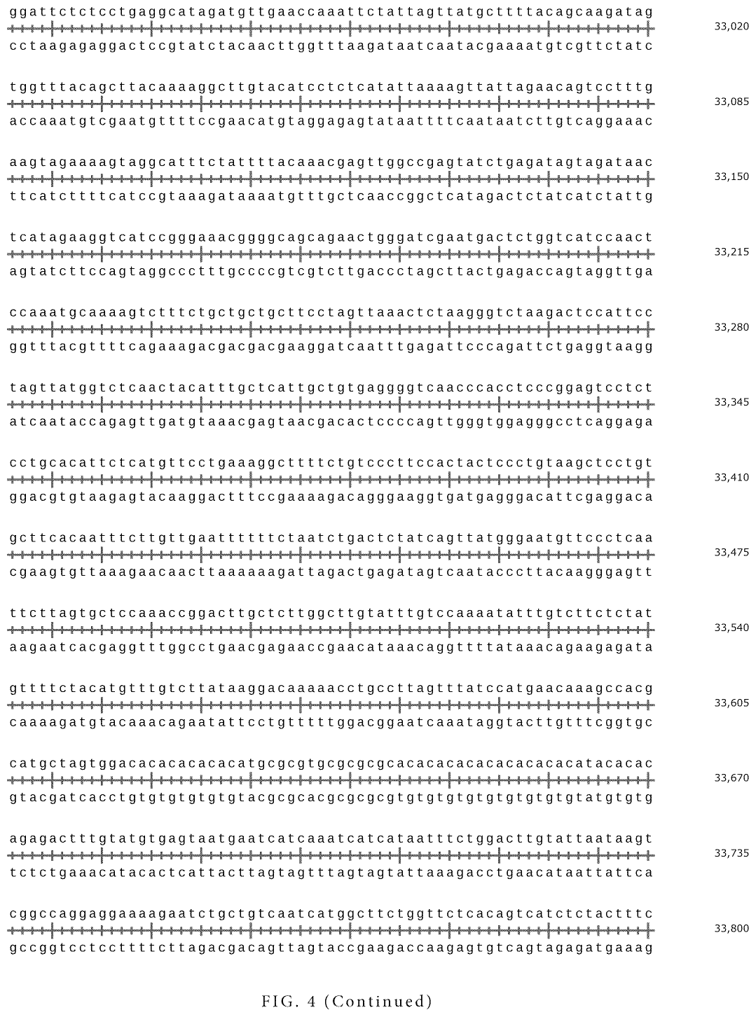

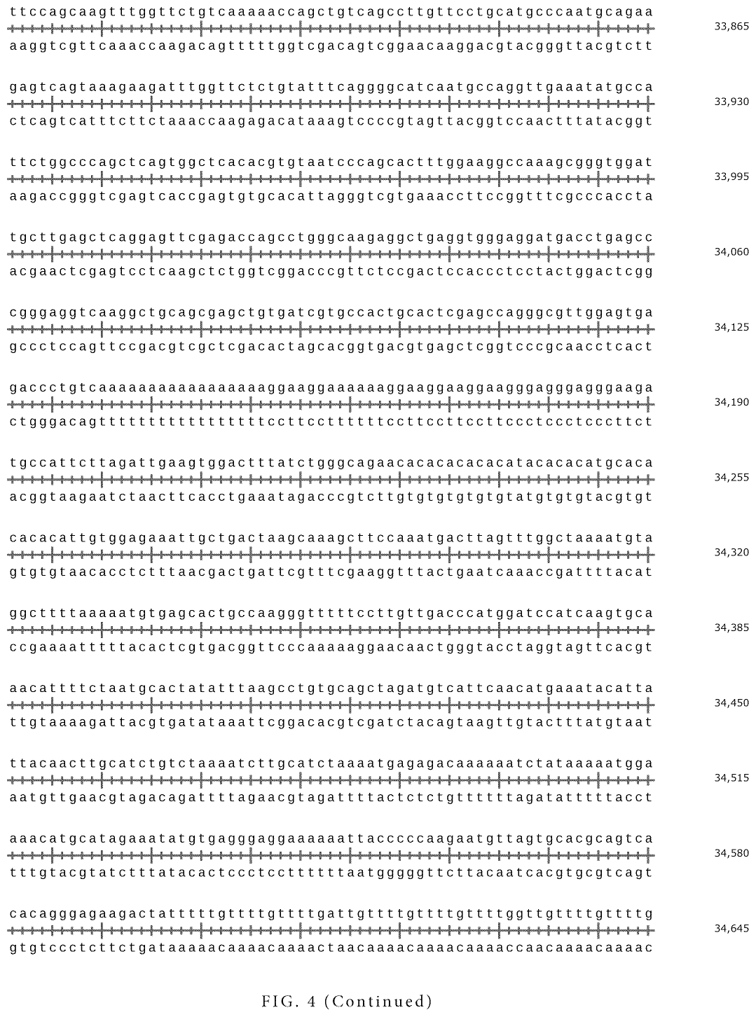

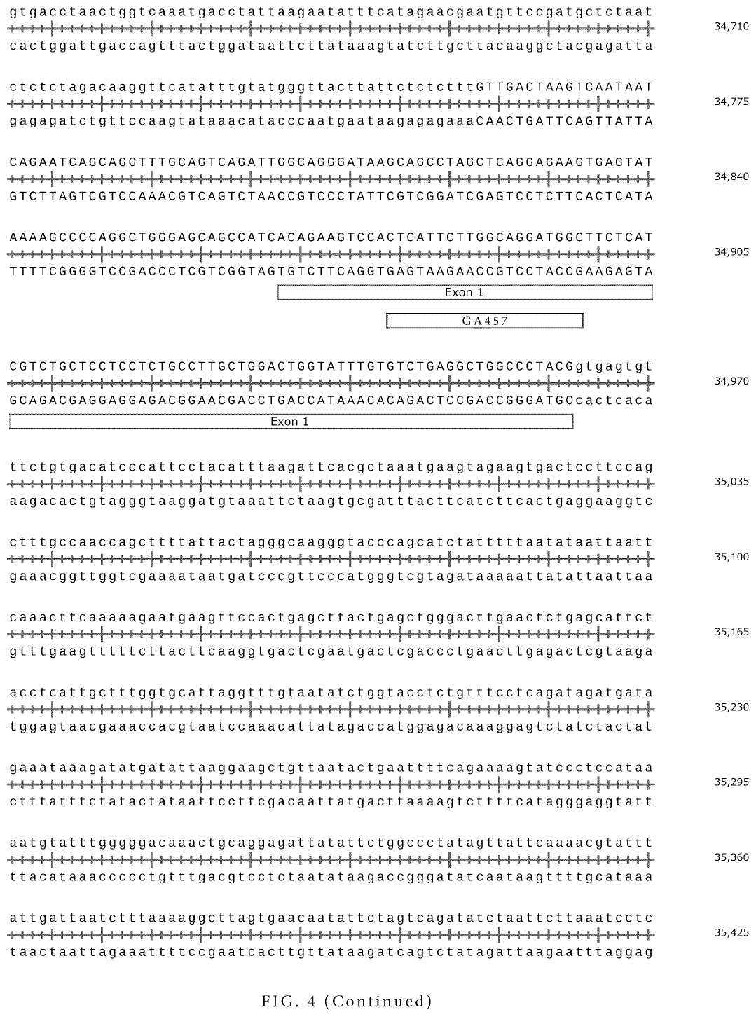

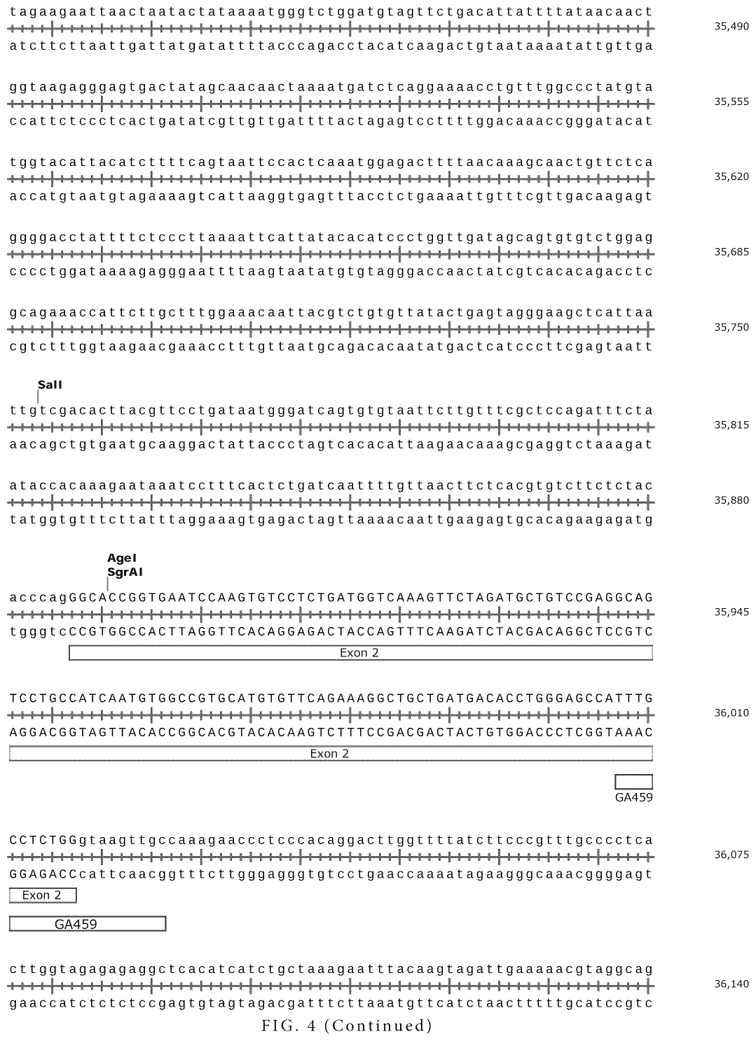

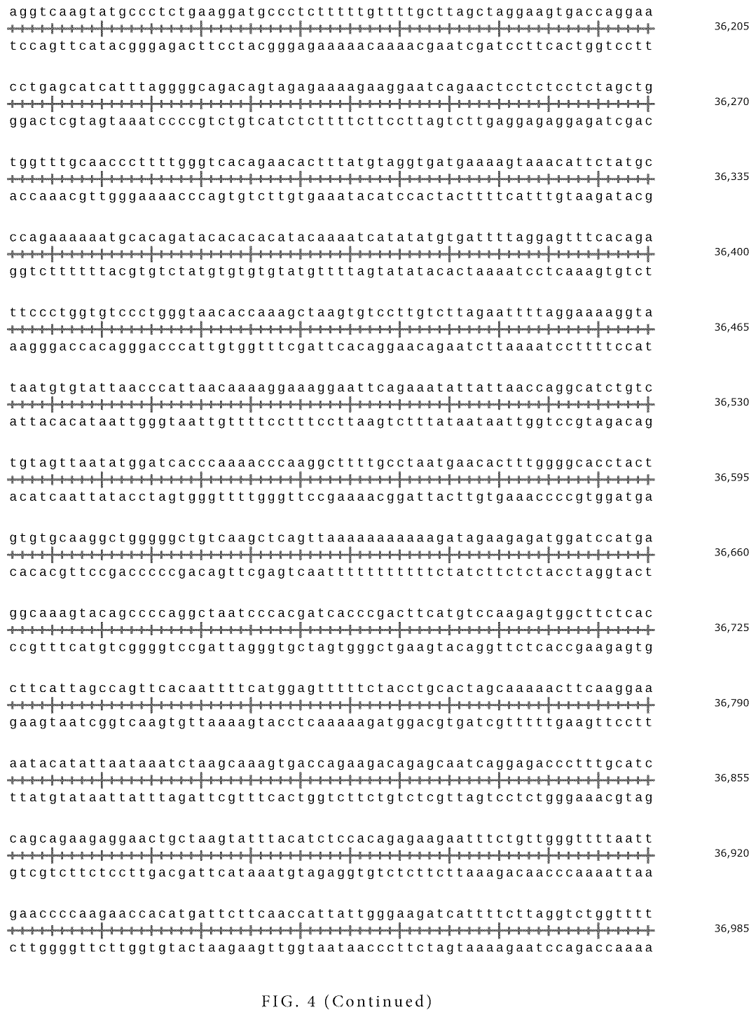

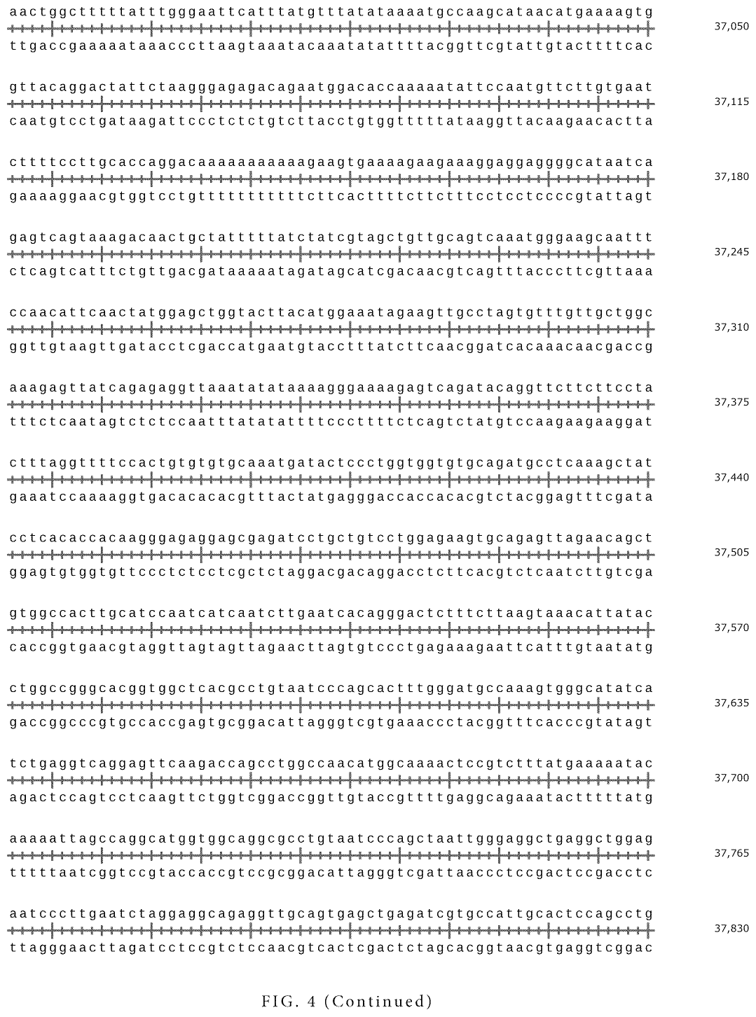

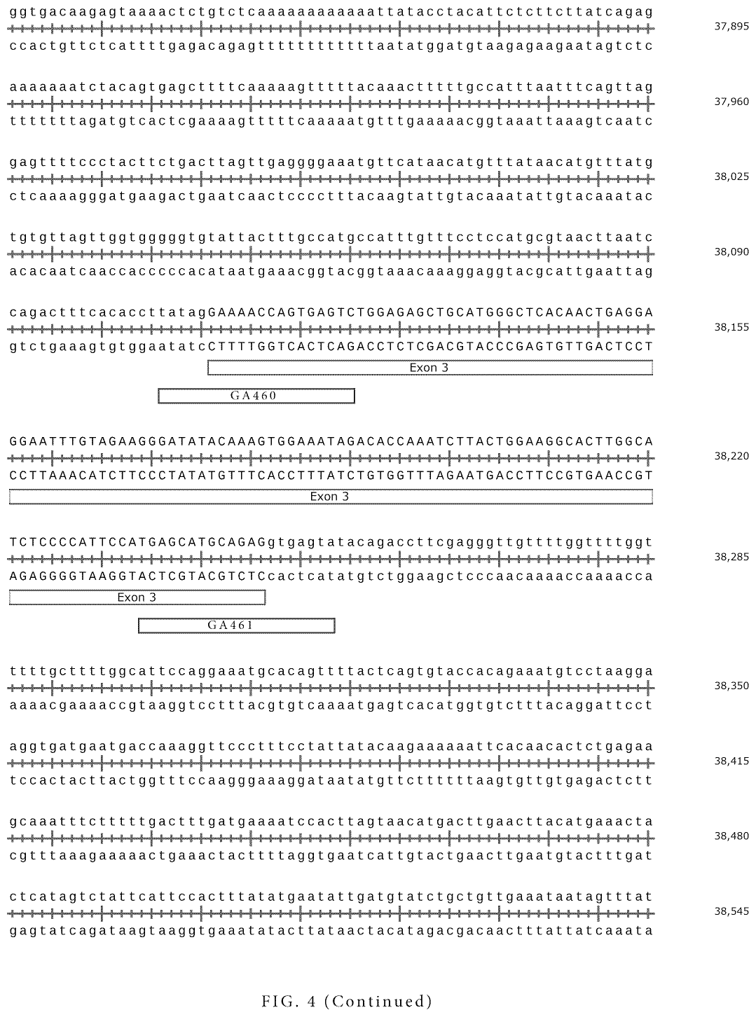

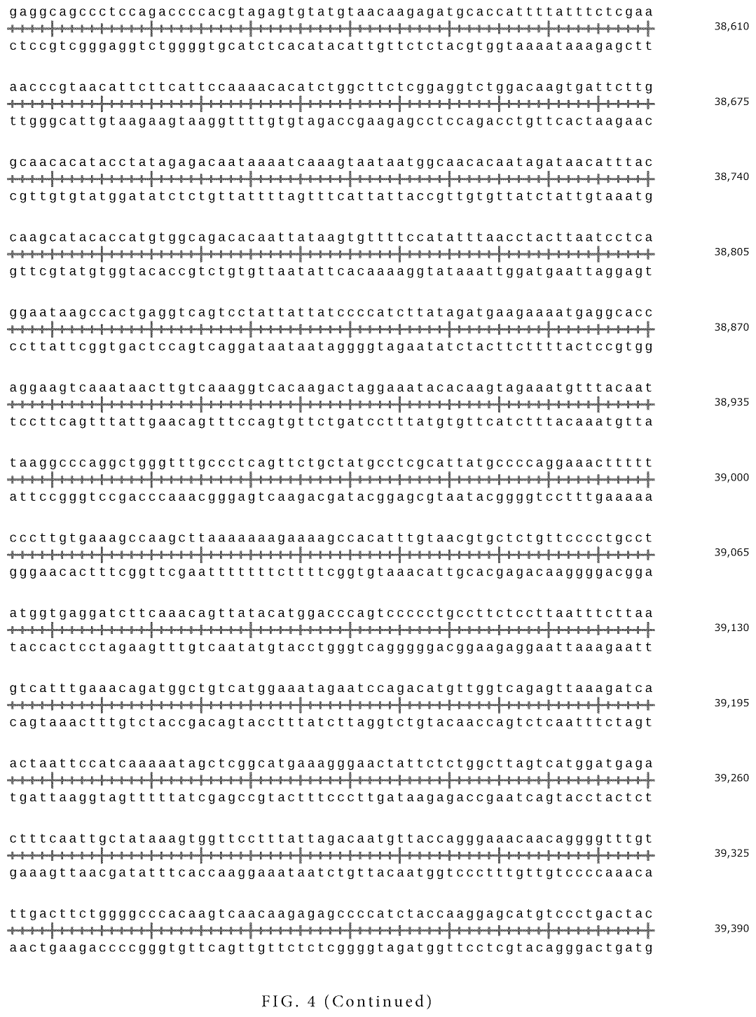

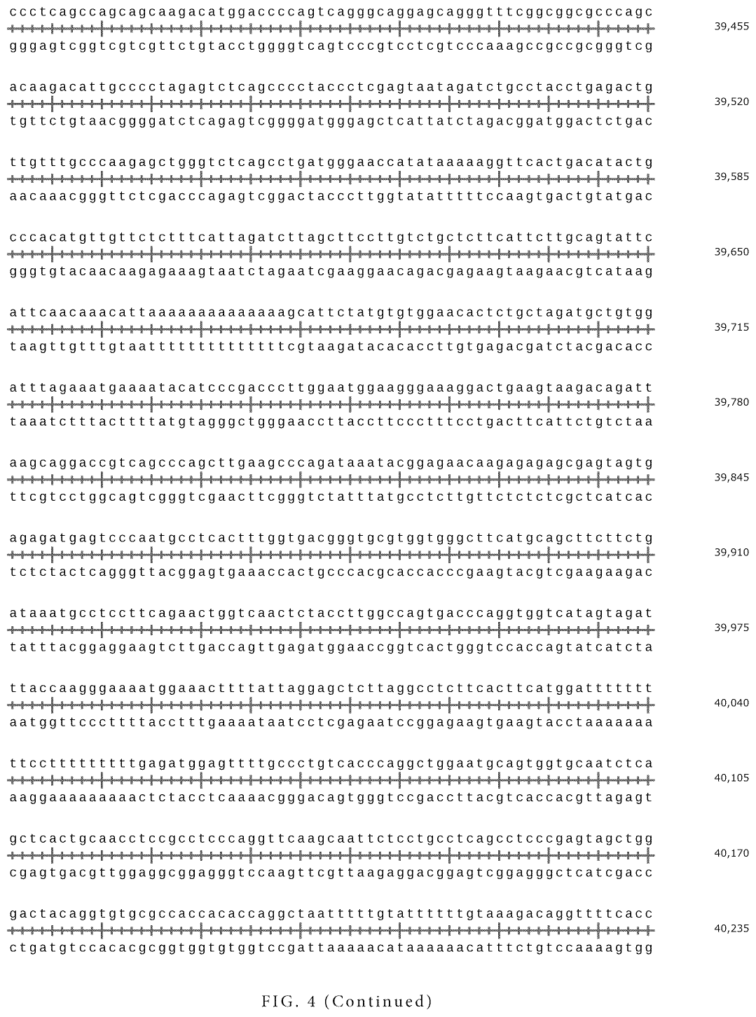

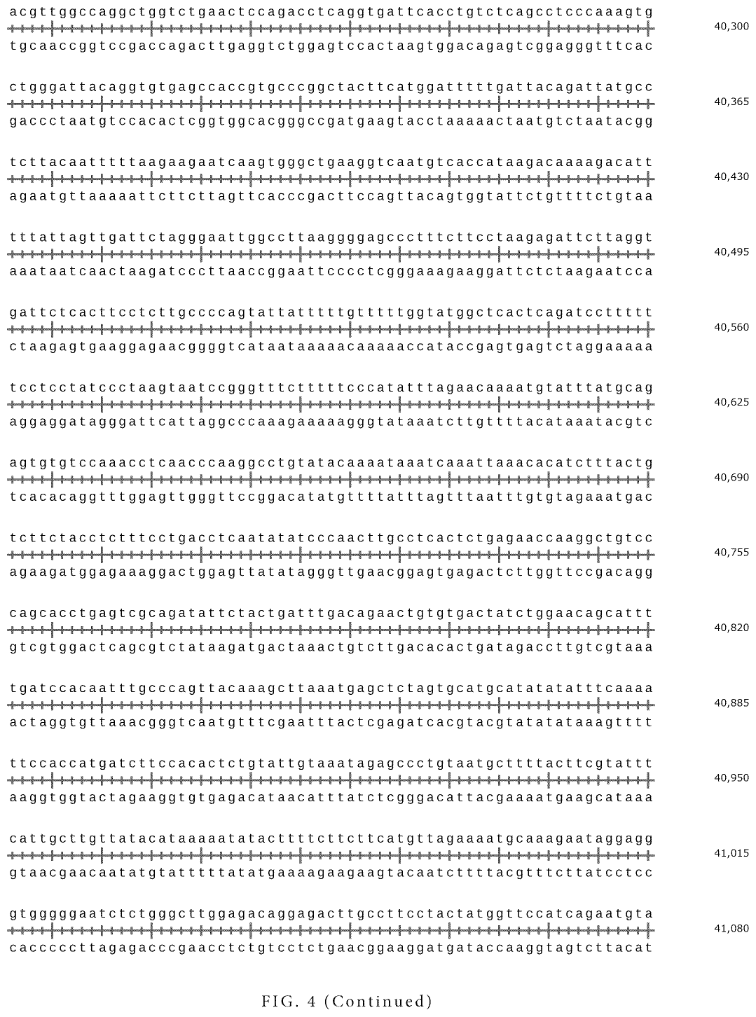

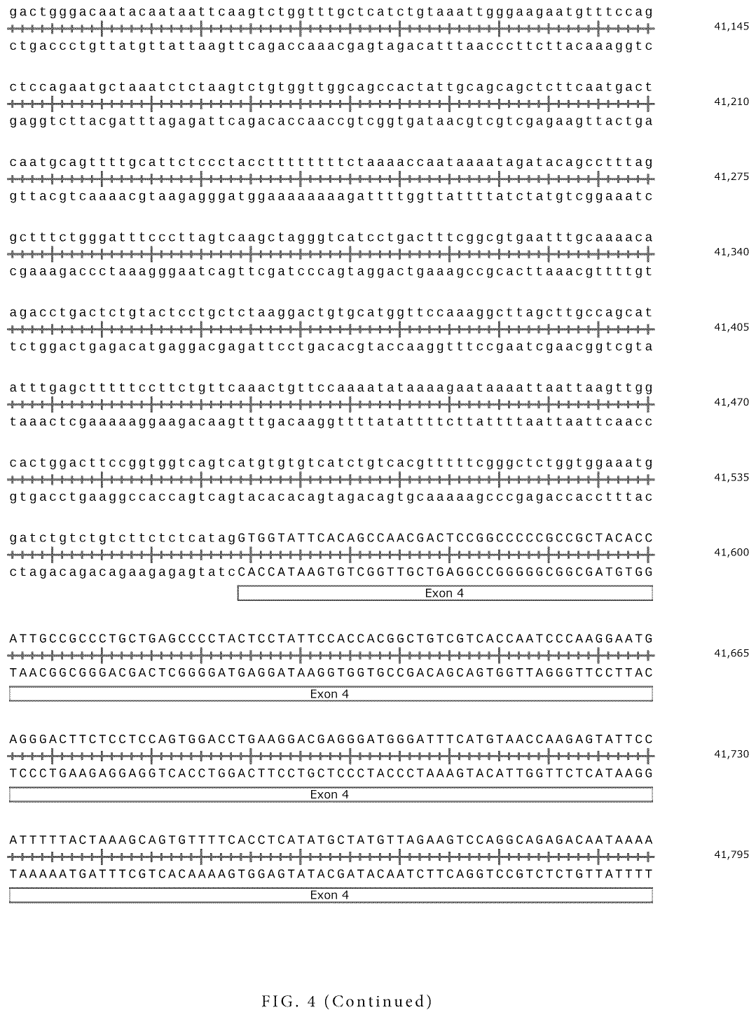

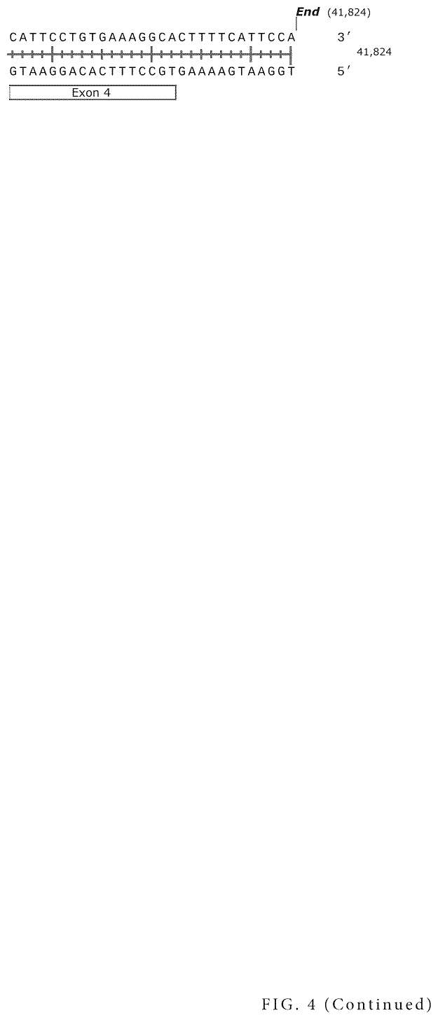

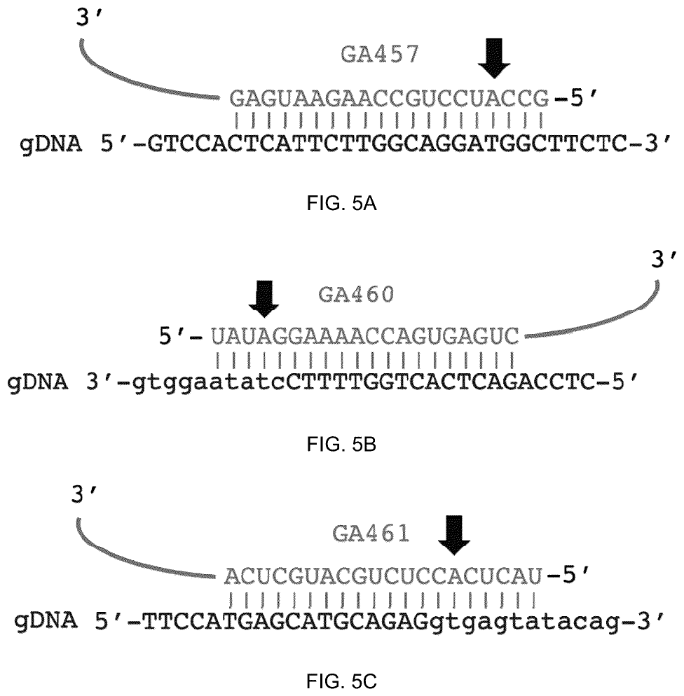

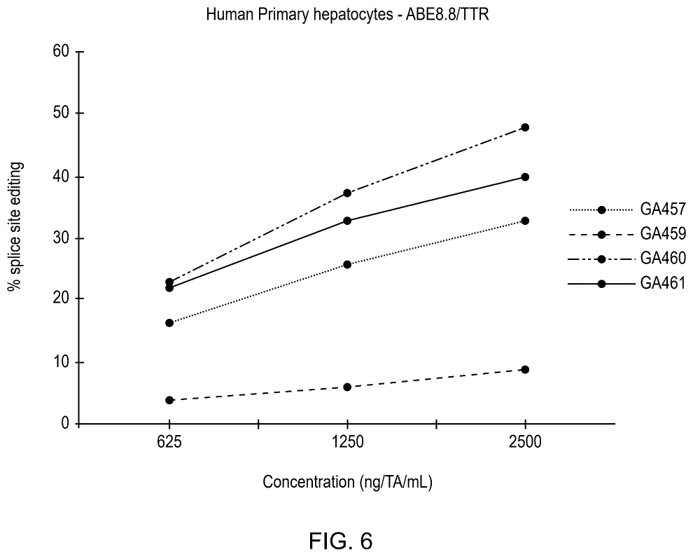

For orthogonal protospacer sequences of the corresponding cynomolgus monkey TTR gene sequence, each gRNA was also transfected with an equivalent amount of ABE8.8 mRNA (1:1 ratio by molecular weight) into primary cynomolgus hepatocytes at 5000, 2500, 1250, 625, 312.5, and 156.25 ng/RNA/mL. The mRNA, and corresponding amino acid, sequence of the ABE8.8 (MA004) used in shown below in Table 11. Three days after transfection, genomic DNA was harvested from the hepatocytes, and assessed for base editing with next-generation sequencing of PCR amplicons generated around the target splice site. Several sites exhibited high editing efficiency. In particular, GA457 (GA458 is the cynomolgus equivalent), GA460, and GA461 showed high editing activity in both human and cynomolgus primary hepatocytes. See FIGS. 5A-5C, FIG. 6, and Tables 2-3.

TABLE 2
Editing activity in human primary hepatocytes

| | | Human hepatocytes- Editing % | | | | | |
|---|---|---|---|---|---|---|---|
| | | 2500, | 2500, | 1250, | 1250, | 625, | 625, |
| gRNA ID | Protospacer (5′-3′) | rep 1 | rep 2 | rep 1 | rep 2 | rep 1 | rep 2 |
| GA457 | GCCATCCTGCCAAGAATGAG | 33.96 | 31.31 | 26.49 | 24.86 | 17.24 | 15.39 |
| | (SEQ ID NO: 24) | | | | | | |
| GA459 | GCAACTTACCCAGAGGCAAA | 8.5 | 8.69 | 5.46 | 6.15 | 3.79 | 3.89 |
| | (SEQ ID NO: 25) | | | | | | |
| GA460 | TATAGGAAAACCAGTGAGTC | 47.61 | 47.79 | 38.33 | 35.8 | 23.77 | 22.27 |
| | (SEQ ID NO: 26) | | | | | | |
| GA461 | TACTCACCTCTGCATGCTCA | 40.42 | 39.43 | 32.82 | 32.38 | 21.81 | 22.31 |
| | (SEQ ID NO: 27) | | | | | | |

TABLE 3
Editing activity in cyno primary hepatocytes

| | | Cyno hepatocytes- Editing % | | | | | |
|---|---|---|---|---|---|---|---|
| gRNA ID | Protospacer (5′-3′) | 5000 | 2500 | 1250 | 625 | 312.5 | 156.25 |
| GA458 | GCCATCCTGCCAAGAACGAG | 35.91 | 27.25 | 22.74 | 16.07 | 10.65 | 6.06 |
| | (SEQ ID NO: 28) | | | | | | |
| GA460 | TATAGGAAAACCAGTGAGTC | 40.16 | 38.25 | 31.71 | 18.44 | 11.22 | 1.62 |
| | (SEQ ID NO: 26) | | | | | | |
| GA461 | TACTCACCTCTGCATGCTCA | 37.53 | 29.8 | 19.46 | 13.16 | 7.45 | 0.07 |
| | (SEQ ID NO: 27) | | | | | | |

Results presented in Tables 2 and 3 are to be understood to be representative of results that may be achieved in accordance with the teachings provided herein. Compositions for editing a TTR gene according to the invention may produce editing activity that varies from the activity set forth in Table 2 or Table 3 by 10% or more, 20% or more, 30% or more, 40% or more, 50% or more, 60% or more, 70% or more, 80% or more, 90% or more, or 100% or more. In some embodiments, the compositions provide editing activity that is within 100%, within 90%, within 80%, with 70%, within 60%, within 50%, within 40%, within 30% or more, within 20% or more, or within 10% of the activity as set forth in Table 2 or Table 3.

### Example 2

### Off Target Analysis

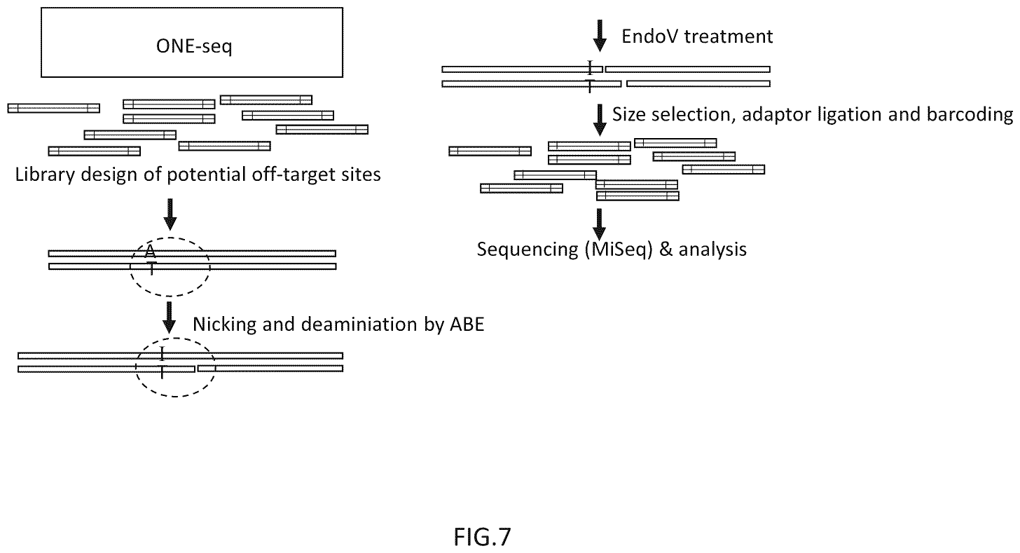

With a view towards establishing the safety of a base-editing therapy knocking down of TTR in the human liver in vivo, off-target mutagenesis analysis is assessed in primary human hepatocytes. Following the ONE-seq procedures detailed in PCT/US19/27788 (“Highly Sensitive in vitro Assays to Define Substrate Preferences and Sites of Nucleic-Acid Binding, Modifying, and Cleaving Agents”), off-target editing in human hepatocytes was assessed. A simplified flowchart of off-target analysis with the ONE-seq procedure is shown in FIG. 7. The in vitro biochemical assay ONE-seq was used to generate a list of candidate off-target sites and to determine the propensity of a ribonucleoprotein comprising the ABE8.8 base editor protein and each of the three protospacer guides sequences (GA457, GA460, and GA461) to cleave oligonucleotides in a library. The results from ONE-seq analysis of libraries generated for GA457, GA460, and GA461 are shown in Tables 8-10, with candidate off-target sites listed.

The methodology for ONE-seq is as follows: the design of a ONE-seq library starts with the computational identification of sites in a reference genome that have sequence homology to the on-target. For human ONE-seq libraries, the reference human genome (GRCh38, Ensembl v98, chromosomes ftp://ftp.ensembl.org/pub/release-98/fasta/homo_sapiens/dna/Homo_sapiens.GRCh38.dna.chromosome.{1-22,X,Y,MT}.fa and ftp://ftp.ensembl.org/pub/release-98/fasta/homo_sapiens/dna/Homo_sapiens.GRCh38.dna.nonchromosomal.fa), was searched for potential off-target sites with up to 6 mismatches to the protospacer sequence above, and sites with up to 4 mismatches plus up to 2 DNA or RNA bulges, using Cas-Designer v1.2 (http://www.rgenome.net/cas-designer/).

Sites with up to 6 mismatches and no bulges are referred to using a X<number of mismatches><number of bulges> code. As such, the on-target site is labelled as X00; a site with 1 mismatch to the on-target and no bulges is labelled as X10, and so on. Sites with DNA bulges are referred to with a similar nomenclature, DNA<number of mismatches><number of bulges>. As such, a site with 4 mismatches to the on-target and 2 DNA bulges is labelled as DNA42. The same nomenclature is used for RNA bulges, but these are coded as RNA<number of mismatches><number of bulges>.

The protospacer sequences identified were extended by 10 nucleotides (nt) on both sides with adjacent sequence from the respective reference genome (these regions are herein referred to as the genomic context). These extended sequences were then padded by additional sequences up to a final length of approximately 200 nt, including 6 predefined constant regions of different nucleotide composition and sequence length; 2 copies of a 14-nt site-specific barcode, one on each side of the central protospacer sequence; and 2 distinct 11-nt unique molecular identifiers (UMIs), one on each side of the central protospacer sequence. The UMIs are used to correct for bias from PCR amplification, and the barcodes allow for the unambiguous identification of each site during analysis. The barcodes are selected from an initial list of 668,420 barcodes, which contain neither a CC nor a GG in their sequences, and each barcode has a Hamming distance of 2 from any other barcode. A custom Python script was used for designing the final library.

The final oligonucleotide libraries are synthesized by a commercial vendor (Agilent Technologies). Each library is PCR-amplified and subjected to 1.25×AMPure XP bead purification (Beckman Coulter). After incubation at 25° C. for 10 minutes in CutSmart buffer (New England Biolabs), RNP comprising 769 nM recombinant ABE8.8-m protein and 1.54 μM gRNA is mixed with 100 ng of the purified library and incubated at 37° C. for 8 hours. The RNP dose is derived from an analysis documenting that it is a super-saturating dose, ie, above the dose that achieves the maximum amount of on-target editing in the biochemical assay.

Proteinase K (New England Biolabs) is added to quench the reaction at 37° C. for 45 minutes, followed by 2×AMPure XP bead purification. The reaction is then serially incubated with EndoV (New England Biolabs) at 37° C. for 30 minutes, Klenow Fragment (New England Biolabs) at 37° C. for 30 minutes, and NEBNext Ultra II End Prep Enzyme Mix (New England Biolabs) at 20° C. for 30 minutes followed by 65° C. for 30 minutes, with 2×AMPure XP bead purification after each incubation. The reaction is ligated with an annealed adaptor oligonucleotide duplex at 20° C. for 1 hour to facilitate PCR amplification of the cleaved library products, followed by 2×AMPure XP bead purification. Size selection of the ligated reaction is performed on a PippinHT system (Sage Sciences) to isolate DNA of 150 to 200 bp on a 3% agarose gel cassette, followed by 2 rounds of PCR amplification to generate a barcoded library, which undergoes paired-end sequencing on an Illumina MiSeq System as described above.

Two cleavage products are obtained in a ONE-seq experiment. The PROTO side includes the part of the oligonucleotide upstream of the cleavage position, whereas the PAM side includes part of the oligonucleotide downstream of the cleavage position. In an ABE experiment, only the PROTO side is informative of editing activity (an A→G substitution); therefore, only this side is sequenced.

Paired-end reads were trimmed for sequencing adapters using trimmomatic v0.39 (Bolger et al., 2014) with custom Nextera adapters (PrefixPE/1: 5′-ACACTCTTTCCCTACACGACGCTCTTCCGATCT-3′ (SEQ ID NO: 30); PrefixPE/2: 5′-GTGACTGGAGTTCAGACGTGTGCTCTTCCGATCT-3′ (SEQ ID NO: 31); as specified in file) and parameters “ILLUMINACLIP:NEB_custom.fa:2:30:10:1:true LEADING:0 TRAILING:0 SLIDINGWINDOW:4:30 MINLEN:36”. For experiments with lower sequencing quality (VOL014), these parameters were set to “ILLUMINACLIP NEB_custom.fa:2:30:10:1:true LEADING:2 TRAILING:0 SLIDINGWINDOW:30:30 MINLEN:36”. Reads were then merged using FLASH v1.2.11 (Magoc and Salzberg, 2011) with parameters “--max-mismatch-density=0.25 --max-overlap=160”. Merged reads were scanned for the constant sequences, barcodes and protospacer sequences unique to each site, and filtered to those with evidence of an A→G substitution in the editing window (defined as the 1-10 most PAM-distal positions of the protospacer). Duplicated reads were discarded.

For each site, the total number of edited reads was normalized to the total number of edited reads assigned to the on-target site, and this ratio defines the ONE-seq score for the site. Sites were ranked by ONE-seq score, and those with a score equal to or larger than 0.001, were selected for validation. A score equal to or larger than 0.001 encompasses sites that have down to 1000-fold less editing activity in the biochemical assay compared to editing of the on-target site. This threshold is based on the premise that in cells, if there is 100% on-target editing, 1/1000-fold less editing activity would translate to <0.1% off-target editing, which falls below the lower limit of detection of editing by NGS. Oligonucleotides with higher sequence counts reflect a higher propensity for Cas9/gRNA cleavage in vitro and hence for greater potential of off-target mutagenesis in cells.

Several candidate off-target sites were analyzed for off-target editing in human primary hepatocytes. Table 4 shows the results from validating 47 candidate off-target sites for guide RNA GA457, from cells co-transfected with gRNA and an equivalent amount of in vitro transcribed ABE8.8 mRNA (1:1 ratio by molecular weight) into primary human hepatocytes via MessengerMax reagent (Lipofectamine). The on-target site has high editing efficiency, while all off-target sites show little to no editing (less than 0.4% net editing).

TABLE 4
GA457 validation against 47 potential off-target
candidate sites in human primary hepatocytes

| | | SEQ | % Editing-human primary | | |
|---|---|---|---|---|---|
| GA457 | | ID | hepatocytes | | |
| ID | Sequence (5′-3′) | NO: | Treated | Untreated | Net |
| On-target | GCCATCCTGCCAAGAATGAG | 24 | 54.21 | 0.11 | 54.1 |
| OT1 | GCCATCCTACCAGGAATGAA | 32 | 0.28 | 0.13 | 0.15 |
| OT2 | GCCATCTTGCCAAGAAAAAG | 33 | 0.12 | 0.09 | 0.03 |
| OT3 | GCCATACCTGCCATGAATGAG | 34 | 0.2 | 0.19 | 0.01 |
| OT4 | GCCATCCTGACAGGAATGAG | 35 | 0.24 | 0.38 | −0.14 |
| OT5 | ACCATCCTGCAAAGAATGAT | 36 | 0.18 | 0.24 | −0.06 |
| OT6 | GCCATCCAATAAGAATGAG | 37 | 0.69 | 0.32 | 0.37 |
| OT7 | GCCATCCTGACAAGTATGAG | 38 | 0.29 | 0.22 | 0.07 |
| OT8 | CCATACCTGCCAAGAATGAA | 39 | 0.18 | 0.15 | 0.03 |
| OT9 | TGCATCCTGCCAAAAATGGG | 40 | 0.06 | 0.08 | −0.02 |
| OT10 | TGCATCCTGCCAAGAAGAAG | 41 | 0.06 | 0.08 | −0.02 |
| OT11 | GCCATCCTCCAAGAATGCT | 42 | 0.13 | 0.13 | 0 |
| OT12 | GCCATCTGCAAGAAGGAG | 43 | 0.11 | 0.19 | −0.08 |
| OT13 | GCCATCCTATCAAGAATAAA | 44 | 0.22 | 0.2 | 0.02 |
| OT14 | TCCATCCTGTAAGAATGAG | 45 | 0.03 | 0.05 | −0.02 |
| OT15 | GGCCATCTGCCAAGAAGGAT | 46 | 0.12 | 0.13 | −0.01 |
| OT16 | ACCATCCTGCCAGCAATGTG | 47 | 0.17 | 0.12 | 0.05 |
| OT17 | TCCATCCTACTAAGAATGAG | 48 | 0.11 | 0.13 | −0.02 |
| OT18 | GGTATCCTGCCAAGAATGGA | 49 | 0.09 | 0.1 | −0.01 |
| OT19 | TCCATCCTGCCAAGAATTGC | 50 | 0.14 | 0.07 | 0.07 |
| OT20 | GCCATCTGCAAGAATGAG | 51 | 0.15 | 0.18 | −0.03 |
| OT21 | GCCATCCTGCAAATATGAG | 52 | 0.04 | 0.07 | −0.03 |
| OT22 | ACCATCCTGTCAAGAATCAA | 53 | 0.2 | 0.15 | 0.05 |
| OT23 | GCCATACTAACAAGAATGAG | 54 | 0.25 | 0.23 | 0.02 |
| OT24 | GTGATCCTGCCAGGAATAAG | 55 | 0.08 | 0.11 | −0.03 |
| OT25 | GCCATCAAGCAAGAATGAG | 56 | 0.3 | 0.27 | 0.03 |
| OT26 | GCCATCCTCACAAGTATGAG | 57 | 0.19 | 0.22 | −0.03 |
| OT27 | ACCATCCAGCAAGAATGAG | 58 | 0.43 | 0.32 | 0.11 |
| OT28 | GCCATATGCCAAAAAGGAG | 59 | 0.24 | 0.24 | 0 |
| OT29 | GACATCCTGTCAAGGATCAG | 60 | 0.21 | 0.16 | 0.05 |
| OT30 | GCCATAGCCAAAAATGAA | 61 | 0.18 | 0.27 | −0.09 |
| OT31 | GCCATAAGCCAAAGAATGAC | 62 | 0.09 | 0 | 0.09 |
| OT32 | GCCATCCTAACAAGTATGAG | 63 | 0.25 | 0.45 | −0.2 |
| OT33 | CATATCCTGCCAGAATGAG | 64 | 0.15 | 0.21 | −0.06 |
| OT34 | TACATCCTACCAAGGAATCAG | 65 | 0.29 | 0.26 | 0.03 |
| OT35 | CCCATCCTGCCAAGAAGTGT | 66 | 0.08 | 0.06 | 0.02 |
| OT36 | GCCATCCTACAAAAATGAG | 67 | 0.16 | 0.21 | −0.05 |
| OT37 | GTCATCCTGCCAGGAATGAA | 68 | 0.09 | 0.07 | 0.02 |
| OT38 | GCCATATCTGCCAAGAATGCG | 69 | 0.16 | 0.16 | 0 |
| OT39 | TCCATCCTGTCAAGAATGTG | 70 | 0.05 | 0.04 | 0.01 |
| OT40 | TCCATCCTCCAGAATGAG | 71 | 0.07 | 0.08 | −0.01 |
| OT41 | GCCATGCTGCCAAGAATGAT | 72 | 0.15 | 0.16 | −0.01 |
| OT42 | GCTATCCTGCCAGAATGAG | 73 | 0.07 | 0.07 | 0 |
| OT43 | TGCATCCTGACAAGAAATAG | 74 | 0.34 | 0.24 | 0.1 |
| OT44 | TCCATAGCCAAGAATGAG | 75 | 0.43 | 0.27 | 0.16 |
| OT45 | ACCATCTGTCAAGAATGAG | 76 | 0.26 | 0.21 | 0.05 |
| OT46 | GCCATCCCGCCAGGAATTAT | 77 | 0.08 | 0.07 | 0.01 |
| OT47 | ACCATCCTTCCAAGAAGATG | 78 | 0.14 | 0.12 | 0.02 |

GA459, GA460, and GA461 were similarly also assessed for off-target editing as shown in Tables 5, 6, and 7, respectively. While the on-target site, for each guide, shows high editing efficiency in the treated groups compared to the control groups, there is little to no off-target editing observed at candidate off-target sites.

TABLE 5
GA459 validation against 6 potential off-target candidate sites
in human primary hepatocytes

| | | SUM Editing %-Human Primary Hepatocytes | | | | |
|---|---|---|---|---|---|---|
| GA459 | | Treat | Treat | Untreated | Untreated | Untreated |
| ID | Sequence (5′-3′) | rep 1 | rep 2 | rep 1 | rep 2 | rep 3 |
| On- | GCAACTTACCCAGAGGCAAA | 14.17 | 14.29 | 0.67 | 0.57 | 0.73 |
| target | (SEQ ID NO: 25) | | | | | |
| OT1 | ACAAATTACCCAGAGGAAAA | 1.19 | 1.25 | 1.31 | 1.28 | 1.27 |
| | (SEQ ID NO: 79) | | | | | |
| OT3 | TCAACTTACCCAGAGTCAAA | 0.98 | 0.82 | 0.63 | 0.68 | 0.8 |
| | (SEQ ID NO: 80) | | | | | |
| OT4 | GCAACTTGCCCAGAGGCACA | 0.92 | 1.07 | 0.87 | 0.99 | 0.79 |
| | (SEQ ID NO: 81) | | | | | |
| OT5 | GCAACATACCCAGTGGCAAA | 1.07 | 0.91 | 0.87 | 0.86 | 0.92 |
| | (SEQ ID NO: 82) | | | | | |
| OT6 | GCAGCCTACCCAGAGGCAAA | 1.02 | 1.1 | 0.97 | 1 | 1.05 |
| | (SEQ ID NO: 83) | | | | | |
| OT7 | GCAACTCCCCCAGAGGCAAA | 1.45 | 1.4 | 1.25 | 1.13 | 1.37 |
| | (SEQ ID NO: 84) | | | | | |

TABLE 6
GA460 validation against 3 potential off-target candidate
sites in human primary hepatocytes

| | | SUM Editing %-Human Primary Hepatocytes | | | | |
|---|---|---|---|---|---|---|
| GA460 | | Treat | Treat | Untreated | Untreated | Untreated |
| ID | Sequence (5′-3′) | rep 1 | rep 2 | rep 1 | rep 2 | rep 3 |
| On- | TATAGGAAAACCAGTGAGTC | 76.4 | 74.3 | 1.54 | 1.17 | 1.41 |
| target | (SEQ ID NO: 26) | | | | | |
| OT1 | TAGAGGAAAACCAGTCAGTC | 1.44 | 1.6 | 1.51 | 1.35 | 1.61 |
| | (SEQ ID NO: 85) | | | | | |
| OT2 | CATAGGAAAACCAGTGAGTT | 5.67 | 6.6 | 1.18 | 0.98 | 1.24 |
| | (SEQ ID NO: 86) | | | | | |
| OT3 | TAAAGGAAAACCAGTGGGTC | 1.26 | 1.29 | 1.31 | 1.02 | 1.53 |
| | (SEQ ID NO: 87) | | | | | |

TABLE 7
GA461 validation against 4 potential off-target candidate
sites in human primary hepatocytes

| | | SUM Editing %- Human Primary Hepatocytes | | | | |
|---|---|---|---|---|---|---|
| GA461 | | Treat | Treat | Untreated | Untreated | Untreated |
| ID | Sequence (5′-3′) | rep 1 | rep 2 | rep 1 | rep 2 | rep 3 |
| On- | TACTCACCTCTGCATGCT | ND | 58.9 | 0.39 | 0.37 | 0.35 |
| target | CA (SEQ ID NO: 27) | | | | | |
| OT1 | TACACAACTGTGCATGCT | 0.93 | 0.95 | 0.89 | 0.86 | 0.82 |
| | CA (SEQ ID NO: 88) | | | | | |
| OT2 | TATTCACCTCTGCATGCT | 0.17 | 0.18 | 0.23 | 0.16 | 0.2 |
| | CT (SEQ ID NO: 89) | | | | | |
| OT3 | TACTTACCTCTGCTTGCT | 0.28 | 0.35 | 0.28 | 0.23 | 0.34 |
| | CA (SEQ ID NO: 90) | | | | | |
| OT4 | TACACACCTCTACATGCT | 0.67 | 0.94 | 0.75 | 0.62 | ND |
| | CA (SEQ ID NO: 91) | | | | | |

Table 8 provides some results for off-target editing with the GA457 guide.

Table 9 provides some results for off-target editing with the GA460 guide.

Table 10 provides some results for off-target editing with the GA461 guide.

Results presented in Tables 4, 6, 7, 8, 9, and 10 are to be understood to be representative of results that may be achieved in accordance with the teachings provided herein. Compositions for editing a TTR gene according to the invention may produce total off-target editing activity that varies from the activity set forth in Table 4, 6, 7, 8, 9, or 10 or discussed regarding GA457, 460, or 461. For example, the compositions may produce total off-target editing activity that varies from the activity set forth in Table 4, 6, 7, 8, 9, or 10 or discussed regarding GA457, 460, or 461 by 10% or more, 20% or more, 30% or more, 40% or more, 50% or more, 60% or more, 70% or more, 80% or more, 90% or more, or 100% or more, for one or more off target site set forth in Table 4, 6, 7, 8, 9, or 10 or discussed regarding GA457, 460, or 461. In some embodiments, the compositions provide total off-target editing activity that is within 100%, within 90%, within 80%, with 70%, within 60%, within 50%, within 40%, within 30% or more, within 20% or more, or within 10% of the activity as set forth in Table 4, 6, 7, 8, 9, or 10 or discussed regarding GA457, 460, or 461 for one or more site set forth in Table 4, 6, 7, 8, 9, or 10 or discussed regarding GA457, 460, or 461. In some embodiments, the compositions produce off-target editing activity that is less than or equal to the activity set forth in Table 4, 6, 7, 8, 9, or 10 or discussed regarding GA457, 460, or 461 for one or more site set forth in Table 4, 6, 7, 8, 9, or 10 or discussed regarding GA457, 460, or 461. In some embodiments, the compositions produce no off-target editing activity for one or more site set forth in Table 4, 6, 7, 8, 9, or 10 or discussed regarding GA457, 460, or 461.

TABLE 8
Some GA457 Candidate Off-target Sites

| | | | SEQ | |
|---|---|---|---|---|
| | | | ID | |
| Chromosome | Location | Sequence (5′-3′) | NO: | Alignment |
| 1 | 214915487 | TGCATCCTGCCAAAAATGGGGAG | 92 | X50 |
| 2 | 162088240 | GCCATCTTGCCAAGAAAAAGGGG | 161 | X30 |
| 3 | 143033429 | AACATCCTGCAAGAATAAGAAG | 255 | RNA41 |
| 4 | 148246005 | GCCATCCAATAAGAATGAGTGG | 314 | RNA31 |
| 5 | 171975758 | GTCATCCTGCCAAAATAAGGAGG | 381 | X60 |
| 6 | 11558428 | CCCATACAGCCAAGAATGAGAAA | 443 | X50 |
| 7 | 136711066 | TCCATCCTACTAAGAATGAGGAG | 490 | X40 |
| 8 | 64649745 | ACCATCCTGACAAGTGTGAGGCA | 601 | X60 |
| 9 | 13461786 | GTGATCCTGCCAGGAATAAGGAG | 602 | X50 |
| 10 | 84701101 | GCCACCCTCCAAGGATCTGAGG | 682 | RNA41 |
| 11 | 36962784 | GCCATACTAACAAGAATGAGGTG | 683 | X40 |
| 12 | 18423002 | GCCATCCTCAAGAATGGGAAA | 774 | RNA32 |
| 13 | 109466404 | ACCATCCTGTCAAGAATCAAGAG | 775 | X50 |
| 14 | 43842189 | GCCAACCTGACAAATGTGAGG | 842 | RNA32 |
| 15 | 38417527 | ACCATCCTTCCAAGAAGATGGGG | 843 | X50 |
| 16 | 19426632 | GCCATCCAGCCAAGCAAGAAGGG | 890 | X40 |
| 17 | 42774058 | GTCATCACTGCCAAGAACAAGAGG | 891 | DNA31 |
| 18 | 6852692 | GCCATCCTGTAAGAATAAGGAT | 941 | RNA41 |
| 19 | 55221349 | ACCATCCTGCCAGCAATGTGAGG | 942 | X40 |
| 20 | 56309168 | TCCATCCTGAAGAATGAATAG | 986 | RNA32 |
| 21 | 40793316 | GCCATATCTGCCAAGAATGCGGAG | 987 | DNA31 |
| 22 | 34535097 | GATATCCTCACAAGAATGAGTGA | 1009 | X50 |
| X | 54383781 | GCTCTACTGCCAAGAAAGTGG | 1068 | RNA32 |
| Y | 6961334 | GCCATCCACCAAGAAAGCGGAG | 331 | RNA41 |

Additional examples of GA457 off-target sites are presented in U.S. Provisional Patent Application No. 63/322,182, filed Mar. 21, 2022. The GA457 off-target sites may include any one of SEQ ID NOS: 92-1073.

TABLE 9
Some GA460 Candidate Off-target Sites

| | | | SEQ | |
|---|---|---|---|---|
| Chromosome | Location | Sequence | ID NO: | Alignment |
| 1 | 114689517 | TATAAGAAAACCAGTGTCTCTGG | 1074 | X30 |
| 2 | 165173430 | TATAGTGAACCAGTGAGGCAGG | 1280 | RNA31 |
| 3 | 56975861 | TGTAGAAAAACCAGTGAATAAGA | 1525 | X50 |
| 4 | 175627437 | GATAGGAAAACCATGAGGGGGT | 1892 | RNA41 |
| 5 | 115033087 | TATAGGAAACCAATGAGTGCTG | 1893 | RNA31 |
| 6 | 132497708 | TATAGGTAAACCAGAGTAGGC | 2232 | RNA32 |
| 7 | 40539970 | AGTAGGAAAACCAGTATATAGGG | 2233 | X60 |
| 8 | 77342779 | AATAGGAAAACCATTTTCAGG | 2494 | RNA32 |
| 9 | 73825991 | AATAGGAAAACCAGTAAAATAGG | 2495 | X50 |
| 10 | 112702031 | CATACGAAGTCCATGAGTCAGG | 2724 | RNA41 |
| 11 | 60264073 | ATATGGAAAACCAGAGAATCAGG | 2725 | X60 |
| 12 | 103497054 | GATAGAAACACCATGAATCAGG | 2957 | RNA41 |
| 13 | 31477698 | TATATGAAACCAGTAAGTTTGG | 2958 | RNA31 |
| 14 | 91758013 | TATAGGTGTAAACCAGTGTGCCTAG | 3137 | DNA42 |
| 15 | 28238271 | TATAGGAGAAACAGTGAATAGGA | 3138 | X50 |
| 16 | 74819495 | AAAATGTAAACCAGTAAGCCCGG | 3289 | X60 |
| 17 | 44943044 | TGTAGGAAGAACCAGTGGATCGGG | 3290 | DNA31 |
| 18 | 1915908 | AAAAGGAAAGCCAGTGACCTGG | 3410 | RNA41 |
| 19 | 44246762 | AAATAGAAAACCAGTAAGTCATG | 3411 | X60 |
| 20 | 24558049 | TATAGGAAAACAGGAACTCTGG | 3517 | RNA31 |
| 21 | 13185396 | TATAATAAAACCAGTGATAAGGG | 3518 | X50 |
| 22 | 38987774 | TGTAGGAAAACATATGATCAGG | 3574 | RNA41 |
| X | 134161037 | AATAGGATAACCAGTCAGTAGGG | 3575 | X40 |
| Y | 15254846 | TATAACATAACCAATAGGTCAGG | 3725 | X60 |

Additional examples of GA460 off-target sites are presented in U.S. Provisional Patent Application No. 63/322,182, filed Mar. 21, 2022. The GA460 off-target sites may include any one of SEQ ID NOS: 1074-3725.

TABLE 10
Some GA461 Candidate Off-target sites

| | | | SEQ ID | |
|---|---|---|---|---|
| Chromosome | Location | Sequence (5′-3′) | NO: | Alignment |
| 1 | 110474222 | TACTCACCTCTACATGCTCAGTG | 3726 | X20 |
| 2 | 136311439 | TACAGCACCTCTGCATTGCCAGGG | 4071 | DNA41 |
| 3 | 159697502 | TATTCACCTCTGCATCTCCAGGG | 4072 | X40 |
| 4 | 3846305 | CTCTCACCTCTGCATGACAAGC | 4326 | RNA41 |
| 5 | 174698295 | TACTCACAATGCATGCTAAAGG | 4327 | RNA31 |
| 6 | 31933576 | TACTCACCTCTGCCTTCCTTTGT | 4551 | X60 |
| 7 | 53373361 | TTATCACCCTGCATGTTCAGGG | 4552 | RNA31 |
| 8 | 77161406 | TACTGACCTTTCCATCTCACTG | 4760 | RNA41 |
| 9 | 101247864 | TACTCACTCAGCATGTTCAGAG | 4761 | RNA31 |
| 10 | 4684859 | GAATAACCTCTGATGGTCAAGG | 4945 | RNA41 |
| 11 | 85210644 | TATTCACCTCTGCATGCTCTGAG | 4946 | X30 |
| 12 | 117261587 | TATTCAGCAATGCATGTCAAGG | 5116 | RNA41 |
| 13 | 94452599 | TACTTACCTTACATGTTCAAGG | 5117 | RNA31 |
| 14 | 64054638 | TACTCAACTCTGCTGCTATAGC | 5243 | RNA41 |
| 15 | 95108931 | TACTCAACTCTGCTGCTCTAGG | 4450 | RNA21 |
| 16 | 56094894 | TACTAACCTTGCCAGCTGAGGG | 5360 | RNA41 |
| 17 | 71161191 | TGCTCACCCCACATGCTCATGG | 5361 | RNA31 |
| 18 | 3284286 | TTTTCTACTCTGCATAATCATGG | 5473 | X60 |
| 19 | 45241812 | ACCTCACCTCTGCCTGCTCTGGG | 5474 | X40 |
| 20 | 13122061 | TACTCAACTGCATTCTCAGGG | 5580 | RNA22 |
| 21 | 45735600 | CACTCACACTACATGCTCTTGG | 5581 | RNA41 |
| 22 | 20392740 | TCCACACCTCTCGGCAAGCTGAGGG | 5645 | DNA42 |
| X | 39842774 | TATATACCTCTGCATGTTCAGAG | 5646 | X50 |
| Y | 20738820 | TAGACACATAAGCATGCTCACAG | 5741 | X60 |

Additional examples of GA461 off-target sites are presented in U.S. Provisional Patent Application No. 63/322,182, filed Mar. 21, 2022. The GA461 off-target sites may include any one of SEQ ID NOS: 3726-5745.

TABLE 11
ABE variant sequences
MA004 mRNA and protein sequences

| Region | Sequence | |
|---|---|---|
| Full | mRNA | Au'GAGCGAGGu'GGAGu'u'CAGCCACGAGu'ACu'GGAu'GCGGCACGCCCu' |
| sequence | | GACCCu'GGCCAAGCGGGCCCGGGACGAGCGGGAGGu'GCCCGu'GGGCGCCG |
| | | u'GCu'GGu'GCu'GAACAACCGGGu'GAu'CGGCGAGGGCu'GGAACCGGGCC |
| | | Au'CGGCCu'GCACGACCCCACCGCCCACGCCGAGAu'CAu'GGCCCu'GCGGC |
| | | AGGGCGGCCu'GGu'GAu'GCAGAACu'ACCGGCu'GAu'CGACGCCACCCu'G |
| | | u'ACGu'GACCu'u'CGAGCCCu'GCGu'GAu'Gu'GCGCCGGCGCCAu'GAu' |
| | | CCACAGCCGGAu'CGGCCGGGu'GGu'Gu'u'CGGCGu'GCGGAACGCCAAGAC |
| | | CGGCGCCGCCGGCAGCCu'GAu'GGACGu'GCu'GCACCACCCCGGCAu'GAAC |
| | | CACCGGGu'GGAGAu'CACCGAGGGCAu'CCu'GGCCGACGAGu'GCGCCGCCC |
| | | u'GCu'Gu'GCCGGu'u'Cu'u'CCGGAu'GCCCCGGCGGGu'Gu'u'CAACGC |
| | | CCAGAAGAAGGCCCAGAGCAGCACCGACAGGAAAu'AAGAGAGAAAAGAAGAGu' |
| | | AAGAAGAAAu'Au'AAGAGCCACCAGCGGCGGCAGCAGCGGCGGCAGCAGCGG |
| | | CAGCGAGACACCCGGCACCAGCGAGAGCGCCACCCCCGAGAGCAGCGGCGGCAG |
| | | CAGCGGCGGCAGCGACAAGAAGu'ACAGCAu'CGGCCu'GGCCAu'CGGCACCA |
| | | ACAGCGu'GGGCu'GGGCCGu'GAu'CACCGACGAGu'ACAAGGu'GCCCAGCA |
| | | AGAAGu'u'CAAGGu'GCu'GGGCAACACCGACCGGCACAGCAu'CAAGAAGAA |
| | | CCu'GAu'CGGCGCCCu'GCu'Gu'u'CGACAGCGGCGAGACAGCCGAGGCCAC |
| | | CCGGCu'GAAGCGGACCGCCCGGCGGCGGu'ACACCCGGCGGAAGAACCGGAu' |
| | | Cu'GCu'ACCu'GCAGGAGAu'Cu'u'CAGCAACGAGAu'GGCCAAGGu'GGAC |
| | | GACAGCu'u'Cu'u'CCACCGGCu'GGAGGAGAGCu'u'CCu'GGu'GGAGGAG |
| | | GACAAGAAGCACGAGCGGCACCCCAu'Cu'u'CGGCAACAu'CGu'GGACGAGG |
| | | u'GGCCu'ACCACGAGAAGu'ACCCCACCAu'Cu'ACCACCu'GCGGAAGAAGC |
| | | u'GGu'GGACAGCACCGACAAGGCCGACCu'GCGGCu'GAu'Cu'ACCu'GGCC |
| | | Cu'GGCCCACAu'GAu'CAAGu'u'CCGGGGCCACu'u'CCu'GAu'CGAGGGC |
| | | GACCu'GAACCCCGACAACAGCGACGu'GGACAAGCu'Gu'u'CAu'CCAGCu' |
| | | GGu'GCAGACCu'ACAACCAGCu'Gu'u'CGAGGAGAACCCCAu'CAACGCCAG |
| | | CGGCGu'GGACGCCAAGGCCAu'CCu'GAGCGCCCGGCu'GAGCAAGAGCCGGC |
| | | GGCu'GGAGAACCu'GAu'CGCCCAGCu'GCCCGGCGAGAAGAAGAACGGCCu' |
| | | Gu'u'CGGCAACCu'GAu'CGCCCu'GAGCCu'GGGCCu'GACCCCCAACu'u' |
| | | CAAGAGCAACu'u'CGACCu'GGCCGAGGACGCCAAGCu'GCAGCu'GAGCAAG |
| | | GACACCu'ACGACGACGACCu'GGACAACCu'GCu'GGCCCAGAu'CGGCGACC |
| | | AGu'ACGCCGACCu'Gu'u'CCu'GGCCGCCAAGAACCu'GAGCGACGCCAu'C |
| | | Cu'GCu'GAGCGACAu'CCu'GCGGGu'GAACACCGAGAu'CACCAAGGCCCCC |
| | | Cu'GAGCGCCAGCAu'GAu'CAAGCGGu'ACGACGAGCACCACCAGGACCU'GA |
| | | CCCu'GCu'GAAGGCCCu'GGu'GCGGCAGCAGCu'GCCCGAGAAGu'ACAAGG |
| | | AGAu'Cu'u'Cu'u'CGACCAGAGCAAGAACGGCu'ACGCCGGCu'ACAu'CGA |
| | | CGGCGGCGCCAGCCAGGAGGAGu'u'Cu'ACAAGu'u'CAu'CAAGCCCAu'CC |
| | | u'GGAGAAGAu'GGACGGCACCGAGGAGCu'GCu'GGu'GAAGCu'GAACCGGG |
| | | AGGACCu'GCu'GCGGAAGCAGCGGACCu'u'CGACAACGGCAGCAu'CCCCCA |
| | | CCAGAu'CCACCu'GGGCGAGCu'GCACGCCAu'CCu'GCGGCGGCAGGAGGAC |
| | | u'u'Cu'ACCCCu'u'CCu'GAAGGACAACCGGGAGAAGAu'CGAGAAGAu'CC |
| | | u'GACCu'u'CCGGAu'CCCCu'ACu'ACGu'GGGCCCCCu'GGCCCGGGGCAA |
| | | CAGCCGGu'u'CGCCu'GGAu'GACCCGCAAGAGCGAGGAGACAAu'CACCCCC |
| | | u'GGAACu'u'CGAGGAGGu'GGu'GGACAAGGGCGCCAGCGCCCAGAGCu'u' |
| | | CAu'CGAGCGGAu'GACCAACu'u'CGACAAGAACCu'GCCCAACGAGAAGGu' |
| | | GCu'GCCCAAGCACAGCCu'GCu'Gu'ACGAGu'ACu'u'CACCGu'Gu'ACAA |
| | | CGAGCu'GACCAAGGu'GAAGu'ACGu'GACCGAGGGCAu'GCGGAAGCCCGCC |
| | | u'u'CCu'GAGCGGCGAGCAGAAGAAGGCCAu'CGu'GGACCu'GCu'Gu'u'C |
| | | AAGACCAACCGGAAGGu'GACCGu'GAAGCAGCu'GAAGGAGGACu'ACu'u'C |
| | | AAGAAGAu'CGAGu'GCu'u'CGACAGCGu'GGAGAu'CAGCGGCGu'GGAGGA |
| | | CCGGu'u'CAACGCCAGCCu'GGGCACCu'ACCACGACCu'GCu'GAAGAu'CA |
| | | u'CAAGGACAAGGACu'u'CCu'GGACAACGAGGAGAACGAGGACAu'CCu'GG |
| | | AGGACAu'CGu'GCu'GACCCu'GACCCu'Gu'u'CGAGGACCGGGAGAu'GAu' |
| | | CGAGGAGCGGCu'GAAGACCu'ACGCCCACCu'Gu'u'CGACGACAAGGu'GA |
| | | u'GAAGCAGCu'GAAGCGGCGGCGGu'ACACCGGCu'GGGGCCGGCu'GAGCCG |
| | | GAAGCu'GAu'CAACGGCAu'CCGGGACAAGCAGAGCGGCAAGACCAu'CCu'G |
| | | GACu'u'CCu'CAAGAGCGACGGCu'u'CGCCAACCGGAACu'u'CAu'GCAGC |
| | | u'GAu'CCACGACGACAGCCu'GACCu'u'CAAGGAGGACAu'CCAGAAGGCCC |
| | | AGGu'GAGCGGCCAGGGCGACAGCCu'GCACGAGCACAu'CGCCAACCu'GGCC |
| | | GGCAGCCCCGCCAu'CAAGAAGGGCAu'CCu'GCAGACCGu'GAAGGu'GGu'G |
| | | GACGAGCu'GGu'GAAGGu'GAu'GGGCCGGCACAAGCCCGAGAACAu'CGu'G |
| | | Au'CGAGAu'GGCCCGGGAGAACCAGACCACCCAGAAGGGCCAGAAGAACAGCC |
| | | GGGAGCGGAu'GAAGCGGAu'CGAGGAGGGCAu'CAAGGAGCu'GGGCAGCCAG |
| | | Au'CCu'GAAGGAGCACCCCGu'GGAGAACACCCAGCu'GCAGAACGAGAAGCu' |
| | | Gu'ACCu'Gu'ACu'ACCu'GCAGAACGGCCGGGACAu'Gu'ACGu'GGACCA |
| | | GGAGCu'GGACAu'CAACCGGCu'GAGCGACu'ACGACGu'GGACCACAu'CGu' |
| | | GCCCCAGAGCu'u'CCu'GAAGGACGACAGCAu'CGACAACAAGGu'GCu'GA |
| | | CCCGGAGCGACAAGAACCGGGGCAAGAGCGACAACGu'GCCCAGCGAGGAGGu' |
| | | GGu'GAAGAAGAu'GAAGAACu'ACu'GGCGGCAGCu'GCu'GAACGCCAAGCu' |
| | | GAu'CACCCAGCGGAAGu'u'CGACAACCu'GACCAAGGCCGAGCGGGGCGGC |
| | | Cu'GAGCGAGCu'GGACAAGGCCGGCu'u'CAu'CAAGCGGCAGCu'GGu'GGA |
| | | GACACGGCAGAu'CACCAAGCACGu'GGCCCAGAu'CCu'GGACAGCCGGAu'G |
| | | AACACCAAGu'ACGACGAGAACGACAAGCu'GAu'CCGGGAGGu'GAAGGu'GA |
| | | u'CACCCu'CAAGAGCAAGCu'GGu'GAGCGACu'u'CCGGAAGGACu'u'CCA |
| | | Gu'u'Cu'ACAAGGu'GCGGGAGAu'CAACAACu'ACCACCACGCCCACGACGC |
| | | Cu'ACCu'GAACGCCGu'GGu'GGGCACCGCCCu'GAu'CAAGAAGu'ACCCCA |
| | | AGCu'GGAGAGCGAGu'u'CGu'Gu'ACGGCGACu'ACAAGGu'Gu'ACGACGu' |
| | | GCGGAAGAu'GAu'CGCCAAGAGCGAGCAGGAGAu'CGGCAAGGCCACCGCCA |
| | | AGu'ACu'u'Cu'u'Cu'ACAGCAACAu'CAu'GAACu'u'Cu'u'CAAGACCG |
| | | AGAu'CACCCu'GGCCAACGGCGAGAu'CCGGAAGCGGCCCCu'GAu'CGAGAC |
| | | AAACGGCGAGACAGGCGAGAu'CGu'Gu'GGGACAAGGGCCGGGACu'u'CGCC |
| | | ACCGu'GCGGAAGGu'GCu'GAGCAu'GCCCCAGGu'GAACAu'CGu'GAAGAA |
| | | GACCGAGGu'GCAGACCGGCGGCu'u'CAGCAAGGAGAGCAu'CCu'GCCCAAG |
| | | CGGAACAGCGACAAGCu'GAu'CGCCCGGAAGAAGGACu'GGGACCCCAAGAAG |
| | | u'ACGGCGGCu'u'CGACAGCCCCACCGu'GGCCu'ACAGCGu'GCu'GGu'GG |
| | | u'GGCCAAGGu'GGAGAAGGGCAAGAGCAAGAAGCu'CAAGAGCGu'GAAGGAG |
| | | Cu'GCu'GGGCAu'CACCAu'CAu'GGAGCGGAGCAGCu'u'CGAGAAGAACCC |
| | | CAu'CGACu'u'CCu'GGAGGCCAAGGGCu'ACAAGGAGGu'GAAGAAGGACCU' |
| | | GAu'CAu'CAAGCu'GCCCAAGu'ACAGCCu'Gu'u'CGAGCu'GGAGAACGG |
| | | CCGGAAGCGGAu'GCu'GGCCAGCGCCGGCGAGCu'GCAGAAGGGCAACGAGCu' |
| | | GGCCCu'GCCCAGCAAGu'ACGu'GAACu'u'CCu'Gu'ACCu'GGCCAGCCA |
| | | Cu'ACGAGAAGCu'GAAGGGCAGCCCCGAGGACAACGAGCAGAAGCAGCu'Gu' |
| | | u'CGu'GGAGCAGCACAAGCACu'ACCu'GGACGAGAu'CAu'CGAGCAGAu'C |
| | | AGCGAGu'u'CAGCAAGCGGGu'GAu'CCu'GGCCGACGCCAACCu'GGACAAG |
| | | Gu'GCu'GAGCGCCu'ACAACAAGCACCGGGACAAGCCCAu'CCGGGAGCAGGC |
| | | CGAGAACAu'CAu'CCACCu'Gu'u'CACCCu'GACCAACCu'GGGCGCCCCCG |
| | | CCGCCu'u'CAAGu'ACu'u'CGACACCACCAu'CGACCGGAAGCGGu'ACACC |
| | | AGCACCAAGGAGGu'GCu'GGACGCCACCCu'GAu'CCACCAGAGCAu'CACCG |
| | | GCCu'Gu'ACGAGACACGGAu'CGACCu'GAGCCAGCu'GGGCGGCGACGAGGG |
| | | CGCCGACAAGCGGACCGCCGACGGCAGCGAGu'u'CGAGAGCCCCAAGAAGAAG |
| | | CGGAAGGu'Gu'GAGCGGCCGCu'u'AAu'u'AAGCu'GCCu'u'Cu'GCGGGG |
| | | Cu'u'GCCu'u'Cu'GGCCAu'GCCCu'u'Cu'u'Cu'Cu'CCCu'u'GCACCu' |
| | | Gu'ACCu'Cu'u'GGu'Cu'u'u'GAAu'AAAGCCu'GAGu'AGGAAGu'Cu' |
| | | AGA (SEQ ID NO: 5746) |
| | protein | MSEVEFSHEYWMRHALTLAKRARDEREVPVGAVLVLNNRVIGEGWNRAIGLHDP |
| | | TAHAEIMALRQGGLVMQNYRLIDATLYVTFEPCVMCAGAMIHSRIGRVVFGVRN |
| | | AKTGAAGSLMDVLHHPGMNHRVEITEGILADECAALLCRFFRMPRRVFNAQKKA |
| | | QSSTDSGGSSGGSSGSETPGTSESATPESSGGSSGGSDKKYSIGLAIGTNSVGW |
| | | AVITDEYKVPSKKFKVLGNTDRHSIKKNLIGALLFDSGETAEATRLKRTARRRY |
| | | TRRKNRICYLQEIFSNEMAKVDDSFFHRLEESFLVEEDKKHERHPIFGNIVDEV |
| | | AYHEKYPTIYHLRKKLVDSTDKADLRLIYLALAHMIKFRGHFLIEGDLNPDNSD |
| | | VDKLFIQLVQTYNQLFEENPINASGVDAKAILSARLSKSRRLENLIAQLPGEKK |
| | | NGLFGNLIALSLGLTPNFKSNFDLAEDAKLQLSKDTYDDDLDNLLAQIGDQYAD |
| | | LFLAAKNLSDAILLSDILRVNTEITKAPLSASMIKRYDEHHQDLTLLKALVRQQ |
| | | LPEKYKEIFFDQSKNGYAGYIDGGASQEEFYKFIKPILEKMDGTEELLVKLNRE |
| | | DLLRKQRTFDNGSIPHQIHLGELHAILRRQEDFYPFLKDNREKIEKILTFRIPY |
| | | YVGPLARGNSRFAWMTRKSEETITPWNFEEVVDKGASAQSFIERMTNFDKNLPN |
| | | EKVLPKHSLLYEYFTVYNELTKVKYVTEGMRKPAFLSGEQKKAIVDLLFKTNRK |
| | | VTVKQLKEDYFKKIECFDSVEISGVEDRFNASLGTYHDLLKIIKDKDFLDNEEN |
| | | EDILEDIVLTLTLFEDREMIEERLKTYAHLFDDKVMKQLKRRRYTGWGRLSRKL |
| | | INGIRDKQSGKTILDFLKSDGFANRNFMQLIHDDSLTFKEDIQKAQVSGQGDSL |
| | | HEHIANLAGSPAIKKGILQTVKVVDELVKVMGRHKPENIVIEMARENOTTQKGQ |
| | | KNSRERMKRIEEGIKELGSQILKEHPVENTQLQNEKLYLYYLQNGRDMYVDQEL |
| | | DINRLSDYDVDHIVPQSFLKDDSIDNKVLTRSDKNRGKSDNVPSEEVVKKMKNY |
| | | WRQLLNAKLITQRKFDNLTKAERGGLSELDKAGFIKRQLVETROITKHVAQILD |
| | | SRMNTKYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIVKKT |
| | | EVQTGGFSKESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKYSLFEL |
| | | ENGRKRMLASAGELQKGNELALPSKYVNFLYLASHYEKLKGSPEDNEQKOLFVE |
| | | QHKHYLDEIIEQISEFSKRVILADANLDKVLSAYNKHRDKPIREQAENIIHLFT |
| | | LTNLGAPAAFKYFDTTIDRKRYTSTKEVLDATLIHQSITGLYETRIDLSQLGGD |
| | | EGADKRTADGSEFESPKKKRKV (SEQ ID NO: 5747) |
| 5'UTR | mRNA | AGGAAAu'AAGAGAGAAAAGAAGAGu'AAGAAGAAAu'Au'AAGAGCCACC |
| | | (SEQ ID NO: 5748) |
| TadA | mRNA | Au'GAGCGAGGu'GGAGu'u'CAGCCACGAGu'ACu'GGAu'GCGGCACGCCCu' |
| | | GACCCu'GGCCAAGCGGGCCCGGGACGAGCGGGAGGu'GCCCGu'GGGCGCCG |
| | | u'GCu'GGu'GCu'GAACAACCGGGu'GAu'CGGCGAGGGCu'GGAACCGGGCC |
| | | Au'CGGCCu'GCACGACCCCACCGCCCACGCCGAGAu'CAu'GGCCCu'GCGGC |
| | | AGGGCGGCCu'GGu'GAu'GCAGAACu'ACCGGCu'GAu'CGACGCCACCCu'G |
| | | u'ACGu'GACCu'u'CGAGCCCu'GCGu'GAu'Gu'GCGCCGGCGCCAu'GAu' |
| | | CCACAGCCGGAu'CGGCCGGGu'GGu'Gu'u'CGGCGu'GCGGAACGCCAAGAC |
| | | CGGCGCCGCCGGCAGCCu'GAu'GGACGu'GCu'GCACCACCCCGGCAu'GAAC |
| | | CACCGGGu'GGAGAu'CACCGAGGGCAu'CCu'GGCCGACGAGu'GCGCCGCCC |
| | | u'GCu'Gu'GCCGGu'u'Cu'u'CCGGAu'GCCCCGGCGGGu'Gu'u'CAACGC |
| | | CCAGAAGAAGGCCCAGAGCAGCACCGAC (SEQ ID NO: 5749) |
| | protein | MSEVEFSHEYWMRHALTLAKRARDEREVPVGAVLVLNNRVIGEGWNRAIGLHDP |
| | | TAHAEIMALRQGGLVMQNYRLIDATLYVTFEPCVMCAGAMIHSRIGRVVFGVRN |
| | | AKTGAAGSLMDVLHHPGMNHRVEITEGILADECAALLCRFFRMPRRVFNAQKKA |
| | | QSSTD (SEQ ID NO: 5750) |
| Linker | mRNA | AGCGGCGGCAGCAGCGGCGGCAGCAGCGGCAGCGAGACACCCGGCACCAGCGAG |
| between | | AGCGCCACCCCCGAGAGCAGCGGCGGCAGCAGCGGCGGCAGC (SEQ ID NO: |
| TadA | | 5751) |
| and Cas9 | protein | SGGSSGGSSGSETPGTSESATPESSGGSSGGS (SEQ ID NO: 5752) |
| nickase | | |
| Cas9 | mRNA | GACAAGAAGu'ACAGCAu'CGGCCu'GGCCAu'CGGCACCAACAGCGu'GGGCu' |
| nickase | | GGGCCGu'GAu'CACCGACGAGu'ACAAGGu'GCCCAGCAAGAAGu'u'CAAG |
| | | Gu'GCu'GGGCAACACCGACCGGCACAGCAu'CAAGAAGAACCu'GAu'CGGCG |
| | | CCCu'GCu'Gu'u'CGACAGCGGCGAGACAGCCGAGGCCACCCGGCu'GAAGCG |
| | | GACCGCCCGGCGGCGGu'ACACCCGGCGGAAGAACCGGAu'Cu'GCu'ACCu'G |
| | | CAGGAGAu'Cu'u'CAGCAACGAGAu'GGCCAAGGu'GGACGACAGCu'u'Cu' |
| | | u'CCACCGGCu'GGAGGAGAGCu'u'CCu'GGu'GGAGGAGGACAAGAAGCACG |
| | | AGCGGCACCCCAu'Cu'u'CGGCAACAu'CGu'GGACGAGGu'GGCCu'ACCAC |
| | | GAGAAGu'ACCCCACCAu'Cu'ACCACCu'GCGGAAGAAGCu'GGu'GGACAGC |
| | | ACCGACAAGGCCGACCu'GCGGCu'GAu'Cu'ACCu'GGCCCu'GGCCCACAu' |
| | | GAu'CAAGu'u'CCGGGGCCACu'u'CCu'GAu'CGAGGGCGACCu'GAACCCC |
| | | GACAACAGCGACGu'GGACAAGCu'Gu'u'CAu'CCAGCu'GGu'GCAGACCu' |
| | | ACAACCAGCu'Gu'u'CGAGGAGAACCCCAu'CAACGCCAGCGGCGu'GGACGC |
| | | CAAGGCCAu'CCu'GAGCGCCCGGCu'GAGCAAGAGCCGGCGGCu'GGAGAACC |
| | | u'GAu'CGCCCAGCu'GCCCGGCGAGAAGAAGAACGGCCu'Gu'u'CGGCAACC |
| | | u'GAu'CGCCCu'GAGCCu'GGGCCu'GACCCCCAACu'u'CAAGAGCAACu'u' |
| | | CGACCu'GGCCGAGGACGCCAAGCu'GCAGCu'GAGCAAGGACACCu'ACGAC |
| | | GACGACCu'GGACAACCu'GCu'GGCCCAGAu'CGGCGACCAGu'ACGCCGACC |
| | | u'Gu'u'CCu'GGCCGCCAAGAACCu'GAGCGACGCCAu'CCu'GCu'GAGCGA |
| | | CAu'CCu'GCGGGu'GAACACCGAGAu'CACCAAGGCCCCCCu'GAGCGCCAGC |
| | | Au'GAu'CAAGCGGu'ACGACGAGCACCACCAGGACCu'GACCCu'GCu'GAAG |
| | | GCCCu'GGu'GCGGCAGCAGCu'GCCCGAGAAGu'ACAAGGAGAu'Cu'u'Cu' |
| | | u'CGACCAGAGCAAGAACGGCu'ACGCCGGCu'ACAu'CGACGGCGGCGCCAGC |
| | | CAGGAGGAGu'u'Cu'ACAAGu'u'CAu'CAAGCCCAu'CCu'GGAGAAGAu'G |
| | | GACGGCACCGAGGAGCu'GCu'GGu'GAAGCu'GAACCGGGAGGACCu'GCu'G |
| | | CGGAAGCAGCGGACCu'u'CGACAACGGCAGCAu'CCCCCACCAGAu'CCACCu' |
| | | GGGCGAGCu'GCACGCCAu'CCu'GCGGCGGCAGGAGGACu'u'Cu'ACCCCu' |
| | | u'CCu'GAAGGACAACCGGGAGAAGAu'CGAGAAGAu'CCu'GACCu'u'CCG |
| | | GAu'CCCCu'ACu'ACGu'GGGCCCCCu'GGCCCGGGGCAACAGCCGGu'u'CG |
| | | CCu'GGAu'GACCCGCAAGAGCGAGGAGACAAu'CACCCCCu'GGAACu'u'CG |
| | | AGGAGGu'GGu'GGACAAGGGCGCCAGCGCCCAGAGCu'u'CAu'CGAGCGGAu' |
| | | GACCAACu'u'CGACAAGAACCu'GCCCAACGAGAAGGu'GCu'GCCCAAGCA |
| | | CAGCCu'GCu'Gu'ACGAGu'ACu'u'CACCGu'Gu'ACAACGAGCu'GACCAA |
| | | GGu'GAAGu'ACGu'GACCGAGGGCAu'GCGGAAGCCCGCCu'u'CCu'GAGCG |
| | | GCGAGCAGAAGAAGGCCAu'CGu'GGACCu'GCu'Gu'u'CAAGACCAACCGGA |
| | | AGGu'GACCGu'GAAGCAGCu'GAAGGAGGACu'ACu'u'CAAGAAGAu'CGAG |
| | | u'GCu'u'CGACAGCGu'GGAGAu'CAGCGGCGu'GGAGGACCGGu'u'CAACG |
| | | CCAGCCu'GGGCACCu'ACCACGACCu'GCu'GAAGAu'CAu'CAAGGACAAGG |
| | | ACu'u'CCu'GGACAACGAGGAGAACGAGGACAu'CCu'GGAGGACAu'CGu'G' |
| | | Cu'GACCCu'GACCCu'Gu'u'CGAGGACCGGGAGAu'GAu'CGAGGAGCGGCu |
| | | GAAGACCu'ACGCCCACCu'Gu'u'CGACGACAAGGu'GAu'GAAGCAGCu'G |
| | | AAGCGGCGGCGGu'ACACCGGCu'GGGGCCGGCu'GAGCCGGAAGCu'GAu'CA |
| | | ACGGCAu'CCGGGACAAGCAGAGCGGCAAGACCAu'CCu'GGACu'u'CCu'CA |
| | | AGAGCGACGGCu'u'CGCCAACCGGAACu'u'CAu'GCAGCu'GAu'CCACGAC |
| | | GACAGCCu'GACCu'u'CAAGGAGGACAu'CCAGAAGGCCCAGGu'GAGCGGCC |
| | | AGGGCGACAGCCu'GCACGAGCACAu'CGCCAACCu'GGCCGGCAGCCCCGCCA |
| | | u'CAAGAAGGGCAu'CCu'GCAGACCGu'GAAGGu'GGu'GGACGAGCu'GGu' |
| | | GAAGGu'GAu'GGGCCGGCACAAGCCCGAGAACAu'CGu'GAu'CGAGAu'GGC |
| | | CCGGGAGAACCAGACCACCCAGAAGGGCCAGAAGAACAGCCGGGAGCGGAu'GA |
| | | AGCGGAu'CGAGGAGGGCAu'CAAGGAGCu'GGGCAGCCAGAu'CCu'GAAGGA |
| | | GCACCCCGu'GGAGAACACCCAGCu'GCAGAACGAGAAGCu'Gu'ACCu'Gu'A |
| | | Cu'ACCu'GCAGAACGGCCGGGACAu'Gu'ACGu'GGACCAGGAGCu'GGACAu' |
| | | CAACCGGCu'GAGCGACu'ACGACGu'GGACCACAu'CGu'GCCCCAGAGCu' |
| | | u'CCu'GAAGGACGACAGCAu'CGACAACAAGGu'GCu'GACCCGGAGCGACAA |
| | | GAACCGGGGCAAGAGCGACAACGu'GCCCAGCGAGGAGGu'GGu'GAAGAAGAu' |
| | | GAAGAACu'ACu'GGCGGCAGCu'GCu'GAACGCCAAGCu'GAu'CACCCAGC |
| | | GGAAGu'u'CGACAACCu'GACCAAGGCCGAGCGGGGGGCCu'GAGCGAGCu' |
| | | GGACAAGGCCGGCu'u'CAu'CAAGCGGCAGCu'GGu'GGAGACACGGCAGAU' |
| | | CACCAAGCACGu'GGCCCAGAu'CCu'GGACAGCCGGAu'GAACACCAAGu'AC |
| | | GACGAGAACGACAAGCu'GAu'CCGGGAGGu'GAAGGu'GAu'CACCCu'CAAG |
| | | AGCAAGCu'GGu'GAGCGACu'u'CCGGAAGGACu'u'CCAGu'u'Cu'ACAAG |
| | | Gu'GCGGGAGAu'CAACAACu'ACCACCACGCCCACGACGCCu'ACCu'GAACG |
| | | CCGu'GGu'GGGCACCGCCCu'GAu'CAAGAAGu'ACCCCAAGCu'GGAGAGCG |
| | | AGu'u'CGu'Gu'ACGGCGACu'ACAAGGu'Gu'ACGACGu'GCGGAAGAu'GA |
| | | u'CGCCAAGAGCGAGCAGGAGAu'CGGCAAGGCCACCGCCAAGu'ACu'u'Cu' |
| | | u'Cu'ACAGCAACAu'CAu'GAACu'u'Cu'u'CAAGACCGAGAu'CACCCu'G |
| | | GCCAACGGCGAGAu'CCGGAAGCGGCCCCu'GAu'CGAGACAAACGGCGAGACA |
| | | GGCGAGAu'CGu'Gu'GGGACAAGGGCCGGGACu'u'CGCCACCGu'GCGGAAG |
| | | Gu'GCu'GAGCAu'GCCCCAGGu'GAACAu'CGu'GAAGAAGACCGAGGu'GCA |
| | | GACCGGCGGCu'u'CAGCAAGGAGAGCAu'CCu'GCCCAAGCGGAACAGCGACA |
| | | AGCu'GAu'CGCCCGGAAGAAGGACu'GGGACCCCAAGAAGu'ACGGCGGCu'u' |
| | | CGACAGCCCCACCGu'GGCCu'ACAGCGu'GCu'GGu'GGu'GGCCAAGGu'G |
| | | GAGAAGGGCAAGAGCAAGAAGCu'CAAGAGCGu'GAAGGAGCu'GCu'GGGCAu' |
| | | CACCAu'CAu'GGAGCGGAGCAGCu'u'CGAGAAGAACCCCAu'CGACu'u'C |
| | | Cu'GGAGGCCAAGGGCu'ACAAGGAGGu'GAAGAAGGACCu'GAu'CAu'CAAG |
| | | Cu'GCCCAAGu'ACAGCCu'Gu'u'CGAGCu'GGAGAACGGCCGGAAGCGGAu' |
| | | GCu'GGCCAGCGCCGGCGAGCu'GCAGAAGGGCAACGAGCu'GGCCCu'GCCCA |
| | | GCAAGu'ACGu'GAACu'u'CCu'Gu'ACCu'GGCCAGCCACu'ACGAGAAGCu' |
| | | GAAGGGCAGCCCCGAGGACAACGAGCAGAAGCAGCu'Gu'u'CGu'GGAGCAG |
| | | CACAAGCACu'ACCu'GGACGAGAu'CAu'CGAGCAGAu'CAGCGAGu'u'CAG |
| | | CAAGCGGGu'GAu'CCu'GGCCGACGCCAACCu'GGACAAGGu'GCu'GAGCGC |
| | | Cu'ACAACAAGCACCGGGACAAGCCCAu'CCGGGAGCAGGCCGAGAACAu'CAu' |
| | | CCACCu'Gu'u'CACCCu'GACCAACCu'GGGCGCCCCCGCCGCCu'u'CAAG |
| | | u'ACu'u'CGACACCACCAu'CGACCGGAAGCGGu'ACACCAGCACCAAGGAGG |
| | | u'GCu'GGACGCCACCCu'GAu'CCACCAGAGCAu'CACCGGCCu'Gu'ACGAG |
| | | ACACGGAu'CGACCu'GAGCCAGCu'GGGCGGCGAC (SEQ ID NO: 5753) |
| | protein | DKKYSIGLAIGTNSVGWAVITDEYKVPSKKFKVLGNTDRHSIKKNLIGALLFDS |
| | | GETAEATRLKRTARRRYTRRKNRICYLQEIFSNEMAKVDDSFFHRLEESFLVEE |
| | | DKKHERHPIFGNIVDEVAYHEKYPTIYHLRKKLVDSTDKADLRLIYLALAHMIK |
| | | FRGHFLIEGDLNPDNSDVDKLFIQLVQTYNQLFEENPINASGVDAKAILSARLS |
| | | KSRRLENLIAQLPGEKKNGLFGNLIALSLGLTPNFKSNFDLAEDAKLQLSKDTY |
| | | DDDLDNLLAQIGDQYADLFLAAKNLSDAILLSDILRVNTEITKAPLSASMIKRY |
| | | DEHHQDLTLLKALVRQQLPEKYKEIFFDQSKNGYAGYIDGGASQEEFYKFIKPI |
| | | LEKMDGTEELLVKLNREDLLRKQRTFDNGSIPHQIHLGELHAILRRQEDFYPFL |
| | | KDNREKIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMRKPAFLS |
| | | GEQKKAIVDLLFKTNRKVTVKQLKEDYFKKIECFDSVEISGVEDRFNASLGTYH |
| | | DLLKIIKDKDFLDNEENEDILEDIVLTLTLFEDREMIEERLKTYAHLFDDKVMK |
| | | QLKRRRYTGWGRLSRKLINGIRDKQSGKTILDFLKSDGFANRNFMQLIHDDSLT |
| | | FKEDIQKAQVSGQGDSLHEHIANLAGSPAIKKGILQTVKVVDELVKVMGRHKPE |
| | | NIVIEMARENQTTQKGQKNSRERMKRIEEGIKELGSQILKEHPVENTQLQNEKL |
| | | YLYYLQNGRDMYVDQELDINRLSDYDVDHIVPQSFLKDDSIDNKVLTRSDKNRG |
| | | KSDNVPSEEVVKKMKNYWRQLLNAKLITQRKFDNLTKAERGGLSELDKAGFIKR |
| | | QLVETRQITKHVAQILDSRMNTKYDENDKLIREVKVITLKSKLVSDFRKDFQFY |
| | | KVREINNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKYGGFD |
| | | SPTVAYSVLVVAKVEKGKSKKLKSVKELLGITIMERSSFEKNPIDFLEAKGYKE |
| | | VKKDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYNKH |
| | | RDKPIREQAENIIHLFTLTNLGAPAAFKYFDTTIDRKRYTSTKEVLDATLIHQS |
| | | ITGLYETRIDLSQLGGD (SEQ ID NO: 5754) |
| Linker | mRNA | GAGGGCGCCGAC (SEQ ID NO: 5755) |
| between | Protein | EGAD (SEQ ID NO: 5756) |
| Cas9 | | |
| nickase | | |
| and NLS | | |
| Nuclear | mRNA | AAGCGGACCGCCGACGGCAGCGAGu'u'CGAGAGCCCCAAGAAGAAGCGGAAGG |
| locali- | | u'Gu'GA (SEQ ID NO: 5757) |
| zation | Protein | KRTADGSEFESPKKKRKV (SEQ ID NO: 5758) |
| sequence | | |
| (NLS) | | |
| 3′UTR | mRNA | GCGGCCGCu'u'AAu'u'AAGCu'GCCu'u'Cu'GCGGGGCu'u'GCCu'u' |
| | | Cu'GGCCAu'GCCCu'u'Cu'u'Cu'Cu'CCCu'u'GCACCu'Gu'ACCu' |
| | | Cu'u'GGu'Cu'u'u'GAAu'AAAGCCu'GAGu'AGGAAGu'Cu'AGA (SEQ |
| | | ID NO: 5759) |

The mutations at amino acid positions 691 and 1135 of the nCas9 component and their correspondingnucleotide sequences are indicated as bold and underlined.
u': N¹-methylpseudouridine
The first nucleotide in the 5′UTR has a 2′-O-methyl modification.

Other ABE variants may be employed to effect editing of human TTR gene. Examples of such ABE variants are described International Patent Application PCT/US21/26729, filed on Apr. 9, 2021, entitled BASE EDITING OF PCSK9 AND METHODS OF USING SAME FOR TREATMENT OF DISEASE, and naming Verve Therapeutics, Inc. as the applicant.

### Example 3

### In Vivo Non-Human Primate (NHP) Base Editing of TTR Gene

In this example, NHP surrogate sgRNAs (GA519 and GA520), corresponding to the human GA457 and GA460 sgRNAs described above, were prepared, and formulated with previously described ABE8.8 mRNA, encapsulated in lipid nanoparticles (LNPs), and intravenously dosed to NHPs. The study involved two distinct aspects.

The first aspect of the NHP in vivo study involved evaluating LNP1 and LNP2, which differed only in that LNP1 was formulated to encapsulate GA519 and ABE8.8 mRNA whereas LNP2 was formulated to encapsulate GA520 and ABE8.8 mRNA. The second aspect of the study involved formulating and evaluating a third LNP (LNP3). LNP3, like LNP1, was formulated to encapsulate GA519 and ABE8.8 mRNA. However, LNP 3 differed from LNP1 in that LNP3 included a GalNAc moiety constituent. In each aspect of the study, base editing efficiency, TTR protein expression, safety profiles, and pharmacokinetics were evaluated at multiple times post-infusion of the NHPs, as is further detailed below and illustrated in the accompanying figures.

### Part A: In Vivo NHP Evaluation of GA519 and GA520 Using Non-GalNAc LNPs

### LNP Preparation

In this first aspect of the NHP study, two LNPs (LNP1 and LNP2) were formulated, with LNP1 encapsulating GA519 and ABE8.8 mRNA and LNP2 encapsulate GA520 and ABE8.8 mRNA. The constituents of each of the LNPs are comprised of an ionizable amino lipid (iLipid), a neutral helper lipid, a PEG-Lipid and a sterol lipid as described in and at the ratios indicated in Table 12 below.

TABLE 12
LNP1/LNP2 Components

| LNP | | | Mol |
|---|---|---|---|
| Component | Lipid names | Lipid structure | % |
| Amino lipid (iLipid) | 3-((4,4-bis(octyloxy)butanoyl)oxy)-2-((((3- (diethylamino)propoxy)carbonyl)oxy)methyl) propyl (9Z,12Z)-octadeca-9,12-dienoate* | 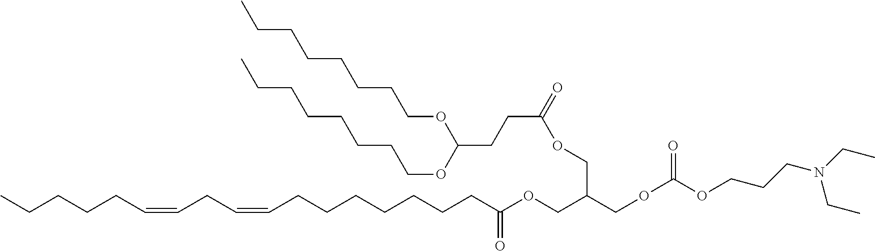 [SMILES: CCCCC/C=C\C/C=C\CCCCCCCC(=O)OCC(COC(=O)CCC(OCCCCCCCC)OCCCCCCCC)COC(=O)OCCCN(CC)CC] | 50 |
| Neutral helper lipid | 1,2-distearoyl-sn-glycero-3- phosphocholine (DSPC) | 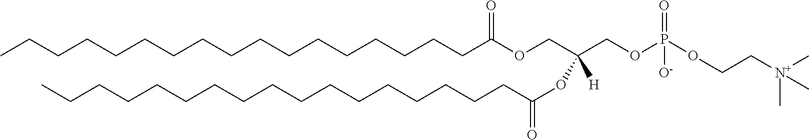 [SMILES: CCCCCCCCCCCCCCCCCC(=O)OC[C@H](COP(=O)([O-])OCC[N+](C)(C)C)OC(=O)CCCCCCCCCCCCCCCCC] | 9 |
| PEG-lipid | 1,2-dimyristoyl-rac-glycero-3- methoxypolyethylene glycol-2000 (PEG₂₀₀₀-DMG) | 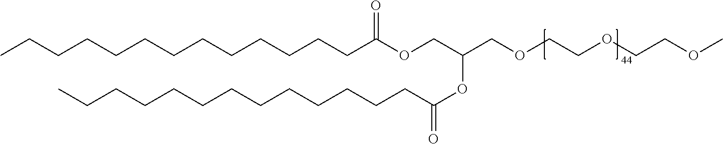 | 3 |
| Sterol lipid | Cholesterol | 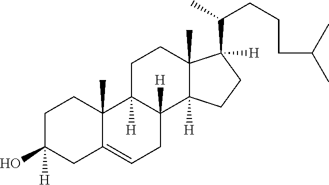 | 38 |

*described in International Published Patent Application WO 2015/095340 A1

It should be understood that the lipids in Table 12 may be substituted for other suitable lipids in the listed class. In some embodiments, for example, the LNP comprises the amino lipid VL422 described in the International published patent application WO 2022/060871 A1. For example, the amino lipid may be VL422, or a pharmaceutically acceptable salt or solvate thereof:

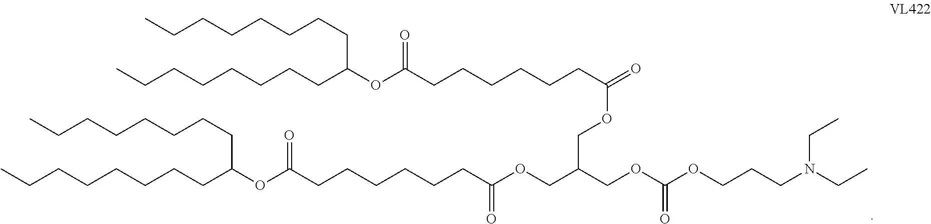 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CC]

It should be further understood that the mol % of lipids in Table 12 may be adjusted and that the mol % included in Table 12 are targeted excipient percentages of the LNP, which is intended to represent the aggregate mol % of all the LNPs formulated in a given batch and that specific LNPs within a batch may have varying mol %. Thus, it is contemplated herein that the mol % of one or more, or all of the LNP components set forth in Table 12 may be adjusted, for example, by +/−1-5%, +/−5-10%, or +/−10%-20%. It is further contemplated herein that the mol % of one or more, or all of the LNP components set forth in Table 12 with respect to a specific LNP formulated in a given batch of LNPs formulated in accordance with desired target excipient percentages, may vary from the targeted mol %, for example, by +/−1-5%, +/−5-10%, or +/−10%-20%, or even greater than +/−20%. Further, it should be understood that additional LNP components, including non-lipid components, may be added to the LNP components set-forth in Table 12. As set forth in Table 13, LNP 1 was formulated with sgRNA GA519 and LNP2 was formulated with GA520, which correspond respectively to the sgRNA GA457 and GA460, previously described. GA519 and GA520 were chemically synthesized and the sequences and chemical modifications of GA519 and GA520 are specified in Table 13.

TABLE 13
GA519 and GA520 TTR Gene Targeted Guides

| | | | | Equivalent | |
|---|---|---|---|---|---|
| | gRNA | | Protospacer | Human | |
| LNP | ID | Species | (5′-3′) | gRNA | gRNA Sequence (5′-3′) |
| 1 | GA519 | Cyno | GCCATCCTGC | GA457 | gscscsAUCCUGCCAAGAACG |
| | | | CAAGAACGAG | | AGgUUUUAGagcuaGaaauag |
| | | | (SEQ ID | | caaGUUaAaAuAaggcuaGUc |
| | | | NO; 28) | | CGUUAucAAcuuGaaaaagug |
| | | | | | Gcaccgagucggugcuususu |
| | | | | | S (SEQ ID NO: 16) |
| 2 | GA520 | Cyno | TATAGGAAAA | GA460 | usasusAGGAAAACCAGUGAG |
| | | | CCAGTGAGTC | | UCgUUUUAGagcuaGaaauag |
| | | | (SEQ ID | | caaGUUaAaAuAaggcuaGUc |
| | | | NO: 26) | | CGUUAucAAcuuGaaaaagug |
| | | | | | Gcaccgagucggugcuususu |
| | | | | | s (SEQ ID NO: 17) |

Letters in the sequences:
A = adenosine; C = cytidine; G = guanosine; U = uridine; a = 2′-O-methyladenosine; c = 2′-O-methylcytidine; g = 2′-O-methylguanosine; u = 2′-O-methyluridine; s = phosphorothioate (PS) backbone linkage.
C = nucleotide that differs in NHP from human TTR sequence.
Bold type in gRNA sequence denotes spacer sequence corresponding to Protospacer.

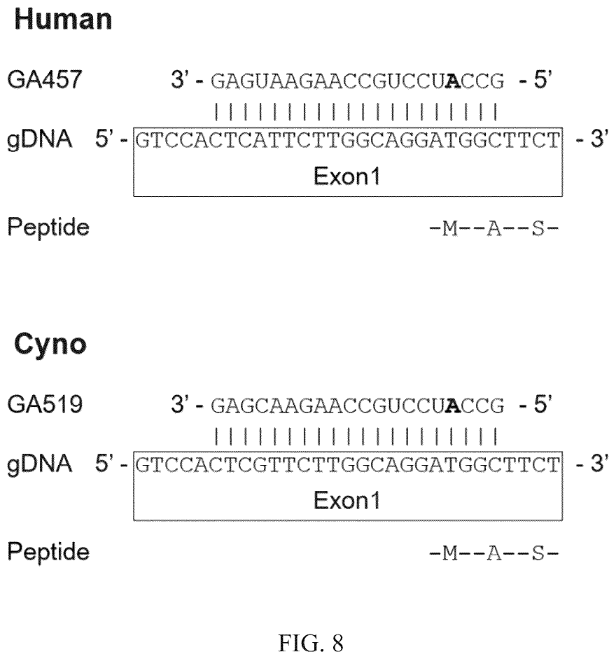

Notably as compared to GA457, GA519 hybridizes between positions 50,681,581 to 50,681,603 in exon 1 of the reference cynomolgus monkey genome (macFas5) and edits the adenosine at position 50,681,584 resulting in disruption of the full length TTR protein sequence by converting a methionine to a threonine amino acid and prohibiting protein translation (FIG. 8). GA519 is the cynomolgus surrogate of the human GA457 gRNA and maps to the analogous region of the human TTR locus as in FIG. 4 as previously described. The cynomolgus GA519 gRNA differs from GA457 by a single nucleotide at position 17 of the protospacer and is highlighted with an underline in Protospacer column of Table 13. Furthermore, GA519 and GA457 differ from one another in that the tracr region of GA519 incorporates chemical modifications (detailed in Table 13). The chemical modifications are designed for, or capable of, improving in vivo stability.

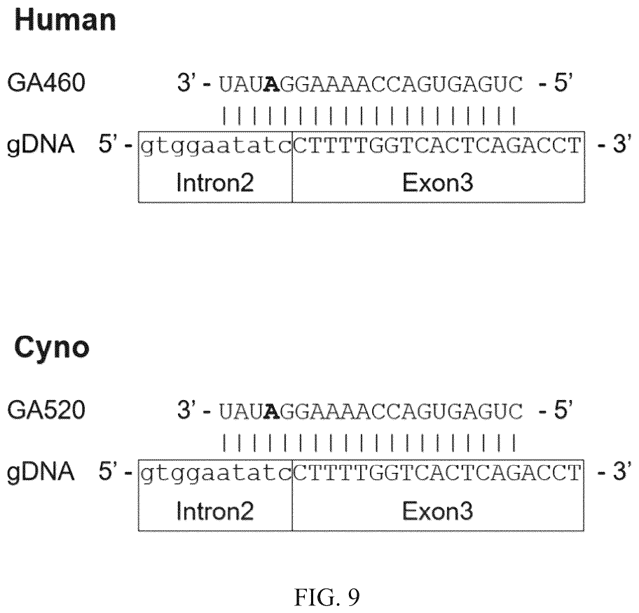

Similarly, as compared to GA460, GA520 hybridizes between positions 50,678,305 to 50,678,327 of exon 3 of the reference cynomolgus monkey genome (macFas5) and edits the adenosine at position 50,678,324 resulting in splicing acceptor disruption producing a truncated non-functional TTR protein (FIG. 9). The protospacer region for GA520 is identical to the human GA460 and maps to the analogous region of the human TTR locus as in FIG. 4 as previously described. GA520 and GA460 differ in the tracr region and incorporate chemical modifications, as detailed in the table above, that are designed for, or capable of, improving in vivo stability.

For reference, the targeted nucleotide for base editing is highlighted in bold in FIGS. 8 and 9. FIGS. 8 and 9 also identify the location of the spacer of GA519 and GA520 relative to the TTR gene as previously described.

LNP 1 and LNP2 were formulated using ABE 8.8 mRNA and GA519 and GA520, respectively, with an sgRNA:mRNA weight ratio of 1:1. In other words, the LNPs were formulated with an equal amount by weight of guide RNA as mRNA. The resulting LNPs encapsulating the sgRNAs and ABE 8.8 mRNA were filtered using 0.2-micron filters and frozen at −80° C. Physical characteristics of the formulated LNPs are summarized in Table 14.

TABLE 14
LNP1/LNP2 Characterization

| | | | RNA entrapment |
|---|---|---|---|
| LNP | Average LNP size (nm) | PDI | (%) |
| 1 | 68.6 | 0.022 | 95.7 |
| 2 | 68.6 | 0.029 | 96.2 |

PDI is Polydispersity Index

One of ordinary skill in the art would understand that the average LNP size, PDI and RNA entrapment values set forth in Table 14 are subject to measurement error or accuracy. It is also contemplated herein that the LNP size, PDI and RNA entrapment values set forth in Table 14 may be varied by +/−1-5%, +/−5-10%, or +/−10%-20%.

### NHP Study Design

In this aspect of the study, female cynomolgus monkeys of Cambodian origin were used as study animals. A premedication regimen comprising dexamethasone and H1 and H2 antihistamines was administered to all animals on day −1 (approximately 24 hours prior to dosing) and day 1 (predose), at 30 to 60 minutes prior to test article dose administration. Three monkeys were dosed with LNP1 and 3 monkey were dosed with LNP 2 on day 1 of the study via a single IV infusion at a dose level of 3 mg of combined sgRNA and mRNA per kg of animal body weight and at a dose volume of 6 mL/kg (n=3/group).

Blood samples were collected from all animals predose for baseline measurement and post-dose at various time points on days 1 through 15 to assess biomarkers, cytokines, plasma iLipid and PEG-Lipid pharmacokinetics, and serum safety parameters.

Necropsies were performed on all animals at day 16. Liver biopsy samples were collected to assess TTR gene editing.

### Analysis of Editing Efficiency

The amount of gene editing in the liver was evaluated by next-generation sequencing (NGS) of targeted polymerase chain reaction (PCR) amplicons at the TTR target site derived from genomic DNA extracted from the liver of the animal using the method described previously (Musunuru et al, Nature 593, no. 7859 (May 2021): 429-34. https://doi.org/10.1038/s41586-021-03534-y). Percent editing was reported as the percent of all reads containing a nonreference allele at the target adenine.

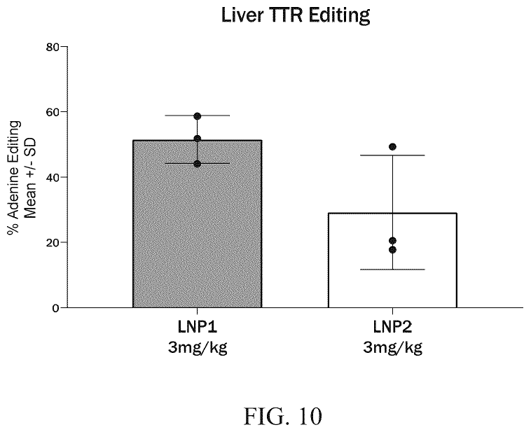

FIG. 10 illustrates TTR editing efficiency of LNP1 as compared to LNP2. Notably, as illustrated in FIG. 10, the average hepatic TTR editing efficiency is higher in NHP treated with LNP1 (52%) compared to LNP2 (29%).

### Quantification of TTR Protein Expression in Serum

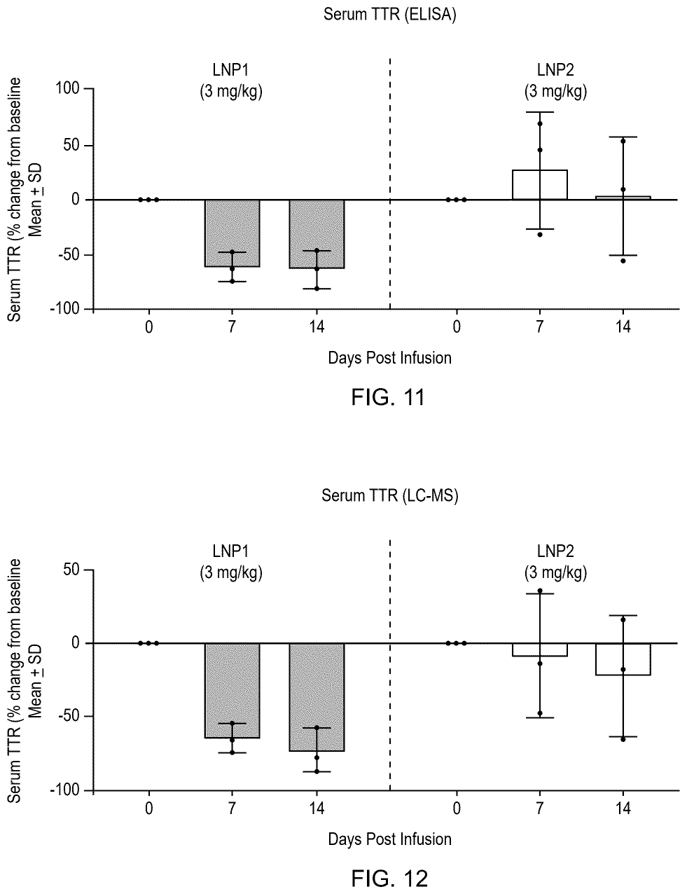

Serum was collected from all animals on days −10, −7, −5 pre-infusion and days 7, and 14 post LNP infusion for TTR protein analysis. Serum TTR was quantified using two methods. TTR protein levels were initially quantified using a custom TTR sandwich ELISA with the data obtained from that analysis presented in FIG. 11. Values for day −10, −7, and −5 were averaged to obtain the baseline value. Notably, LNP1 treated animals showed greater liver TTR editing, also showed greater plasma TTR reductions (−63% change from baseline on Day 14) when compared to LNP2 treated animals (3% change from baseline on Day 14). TTR protein collected from serum were also quantitated using liquid chromatography mass-spectrometry (LC-MS), in which four unique serum TTR peptide fragments were quantitated from each sample time point and the average of the results is reported. LC-MS serum TTR quantitation analysis using LC-MS is set forth in FIG. 12 and was notably consistent with the data obtained from the ELISA quantification in that it also demonstrated that LNP1 showed greater plasma TTR reductions (−73% change from baseline on day 14) when compared to LNP2 (−21% change from baseline on day 14).

Thus, as illustrated in FIGS. 10, 11 and 12, infusion of LNP1 and LNP2 in NHPs resulted in editing of the TTR gene in the liver, with LNP1 demonstrating greater editing than LNP2. The greater editing of LNP1 NHPs corresponded to a commensurate increase in the reduction in serum TTR concentrations in serum.

### Safety Analysis

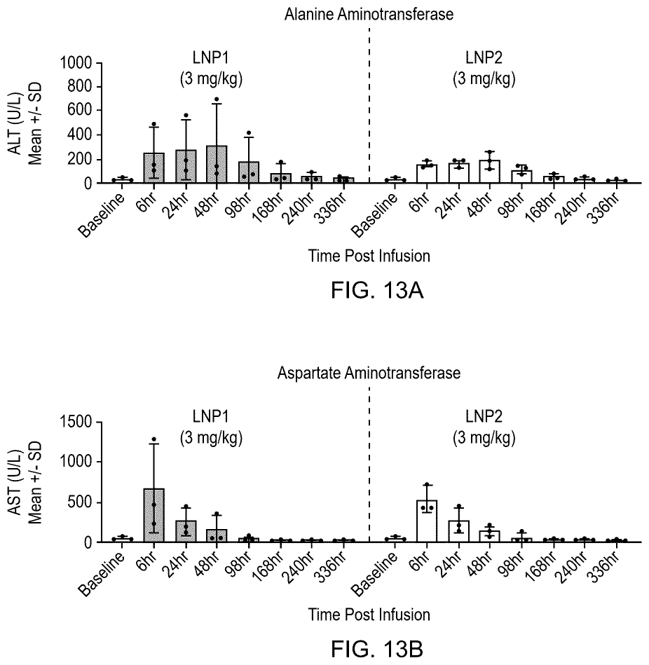

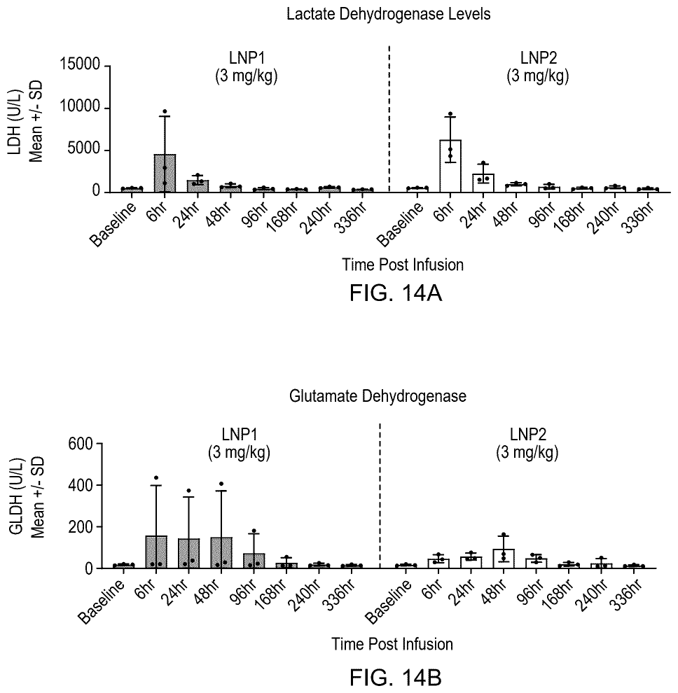

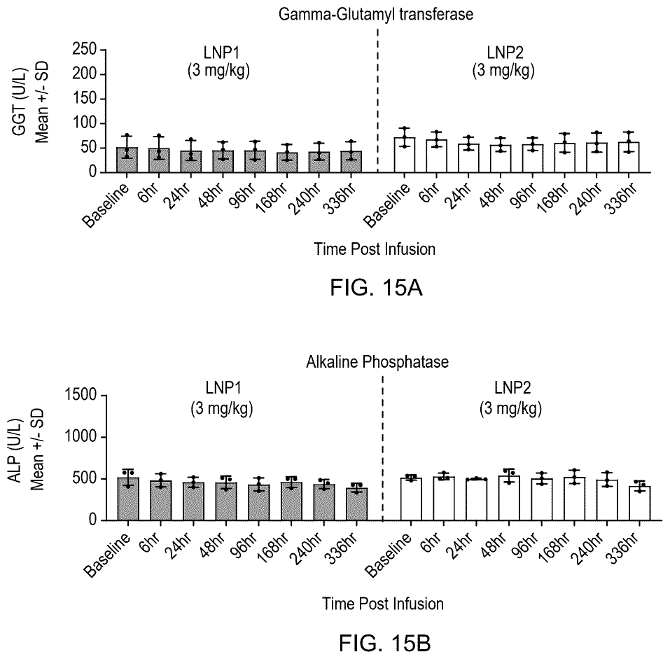

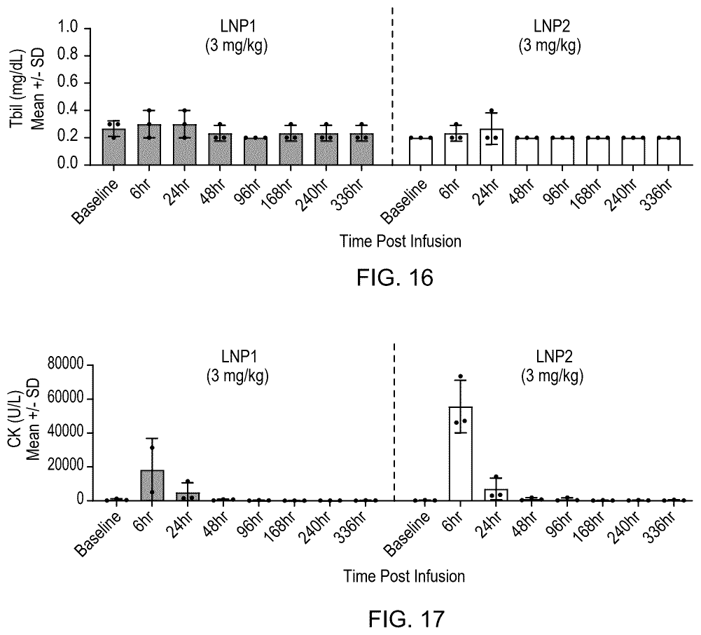

Blood serum was collected from all animals at day −10, −7, −5 pre-infusion and 6, 24, 48, 96, 168, 240, and 336 hours post LNP infusion for safety analysis and specifically directed to observing changes in liver enzymes and cytokine levels. Serum chemistry parameters were directly measured from blood serum samples on a Beckman Coulter AU680 analyzer. Values for day −10, −7, and −5 were averaged to obtain the baseline value. Both LNP1 and LNP2 dosed animals showed transient alanine aminotransferase (FIG. 13A) elevations that peaked at 48 hours post end of infusion and returned to baseline levels 168 hours post end of infusion. Aspartate aminotransferase levels, illustrated in FIG. 13B, were also elevated by both LNP1 and LNP2 treatments, peaking at 6 hours post end of infusion and returning to baseline levels 96 hours post end of infusion. Serum lactate dehydrogenase concentrations, as illustrated in FIG. 14A, and glutamate dehydrogenase concentrations, as illustrated in FIG. 14B, were also found to be elevated shortly following administration of either LNP1 or LNP2 that returned to baseline levels 96-168 hours post end of infusion. Serum concentrations of gamma-glutamyl transferase, illustrated in FIG. 15A, and alkaline phosphatase, FIG. 15B, were not changed by either LNP1 or LNP2 infusion. In addition, LNP1 and LNP2 treatment did not affect serum total bilirubin concentrations, as illustrated in FIG. 16. LNP1 and LNP2 dosed animals, each also showed elevated serum creatine kinase concentrations, as illustrated in FIG. 17, which in each case peaked at 6 hours and returned fully to baseline levels by 168 hours post end of infusion.

Serum was collected from all animals at day −10, −7, −5 pre-treatment and 24, 168, and 336 hours post LNP infusion for serum cytokine analysis. Cytokines were measured using a multiplexed sandwich immunoassay, where four (MCP-1, IL-6, IP-10, IL-1RA) cytokines are quantitated simultaneously from serum samples using the U-PLEX Biomarker Group 1 (monkey) Assay from Meso Scale Diagnostics (Rockville, MD). Values for day −10, −7, and −5 were averaged to obtain the baseline value. Both LNP1 and LNP2 dosed animals showed elevated serum IL-6 concentrations, as illustrated in FIG. 18, to a similar extent, peaking at 6 hours and returning to baseline by 24 hours post end of infusion. As further illustrated in FIG. 18, both LNP1 and LNP2 dosed animals showed increased serum IL-1RA that peaked at 6 hours and returned fully to baseline by 336 hours. Also, as illustrated in FIG. 18, neither LNP1 nor LNP2 had any measurable significant effect on serum MCP-1 or IP-10 concentrations.

Overall, the analysis of foregoing parameters showed that infusion of either LNP1 and LNP2 in monkeys produced a transient increase in liver enzymes and cytokines that resolves rapidly.

### Pharmacokinetics (PK) Evaluation

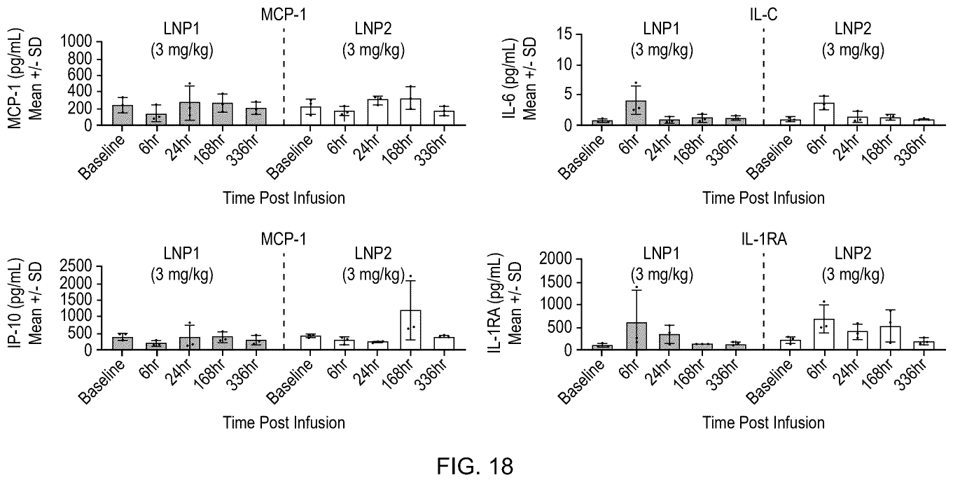

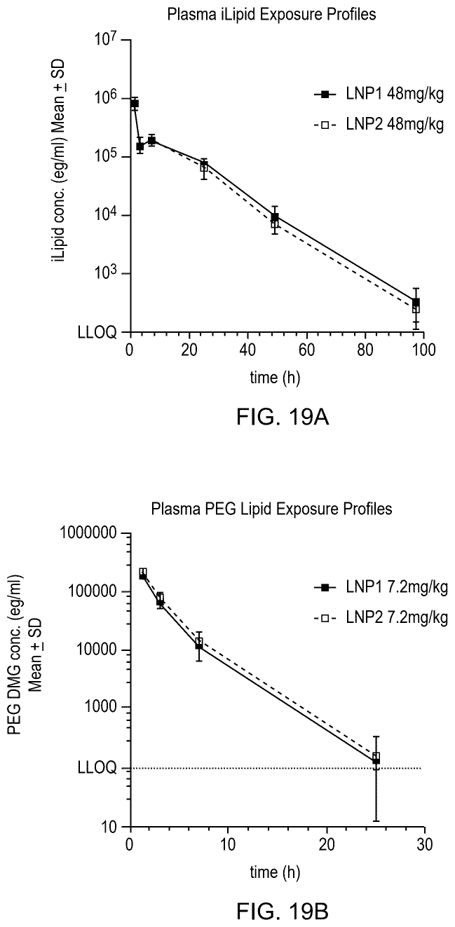

Blood samples were obtained (K₂EDTA) for plasma PK analysis and determination of concentrations of the iLipid and PEGLipid excipients that comprised LNP1 and LNP2. After the end of the infusion, plasma samples were collected at 0.25, 2, 6, 24, 48, 96, 168, 240, and 336 hours post LNP infusion. Concentrations of the iLipid and PEG Lipids were measured using qualified LC-MS assays and are shown in FIG. 19A. Timepoints in which the lipids were below the limit of quantitation are not included in the figure. As illustrated in FIG. 19A, serum iLipid concentrations for LNP1 and LNP2 dosed animals continuously declined until approaching lower limit of quantitation (LLOQ) at 96 hour post LNP infusion. Similarly, as illustrated in FIG. 19B, serum PEG-Lipid concentrations for LNP1 and LNP2 dosed animals also rapidly declined reaching an LLOQ at 24 hours post end of infusion.

Part B: In Vivo NHP Evaluation of GA519 with GalNAc LNP

In further evaluation of GA519, an additional LNP (LNP3) was formulated to encapsulate the same GA519 and ABE8.8 mRNA at the 1:1 weight ratio and dosed intravenously to NHPs as previously described. LNP3 differs from LNP1 in that LNP3 was formulated with an additional GalNAc ligand excipient, as described in more detail below.

### LNP Preparation

The GalNAc LNPs (LNP3) formulated for this aspect of the study were comprised of the same iLipid, neutral helper lipid, PEG-Lipid and sterol lipid as described in connection with LNP1/LNP2, but unlike LNP1/LNP2, LNP3 also is comprised of a GalNAc conjugated lipid. The molar ratios of each constituent component of LNP3 are described in Table 15.

TABLE 15
LNP3 Components

| LNP | | | Mol |
|---|---|---|---|
| Component | Lipid names | Lipid structure | % |
| Amino lipid (iLipid) | 3-((4,4-bis(octyloxy)butanoyl)oxy)- 2-((((3- (diethylamino)propoxy)carbonyl) oxy)methyl)propyl (9Z,12Z)- octadeca-9,12-dienoate* | 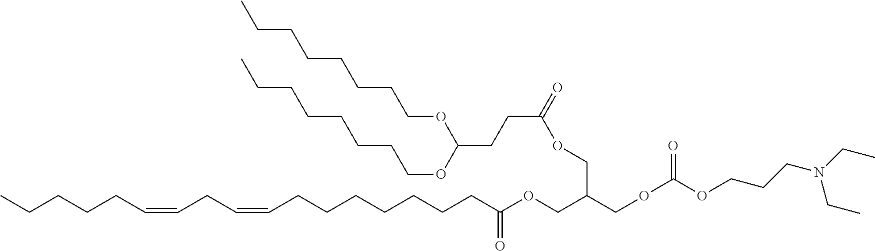 [SMILES: CCCCC/C=C\C/C=C\CCCCCCCC(=O)OCC(COC(=O)CCC(OCCCCCCCC)OCCCCCCCC)COC(=O)OCCCN(CC)CC] | 50 |
| Neutral helper lipid | 1,2-distearoyl-sn-glycero-3- phosphocholine (DSPC) | 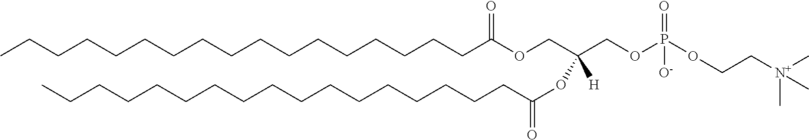 [SMILES: CCCCCCCCCCCCCCCCCC(=O)OC[C@H](COP(=O)([O-])OCC[N+](C)(C)C)OC(=O)CCCCCCCCCCCCCCCCC] | 9 |
| PEG-lipid | 1,2-dimyristoyl-rac-glycero-3- methoxypolyethylene glycol-2000 (PEG₂₀₀₀-DMG) | 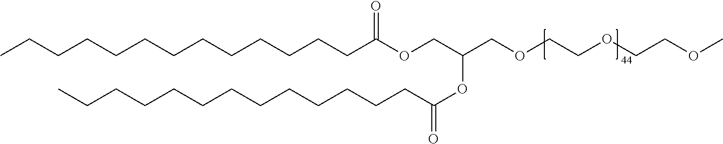 | 3 |
| Sterol lipid | Cholesterol | 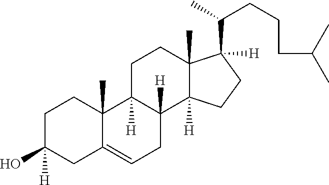 | 37.95 |
| GalNAc- lipid | N²-(PEG-DSG)-N⁶-((C5- GalNAc)amido)-Lys-[bis((C5- GalNAc)propylamido)]amide or DSG-PEG-Lys-tris(GalNAc)** | 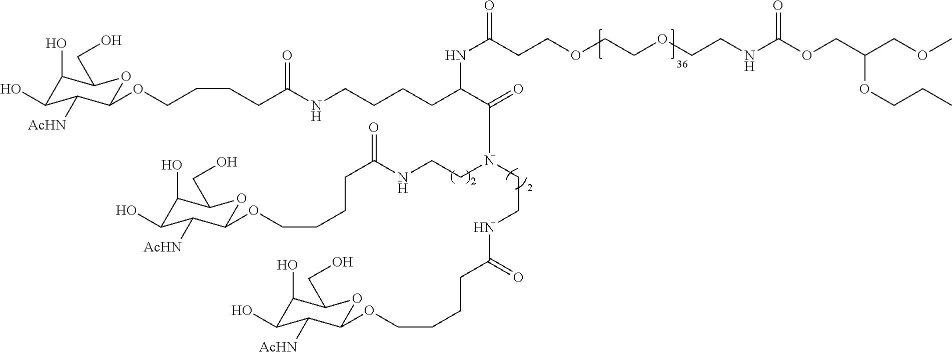 | 0.05 |

*described in International Published Patent Application WO 2015 095340 A1
**described in International Published Patent Application WO 2021/178725 A1

It should be understood that the lipids in Table 15 may be substituted for other suitable lipids in the listed class. For example, the amino lipid may be the following amino lipid, or a salt thereof:

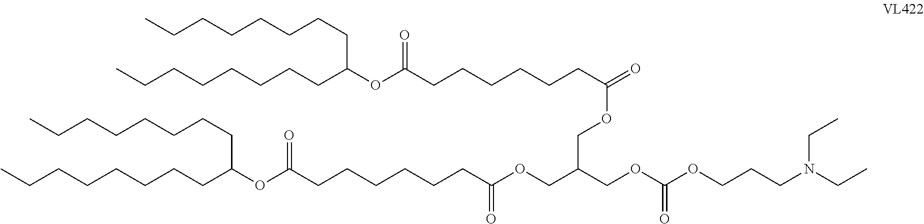 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CC]

It should be further understood that the mol % of lipids in Table 13 may be adjusted and that the mol % included in Table 13 are targeted excipient percentages of the LNP, which is intended to represent the aggregate mol % of all the LNPs formulated in a given batch and that specific LNPs within a batch may have varying mol %. Thus, it is contemplated herein that the mol % of one or more, or all of the LNP components set forth in Table 13 may be adjusted, for example, by +/−1-5%, +/−5-10%, or +/−10%-20%. It is further contemplated herein that the mol % of one or more, or all of the LNP components set forth in Table 13 with respect to a specific LNP formulated in a given batch of LNPs formulated in accordance with desired target excipient percentages, may vary from the targeted mol %, for example, by +/−1-5%, +/−5-10%, or +/−10%-20%, or even greater than +/−20%. Further, it should be understood that additional LNP components, including non-lipid components, may be added to the LNP components set-forth in Table 13.

In formulating LNP3, the GalNAc-Lipid was premixed with other LNP excipients referenced in Table 15 prior to in-line mixing with GA519 sgRNA and ABE 8.8 mRNA (at 1:1 weight ratio) to form LNP3. Rajeev et al., WO2021178725, includes a description of the synthesis and characterization of the GalNAc lipid. As with LNP1/LNP2, the resulting GalNAc-LNPs, LNP3, were filtered using 0.2-micron filters and frozen at −80° C. The physical characteristics of the formulated LNP3 is summarized in Table 16.

TABLE 16
LNP3 Characterization.

| LNP | Average LNP size (nm) | PDI | RNA entrapment |
|---|---|---|---|
| 3 | 61.92 | 0.055 | 98.7 |

One of ordinary skill in the art would understand that the average LNP size, PDI and RNA entrapment values set forth in Table 16 are subject to measurement error or accuracy. It is also contemplated herein that the LNP size, PDI and RNA entrapment values set forth in Table 16 may be varied by +/−1-5%, +/−5-10%, or +/−10%-20%.

### NHP Study Design

Male cynomolgus monkeys of Cambodian origin were used in this aspect of the study. A premedication regimen comprising dexamethasone and H1 and H2 antihistamines were administered to all animals on day −1 (approximately 24 hours prior to dosing) and day 1 (predose), between 30 and 60 minutes before test article dose administration. The LNP3 dosing formulations were administered once on day 1 of the study by IV infusion of two groups of 3 monkeys at dose levels of (i) 2 mg of combined sgRNA and mRNA per kg of animal body weight and at a dose volume of 6 mL/kg (n=3/group) for the first group of three monkeys and (ii) 3 mg of combined sgRNA and mRNA per kg of animal body weight and at a dose volume of 6 mL/kg (n=3/group) for the second group of three monkeys.

Blood samples were collected from all animals predose for baseline measurement and post infusion at various time points from days 1 through 35 to assess biomarkers, plasma iLipid and PEG pharmacokinetics, and serum safety parameters.

Necropsies were performed on day 36. Liver tissue samples were collected from all animals to assess TTR gene editing in the liver.

### Analysis of Editing Efficiency

The amount of gene editing in the liver was evaluated by next-generation sequencing (NGS) of targeted polymerase chain reaction (PCR) amplicons at the TTR target site derived from genomic DNA extracted from the liver as described previously (Musunuru et al., Nature 593, no. 7859 (May 2021): 429-34. https://doi.org/10.1038/s41586-021-03534-y). Percent editing was reported as the percent of all reads containing a nonreference allele at the target adenine.

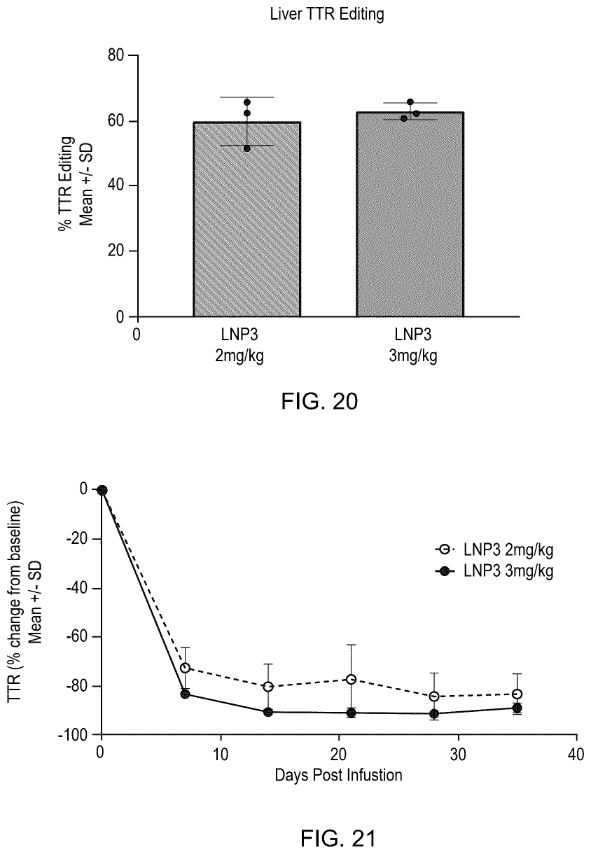

As set forth in FIG. 20, LNP3 led to similar levels of hepatic TTR editing efficiency at 2 mg/kg dosed monkeys (60%) as compared to 3 mg/kg dosed monkeys (63%).

### Quantification of TTR Protein Expression in Serum

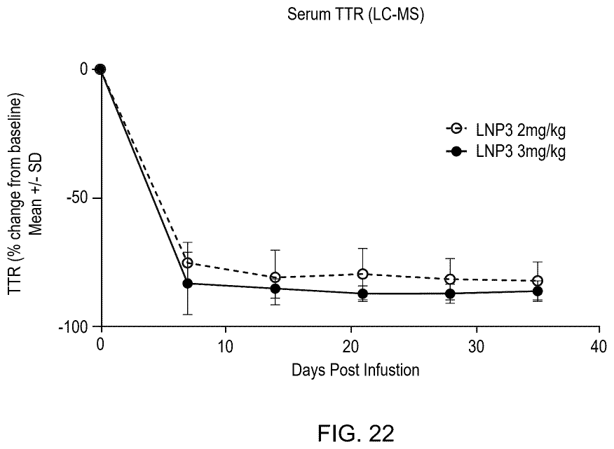

Serum was collected at day −10, −7, −5 pre-infusion and 7, 14, 21, 28, and 35 days post end of infusion for TTR protein analysis. Serum TTR was initially quantified using a custom TTR sandwich ELISA with the data obtained from that analysis presented in FIG. 21. Values for day −10, −7, and −5 were averaged to obtain the baseline value. As illustrated in FIG. 21, both groups of LNP3 dosed animals showed marked reductions in serum TTR protein at the first timepoint (day 7) after dosing. These reductions were maintained for the duration of the study, reaching maximal reductions on day 28 of −84% and −91% change from baseline for the 2 mg/kg and 3 mg/kg monkey groups, respectively. To confirm the ELISA results, TTR protein was also quantitated by LC-MS, in which 4 unique TTR peptide fragments were quantitated in serum at each time point and the average of the 4 results is reported. LC-MS serum TTR quantitation, as illustrated in FIG. 22, confirmed that TTR was reduced at the first timepoint after infusion of the animals on day 7 and was maintained until necropsy on day 35. For the 2 mg/kg LNP3 dosed animals, maximal reduction of TTR protein was reached on day 35 (−82% change from baseline), while for the 3 mg/kg group maximal of TTR protein was reached on day 28 (−87% change from baseline).

Therefore, as described above and illustrated in the foregoing referenced figures, both the 2 mg/kg and 3 mg/kg LNP3 dosed NHPs resulted in marked relatively rapid liver TTR gene editing and corresponding reductions in serum TTR concentrations in protein.

### Safety Analysis

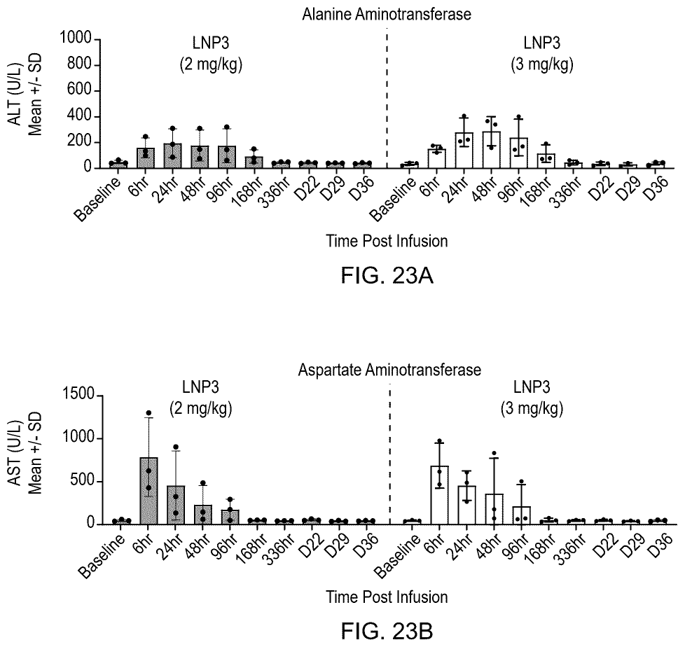

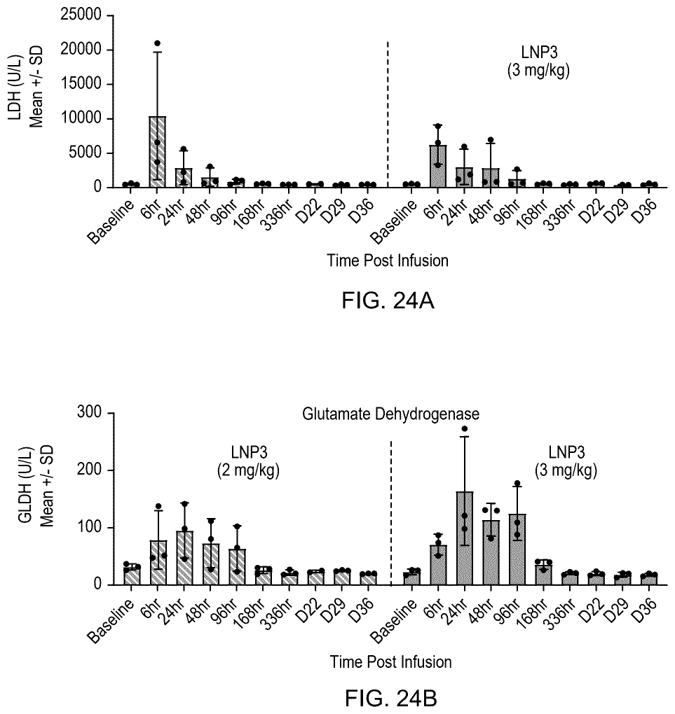

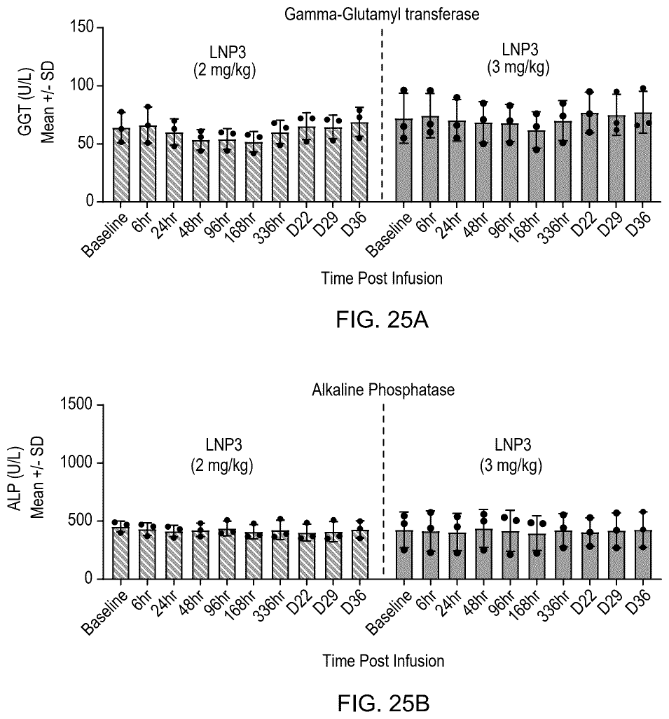

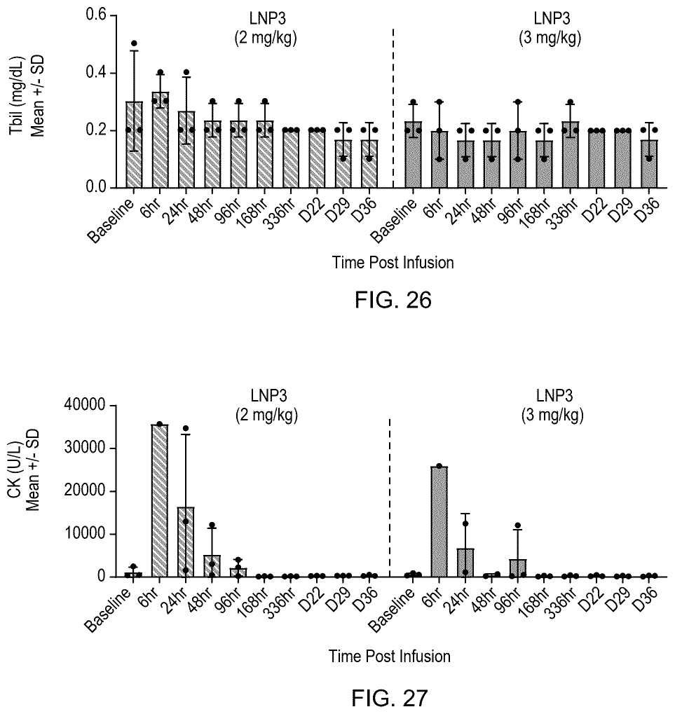

Blood serum was collected from each of the animals in the study at day −10, −7, −5 pre-infusion and 6, 24, 48, 96, 168, 336 hours, day 21, day 28, and day 35 post end of infusion for safety analysis and specifically directed at observing changes in liver enzymes and cytokine levels. Serum chemistry parameters were directly measured from blood serum samples on a Beckman Coulter AU680 analyzer. Values for day −10, −7, and −5 were averaged to obtain the baseline value. LNP3 dosed animals showed dose-dependent, transient alanine aminotransferase elevations as illustrated in FIG. 23A, which peaked at 24-48 hours post end of infusion and returned to baseline levels 336 hours post end of infusion. Aspartate aminotransferase levels, as illustrated in FIG. 23B, were elevated to a similar extent by both the 2 mg/kg and 3 mg/kg LNP3 doses, peaking at 6 hours post end of infusion and returning to baseline levels 168 hours post end of infusion. As illustrated in FIG. 24A, both the 2 mg/k and 3 mg/kg LNP3 doses elevated serum lactate dehydrogenase concentrations that returned to baseline levels by 168 hours post end of infusion. LNP3 also elevated glutamate dehydrogenase concentrations, as illustrated in FIG. 24B, in a dose-dependent manner, peaking at 24-hours, and returning to baseline levels 336 hours post end of infusion. Serum concentrations of gamma-glutamyl transferase and alkaline phosphatase, illustrated in FIGS. 25A and 25B, respectively, were not significantly changed by either LNP dose. In addition, LNP3 treatment did not significantly affect serum total bilirubin concentrations, as illustrated in FIG. 26. LNP3 elevated serum creatine kinase concentrations, as illustrated in FIG. 27, peaking at 6 hours post end of infusion then returning to baseline levels by 168 hours post end of infusion.

The analysis of the foregoing safety parameters in this aspect of the in vivo NHP study were consistent the prior aspect of the study in that they demonstrated that both doses of LNP3 produced a transient increase in liver enzymes that resolved rapidly within 2 weeks following dosing of the subjects.

### Pharmacokinetics (PK) Evaluation

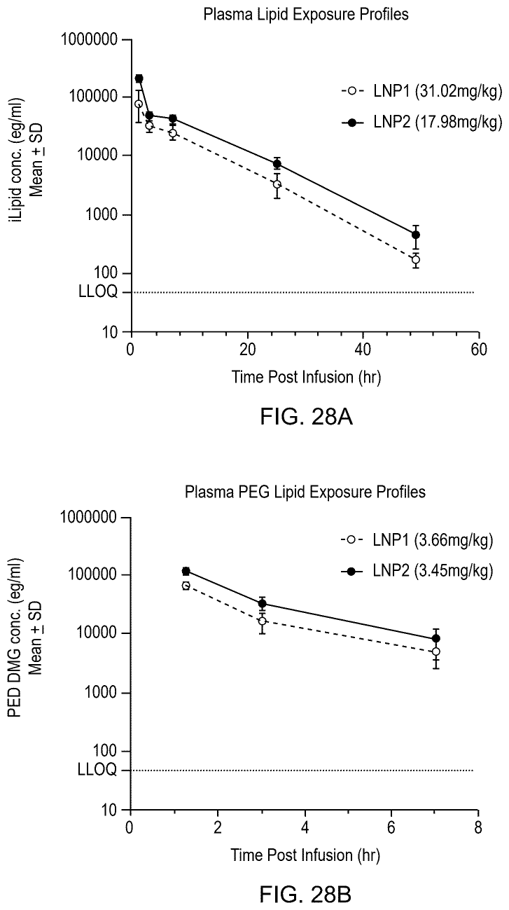

Blood samples were obtained from all animals (K₂EDTA) for plasma PK analysis and determination of concentrations of the ionizable amino lipid (iLipid) and PEGLipid that comprised LNP3. After the end of the infusion, plasma samples were collected at 0.25, 2, 6, 24, 48, 96, 168, 240, and 336 hours post LNP3 infusion. Concentrations of iLipid and PEG-Lipid were measured using qualified LC-MS assays. Dose-dependent iLipid plasma exposure was observed, as illustrated in FIG. 28A, declining below the LLOQ by 96 hours post end of infusion. Dose dependent plasma exposure of PEG lipid was also observed, as illustrated in FIG. 28B, reaching the LLOQ by 24 hours post end of infusion.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### Example 4

### TTR Gene Editing by GA521 Guide RNA

This example illustrates gene editing by an exemplary modified guide RNA, GA521.

Exemplary guide RNA GA521 was transfected into primary human hepatocytes using MessengerMax transfection. GA521 disrupts the start codon AUG of the TTR gene by editing it to ACG with an A-to-G base editor (e.g., ABE8.8; ABE8.8-m).

Three days after transfection, genomic DNA was harvested from the hepatocytes, and assessed for base editing with next-generation sequencing of PCR amplicons generated around the target splice site.

The human TTR locus primers used for NGS analysis are listed below:

With NGS adapters:

Forward (F):
SEQ ID NO: 5769)
TCGTCGGCAGCGTCAGATGTGTATAAGAGACAGGATAAGCAGCCTAGCTC
AGGAGA
Reverse (R):
(SEQ ID NO: 5770)
GTCTCGTGGGCTCGGAGATGTGTATAAGAGACAGGGGCCAGCCTCAGACA
CAAA

Without NGS adapters:

```
  (SEQ ID NO: 5771))
Forward (F): GATAAGCAGCCTAGCTCAGGAGA
  (SEQ ID NO: 5772))
Reverse (R): GGGCCAGCCTCAGACACAAA
```

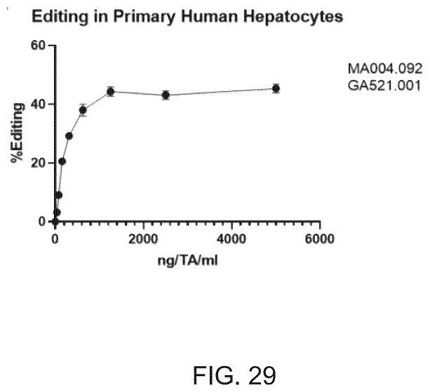

FIG. 29 depicts dose response for human gRNA GA521 in primary human hepatocytes. Percent base editing at various doses (ng/ml) total RNA was determined from NGS analysis. GA521 was the guide RNA.

Overall, GA521 showed an increase in base editing with increasing dose (ng/ml) total RNA and high and sustained editing activity of greater than 40% in human cells.

### OTHER EMBODIMENTS

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment, any portion of the embodiment, or in combination with any other embodiments or any portion thereof.

As is set forth herein, it will be appreciated that the disclosure comprises specific embodiments and examples of base editing systems to effect a nucleobase alteration in a gene and methods of using same for treatment of disease including compositions that comprise such base editing systems, designs and modifications thereto; and specific examples and embodiments describing the synthesis, manufacture, use, and efficacy of the foregoing individually and in combination including as pharmaceutical compositions for treating disease and for in vivo and in vitro delivery of active agents to mammalian cells under described conditions.

While specific examples and numerous embodiments have been provided to illustrate aspects and combinations of aspects of the foregoing, it should be appreciated and understood that any aspect, or combination thereof, of an exemplary or disclosed embodiment may be excluded therefrom to constitute another embodiment without limitation and that it is contemplated that any such embodiment can constitute a separate and independent claim. Similarly, it should be appreciated and understood that any aspect or combination of aspects of one or more embodiments may also be included or combined with any aspect or combination of aspects of one or more embodiments and that it is contemplated herein that all such combinations thereof fall within the scope of this disclosure and can be presented as separate and independent claims without limitation. Accordingly, it should be appreciated that any feature presented in one claim may be included in another claim; any feature presented in one claim may be removed from the claim to constitute a claim without that feature; and any feature presented in one claim may be combined with any feature in another claim, each of which is contemplated herein.

TABLE 17
gRNA spacer sequence with PS linkage at 5′ end
gRNA spacer sequence (5′-3′)

gscscsAUCCUGCCAAGAAUGAG (SEQ ID NO: 6)
gscscsAUCCUGCCAAGAACGAG (SEQ ID NO: 7)
gscsasACUUACCCAGAGGCAAA (SEQ ID NO: 8)
usasusAGGAAAACCAGUGAGUC (SEQ ID NO: 9)
usascsUCACCUCUGCAUGCUCA (SEQ ID NO: 10)
gscscsAUCCUGCCAAGAACGAG (SEQ ID NO: 7)
wherein:
A is adenosine; C is cytidine; G is guanosine; U is uridine; a is 2′-O-methyladenosine; c is 2′-O-methylcytidine; g is 2′-O-methylguanosine; u is 2′-O-methyluridine and s is phosphorothioate (PS) backbone linkage.

TABLE 18
gRNA spacer sequence without PS linkage
gRNA spacer sequence (5′-3′)

GCCAUCCUGCCAAGAAUGAG (SEQ ID NO: 1)
GCCAUCCUGCCAAGAACGAG (SEQ ID NO: 2)
GCAACUUACCCAGAGGCAAA (SEQ ID NO: 3)
UAUAGGAAAACCAGUGAGUC (SEQ ID NO: 4)
UACUCACCUCUGCAUGCUCA (SEQ ID NO: 5)
GCCAUCCUGCCAAGAACGAG (SEQ ID NO: 2)
wherein A is a modified or unmodified adenosine; C is a modified or unmodified cytidine; G is modified or unmodified guanosine; and U is a modified or unmodified uridine.

TABLE 19
Guide RNA sequence
Guide RNA sequence (5′-3′)

gscscsAUCCUGCCAAGAAUGAGGUUUUAGAGCUAGAAAUAGCAAGUUAAAAUAAGGCUAGUC
CGUUAUCAACUUGAAAAAGUGGCACCGAGUCGGUGCUsususu (SEQ ID NO: 11)
AUCCUGCCAAGAACGAGGUUUUAGAGCUAGAAAUAGCAAGUUAAAAUAAGGCUAGUCCGUUAU
CAACUUGAAAAAGUGGCACCGAGUCGGUGCUsususu (SEQ ID NO: 12)
gscsasACUUACCCAGAGGCAAAGUUUUAGAGCUAGAAAUAGCAAGUUAAAAUAAGGCUAGUC
CGUUAUCAACUUGAAAAAGUGGCACCGAGUCGGUGCUsususu (SEQ ID NO: 13)
usasusAGGAAAACCAGUGAGUCGUUUUAGAGCUAGAAAUAGCAAGUUAAAAUAAGGCUAGUC
CGUUAUCAACUUGAAAAAGUGGCACCGAGUCGGUGCUsususu (SEQ ID NO: 14)
usascsUCACCUCUGCAUGCUCAGUUUUAGAGCUAGAAAUAGCAAGUUAAAAUAAGGCUAGUC
CGUUAUCAACUUGAAAAAGUGGCACCGAGUCGGUGCUsususu (SEQ ID NO: 15)
gscscsAUCCUGCCAAGAACGAGgUUUUAGagcuaGaaauagcaaGUUaAaAuAaggcuaGUc
GUUAucAAcuuGaaaaagugGcaccgagucggugcuususus (SEQ ID NO: 16)
usasusAGGAAAACCAGUGAGUCgUUUUAGagcuaGaaauagcaaGUUaAaAuAaggcuaGUc
CGUUAucAAcuuGaaaaagugGcaccgagucggugcuususus (SEQ ID NO: 17)
wherein A is adenosine; C is cytidine; G is guanosine; U is uridine; a is 2′-O-methyladenosine; c is 2′-O-methylcytidine; g is 2′-O-methylguanosine; u is 2′-O-methyluridine and s isphosphorothioate (PS) backbone linkage and wherein bold type represents the spacer sequence.

TABLE 20
Guide RNA sequence
gRNA sequence (5′-3′)

GCCAUCCUGCCAAGAAUGAGGUUUUAGAGCUAGAAAUAGCAAGUUAAAAUAAGGCUAGUCCGU
UAUCAACUUGAAAAAGUGGCACCGAGUCGGUGCUUUU (SEQ ID NO: 18)
GCCAUCCUGCCAAGAACGAGGUUUUAGAGCUAGAAAUAGCAAGUUAAAAUAAGGCUAGUCCGU
UAUCAACUUGAAAAAGUGGCACCGAGUCGGUGCUUUU (SEQ ID NO: 19)
GCAACUUACCCAGAGGCAAAGUUUUAGAGCUAGAAAUAGCAAGUUAAAAUAAGGCUAGUCCGU
UAUCAACUUGAAAAAGUGGCACCGAGUCGGUGCUUUU (SEQ ID NO: 20)
UAUAGGAAAACCAGUGAGUCGUUUUAGAGCUAGAAAUAGCAAGUUAAAAUAAGGCUAGUCCGU
UAUCAACUUGAAAAAGUGGCACCGAGUCGGUGCUUUU (SEQ ID NO: 21)
UACUCACCUCUGCAUGCUCAGUUUUAGAGCUAGAAAUAGCAAGUUAAAAUAAGGCUAGUCCGU
UAUCAACUUGAAAAAGUGGCACCGAGUCGGUGCUUUU (SEQ ID NO: 22)
GCCAUCCUGCCAAGAACGAGGUUUUAGAGCUAGAAAUAGCAAGUUAAAAUAAGGCUAGUCCGU
UAUCAACUUGAAAAAGUGGCACCGAGUCGGUGCUUUU (SEQ ID NO: 19)

It will also be appreciated from reviewing the present disclosure, that it is contemplated that the one or more aspects or features presented in one of or a group of related clauses may also be included in other clauses or in combination with the one or more aspects or features in other clauses.

What is claimed is:

1. A base editor system for modifying a target Transthyretin (TTR) gene, comprising a guide RNA, comprising a sequence defined by
(GA521, SEQ ID NO: 11)
mG*mC*mC*AUCCUGCCAAGAAUGAGmGUUUUAGmAmGmCmUmAGmA
mAmAmUmAmGmCmAmAGUUmAAmAAmUAmAmGmGmCmUmAGUmC
mCGUUAmUmCAAmCmUmUGmAmAmAmAmAmGmUmGGmCmAmCmC
mGmAmGmUmCmGmGmUmGmCmU*mU*mU*mU,
wherein A is adenosine, C is cytidine, G is guanosine, U is uridine, mA* is 2′-O-methyladenosine, mC* is 2′-O-methylcytidine, mG* is 2′-O-methylguanosine, mU* is 2′-O-methyluridine, and wherein nucleotides represented in bold are linked by a phosphorothioate (PS) backbone linkage,
the guide RNA directing the base editor system to effect a nucleobase alteration in the TTR gene.

2. An engineered, non-naturally occurring base editing system for modifying a target Transthyretin (TTR) gene, comprising (a) a guide RNA molecule having a sequence defined by
(SEQ ID NO: 11)
mG*mC*mC*AUCCUGCCAAGAAUGAGmGUUUUAGmAmGmCmUmAGmA
mAmAmUmAmGmCmAmAGUUmAAmAAmUAmAmGmGmCmUmAGUmC
mCGUUAmUmCAAmCmUmUGmAmAmAmAmAmGmUmGGmCmAmCmC
mGmAmGmUmCmGmGmUmGmCmU*mU*mU*mU,
and (b) a codon-optimized nucleic acid encoding a Cas9 protein fused to a deaminase, wherein the Cas9 protein fusion is capable of binding to the guide RNA and of editing the target TTR sequence complementary to the guide RNA.

3. The composition of claim 2, wherein the deaminase comprises a cytosine deaminase or an adenine deaminase.

4. The composition of claim 2, wherein the Cas9 protein is a catalytically impaired Cas9 protein.

5. The composition of claim 4, wherein the Cas9 protein is a dead Cas9 or nickase Cas9.

6. The composition of claim 3, wherein the cytosine or adenine deaminase is a deoxycytosine or a deoxyadenosine deaminase.

7. The composition of claim 3, wherein the Cas9 is fused to ABE8.8.

8. A lipid nanoparticle (LNP) comprising the system of claim 2.

9. The LNP of claim 8, wherein the LNP comprises an ionizable amino lipid, a neutral helper lipid, a PEG-Lipid, a sterol lipid and/or a GalNAc lipid.

10. The LNP of claim 9, wherein the ionizable amino lipid is VL422 or LP-01, the neutral helper lipid is DSPC, the PEG lipid is PEG₂₀₀₀-DMG, the sterol lipid is cholesterol and the GalNAc lipid is DSG-PEG-Lys-tris (GalNAc).

11. The LNP of claim 10, wherein the ionizable lipid is LP-01 (or CIN16645) defined by the structure,

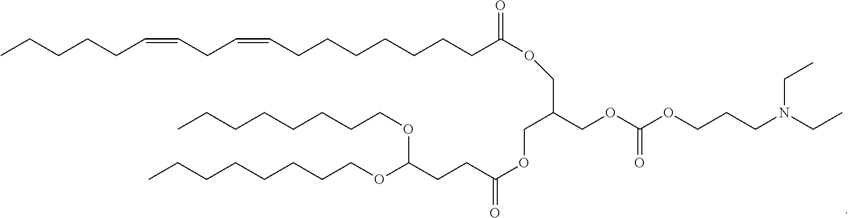 [SMILES: CCCCC/C=C\C/C=C\CCCCCCCC(=O)OCC(COC(=O)CCC(OCCCCCCCC)OCCCCCCCC)COC(=O)OCCCN(CC)CC]

12. The LNP of claim 1, wherein the LNP comprises an N:P ratio of between about 1:40 to about 1:1.

13. The LNP of claim 12, wherein the LNP comprises an N:P ratio of about 1:6.

14. A pharmaceutical composition comprising the LNP of claim 1.

15. A method of editing a TTR gene in a cell, the method comprising contacting the cell with an LNP comprising (a) a guide RNA molecule having a sequence defined by
(SEQ ID NO: 11)
mG*mC*mC*AUCCUGCCAAGAAUGAGmGUUUUAGmAmGmCmUmAGmA
mAmAmUmAmGmCmAmAGUUmAAmAAmUAmAmGmGmCmUmAGUmC
mCGUUAmUmCAAmCmUmUGmAmAmAmAmAmGmUmGGmCmAmCmC
mGmAmGmUmCmGmGmUmGmCmU*mU*mU*mU,
and (b) a base editor system comprising a codon-optimized nucleic acid encoding a Cas9 protein fused to a deaminase, wherein the Cas9 protein fusion is capable of binding to the guide RNA and of editing the target nucleic acid sequence complementary to the guide RNA.

16. A method of treating a disease or disorder, comprising administering to a subject in need thereof, the pharmaceutical composition of claim 14.

17. The method of claim 16, wherein the disease or disorder is hereditary transthyretin amyloidosis, cardiomyopathy, polyneuropathy or senile cardiac amyloidosis.

18. The method of claim 16, wherein the pharmaceutical composition is administered by a route selected from intravenous, intradermal, transdermal, intranasal, intramuscular, subcutaneous, transmucosal or oral.

19. The method of claim 1, wherein the LNP is delivered to liver.
